SEQUENCE LISTING (85 sequences)

SEQ ID NO: 1 - protein, 19 aa, Artificial sequence
  Synthetic
KRRTHNVLERQRRNELKRS

SEQ ID NO: 2 - protein, 19 aa, Artificial sequence
  Synthetic
KRAHHNALERKRRDHIKDS

SEQ ID NO: 3 - protein, 19 aa, Artificial sequence
  Synthetic
KRAHHNALERKRRDHIKDS

SEQ ID NO: 4 - protein, 19 aa, Artificial sequence
  Synthetic
  MISC_FEATURE (7)..(7): Xaa=2-amino-2-methylhept-6-enoic acid
  MISC_FEATURE (11)..(1): Xaa=2-amino-2-methylhept-6-enoic acid
  misc_feature (11)..(11): Xaa can be any naturally occurring amino acid
KRRTHNXLERXRRNELKRS

SEQ ID NO: 5 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRRTHNVLERXRRNXLKRS

SEQ ID NO: 6 - protein, 19 aa, Artificial sequence
  Synthetic
  MISC_FEATURE (3)..(3): Xaa=2-amino-2-methylhept-6-enoic acid
  MISC_FEATURE (7)..(7): Xaa=2-amino-2-methylhept-6-enoic acid
KRXTHNXLERQRRNELKRS

SEQ ID NO: 7 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRAHHNXLERXRRDHIKDS

SEQ ID NO: 8 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRAHHNALERXRRDXIKDS

SEQ ID NO: 9 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRAHHNALERXRRDXIKDS

SEQ ID NO: 10 - protein, 19 aa, Artificial sequence
  Synthetic
  MISC_FEATURE (3)..(3): Xaa=2-amino-2-methylhept-6-enoic acid
  MISC_FEATURE (7)..(7): Xaa=2-amino-2-methylhept-6-enoic acid
KRXHHNXLERKRRDHIKDS

SEQ ID NO: 11 - protein, 21 aa, Artificial sequence
  Synthetic
  MISC_FEATURE (8)..(8): Xaa=2-amino-2-methylhept-6-enoic acid
  MISC_FEATURE (12)..(12): Xaa=2-amino-2-methylhept-6-enoic acid
GKRRTHNXLERXRRNELKRSG

SEQ ID NO: 12 - protein, 21 aa, Artificial sequence
  Synthetic
  MISC_FEATURE (4)..(4): Xaa=2-amino-2-methylhept-6-enoic acid
  MISC_FEATURE (8)..(8): Xaa=2-amino-2-methylhept-6-enoic acid
GKRXHHNXLERKRRDHIKDSG

SEQ ID NO: 13 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRAHHNALERXRRDXIKDS

SEQ ID NO: 14 - protein, 19 aa, Artificial sequence
  Synthetic
  MISC_FEATURE (3)..(3): Xaa=2-amino-2-methylhept-6-enoic acid
  MISC_FEATURE (7)..(7): Xaa=2-amino-2-methylhept-6-enoic acid
KRXHHNXLERKRRDHIKDS

SEQ ID NO: 15 - protein, 19 aa, Artificial sequence
  Synthetic
  MISC_FEATURE (3)..(3): Xaa=2-amino-2-methylhept-6-enoic acid
  MISC_FEATURE (7)..(7): Xaa=2-amino-2-methylhept-6-enoic acid
KRXHHNXLERKRRDHIKDS

SEQ ID NO: 16 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRS

SEQ ID NO: 17 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRRTHNVLERXRRNXLKRS

SEQ ID NO: 18 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRAHHNALERXRRDXIKDS

SEQ ID NO: 19 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRAHHNALERXRRDXIKDS

SEQ ID NO: 20 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRAHHNALERXRRDXIKDS

SEQ ID NO: 21 - protein, 19 aa, Artificial sequence
  Synthetic
KRRTHNVLERQRRNELKRS

SEQ ID NO: 22 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRS

SEQ ID NO: 23 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRRTHNVLERXRRNXLKRS

SEQ ID NO: 24 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRS

SEQ ID NO: 25 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRS

SEQ ID NO: 26 - protein, 19 aa, Artificial sequence
  Synthetic
KRRTHNVLERQRRNELKRS

SEQ ID NO: 27 - protein, 19 aa, Artificial sequence
  Synthetic
KRAHHNALERKRRDHIKDS

SEQ ID NO: 28 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRS

SEQ ID NO: 29 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRS

SEQ ID NO: 30 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRRTHNVLERXRRNXLKRS

SEQ ID NO: 31 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRRTHNVLERXRRNXLKRS

SEQ ID NO: 32 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRAHHNALERXRRDXIKDS

SEQ ID NO: 33 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRAHHNALERXRRDXIKDS

SEQ ID NO: 34 - protein, 19 aa, Artificial sequence
  Synthetic
  MISC_FEATURE (3)..(3): Xaa=2-amino-2-methylhept-6-enoic acid
  MISC_FEATURE (7)..(7): Xaa=2-amino-2-methylhept-6-enoic acid
KRXHHNXLERKRRDHIKDS

SEQ ID NO: 35 - protein, 19 aa, Artificial sequence
  Synthetic
  MISC_FEATURE (3)..(3): Xaa=2-amino-2-methylhept-6-enoic acid
  MISC_FEATURE (7)..(7): Xaa=2-amino-2-methylhept-6-enoic acid
KRXHHNXLERKRRDHIKDS

SEQ ID NO: 36 - protein, 20 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRSG

SEQ ID NO: 37 - protein, 20 aa, Artificial sequence
  Synthetic
  \MISC_FEATURE (7)..(7): Xaa=2-amino-2-methylhept-6-enoic acid
  \MISC_FEATURE (11)..(11): Xaa=2-amino-2-methylhept-6-enoic acid
KRRTHNXLERXRRNELKRSG

SEQ ID NO: 38 - protein, 20 aa, Artificial sequence
  Synthetic
  MISC_FEATURE (3)..(3): Xaa=2-amino-2-methylhept-6-enoic acid
  MISC_FEATURE (7)..(7): Xaa=2-amino-2-methylhept-6-enoic acid
KRXHHNXLERKRRDHIKDSG

SEQ ID NO: 39 - protein, 20 aa, Artificial sequence
  Synthetic
  MISC_FEATURE (3)..(3): Xaa=2-amino-2-methylhept-6-enoic acid
  MISC_FEATURE (7)..(7): Xaa=2-amino-2-methylhept-6-enoic acid
KRXHHNXLERKRRDHIKDSG

SEQ ID NO: 40 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRAHHNALERXRRDXIKDS

SEQ ID NO: 41 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRAHHNALERXRRDXIKDS

SEQ ID NO: 42 - protein, 19 aa, Artificial sequence
  Synthetic
  MISC_FEATURE (3)..(3): Xaa=2-amino-2-methylhept-6-enoic acid
  MISC_FEATURE (7)..(7): Xaa=2-amino-2-methylhept-6-enoic acid
KRXHHNXLERKRRDHIKDS

SEQ ID NO: 43 - protein, 19 aa, Artificial sequence
  Synthetic
  MISC_FEATURE (3)..(3): Xaa=2-amino-2-methylhept-6-enoic acid
  MISC_FEATURE (7)..(7): Xaa=2-amino-2-methylhept-6-enoic acid
KRXHHNXLERKRRDHIKDS

SEQ ID NO: 44 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRS

SEQ ID NO: 45 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRS

SEQ ID NO: 46 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRRTHNVLERXRRNXLKRS

SEQ ID NO: 47 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRRTHNVLERXRRNXLKRS

SEQ ID NO: 48 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRS

SEQ ID NO: 49 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRRTHNVLERXRRNXLKRS

SEQ ID NO: 50 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNELKRS

SEQ ID NO: 51 - protein, 19 aa, Artificial sequence
  Synthetic
  MISC_FEATURE (11)..(11): Xaa=2-amino-2-methylhept-6-enoic acid
  MISC_FEATURE (15)..(15): Xaa=2-amino-2-methylhept-6-enoic acid
KRRTHNVLERXRRNXLKRS

SEQ ID NO: 52 - protein, 23 aa, Artificial sequence
  Synthetic
  MISC_FEATURE (1)..(1): Xaa=W
  MISC_FEATURE (2)..(2): Xaa= a natural amino acid or unnatural amino acid
  MISC_FEATURE (3)..(3): Xaa= K
  MISC_FEATURE (4)..(4): Xaa= R
  MISC_FEATURE (5)..(5): Xaa= an amino acid selected from the group consisting of A, R, and an amino acid of Formula (i)
  MISC_FEATURE (6)..(6): Xaa= H and T
  MISC_FEATURE (7)..(7): Xaa= H
  MISC_FEATURE (8)..(8): Xaa= N
  MISC_FEATURE (9)..(9): Xaa= an amino acid selected from the group consisting of A, V, and an amino acid of Formula (i) or (ii)
  MISC_FEATURE (10)..(10): Xaa= L
  MISC_FEATURE (11)..(11): Xaa= E
  MISC_FEATURE (12)..(12): Xaa= R
  MISC_FEATURE (13)..(13): Xaa= an amino acid selected from the group consisting of K, Q, and an amino acid of Formula (i) or (ii)
  MISC_FEATURE (14)..(14): Xaa= R
  MISC_FEATURE (15)..(15): Xaa= R
  MISC_FEATURE (16)..(16): Xaa= D and N
  MISC_FEATURE (17)..(17): Xaa= an amino acid selected from the group consisting of H, E, and an amino acid of Formula (i) or (ii)
  MISC_FEATURE (18)..(18): Xaa= I and L
  MISC_FEATURE (19)..(19): Xaa= K
  MISC_FEATURE (20)..(20): Xaa= D and R
  MISC_FEATURE (21)..(21): Xaa= S
  MISC_FEATURE (22)..(22): Xaa= is independently a natural or unnatural amino acid
  MISC_FEATURE (23)..(23): Xaa= is a natural or unnatural amino acid of the Formula (v) or (vi)
XXXXXXXXXXXXXXXXXXXXXXX

SEQ ID NO: 53 - protein, 23 aa, Artificial sequence
  Synthetic
  MISC_FEATURE (1)..(1): Xaa = W
  MISC_FEATURE (2)..(2): Xaa = is absent or is a natural or unnatural amino acid
  MISC_FEATURE (3)..(3): Xaa = K
  MISC_FEATURE (4)..(4): Xaa = R
  MISC_FEATURE (5)..(5): Xaa = an amino acid selected from the group consisting of A and R, or X5 and X9 are stapled amino acids of Formula (iii), or X5, X9, and X13 are stapled amino acids of Formula (iv)
  MISC_FEATURE (6)..(6): Xaa = H and T
  MISC_FEATURE (7)..(7): Xaa = H
  MISC_FEATURE (8)..(8): Xaa = N
  MISC_FEATURE (9)..(9): Xaa = an amino acid selected from the group consisting of A and V
  MISC_FEATURE (10)..(10): Xaa = L
  MISC_FEATURE (11)..(11): Xaa = E
  MISC_FEATURE (12)..(12): Xaa = R
  MISC_FEATURE (13)..(13): Xaa = an amino acid selected from the group consisting of K and Q
  MISC_FEATURE (14)..(14): Xaa = R
  MISC_FEATURE (15)..(15): Xaa = R
  MISC_FEATURE (16)..(16): Xaa = D and N
  MISC_FEATURE (17)..(17): Xaa = H and E
  MISC_FEATURE (18)..(18): Xaa = I and L
  MISC_FEATURE (19)..(19): Xaa = K
  MISC_FEATURE (20)..(20): Xaa = D and R
  MISC_FEATURE (21)..(21): Xaa = S
  MISC_FEATURE (22)..(22): Xaa = is independently a natural amino acid or an unnatural amino acid
  MISC_FEATURE (23)..(23): Xaa = an amino acid of the Formula (iii) or (iv)
XXXXXXXXXXXXXXXXXXXXXXX

SEQ ID NO: 54 - protein, 23 aa, Artificial sequence
  Synthetic
  MISC_FEATURE (1)..(1): Xaa = W
  MISC_FEATURE (2)..(2): Xaa = R
  MISC_FEATURE (3)..(3): Xaa = K
  MISC_FEATURE (4)..(4): Xaa = R
  MISC_FEATURE (5)..(5): Xaa = is independently an amino acid selected from the group consisting of A and R
  MISC_FEATURE (6)..(6): Xaa = H and T
  MISC_FEATURE (7)..(7): Xaa = T
  MISC_FEATURE (8)..(8): Xaa = N
  MISC_FEATURE (9)..(9): Xaa = is independently an amino acid selected from the group consisting of A and V
  MISC_FEATURE (10)..(10): Xaa = L
  MISC_FEATURE (11)..(11): Xaa = E
  MISC_FEATURE (12)..(12): Xaa = R
  MISC_FEATURE (13)..(13): Xaa =  is independently an amino acid selected from the group consisting of K and Q
  MISC_FEATURE (14)..(14): Xaa =  R
  MISC_FEATURE (15)..(15): Xaa =  R
  MISC_FEATURE (16)..(16): Xaa = D and N
  MISC_FEATURE (17)..(16): Xaa =  is independently an amino acid selected from the group consisting of H and E
  misc_feature (17)..(17): Xaa can be any naturally occurring amino acid
  MISC_FEATURE (18)..(18): Xaa =  I and L
  MISC_FEATURE (19)..(19): Xaa = K
  MISC_FEATURE (20)..(20): Xaa = D and R
  MISC_FEATURE (21)..(21): Xaa = S
  MISC_FEATURE (22)..(22): Xaa = is independently a natural amino acid or an unnatural amino acid
  MISC_FEATURE (23)..(23): Xaa = first stapled amino acid sequence of Formula (IIIa) and amino acid X23 of the second stapled amino acid sequence
XXXXXXXXXXXXXXXXXXXXXXX

SEQ ID NO: 55 - protein, 23 aa, Artificial sequence
  Synthetic
  MISC_FEATURE (1)..(1): Xaa= W
  MISC_FEATURE (2)..(2): Xaa= is absent or is a natural amino acid or unnatural amino acid
  MISC_FEATURE (3)..(3): Xaa= K
  MISC_FEATURE (4)..(4): Xaa= R
  MISC_FEATURE (5)..(5): Xaa= an amino acid selected from the group consisting of A, R, and an amino acid of Formula (i);
  MISC_FEATURE (6)..(6): Xaa= H and T
  MISC_FEATURE (7)..(7): Xaa= H
  MISC_FEATURE (8)..(8): Xaa= N
  MISC_FEATURE (9)..(9): Xaa= an amino acid selected from the group consisting of A, V, and an amino acid of Formula (i) or (ii)
  MISC_FEATURE (10)..(10): Xaa= L
  MISC_FEATURE (11)..(11): Xaa= E
  MISC_FEATURE (12)..(12): Xaa= R
  MISC_FEATURE (13)..(13): Xaa= an amino acid selected from the group consisting of K, Q, and an amino acid of Formula (i) or (ii)
  MISC_FEATURE (14)..(14): Xaa= R
  MISC_FEATURE (15)..(15): Xaa= R
  MISC_FEATURE (16)..(16): Xaa= D and N
  MISC_FEATURE (17)..(17): Xaa= an amino acid selected from the group consisting of H, E, and an amino acid of Formula (i) or (ii);
  MISC_FEATURE (18)..(18): Xaa= an amino acid selected from the group consisting of C and an amino acid of the Formula (v) or (vi)
  MISC_FEATURE (19)..(19): Xaa= K
  MISC_FEATURE (20)..(20): Xaa= D and R
  MISC_FEATURE (21)..(21): Xaa= S
  MISC_FEATURE (22)..(22): Xaa= is independently a natural or unnatural amino acid
  MISC_FEATURE (23)..(23): Xaa= a natural or unnatural amino acid of the Formula (v) or (vi)
XXXXXXXXXXXXXXXXXXXXXXX

SEQ ID NO: 56 - protein, 23 aa, Artificial sequence
  Synthetic
  MISC_FEATURE (1)..(1): Xaa= W
  MISC_FEATURE (2)..(2): Xaa= is absent or is a natural or unnatural amino acid
  MISC_FEATURE (3)..(3): Xaa= K
  MISC_FEATURE (4)..(4): Xaa= R
  MISC_FEATURE (5)..(5): Xaa= an amino acid selected from the group consisting of A and R, or X5 and X9 are stapled amino acids of Formula (iii), or X5, X9, and X13 are stapled amino acids of Formula (iv)
  MISC_FEATURE (6)..(6): Xaa= H and T
  MISC_FEATURE (7)..(7): Xaa= H
  MISC_FEATURE (8)..(8): Xaa= N
  MISC_FEATURE (9)..(8): Xaa= an amino acid selected from the group consisting of A and V
  misc_feature (9)..(9): Xaa can be any naturally occurring amino acid
  MISC_FEATURE (10)..(10): Xaa= L
  MISC_FEATURE (11)..(11): Xaa= E
  MISC_FEATURE (12)..(12): Xaa= R
  MISC_FEATURE (13)..(13): Xaa= an amino acid selected from the group consisting of K and Q
  MISC_FEATURE (14)..(14): Xaa= R
  MISC_FEATURE (15)..(15): Xaa= R
  MISC_FEATURE (16)..(16): Xaa= D and N
  MISC_FEATURE (17)..(17): Xaa= an amino acid selected from the group consisting of H and E
  MISC_FEATURE (18)..(18): Xaa= an amino acid selected from the group consisting of C and an amino acid of the Formula (iii) or (iv)
  MISC_FEATURE (19)..(19): Xaa= K
  MISC_FEATURE (20)..(20): Xaa=D and R
  MISC_FEATURE (21)..(21): Xaa=S
  MISC_FEATURE (22)..(22): Xaa=  is independently a natural amino acid or an unnatural amino acid
  MISC_FEATURE (23)..(23): Xaa= an amino acid of the Formula (iii) or (iv)
XXXXXXXXXXXXXXXXXXXXXXX

SEQ ID NO: 57 - protein, 23 aa, Artificial sequence
  Synthetic
  MISC_FEATURE (1)..(1): Xaa= W
  MISC_FEATURE (2)..(2): Xaa= is independently absent or is a natural amino acid or unnatural amino acid
  MISC_FEATURE (3)..(3): Xaa= K
  MISC_FEATURE (4)..(4): Xaa= R
  MISC_FEATURE (5)..(5): Xaa= is independently an amino acid selected from the group consisting of A and R
  MISC_FEATURE (6)..(6): Xaa= H and T
  MISC_FEATURE (7)..(7): Xaa= H
  MISC_FEATURE (8)..(8): Xaa= N
  MISC_FEATURE (9)..(9): Xaa= is independently an amino acid selected from the group consisting of A and V
  MISC_FEATURE (10)..(10): Xaa= L
  MISC_FEATURE (11)..(11): Xaa= E
  MISC_FEATURE (12)..(12): Xaa= R
  MISC_FEATURE (13)..(13): Xaa= is independently an amino acid selected from the group consisting of K and Q
  MISC_FEATURE (14)..(14): Xaa= R
  MISC_FEATURE (15)..(15): Xaa= R
  MISC_FEATURE (16)..(16): Xaa= D and N
  MISC_FEATURE (17)..(17): Xaa= is independently an amino acid selected from the group consisting of H and E
  MISC_FEATURE (18)..(18): Xaa= the first stapled amino acid sequence of Formula (VIa) and amino acid X18 of the second stapled amino acid sequence
  MISC_FEATURE (19)..(19): Xaa= K
  MISC_FEATURE (20)..(20): Xaa= D and R
  MISC_FEATURE (21)..(21): Xaa= S
  MISC_FEATURE (22)..(22): Xaa= is independently a natural amino acid or an unnatural amino acid
  MISC_FEATURE (23)..(23): Xaa= the first stapled amino acid sequence of Formula (VIa) and amino acid X23 of the second stapled amino acid sequence of Formula (VIb)
XXXXXXXXXXXXXXXXXXXXXXX

SEQ ID NO: 58 - protein, 11 aa, Artificial sequence
  Synthetic
  MISC_FEATURE (1)..(1): Xaa= is independently a natural or unnatural amino acid
  MISC_FEATURE (2)..(2): Xaa= is selected from the group consisting of a natural or unnatural amino acid, and an amino acid of Formula (i) or (ii)
  MISC_FEATURE (3)..(6): Xaa= any a natural or unnatural amino acid
  MISC_FEATURE (7)..(7): Xaa= any a natural or unnatural amino acid, and an amino acid of Formula (i) or (ii)
  MISC_FEATURE (8)..(8): Xaa= is independently a natural or unnatural amino acid
  MISC_FEATURE (9)..(9): Xaa= an amino acid selected from the group consisting of C and an amino acid of the Formula (v) or (vi)
  MISC_FEATURE (10)..(10): Xaa=  is independently a natural or unnatural amino acid
  MISC_FEATURE (11)..(11): Xaa=  an amino acid selected from the group consisting of C and an amino acid of the Formula (v) or (vi)
XXXXXXXXXXX

SEQ ID NO: 59 - protein, 11 aa, Artificial sequence
  Synthetic
  MISC_FEATURE (1)..(1): Xaa=is independently a natural or unnatural amino acid
  MISC_FEATURE (2)..(2): Xaa= stapled amino acids of Formula (iii);
  MISC_FEATURE (3)..(6): Xaa= any a natural or unnatural amino acid
  MISC_FEATURE (7)..(7): Xaa= stapled amino acids of Formula (iii);
  MISC_FEATURE (8)..(8): Xaa= any a natural or unnatural amino acid
  MISC_FEATURE (9)..(9): Xaa= is an amino acid selected from the group consisting of C and an amino acid of the Formula (v) or (vi)
  MISC_FEATURE (10)..(10): Xaa= is independently a natural or unnatural amino acid
  MISC_FEATURE (11)..(11): Xaa= is an amino acid selected from the group consisting of C and an amino acid of the Formula (v) or (vi)
XXXXXXXXXXX

SEQ ID NO: 60 - protein, 11 aa, Artificial sequence
  Synthetic
  MISC_FEATURE (1)..(1): Xaa= is independently a natural or unnatural amino acid
  MISC_FEATURE (2)..(2): Xaa= stapled amino acids of Formula (iii)
  MISC_FEATURE (3)..(6): Xaa= is independently a natural or unnatural amino acid
  MISC_FEATURE (7)..(7): Xaa= stapled amino acids of Formula (iii)
  MISC_FEATURE (8)..(8): Xaa= is independently a natural or unnatural amino acid
  MISC_FEATURE (9)..(9): Xaa= the first stapled amino acid sequence of Formula (IXa) and amino acid X9 of the second stapled amino acid sequence of Formula (IXb)
  MISC_FEATURE (10)..(10): Xaa= independently a natural or unnatural amino acid
  MISC_FEATURE (11)..(11): Xaa= the first stapled amino acid sequence of Formula (IXa) and amino acid X11 of the second stapled amino acid sequence of Formula (IXb)
XXXXXXXXXXX

SEQ ID NO: 61 - protein, 13 aa, Artificial sequence
  Synthetic
  MISC_FEATURE (1)..(1): Xaa= is independently a natural or unnatural amino acid
  MISC_FEATURE (2)..(2): Xaa= a modified or unnatural amino acid comprising hindered alpha-methyl, alpha-alkenyl substituents
  MISC_FEATURE (3)..(6): Xaa= any a natural or unnatural amino acid
  MISC_FEATURE (7)..(7): Xaa= a modified or unnatural amino acid comprising hindered alpha-methyl, alpha-alkenyl substituents
  MISC_FEATURE (8)..(8): Xaa= is independently a natural or unnatural amino acid
  MISC_FEATURE (9)..(9): Xaa= is absent or a modified or unnatural amino acid comprising hindered alpha-methyl, alpha-alkenyl substituents
  MISC_FEATURE (10)..(10): Xaa= is independently a natural or unnatural amino acid
  MISC_FEATURE (11)..(11): Xaa= a modified or unnatural amino acid displaying a linker group
  MISC_FEATURE (12)..(12): Xaa= is independently a natural or unnatural amino acid
  MISC_FEATURE (13)..(13): Xaa= absent or is a modified or unnatural amino acid displaying a linker group
XXXXXXXXXXXXX

SEQ ID NO: 62 - protein, 13 aa, Artificial sequence
  Synthetic
  MISC_FEATURE (1)..(1): Xaa= is independently a natural or unnatural amino acid
  MISC_FEATURE (2)..(2): Xaa= a modified or unnatural amino acid linked to X7 via a hydrocarbon staple resulting from a ring-closing olefin metathesis (RCM) of hindered alpha-methyl, alpha-alkenyl amino acids
  MISC_FEATURE (3)..(6): Xaa= a natural or unnatural amino acid
  MISC_FEATURE (7)..(7): Xaa= a modified or unnatural amino acid linked to X7 via a hydrocarbon staple resulting from a ring-closing olefin metathesis (RCM) of hindered alpha-methyl, alpha-alkenyl amino acids
  MISC_FEATURE (8)..(8): Xaa= is independently a natural or unnatural amino acid
  misc_feature (9)..(9): Xaa can be any naturally occurring amino acid
  MISC_FEATURE (10)..(10): Xaa= is independently a natural or unnatural amino acid
  MISC_FEATURE (11)..(11): Xaa= a modified or unnatural amino acid displaying a linker group
  MISC_FEATURE (12)..(12): Xaa= is independently a natural or unnatural amino acid
  MISC_FEATURE (13)..(13): Xaa= a modified or unnatural amino acid displaying a linker group
XXXXXXXXXXXXX

SEQ ID NO: 63 - protein, 13 aa, Artificial sequence
  Synthetic
  MISC_FEATURE (1)..(1): Xaa= a natural or unnatural amino acid
  MISC_FEATURE (2)..(2): Xaa= modified or unnatural amino acid linked to X7 and X7, respectively, via a hydrocarbon staple resulting from a ring-closing olefin metathesis (RCM) of hindered alpha-methyl, alpha-alkenyl amino acids
  MISC_FEATURE (3)..(6): Xaa= a natural or unnatural amino acid
  MISC_FEATURE (7)..(7): Xaa= modified or unnatural amino acid linked to X2 and X2, respectively, via a hydrocarbon staple resulting from a ring-closing olefin metathesis (RCM) of hindered alpha-methyl, alpha-alkenyl amino acids
  MISC_FEATURE (8)..(8): Xaa=  a natural or unnatural amino acid
  MISC_FEATURE (9)..(9): Xaa= absent or a modified or unnatural amino acid comprising hindered alpha-methyl, alpha-alkenyl substituents
  MISC_FEATURE (10)..(10): Xaa= a natural or unnatural amino acid
  MISC_FEATURE (11)..(11): Xaa= modified or unnatural amino acid displaying a linker group
  MISC_FEATURE (12)..(12): Xaa= a natural or unnatural amino acid
  MISC_FEATURE (13)..(13): Xaa= modified or unnatural amino acid displaying a linker group
XXXXXXXXXXXXX

SEQ ID NO: 64 - DNA, 19 bp, Artificial sequence
  Synthetic
accccaccacgtggtgcct

SEQ ID NO: 65 - DNA, 19 bp, Artificial sequence
  Synthetic
aggcaccacgtggtggggt

SEQ ID NO: 66 - DNA, 19 bp, Artificial sequence
  Synthetic
accccaccatatggtgcct

SEQ ID NO: 67 - DNA, 19 bp, Artificial sequence
  Synthetic
aggcaccatatggtggggt

SEQ ID NO: 68 - DNA, 19 bp, Artificial sequence
  Synthetic
accccacatatatgtgcct

SEQ ID NO: 69 - DNA, 19 bp, Artificial sequence
  Synthetic
aggcacatatatgtggggt

SEQ ID NO: 70 - protein, 19 aa, Artificial sequence
  Synthetic
  MISC_FEATURE (7)..(7): Xaa=2-amino-2-methylhept-6-enoic acid
  MISC_FEATURE (11)..(11): Xaa=2-amino-2-methylhept-6-enoic acid
KRRTHNXLERXRRNEKKRS

SEQ ID NO: 71 - protein, 19 aa, Artificial sequence
  Synthetic
  MISC_FEATURE (11)..(11): Xaa= 2-amino-2-methylhept-6-enoic acid
  misc_feature (15)..(15): Xaa can be any naturally occurring amino acid
  MISC_FEATURE (158)..(15): Xaa= 2-amino-2-methylhept-6-enoic acid
KRRTHNVLERXRRNXKKRS

SEQ ID NO: 72 - protein, 19 aa, Artificial sequence
  Synthetic
  MISC_FEATURE (11)..(11): Xaa= 2-amino-2-methylhept-6-enoic acid
  MISC_FEATURE (15)..(15): Xaa= 2-amino-2-methylhept-6-enoic acid
KRAHHNALERXRRDXCKDS

SEQ ID NO: 73 - protein, 19 aa, Artificial sequence
  Synthetic
  MISC_FEATURE (3)..(3): Xaa= 2-amino-2-methylhept-6-enoic acid
  MISC_FEATURE (7)..(7): Xaa= 2-amino-2-methylhept-6-enoic acid
KRXHHNXLERKRRDHCKDS

SEQ ID NO: 74 - protein, 19 aa, Artificial sequence
  Synthetic
  MISC_FEATURE (7)..(7): Xaa= 2-amino-2-methylhept-6-enoic acid
  MISC_FEATURE (11)..(11): Xaa= 2-amino-2-methylhept-6-enoic acid
KRRTHNXLERXRRNECKRS

SEQ ID NO: 75 - protein, 19 aa, Artificial sequence
  Synthetic
  MISC_FEATURE (11)..(11): Xaa= 2-amino-2-methylhept-6-enoic acid
  MISC_FEATURE (15)..(15): Xaa= 2-amino-2-methylhept-6-enoic acid
KRRTHNVLERXRRNXCKRS

SEQ ID NO: 76 - protein, 19 aa, Artificial sequence
  Synthetic
  MISC_FEATURE (11)..(11): Xaa= 2-amino-2-methylhept-6-enoic acid
  MISC_FEATURE (15)..(15): Xaa= 2-amino-2-methylhept-6-enoic acid
KRAHHNALERXRRDXKKDS

SEQ ID NO: 77 - protein, 19 aa, Artificial sequence
  Synthetic
  MISC_FEATURE (3)..(3): Xaa= 2-amino-2-methylhept-6-enoic acid
  MISC_FEATURE (7)..(7): Xaa= 2-amino-2-methylhept-6-enoic acid
KRXHHNXLERKRRDHKKDS

SEQ ID NO: 78 - protein, 20 aa, Artificial sequence
  Synthetic
IRRERNKMAAAKSRNRRREL

SEQ ID NO: 79 - protein, 20 aa, Artificial sequence
  Synthetic
  MISC_FEATURE (4)..(4): Xaa= 2-amino-2-methylhept-6-enoic acid
  MISC_FEATURE (8)..(8): Xaa= 2-amino-2-methylhept-6-enoic acid
IRRXRNKXAAAKSRNRRREL

SEQ ID NO: 80 - protein, 20 aa, Artificial sequence
  Synthetic
  MISC_FEATURE (11)..(11): Xaa= 2-amino-2-methylhept-6-enoic acid
  MISC_FEATURE (15)..(15): Xaa= 2-amino-2-methylhept-6-enoic acid
IRRERNKMAAXKSRXRRREL

SEQ ID NO: 81 - protein, 20 aa, Artificial sequence
  Synthetic
RKRMRNRIAASKSRKRKLER

SEQ ID NO: 82 - protein, 20 aa, Artificial sequence
  Synthetic
  MISC_FEATURE (4)..(4): Xaa= 2-amino-2-methylhept-6-enoic acid
  MISC_FEATURE (8)..(8): Xaa= 2-amino-2-methylhept-6-enoic acid
RKRXRNRXAASKSRKRKLER

SEQ ID NO: 83 - protein, 20 aa, Artificial sequence
  Synthetic
  MISC_FEATURE (11)..(11): Xaa= 2-amino-2-methylhept-6-enoic acid
  MISC_FEATURE (15)..(15): Xaa= 2-amino-2-methylhept-6-enoic acid
RKRMRNRIAAXKSRXRKLER

SEQ ID NO: 84 - DNA, 21 bp, Artificial sequence
  Synthetic
cgcttgatgactcagccggaa

SEQ ID NO: 85 - DNA, 21 bp, Artificial sequence
  Synthetic
ttccggctgagtcatcaagcg

(19) United States
(12) Patent Application Publication
(10) Pub. No.: US 2019/0135868 A1
(43) Pub. Date: May 9, 2019

(54) SYNTHETIC DNA BINDING DOMAIN PEPTIDES AND USES THEREOF

(71) Applicant: The University of Chicago, Chicago, IL (US)

(72) Inventors: Raymond E. Moellering, Chicago, IL (US); Xianghang Shangguan, Chicago, IL (US)

(21) Appl. No.: 16/096,609

(22) PCT Filed: Apr. 28, 2017
(86) PCT No.: PCT/US17/30217

§ 371 Date: Oct. 25, 2018

Related U.S. Application Data
(60) Provisional application No. 62/329,497, filed on Apr. 29, 2016.

(51) Int. Cl. C07K 14/00; A61P 35/00; A61K 47/64

(52) U.S. Cl. CPC C07K 14/001; A61K 38/00; A61K 47/64; A61P 35/00

(57) ABSTRACT

The present invention relates to peptides and protein mimetics and their therapeutic and research use. In particular, the present invention provides synthetic, stabilized DNA binding domain peptides and methods of using such peptides as therapeutic agents.

### CROSS-REFERENCE TO RELATED APPLICATIONS

The present invention claims the priority benefit of U.S. Provisional Patent Application 62/329,497, filed Apr. 29, 2016, which is incorporated by reference in its entirety.

### FIELD OF THE INVENTION

The present invention relates to peptides and protein mimetics and their therapeutic and research use. In particular, the present invention provides synthetic, stabilized DNA binding domain peptides and methods of using such peptides as therapeutic agents.

### BACKGROUND OF THE INVENTION

The important biological roles that peptides and proteins play as hormones, enzyme inhibitors, substrates, gene expression regulators and neurotransmitters has led to the use of peptides and/or peptide mimetics as therapeutic agents. The bioactive conformation of a peptide, combining structural elements such as alpha-helices, beta-sheets, turns, and/or loops, is important as it allows for selective recognition of biological molecules such as receptors, enzymes, and nucleic acids, thereby influencing cell-cell communication and/or controlling vital cellular functions, such as metabolism, immune defense, and cell division (see, e.g., Babine et al., Chem. Rev. (1997) 97:1359; incorporated by reference in its entirety). Unfortunately, the utility of peptides as drugs is severely limited by several factors, including their rapid degradation by proteases under physiological conditions, their poor cell permeability, and their lack of binding specificity resulting from conformational flexibility. Moreover, alpha-helical peptides have a propensity for unraveling and forming random coils, which are biologically less capable, or even incapable, of binding their target(s) with suitable affinity. Additionally, unstructured peptides are highly susceptible to proteolytic degradation.

Several strategies have been devised to design and synthesize more robust peptides as therapeutics. As an example, one strategy has been to incorporate more non-natural or more robust functionalities into the peptide chain while still maintaining the peptide's unique conformation and secondary structure (see, e.g., Gante, Angew. Chem. Int. Ed. Engl. (1994) 33:1699-1720; Liskamp, Recl. Tray. Chim. Pays-Bas (1994) 113:1; Giannis, Angew. Chem. Int. Ed. Engl. (1993) 32:1244; Bailey, Peptide Chemistry, Wiley, New York (1990), 182; and references cited therein: incorporated by reference in their entireties). Another approach has been to stabilize the peptide via covalent cross-links (see, e.g., Phelan et al., J. Am. Chem. Soc. (1997) 119:455; Leuc et al., Proc. Natl. Acad. Sci. USA (2003) 100: 11273; Bracken et al., J. Am. Chem. Soc. (1994) 116:6432; Yan et al., Bioorg. Med. Chem. (2004) 14:1403; incorporated by reference in their entireties). However, the majority of reported approaches involved the use of polar and/or labile cross-linking groups.

“Peptide stapling” is a term coined for a synthetic methodology used to covalently join two olefin-containing side chains present in a polypeptide chain by ring closing metathesis (RCM) (see, e.g., Blackwell et al., J. Org. Chem. (2001) 66:5291-5302; Blackwell et al., Angew. Chem. Int. Ed. (1998) 37:3281; incorporated by reference in their entireties). Stapling of a polypeptide using a hydrocarbon cross-linker created from an olefin metathesis reaction has been shown to help maintain a peptide's native conformation, particularly under physiological conditions (see, e.g., U.S. Pat. Nos. 7,192,713; 7,723,469; 7,786,072; U.S. Patent Application Publication Nos: 2010-0184645; 2010-0168388; 2010-0081611; 2009-0176964; 2009-0149630; 2006-0008848; PCT Application Publication Nos: WO 2010/011313; WO 2008/121767; WO 2008/095063; WO 2008/061192; WO 2005/044839; Schafmeister et al., J. Am. Chem. Soc. (2000) 122:5891-5892; Walensky et al., Science (2004) 305:1466-1470, Moellering et al., Nature (2009) 462:182-188; incorporated by reference in their entireties). The stapled polypeptide strategy in which an all-hydrocarbon cross-link is generated by olefin metathesis is an efficient approach to increase the helical character of polypeptides to target α-helical binding motifs. Unlike their unstapled analogues, these hydrocarbon-stapled polypeptides have shown to be α-helical, protease-resistant, and cell permeable.

Transcription factors (TFs) regulate cell state by binding specific DNA sequences, thereby recruiting transcriptional machinery that either activate or repress gene expression. Given the central role served by TFs in all aspects of cellular function, it is unsurprising that aberrant TF activity is widely and unambiguously implicated in human disease. Cancer in particular is hallmarked by direct or upstream deregulation of TFs, among them notorious oncogenes (e.g. MYC) and tumor suppressors (e.g. TP53). Modulation of TF activity therefore offers exceptionally compelling opportunities for the treatment of cancer and other forms of human disease. Notwithstanding their great promise, TFs have proven to be particularly difficult to manipulate pharmacologically(1). Only a small subset of TFs (e.g., nuclear hormone receptors), which possess binding sites for endogenous effector metabolites, have been successfully targeted by cell-permeable small molecules. The remainder of TFs are commonly referred to as being “undruggable.” Additional synthetic strategies and treatments that target TFs are needed.

A distinct family of DNA-binding proteins is characterized by the presence of adjacent “basic” helix-loop-helix, and leucine zipper domains. Members of this family include the Myc oncoproteins, their binding partner Max, and the mammalian transcription factors USF, TFE3, and TFEB (see, e.g., Fisher et al., PNAS (1992) 89:11779-11783; incorporated by reference in its entirety). A large body of evidence has accumulated that demonstrates dominant effects of Myc proto-oncoproteins on different aspects of cellular growth (see, e.g., Luscher et al., Oncogene (1999) 18:2955-29660; incorporated by reference in its entirety). Myc is one of the few proteins that is sufficient to drive resting cells into the cell cycle and promote DNA synthesis. These growth-stimulating properties are most likely responsible for Myc's ability to initiate and promote tumor formation. Interestingly Myc can also sensitize cells to apoptosis, suggesting that this protein is part of a life-and-death switch. Myc is a highly validated target in numerous diseases, including cancer. However, despite being attractive targets for drug discovery, no drugs exist that target non-nuclear hormone receptor transcription factors. Furthermore, there does not currently exist a modular, general method to generate inhibitors of basic-helix-loop-helix transcription factors or related bZIP transcription factors.

### SUMMARY OF THE INVENTION

Provided herein are stapled polypeptides comprising synthetic DNA-binding domains (sDBDs) that mimic the DNA-binding domains of basic helix-loop-helix (bHLH) transcription factors and methods of using such peptides as therapeutic agents. For example, in some embodiments, provided herein is a synthetic DNA binding domain peptide, comprising: a synthetically modfied peptide that binds to a DNA molecule (e.g., a DNA comprising an E-box transcription factor binding site or other DNA binding site), wherein the peptide comprises a dimerization moiety configured to form a dimer with a second modified peptide. The present disclosure is not limited to particular dimerization moieties. Examples include, but are not limited to thiol, maliemide, an alkyne, azide, SMCC, AMAS, EMCS, or MBS. In some embodiments, the E-box transcription factor binding domain has the sequence 5′-CACGTG-3′. In some embodiments, the stapled polypeptides mimic binding of transcription factors that comprises a leucine zipper (LZ) domain (e.g., bZIP transcription factors). In some embodiments, the transcription factor comprises a bHLH and an LZ domain (bHLH-LZ transcription factors). In certain embodiments, binding of the synthetic DNA-binding domains to transcription factor targets interferes with transcription factor function. In particular embodiments, binding of a stapled polypeptide inhibits transcription of the target nucleic acid. Herein, “target nucleic acid” or “target DNA” refers to the nucleic acid to which the bHLH transcription factor (and the DBD mimetic) binds. In certain embodiments, the stapled polypeptide comprises two stapled alpha-helical polypeptides which are covalently conjugated in a specific stereochemical orientation, and which are derived from bHLH transcription factor basic domains of the Myc and Max transcription factors. In certain embodiments, the stapled polypeptides inhibit Myc/Max function, for example, by interfering with Myc/Max binding to DNA. In some embodiments, the peptide is derived from a basic helix-loop-helix leucine-zipper (bHLH-LZ) transcription factor (e.g., AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, CLOCK, EPAS1, FERD3L, FIGLA, HAND1, HAND2, HES1, HES2, HES3, HES4, HES5, HES6, HES7, HEY1, HEY2, HIF1A, ID1, ID2, ID3, ID4, KIAA2018, LYL1, MASH1, MATH2, MAX, MESP1, MESP2, MIST1, MITF, MLX, MLXIP, MLXIPL, MNT, MSC, MSGN1, MXD1, MXD3, MXD4, MXI1, MYC, MYCL1, MYCL2, MYCN, MYF5, MYF6, MYOD1, MYOG, NCOA1, NCOA3, NEUROD1, NEUROD2, NEUROD4, NEUROD6, NEUROG1, NEUROG2, NEUROG3, NHLH1, NHLH2, NPAS1, NPAS2, NPAS3, OAF1, OLIG1, OLIG2, OLIG3, PTF1A, SCL, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, or USF2).

In some embodiments, the polypeptides mimic binding of bZIP transcription factors ATF1, ATF2, ATF4, ATF5, ATF6, ATF7, BACH1, BACH2, BATF, BATF2, CREB1, CREB3, CREB3L1, CREB3L2, CREB3L3, CREB3L4, CREB5, CREBL1, CREM, E4BP4, FOSL1, FOSL2, JUN, JUNB, JUND, NFE2, NFE2L2, NFE2L3, OPAQUE2, SNFT, or CREM.

In specific embodiments, the two amino acid sequences of the stapled polypeptide may be derived from Myc and Max. In yet other embodiments, the stapled polypeptides may be derived from a peptide library screening approach. In certain embodiments, the stapled polypeptides may be modified further, e.g. to substitute non-natural amino acids for natural amino acids, to add or substitute positively charged amino acids for uncharged or negatively charged amino acids, or to add N-terminal or C-terminal moieties, such as tags or labels (e.g., fluorophores, fatty acids, biotin, polyethylene glycol, and acetylation).

In some embodiments, the peptide is a monomeric peptide or a dimeric peptide linked by the dimerization moiety. Examples of specific peptides include, but are not limited to, AcW-βKRRTHNVLERQRRNELKRSβ-C(SEQ ID NO: 1), AcW-βKRAHHNALERKRRDHIKDSβ-K(Mmt) (SEQ ID NO: 2), AcW-βKRAHHNALERKRRDHIKDSβ-K(Mmt) (SEQ ID NO: 3), AcW-βKRRTHN*LER*RRNELKRSβ-C(SEQ ID NO: 4), AcW-βKRRTHNVLER*RRN*LKRSβ-C(SEQ ID NO: 5), AcW-βKR*THN*LERQRRNELKRSβ-C(SEQ ID NO: 6), AcW-βKRAHHN*LER*RRDHIKDSβ-K(Mmt) (SEQ ID NO: 7), AcW-βKRAHHNALER*RRD*IKDSβ-K(Mmt) (SEQ ID NO: 8), AcW-βKRAHHNALER*RRD*IKDSβ-K(Mmt) (SEQ ID NO: 9), AcW-βKR*HHN*LERKRRDHIKDSβ-K(Mmt) (SEQ ID NO: 10), AcW-GKRRTHN*LER*RRNELKRSG-C(SEQ ID NO: 11), AcW-GKR*HHN*LERKRRDHIKDSG-K(Mmt) (SEQ ID NO: 12), AcW-βKRAHHNALER*RRD*IKDS-K(Mmt) (SEQ ID NO: 13), AcW-βKR*HHN*LERKRRDHIKDS-K(Mmt) (SEQ ID NO: 14), AcW-βKR*HHN*LERKRRDHIKDS-K(Mmt) (SEQ ID NO: 15), AcW-βKRRTHN*LER*RRNELKRS-C(SEQ ID NO: 16), AcW-βKRRTHNVLER*RRN*LKRS-C (SEQ ID NO: 17), AcW-βKRAHHNALER*RRD*IKDS-K(Mmt) (SEQ ID NO: 18), AcW-βKRAHHNALER*RRD*IKDS-K(Mmt) (SEQ ID NO: 19), AcW-βKRAHHNALER*RRD*IKDS-K(Mmt) (SEQ ID NO: 20), FITC-PEG3-βKRRTHNVLERQRRNELKRSβ-C(SEQ ID NO: 21), FITC-PEG3-βKRRTHN*LER*RRNELKRSβ-C(SEQ ID NO: 22), FITC-PEG3-βKRRTHNVLER*RRN*LKRSβ-C(SEQ ID NO: 23), Biotin-PEG3-W-βKRRTHN*LER*RRNELKRSβ-C(SEQ ID NO: 24), Biotin-PEG3-W-βKRRTHN*LER*RRNELKRS-C(SEQ ID NO: 25), Fmoc-βKRRTHNVLERQRRNELKRSβ-C(SEQ ID NO: 26), Fmoc-βKRAHHNALERKRRDHIKDSβ-K(Mmt) (SEQ ID NO: 27), Fmoc-βKRRTHN*LER*RRNELKRSβ-C(SEQ ID NO: 28), Fmoc-βKRRTHN*LER*RRNELKRSβ-C(SEQ ID NO: 29), Fmoc-βKRRTHNVLER*RRN*LKRSβ-C(SEQ ID NO: 30), Fmoc-βKRRTHNVLER*RRN*LKRSβ-C(SEQ ID NO: 31), Fmoc-βKRAHHNALER*RRD*IKDSβ-K(Mmt) (SEQ ID NO: 32), Fmoc-βKRAHHNALER*RRD*IKDSβ-K(Mmt) (SEQ ID NO: 33), Fmoc-βKR*HHN*LERKRRDHIKDSβ-K(Mmt) (SEQ ID NO: 34), Fmoc-βKR*HHN*LERKRRDHIKDSβ-K(Mmt) (SEQ ID NO: 35), Fmoc-βKRRTHN*LER*RRNELKRSG-C(SEQ ID NO: 36), Fmoc-βKRRTHN*LER*RRNELKRSG-C(SEQ ID NO: 37), Fmoc-βKR*HHN*LERKRRDHIKDSG-K(Mmt) (SEQ ID NO: 38), Fmoc-βKR*HHN*LERKRRDHIKDSG-K(Mmt) (SEQ ID NO: 39), Fmoc-βKRAHHNALER*RRD*IKDS-K(Mmt) (SEQ ID NO: 40), Fmoc-βKRAHHNALER*RRD*IKDS-K(Mmt) (SEQ ID NO: 41), Fmoc-βKR*HHN*LERKRRDHIKDS-K(Mmt) (SEQ ID NO: 42), Fmoc-βKR*HHN*LERKRRDHIKDS-K(Mmt) (SEQ ID NO: 43), Fmoc-βKRRTHN*LER*RRNELKRS-C (SEQ ID NO: 44), Fmoc-βKRRTHN*LER*RRNELKRS C (SEQ ID NO: 45), Fmoc-βKRRTHNVLER*RRN*LKRS-C (SEQ ID NO: 46), Fmoc-βKRRTHNVLER*RRN*LKRS-C (SEQ ID NO: 47), Fmoc-βKRRTHN*LER*RRNELKRSβ-K(Mmt) (SEQ ID NO: 48), Fmoc-βKRRTHNVLER*RRN*LKRSβ-K(Mmt) (SEQ ID NO: 49), Fmoc-βKRRTHN*LER*RRNELKRSβ-K(Mmt) (SEQ ID NO: 50), Fmoc-βKRRTHNVLER*RRN*LKRSβ-K(Mmt) (SEQ ID NO: 51), the peptides shown in FIGS. 14 and 15, and variants (e.g., variants that are at least 80%, 85%, 90%, 95%, or 99% identical to such peptides), mimetics, or modified versions thereof. In some embodiments, S5 and S5 amino acids are utilzed to create a single alpha-helical turn cross-link. In some embodiments, the crosslinking is two turns (e.g., R8/S5), triazole “click” crosslinks, or thioether crosslinks. In some embodiments, the peptide inhibits the activity of the transcription factor.

Further embodiments provide a complex, comprising: at least one peptide described herein bound to an E-box transcription factor binding domain (e.g., 5′-CACGTG-3′). In some embodiments, the at least one peptide is two peptides, wherein each of the two peptides has a different dimerization moiety, and wherein the different dimerization moieties form a covalent bond when contacted. In some embodiments, the at least one peptide comprises two peptides covalently linked by one or more dimerization moieties.

In some embodiments, provided herein are compositions, comprising: (a) a first synthetic peptide comprising: (i) at least one internal hydrocarbon staple, and (ii) a first dimerization moiety; and (b) a second synthetic peptide comprising: (i) at least one internal hydrocarbon staple, and (ii) a second dimerization moiety; wherein the first and second dimerization moieties are capable of interacting to form a stable bond, thereby forming a dimer of the first and second synthetic peptides. In some embodiments, the hydrocarbon staples are the result of ring-closing olefin metathesis (RCM) of hindered α-methyl, α-alkenyl amino acids. In some embodiments, the first and second dimerization moieties are attached to a side chain of N-terminal amino acids. In some embodiments, the first dimerization moiety comprises a thiol and the second dimerization moiety comprises a maleimide. In some embodiments, the first dimerization moiety comprises a azide and the second dimerization moiety comprises an alkyne. In some embodiments, the first synthetic peptide further comprises a third dimerization moiety and the second synthetic peptide further comprises a fourth dimerization moiety; wherein the third and fourth dimerization moieties are capable of interacting to form a stable bond, thereby forming a dimer of the first and second synthetic peptides. In some embodiments, the third and fourth dimerization moieties are attached to a side chain of an amino acid within 5 positions (e.g., 5, 4, 3, 2, 1, or ranges therebetween) of the N-terminal amino acid. In some embodiments, the first dimerization moiety comprises a thiol, the second dimerization moiety comprises a maleimide, the third dimerization moiety comprises an azide, and the fourth dimerization moiety comprises an alkyne. In some embodiments, provided herein are dimers of the synthetic peptides described herein.

In some embodiments, provided herein is a peptide or conjugate of peptides of one of Formulas I-XII.

Additional embodiments provide a pharmaceutical composition, comprising at least one of the peptides described herein.

Yet other embodiments provide a method of inhibiting the activity of a transcription factor, comprising: contacting the transcription factor with at least one of the peptides described herein, wherein the contacting inhibits the activity of the transcription factor. In some embodiments, the inhibiting treats a disease (e.g., cancer).

Still other embodiments provide a method of treating a disease, comprising: administering the pharmaceutical composition or peptides described herein to a subject in need thereof, wherein the administering treats the disease (e.g., cancer).

Also provided herein is the use of the described peptides to inhibit at least one activity of a transcription factor.

Some embodiments provide the use of the described peptides or pharmaceutical compositions to treat a disease (e.g., cancer).

Additional embodiments are described herein.

### DESCRIPTION OF THE FIGURES

FIG. 1 shows that sDBDs bind E-box DNA sites and antagonize Myc. a) Structure of Myc/Max bound to an E-box (5′-CACGTG-3′) consensus site. b-c) Libraries of sDBDs can be synthesized (b) and purified (c) by conventional techniques. d) EMSA gel-shift assays of E-box DNA show comparable binding by Myc/Max and a representative sDBD. e-f) ALPHAscreen proximity assay reveals potent binding of E-box DNA by Myc/Max (e), which is competed by soluble sDBD1 (f).

FIG. 2 shows design and synthesis of optimized sDBDs a) Circular dichroism spectra of unmodified wild type (WT) Myc and Max basic peptides and the corresponding stapled counterparts. Absorbance minima at 208 and 222 nm are characteristic for helical character. b) Optimization strategies for improved sDBD binding affinity and specificity. c) Schematic of the structure of i−i+4, i−i+7 and i−i+4−i+11 hyrdocarbon macrocycles. d) Representative alternative linker structures to be compared with the current cyclohexyl-maleimide scaffold. e) Representative secondary linker structures to mediate non-covalent ionic and hydrophobic contacts, which mimic natural bHLH proteins, or chemically orthogonal covalent linkers.

FIG. 3 shows biochemical assays to study sDBD and Myc/Max DNA binding a) Representative biacore binding curves derived from immobilized, biotinylated E-box DNA binding soluble Myc/Max heterodimer. Kinetic fitting of association and dissociation yields both thermodynamic and kinetic binding constants. b) Schematic of a Myc/Max-E-box ALPHAscreen proximity assay. Incubation of biotinylated-E-box DNA with His6-tagged Myc/Max leads to the formation of heterotrimeric complex and subsequent association of streptavidin-coated donor beads with Ni²⁺-NTA-functionalized acceptor beads. This highly specific proximity assay yields excellent signal to noise values and can be used to compare sDBDs for the ability to inhibit Myc/Max DNA binding (FIGS. 1e, f).

FIG. 4 Shows bHLH domains of Myc and Max proteins bound to E-box DNA. Leucine zipper and loop domains enable juxtaposition and orientation of individual DNA-binding helices. Mimics, such as sDBDs, must account for both secondary and tertiary structural aspects.

FIG. 5 shows Minimized DNA binding helices from Myc and Max, which are equivalent to the same region in other bHLH proteins and related transcription factors.

FIG. 6 shows sDBD design: Schematic depicting solid-phase synthesis of individual sDBD monomer stapled peptides with dimerization motifs. In the example shown, the top peptide is stapled, an orthogonally protected lysine at the C-terminus is deprotected and a modular reactive group used for subsequent dimerization is covalently attached, in this case this is a maleimide. The corresponding monomer stapled helix containing a C-terminal reactive dimerization moeity also synthesized, in this case a thiol.

FIG. 7 shows sDBD design: Schematic depicting solution-based conjugation of two stapled monomer peptides to synthesize an sDBD dimer with specific secondary structure stabilization (defined hydrocarbon staples) and intermolecular covalent dimerization. Individual monomers and conjugated dimers can be analyzed and purified by conventional liquid chromatography-mass spectrometry. Also shown are design elements for individual monomer DNA-binding peptides from Myc (SEQ ID NO: 1) and Max (SEQ ID NO: 2). Contacts to specific nucleobases in the consensus E-box motif, van der waals contacts and contacts to the phosphodiester backbone are indicated. Representative non-binding residues that may be amenable to non-natural amino acid incorporation are shown in white.

FIG. 8 shows Design elements of synthetic DNA-binding domains (sDBDs) targeting bHLH-LZ TFs. Highlighted residues in individual DNA binding helices from Myc and Max were tested, including others, for incorporation of hydrocarbon stapling amino acids. Also shown are representative CD spectra from non-modified Myc and Max basic helices (black) and a corresponding hydrocarbon stapled version (grey), with signal at 208 and 222 nm indicated.

FIG. 9 Representative electrophoretic mobility shift assay (EMSA, referred to as “Gel Shift) images of full length Myc/Max protein binding a fluorophore-labled oligonucleotide containing an E-box binding site. Full-length Myc/Max binds in this assay with an affinity of ˜10 nM. A synthetic dimer of the two unmodified DNA binding helices from Myc/Max (RTD31) does not bind this E-box site with appreciable stability or affinity. This demonstrates the importance of secondary structure stabilization as well as tertiary structure stabilization in sDBDs, as joining of the individual helices alone does not result in appreciable binding.

FIG. 10 shows that sDBDs potently bind E-box sequences. A representative sDBD (RTD84), containing specific sites of hydrocarbon helix stabilization, a beta-alanine linker within each helix, and an SMCC-thiol dimerization motif, binds E-box DNA with Kd=50 nM. Changing only the beta-alanine linker to a glycine (one methylene change) abrogates binding, due to improper orientation of DNA-binding residues in the resulting compound (RTD84G). Removal of the in-helix linker altogether keeps the helix in proper register but leads to less-stable binding of a 1:1 sDBD-DNA complex in the compound RTD913. These data show the sensitivity and non-obvious nature of incorporating secondary and tertiary stabilization elements in sDBDs.

FIG. 11 shows thaT sDBDs are cell permeable. Direct comparison of RTD31, the dimer of Myc and Max basic helices without hydrocarbon staples, and RTD84, a fully stabilized sDBD, for cellular uptake. FITC-conjugated compounds were incubated with HeLa cells in media containing 10% FBS for six hours. Microscopy showed increased overall cellular fluorescence and localization to the nucleus.

FIG. 12 shows exemplary monomer structures.

FIG. 13 shows exemplary dimer structures.

FIG. 14 shows exemplary conjugation methods. Various biocompatible, high-efficiency ligation strategies are listed, such as thiol-maleimide Michael addition, azide-alkyne cycloaddition, amide bond formation, and hydrazone formation.

FIG. 15 shows exemplary conjugation methods.

FIG. 16 shows alkyne-azide huisgen conjugation. Monomeric stapled peptides containing either C-terminal alkyne or azide are synthesized by orthogonal chemistry.

FIG. 17 shows alkyne-azide huisgen conjugation to form triazole-linker sDBDs.

FIG. 18 shows circular dichroism Myc Peptides RTD-1 (SEQ ID NO: 21), RTD-4 (SEQ ID NO: 22), RTD-5 (SEQ ID NO: 23), and RTD-6 (SEQ ID NO: 6); and Max peptides RTD-3 (SEQ ID NO: 2), RTD-7 (SEQ ID NO: 8), RTD-2 (SEQ ID NO: 7), and RTD-8 (SEQ ID NO: 10).

FIG. 19 shows recombinant Myc/Max DNA-binding activity measured by gel-shift assay. Unlabeled competitor DNA oligos containing either consensus or mutant sequences are added to reveal sequence-specific binding.

FIG. 20 shows sDBD DNA-binding activity measured by gel-shift assay.

FIG. 21 shows DNA-binding activity of peptides comprising linkers measured by gel-shift assay. Beta-alanine helix cap promotes tight, stable binding. Glycine insertion over-rotates helix, leads to reduced binding. No helix cap (continuous helix) leads to potent but unstable binding.

FIG. 22 shows dimerization moiety effect on stable DNA-binding as measured by gel-shift assay. RTD913 EC50 is ˜20 nM for stable complex formation. RTD913 represents contains no helix-cap and therefore both DNA-binding helices are continuous, this promotes tight, but somewhat unstable binding. Incorporation of a rigid linker (MBS) in this design results in both tight and stable binding (913-MBS).

FIG. 23 shows sDBD structure-activity relationship. The binding affinity is dependent on average helical content of the sDBDs (left) and the dimerization motif that alters the helix distance/orientation (right).

FIG. 24 shows the results of the ALPHAscreen assay. Titration of the biotinylated-E-box probe with various concentration of the Myc/Max heterodimer reveals specific binding and a robust signal-to-noise ratio of >60-fold. In a competitive assay, a lead first generation RTD compound (RTD84) was co-incubated with a constant amount of E-box probe and Myc/Max heterodimer. RTD84 shows dose-dependent inhibition of complex formation, with an IC50 value in the low micromolar range. A positive control compound, 10058-F4, which has been shown to inhibit Myc/Max dimerization, also inhibited DNA-binding competitively, but with a higher IC50 value (˜8-10 micromolar). This indicates that sDBDs derived from Myc/Max show competitive inhibition of Myc/Max binding to a consensus E-box oligonucleotide.

FIG. 25 shows that sDBDs are cell permeable, localize to cytoplasm and nucleus.

FIG. 26 shows that RTD84 shows strong cytosolic and nuclear localization at 6 and 10 hrs.

FIG. 27 show a schematic of an exemplary synthesis of sDBDs.

FIG. 28 show a schematic of an exemplary synthesis of sDBDs by click chemistry.

FIG. 29 show a schematic of an exemplary synthesis of dual-linker sDBDs.

FIG. 30A-D shows graphs depicting the results of competition ChIP-qPCR assays. Myc-ChIP was performed with HeLa cells. HeLa cells were seeded in 150-mm plates in RPMI-1640 (containing 10% FBS) and incubated at 37° C. and 5% CO2 until confluent. Each plate was then treated with media containing either DMSO or sDBD for specified time. After crosslinked with 1% formaldehyde and harvested, cells were lyzed and chromatins were isolated and sonicated to an average size of ˜200 bp. ChIP was performed using rabbit polyclonal c-Myc antibody N-262 (sc-764) and protein G beads. After reverse-crosslinking at 65° C. overnight, ChIP DNA was purified and quantified by qPCR. (A) Primer validation; (B) Dose-dependent competition (RTD-84, 4 hr); (C) Time-dependent competition (RTD-84, 10 μM); (D) Compound-dependent competition (10 μM, 4 hr).

FIG. 31 shows graphs depicting the results of cell viability assays. A variety of cancer cell lines were treated with sDBDs for 24 hrs. Viable cells were quantified by CELLTITER-GLO reagent. 2500 cells were seeded in flat-bottom 96-well white plates in RPMI-1640 (containing 10% FBS) and allowed to settle at 37° C. and 5% CO₂ overnight. Each well was then treated with media containing varied concentrations of sDBD for 24 hours. After incubation was complete, cells were lyzed with CELLTITER-GLO reagent and viable cell numbers were quantified using a luminescence plate reader.

FIG. 32 shows exemplary circular dichroism spectra of peptide monomers. All stapled peptides show significantly increased helical content compared to their unmodified counterparts.

FIG. 33A-F shows exemplary cell penetration data. (A) Fluorescent microscopic images of HeLa cells treated with either DMSA, unmodified FITC-DBD or FITC-sDBD. (B) Quantification of FITC channel fluorescence intensity. (C) Fluorescent microscopic images of HeLa cells treated with FITC-sDBD for different time periods. (D) Channel overlays demonstrating colocalization of fluorescent signal. (E) Plot of time-dependent cell penetration. (F) Quantification of colocalization percentage indicates substantial concurrence of FITC and DAPI fluorescent signals.

FIG. 34 shows exemplary bifunctional monomers. Featuring two orthogonal reactive groups on each single monomer, they can be further ligated to form the dual-linker sDBDs, which are constrained to the binding conformation due to the presence of the secondary linker, maximizing binding affinity.

FIG. 35 shows exemplary bifunctional monomers with swapped linker positions. The structure of maleimide linker can be varied to alter the distance of the two helices.

FIG. 36 shows the exemplary sequences of Fos/Jun sDBD library. Fos/Jun is a proto-oncogenic bZIP TF whose inhibitors can be designed by applying the similar strategy.

FIG. 37 shows a graph depicting the results of electrophoretic mobility shift assays using various combinations of the Fos/Jun sDBD monomers depicted in FIG. 38. An infrared dye labeled DNA oligo containing Fos/Jun binding consensus 5′-TGACTCA-3′ is used as a florescent probe. It is shown that some candidates show enhanced DNA binding affinity compared to unmodified dimeric peptide FJ-WT.

FIG. 38 shows exemplary monomers derived from Fos.

FIG. 39 shows exemplary monomers derived from Jun.

FIG. 40A-B shows exemplary dimeric sDBDs targeting Fos/Jun.

### DEFINITIONS

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a stapled polypeptide” is a reference to one or more stapled polypeptides and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” refers to organisms to be treated by the methods of embodiments of the present invention. Such organisms preferably include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of a peptide of the present invention and optionally one or more other agents) for disease (e.g., cancer) or other condition requiring treatment. As used herein, the term “patient” typically refers to a human subject that is being treated for a disease or condition.

“Stapling” or “hydrocarbon-stapling,” as used herein, is a process by which two terminally unsaturated amino acid side chains in a polypeptide chain react with each in the presence of a ring closing metathesis catalyst to generate a C—C double bonded cross-link between the two amino acids (a “staple”). Stapling engenders constraint on a secondary structure, such as an alpha helical structure. The length and geometry of the cross-link can be optimized to improve the yield of the desired secondary structure content. The constraint provided can, for example, prevent the secondary structure to unfold and/or can reinforce the shape of the secondary structure, and thus makes the secondary structure more stable.

Multiple stapling is also referred to herein as “stitching.” In certain embodiments, hydrocarbon staples are the result of ring-closing olefin metathesis (RCM) of hindered α-methyl, α-alkenyl amino acids.

It will be appreciated that the compounds of the present invention, as described herein, may be substituted with any number of substituents or functional moieties. As used herein, “optionally substituted” refers to a group as substituted or unsubstituted. In general, the term “substituted” whether preceded by the term “optionally” or not, and substituents contained in Formulas of this invention, refer to the replacement of hydrogen radicals in a given structure with the radical of a specified substituent. When more than one position in any given structure may be substituted with more than one substituent selected from a specified group, the substituent may be either the same or different at every position.

As used herein, the term “substituted” is contemplated to include substitution with all permissible substituents of organic compounds, any of the substituents described herein (for example, aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, etc.), and any combination thereof (for example, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like) that results in the formation of a stable moiety.

The term “stable moiety,” as used herein, preferably refers to a moiety which possess stability sufficient to allow manufacture, and which maintains its integrity for a sufficient period to be useful for the purposes detailed herein. The present invention contemplates any and all such combinations in order to arrive at a stable substituent/moiety. Additional examples of generally applicable substituents are illustrated by the specific embodiments shown in the Examples, which are described herein. For purposes of this invention, heteroatoms such as nitrogen may have hydrogen substituents and/or any suitable substituent as described herein which satisfy the valencies of the heteroatoms and results in the formation of a stable moiety

The term “diagnosed,” as used herein, refers to the recognition of a disease by its signs and symptoms (e.g., resistance to conventional therapies), or genetic analysis, pathological analysis, histological analysis, diagnostic assay (e.g., for disease) and the like.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “host cell” refers to any eukaryotic or prokaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the term “effective amount” refers to the amount of a therapeutic agent (e.g., a peptide of the present invention) sufficient to effect beneficial or desired results. An effective amount can be administered in one or more administrations, applications or dosages and is not intended to be limited to a particular formulation or administration route.

As used herein, the term “co-administration” refers to the administration of at least two agent(s) (e.g., a peptide of the present invention) or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

The term “sample” as used herein is used in its broadest sense. A sample may be biological or environmental in origin, and may comprise a cell, tissue, or fluids, nucleic acids or polypeptides isolated from a cell, and the like.

As used herein, the terms “purified” or “to purify” refer, to the removal of undesired components from a sample. As used herein, the term “substantially purified” refers to molecules that are at least 60% free, preferably 75% free, and most preferably 90%, or more, free from other components with which they usually associated.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein, the term “wild-type,” refers to a gene or gene product (e.g., protein) that has the characteristics (e.g., sequence) of that gene or gene product isolated from a naturally occurring source, and is most frequently observed in a population. In contrast, the term “mutant” refers to a gene or gene product that displays modifications in sequence when compared to the wild-type gene or gene product. It is noted that “naturally-occurring mutants” are genes or gene products that occur in nature, but have altered sequences when compared to the wild-type gene or gene product; they are not the most commonly occurring sequence. “Synthetic mutants” are genes or gene products that have altered sequences when compared to the wild-type gene or gene product and do not occur in nature. Mutant genes or gene products may be naturally occurring sequences that are present in nature, but not the most common variant of the gene or gene product, or “synthetic,” produced by human or experimental intervention.

As used herein the term “portion” when in reference to a protein (as in “a portion of a given protein”) refers to fragments of that protein. The fragments may range in size from four amino acid residues to the entire amino acid sequence minus one amino acid.

The term “test compound” refers to any chemical entity, pharmaceutical, drug, and the like, that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention. In some embodiments, “test compounds” are agents that treat or prevent disease (e.g., cancer).

As used herein, substituent names which end in the suffix “ene” refer to a biradical derived from the removal of two hydrogen atoms from the substituent. Thus, for example, acyl is acylene; alkyl is alkylene; alkeneyl is alkenylene; alkynyl is alkynylene; heteroalkyl is heteroalkylene, heteroalkenyl is heteroalkenylene, heteroalkynyl is heteroalkynylene, aryl is arylene, and heteroaryl is heteroarylene.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms (C₁₋₂₀ aliphatic). In certain embodiments, the aliphatic group has 1-10 carbon atoms (C₁₋₁₀ aliphatic). In certain embodiments, the aliphatic group has 1-6 carbon atoms (C₁₋₆ aliphatic). In certain embodiments, the aliphatic group has 1-5 carbon atoms (C₁₋₅ aliphatic). In certain embodiments, the aliphatic group has 1-4 carbon atoms (C₁₋₄ aliphatic). In certain embodiments, the aliphatic group has 1-3 carbon atoms (C₁₋₃ aliphatic). In certain embodiments, the aliphatic group has 1-2 carbon atoms (C₁₋₂ aliphatic). Aliphatic group substituents include, but are not limited to, any of the substituents described herein, that result in the formation of a stable moiety.

The term “alkyl,” as used herein, refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-20 carbon atoms (C₁₋₂₀alkyl). In another embodiment, the alkyl group employed contains 1-15 carbon atoms (C₁₋₁₅alkyl). In another embodiment, the alkyl group employed contains 1-10 carbon atoms (C₁₋₁₀alkyl). In another embodiment, the alkyl group employed contains 1-8 carbon atoms (C₁₋₈alkyl). In another embodiment, the alkyl group employed contains 1-6 carbon atoms (C₁₋₆alkyl). In another embodiment, the alkyl group employed contains 1-5 carbon atoms (C₁₋₅alkyl). In another embodiment, the alkyl group employed contains 1-4 carbon atoms (C₁₋₄alkyl). In another embodiment, the alkyl group employed contains 1-3 carbon atoms (C₁₋₃alkyl). In another embodiment, the alkyl group employed contains 1-2 carbon atoms (C₁₋₂alkyl). Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents. Alkyl group substituents include, but are not limited to, any of the substituents described herein, that result in the formation of a stable moiety. The term “alkylene,” as used herein, refers to a biradical derived from an alkyl group, as defined herein, by removal of two hydrogen atoms. Alkylene groups may be cyclic or acyclic, branched or unbranched, substituted or unsubstituted. Alkylene group substituents include, but are not limited to, any of the substituents described herein, that result in the formation of a stable moiety.

The term “alkenyl,” as used herein, denotes a monovalent group derived from a straight- or branched-chain hydrocarbon moiety having at least one carbon-carbon double bond by the removal of a single hydrogen atom. In certain embodiments, the alkenyl group employed in the invention contains 2-20 carbon atoms (C₂₋₂₀alkenyl). In some embodiments, the alkenyl group employed in the invention contains 2-15 carbon atoms (C₂₋₁₅alkenyl). In another embodiment, the alkenyl group employed contains 2-10 carbon atoms (C₂₋₁₀alkenyl). In still other embodiments, the alkenyl group contains 2-8 carbon atoms (C₂₋₈alkenyl). In yet other embodiments, the alkenyl group contains 2-6 carbons (C₂₋₆alkenyl). In yet other embodiments, the alkenyl group contains 2-5 carbons (C₂₋₅alkenyl). In yet other embodiments, the alkenyl group contains 2-4 carbons (C₂₋₄alkenyl). In yet other embodiments, the alkenyl group contains 2-3 carbons (C₂₋₃alkenyl). In yet other embodiments, the alkenyl group contains 2 carbons (C₂alkenyl). Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety. The term “alkenylene,” as used herein, refers to a biradical derived from an alkenyl group, as defined herein, by removal of two hydrogen atoms. Alkenylene groups may be cyclic or acyclic, branched or unbranched, substituted or unsubstituted. Alkenylene group substituents include, but are not limited to, any of the substituents described herein, that result in the formation of a stable moiety.

The term “alkynyl,” as used herein, refers to a monovalent group derived from a straight- or branched-chain hydrocarbon having at least one carbon-carbon triple bond by the removal of a single hydrogen atom. In certain embodiments, the alkynyl group employed in the invention contains 2-20 carbon atoms (C₂₋₂₀alkynyl). In some embodiments, the alkynyl group employed in the invention contains 2-15 carbon atoms (C₂₋₁₅alkynyl). In another embodiment, the alkynyl group employed contains 2-10 carbon atoms (C₂₋₁₀alkynyl). In still other embodiments, the alkynyl group contains 2-8 carbon atoms (C₂₋₈alkynyl). In still other embodiments, the alkynyl group contains 2-6 carbon atoms (C₂₋₆alkynyl). In still other embodiments, the alkynyl group contains 2-5 carbon atoms (C₂₋₅alkynyl). In still other embodiments, the alkynyl group contains 2-4 carbon atoms (C₂₋₄alkynyl). In still other embodiments, the alkynyl group contains 2-3 carbon atoms (C₂₋₃alkynyl). In still other embodiments, the alkynyl group contains 2 carbon atoms (C₂alkynyl). Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety. The term “alkynylene,” as used herein, refers to a biradical derived from an alkynylene group, as defined herein, by removal of two hydrogen atoms. Alkynylene groups may be cyclic or acyclic, branched or unbranched, substituted or unsubstituted. Alkynylene group substituents include, but are not limited to, any of the substituents described herein, that result in the formation of a stable moiety.

The term “carbocyclic” or “carbocyclyl” as used herein, refers to an as used herein, refers to a cyclic aliphatic group containing 3-10 carbon ring atoms (C₃₋₁₀carbocyclic). Carbocyclic group substituents include, but are not limited to, any of the substituents described herein, that result in the formation of a stable moiety.

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that further contains one or more heteroatoms (e.g., oxygen, sulfur, nitrogen, phosphorus, or silicon atoms) between carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl,” “heteroalkynyl,” and the like. Furthermore, as used herein, the terms “heteroalkyl,” “heteroalkenyl,” “heteroalkynyl,” and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms and 1-6 heteroatoms (C₁₋₂₀heteroaliphatic). In certain embodiments, the heteroaliphatic group contains 1-10 carbon atoms and 1-4 heteroatoms (C₁₋₁₀heteroaliphatic). In certain embodiments, the heteroaliphatic group contains 1-6 carbon atoms and 1-3 heteroatoms (C₁₋₆heteroaliphatic). In certain embodiments, the heteroaliphatic group contains 1-5 carbon atoms and 1-3 heteroatoms (C₁₋₅heteroaliphatic). In certain embodiments, the heteroaliphatic group contains 1-4 carbon atoms and 1-2 heteroatoms (C₁₋₄heteroaliphatic). In certain embodiments, the heteroaliphatic group contains 1-3 carbon atoms and 1 heteroatom (C₁₋₃heteroaliphatic). In certain embodiments, the heteroaliphatic group contains 1-2 carbon atoms and 1 heteroatom (C₁₋₂heteroaliphatic). Heteroaliphatic group substituents include, but are not limited to, any of the substituents described herein, that result in the formation of a stable moiety.

The term “heteroalkyl,” as used herein, refers to an alkyl moiety, as defined herein, which contain one or more heteroatoms (e.g., oxygen, sulfur, nitrogen, phosphorus, or silicon atoms) in between carbon atoms. In certain embodiments, the heteroalkyl group contains 1-20 carbon atoms and 1-6 heteroatoms (C₁₋₂₀ heteroalkyl). In certain embodiments, the heteroalkyl group contains 1-10 carbon atoms and 1-4 heteroatoms (C₁₋₁₀ heteroalkyl). In certain embodiments, the heteroalkyl group contains 1-6 carbon atoms and 1-3 heteroatoms (C₁₋₆ heteroalkyl). In certain embodiments, the heteroalkyl group contains 1-5 carbon atoms and 1-3 heteroatoms (C₁₋₅ heteroalkyl). In certain embodiments, the heteroalkyl group contains 1-4 carbon atoms and 1-2 heteroatoms (C₁₋₄ heteroalkyl). In certain embodiments, the heteroalkyl group contains 1-3 carbon atoms and 1 heteroatom (C₁₋₃ heteroalkyl). In certain embodiments, the heteroalkyl group contains 1-2 carbon atoms and 1 heteroatom (C₁₋₂ heteroalkyl). The term “heteroalkylene,” as used herein, refers to a biradical derived from an heteroalkyl group, as defined herein, by removal of two hydrogen atoms. Heteroalkylene groups may be cyclic or acyclic, branched or unbranched, substituted or unsubstituted. Heteroalkylene group substituents include, but are not limited to, any of the substituents described herein, that result in the formation of a stable moiety.

The term “heteroalkenyl,” as used herein, refers to an alkenyl moiety, as defined herein, which further contains one or more heteroatoms (e.g., oxygen, sulfur, nitrogen, phosphorus, or silicon atoms) in between carbon atoms. In certain embodiments, the heteroalkenyl group contains 2-20 carbon atoms and 1-6 heteroatoms (C₂₋₂₀ heteroalkenyl). In certain embodiments, the heteroalkenyl group contains 2-10 carbon atoms and 1-4 heteroatoms (C₂₋₁₀ heteroalkenyl). In certain embodiments, the heteroalkenyl group contains 2-6 carbon atoms and 1-3 heteroatoms (C₂₋₆ heteroalkenyl). In certain embodiments, the heteroalkenyl group contains 2-5 carbon atoms and 1-3 heteroatoms (C₂₋₅ heteroalkenyl). In certain embodiments, the heteroalkenyl group contains 2-4 carbon atoms and 1-2 heteroatoms (C₂₋₄ heteroalkenyl). In certain embodiments, the heteroalkenyl group contains 2-3 carbon atoms and 1 heteroatom (C₂₋₃ heteroalkenyl). The term “heteroalkenylene,” as used herein, refers to a biradical derived from an heteroalkenyl group, as defined herein, by removal of two hydrogen atoms. Heteroalkenylene groups may be cyclic or acyclic, branched or unbranched, substituted or unsubstituted.

The term “heteroalkynyl,” as used herein, refers to an alkynyl moiety, as defined herein, which further contains one or more heteroatoms (e.g., oxygen, sulfur, nitrogen, phosphorus, or silicon atoms) in between carbon atoms. In certain embodiments, the heteroalkynyl group contains 2-20 carbon atoms and 1-6 heteroatoms (C₂₋₂₀ heteroalkynyl). In certain embodiments, the heteroalkynyl group contains 2-10 carbon atoms and 1-4 heteroatoms (C₂₋₁₀ heteroalkynyl). In certain embodiments, the heteroalkynyl group contains 2-6 carbon atoms and 1-3 heteroatoms (C₂₋₆ heteroalkynyl). In certain embodiments, the heteroalkynyl group contains 2-5 carbon atoms and 1-3 heteroatoms (C₂₋₅ heteroalkynyl). In certain embodiments, the heteroalkynyl group contains 2-4 carbon atoms and 1-2 heteroatoms (C₂₋₄ heteroalkynyl). In certain embodiments, the heteroalkynyl group contains 2-3 carbon atoms and 1 heteroatom (C₂₋₃ heteroalkynyl). The term “heteroalkynylene,” as used herein, refers to a biradical derived from an heteroalkynyl group, as defined herein, by removal of two hydrogen atoms. Heteroalkynylene groups may be cyclic or acyclic, branched or unbranched, substituted or unsubstituted.

The term “heterocyclic,” “heterocycles,” or “heterocyclyl,” as used herein, refers to a cyclic heteroaliphatic group. A heterocyclic group refers to a nonaromatic, partially unsaturated or fully saturated, 3-to 10-membered ring system, which includes single rings of 3 to 8 atoms in size, and bi- and tri-cyclic ring systems which may include aromatic five- or six-membered aryl or heteroaryl groups fused to a nonaromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety.

The term “aryl,” as used herein, refers to an aromatic mono- or polycyclic ring system having 3-20 ring atoms, of which all the ring atoms are carbon, and which may be substituted or unsubstituted. In certain embodiments of the present invention, “aryl” refers to a mono, bi, or tricyclic C₄-C₂₀ aromatic ring system having one, two, or three aromatic rings which include, but are not limited to, phenyl, biphenyl, naphthyl, and the like, which may bear one or more substituents. Aryl substituents include, but are not limited to, any of the substituents described herein, that result in the formation of a stable moiety. The term “arylene,” as used herein refers to an aryl biradical derived from an aryl group, as defined herein, by removal of two hydrogen atoms. Arylene groups may be substituted or unsubstituted. Arylene group substituents include, but are not limited to, any of the substituents described herein, that result in the formation of a stable moiety. Additionally, arylene groups may be incorporated as a linker group into an alkylene, alkenylene, alkynylene, heteroalkylene, heteroalkenylene, or heteroalkynylene group, as defined herein.

The term “heteroaryl,” as used herein, refers to an aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety. The term “heteroarylene,” as used herein, refers to a biradical derived from an heteroaryl group, as defined herein, by removal of two hydrogen atoms. Heteroarylene groups may be substituted or unsubstituted. Additionally, heteroarylene groups may be incorporated as a linker group into an alkylene, alkenylene, alkynylene, heteroalkylene, heteroalkenylene, or heteroalkynylene group, as defined herein. Heteroarylene group substituents include, but are not limited to, any of the substituents described herein, that result in the formation of a stable moiety.

The term “acyl,” as used herein, is a subset of a substituted alkyl group, and refers to a group having the general formula —C(═O)R^{A}, —C(═O)OR^{A}, —C(═O)—O—C(═O)R^{A}, —C(═O)SR^{A}, —C(═O)N(R^{A})₂, —C(═S)R^{A}, —C(═S)N(R^{A})₂, and —C(═S)S(R^{A}), —C(═NR^{A})R^{A}, —C(═NR^{A})OR^{A}, —C(═NR^{A})SR^{A}, and —C(═NR^{A})N(R^{A})₂, wherein R^{A} is hydrogen; halogen; substituted or unsubstituted hydroxyl; substituted or unsubstituted thiol; substituted or unsubstituted amino; acyl; optionally substituted aliphatic; optionally substituted heteroaliphatic; optionally substituted alkyl; optionally substituted alkenyl; optionally substituted alkynyl; optionally substituted aryl, optionally substituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{A} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety.

The term “acylene,” as used herein, is a subset of a substituted alkylene, substituted alkenylene, substituted alkynylene, substituted heteroalkylene, substituted heteroalkenylene, or substituted heteroalkynylene group, and refers to an acyl group having the general formulae: —R⁰—(C═X¹)—R⁰—, —R⁰—X²(C═X¹)—R⁰—, or —R⁰—X²(C═X¹)X³—R⁰—, where X¹, X², and X³ is, independently, oxygen, sulfur, or NR^{r}, wherein R^{r} is hydrogen or optionally substituted aliphatic, and R⁰ is an optionally substituted alkylene, alkenylene, alkynylene, heteroalkylene, heteroalkenylene, or heteroalkynylene group, as defined herein. Exemplary acylene groups wherein R⁰ is alkylene includes —(CH₂)_{T}—O(C═O)—(CH₂)_{T}—; —(CH₂)_{T}—NR^{r}(C═O)—(CH₂)_{T}—; —(CH₂)_{T}—O(C═NR^{r})—(CH₂)_{T}—; —(CH₂)_{T}—NR^{r}(C═NR^{r})—(CH₂)_{T}—; —(CH₂)_{T}—(C═O)—(CH₂)_{T}—; —(CH₂)_{T}—(C═NR^{r}—(CH₂)_{T}—; —(CH₂)_{T}—S(C═S)—(CH₂)_{T}—; —(CH₂)_{T}—NR^{r}(C═S)—(CH₂)_{T}; —(CH₂)_{T}—S(S═NR^{r})—(CH₂)_{T}—; —(CH₂)_{T}—O(C═S)—(CH₂)_{T}—; —(CH₂)_{T}—(C═S)—(CH₂)_{T}—; or —(CH₂)_{T}—S(C═O)—(CH₂)_{T}—, and the like, which may bear one or more substituents; and wherein each instance of T is, independently, an integer between 0 to 20.

Acylene groups may be cyclic or acyclic, branched or unbranched, substituted or unsubstituted. Acylene substituents include, but are not limited to, any of the substituents described herein, that result in the formation of a stable moiety.

The term “amino,” as used herein, refers to a group of the formula (—NH₂). A “substituted amino” refers either to a mono-substituted amine (—NHR^{h}) of a disubstituted amine (—NR^{h}₂), wherein the R^{h} substituent is any substituent as described herein that results in the formation of a stable moiety (e.g., an amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). In certain embodiments, the R^{h} substituents of the di-substituted amino group(—NR^{h}₂) form a 5- to 6-membered heterocyclic ring.

The term “hydroxy” or “hydroxyl,” as used herein, refers to a group of the formula (—OH). A “substituted hydroxyl” refers to a group of the formula (—ORⁱ), wherein Rⁱ can be any substituent which results in a stable moiety (e.g., a hydroxyl protecting group; aliphatic, alkyl, alkenyl, alkynyl, heteroaliphatic, heterocyclic, aryl, heteroaryl, acyl, nitro, alkylaryl, arylalkyl, and the like, each of which may or may not be further substituted).

The term “thio” or “thiol,” as used herein, refers to a group of the formula (—SH). A “substituted thiol” refers to a group of the formula (—SR^{r}), wherein R^{r} can be any substituent that results in the formation of a stable moiety (e.g., a thiol protecting group; aliphatic, alkyl, alkenyl, alkynyl, heteroaliphatic, heterocyclic, aryl, heteroaryl, acyl, sulfinyl, sulfonyl, cyano, nitro, alkylaryl, arylalkyl, and the like, each of which may or may not be further substituted).

The term “imino,” as used herein, refers to a group of the formula (═NR^{r}), wherein R^{r} corresponds to hydrogen or any substituent as described herein, that results in the formation of a stable moiety (for example, an amino protecting group; aliphatic, alkyl, alkenyl, alkynyl, heteroaliphatic, heterocyclic, aryl, heteroaryl, acyl, amino, hydroxyl, alkylaryl, arylalkyl, and the like, each of which may or may not be further substituted).

The term “azido,” as used herein, refers to a group of the formula (—N₃).

The term “cyano,” as used herein, refers to a group of the formula (—CN).

The terms “halo” and “halogen,” as used herein, refer to an atom selected from fluorine (fluoro, —F), chlorine (chloro, —Cl), bromine (bromo, —Br), and iodine (iodo, —I). The term “isocyano,” as used herein, refers to a group of the formula (—NC).

The term “nitro,” as used herein, refers to a group of the formula (—NO₂).

The term “oxo,” as used herein, refers to a group of the formula (═O).

The term “thiooxo,” as used herein, refers to a group of the formula (═S).

An “amino-protecting group,” as used herein includes, for example, those described in detail in Protecting Groups in Organic Synthesis, T. W. Greene and P. G. M. Wuts, 3^{rd} edition, John Wiley & Sons, 1999, the entirety of which is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N (1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

A “carboxylic acid protecting group” or “protected carboxylic acid,” as used herein, includes, for example, those described in detail in Greene (1999). Examples of protected carboxylic acids further include, but are not limited to, silyl-, alkyl-, alkenyl-, aryl-, and arylalkyl-protected carboxylic acids. Examples of silyl groups include trimethylsilyl, triethylsilyl, t-butyldimethylsilyl, t-butyldiphenylsilyl, triisopropylsilyl, and the like. Examples of alkyl groups include methyl, benzyl, p-methoxybenzyl, 3,4-dimethoxybenzyl, trityl, t-butyl, tetrahydropyran-2-yl. Examples of alkenyl groups include allyl. Examples of aryl groups include optionally substituted phenyl, biphenyl, or naphthyl. Examples of arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

A “hydroxyl protecting group,” as used herein, includes, for example, those described in detail in Protecting Groups in Organic Synthesis, T. W. Greene and P. G. M. Wuts, 3^{rd} edition, John Wiley & Sons, 1999, the entirety of which is incorporated herein by reference. Hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

A “thiol protecting group,” as used herein, includes, for example, those described in detail in Protecting Groups in Organic Synthesis, T. W. Greene and P. G. M. Wuts, 3^{rd} edition, John Wiley & Sons, 1999, the entirety of which is incorporated herein by reference. Examples of protected thiol groups include, but are not limited to, thioesters, carbonates, sulfonates allyl thioethers, thioethers, silyl thioethers, alkyl thioethers, arylalkyl thioethers, and alkyloxyalkyl thioethers. Examples of ester groups include formates, acetates, proprionates, pentanoates, crotonates, and benzoates. Specific examples of ester groups include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benylbenzoate, 2,4,6-trimethylbenzoate. Examples of carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of silyl groups include trimethylsilyl, triethylsilyl, t-butyldimethylsilyl, t-butyldiphenylsilyl, triisopropylsilyl ether, and other trialkylsilyl ethers. Examples of alkyl groups include methyl, benzyl, p-methoxybenzyl, 3,4-dimethoxybenzyl, trityl, t-butyl, and allyl ether, or derivatives thereof. Examples of arylalkyl groups include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

A “leaving group” refers to a molecular fragment that departs with a pair of electrons in heterolytic bond cleavage, wherein the molecular fragment is an anion or neutral molecule. See, for example, Smith, March's Advanced Organic Chemistry 6th ed. (501-502). Exemplary leaving groups include, but are not limited to, halo (e.g., chloro, bromo, iodo) and activated substituted hydroxyl groups, e.g, of the formula —OC(═O)SR^{aa}, —OC(═O)R^{aa}, —OCO₂R^{aa}, —OC(═O)N(R^{bb})₂, —OC(═NR^{bb})R^{aa}, OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, or —OP(═O)(NR^{bb})₂ wherein R^{aa} is optionally substituted aliphatic, optionally substituted heteroaliphatic, optionally substituted aryl, or optionally substituted heteroaryl; R^{bb} is hydrogen, an amino protecting group, optionally substituted aliphatic, optionally substituted heteroaliphatic, optionally substituted aryl, or optionally substituted heteroaryl; and R^{cc} is hydrogen, optionally substituted aliphatic, optionally substituted heteroaliphatic, optionally substituted aryl, or optionally substituted heteroaryl.

The term “amino acid” refers to natural amino acids, unnatural amino acids, and amino acid analogs, all in their D and L stereoisomers, unless otherwise indicated, if their structures allow such stereoisomeric forms.

As used herein, the symbol —[X_{AA}]— refers to an amino acid, e.g, of the formula:

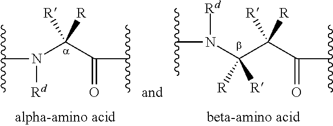

wherein each instance of R and R′ independently are selected from the group consisting of hydrogen, optionally substituted aliphatic, optionally substituted heteroaliphatic, optionally substituted aryl, optionally substituted heteroaryl, and R^{d} is hydrogen or an amino protecting group. Amino acids encompassed by the above two formulae include, without limitation, natural alpha-amino acids such as D- and L-isomers of the 20 common naturally occurring alpha-amino acids found in polypeptides and proteins (e.g., A, R, N, C, D, Q, E, G, H, I, L, K, M, F, P, S, T, W, Y, V, as depicted in Table 1 below), unnatural alpha-amino acids (examples of which are depicted in Table 2 below), natural beta-amino acids (e.g., beta-alanine), and unnatural beta-amino acids.

TABLE 1
Natural alpha-amino acids

| | R | R′ |
|---|---|---|
| L-Alanine (A) | —CH₃ | —H |
| L-Arginine (R) | —CH₂CH₂CH₂—NHC(═NH)NH₂ | —H |
| L-Asparagine (N) | —CH₂C(═O)NH₂ | —H |
| L-Aspartic acid (D) | —CH₂CO₂H | —H |
| L-Cysteine (C) | —CH₂SH | —H |
| L-Glutamic acid (E) | —CH₂CH₂CO₂H | —H |
| L-Glutamine (Q) | —CH₂CH₂C(═O)NH₂ | —H |
| Glycine (G) | —H | —H |
| L-Histidine (H) | —CH₂-2-(1H-imidazole) | —H |
| L-Isoleucine (I) | -sec-butyl | —H |
| L-Leucine (L) | -iso-butyl | —H |
| L-Lysine (K) | —CH₂CH₂CH₂CH₂NH₂ | —H |
| L-Methionine (M) | —CH₂CH₂SCH₃ | —H |
| L-Phenylalanine (F) | —CH₂Ph | —H |
| L-Proline (P) | -2-(pyrrolidine) | —H |
| L-Serine (S) | —CH₂OH | —H |
| L-Threonine (T) | —CH₂CH(OH)(CH₃) | —H |
| L-Tryptophan (W) | —CH₂-3-(1H-indole) | —H |
| L-Tyrosine (Y) | —CH₂-(p-hydroxyphenyl) | —H |
| L-Valine (V) | -isopropyl | —H |

TABLE 2
Unnatural alpha-amino acids

| | R | R′ |
|---|---|---|
| D-Alanine | —H | —CH₃ |
| D-Arginine | —H | —CH₂CH₂CH₂—NHC(═NH)NH₂ |
| D-Asparagine | —H | —CH₂C(═O)NH₂ |
| D-Aspartic acid | —H | —CH₂CO₂H |
| D-Cysteine | —H | —CH₂SH |
| D-Glutamic acid | —H | —CH₂CH₂CO₂H |
| D-Glutamine | —H | —CH₂CH₂C(═O)NH₂ |
| D-Histidine | —H | —CH₂-2-(1H-imidazole) |
| D-Isoleucine | —H | -sec-butyl |
| D-Leucine | —H | -iso-butyl |
| D-Lysine | —H | —CH₂CH₂CH₂CH₂NH₂ |
| D-Methionine | —H | —CH₂CH₂SCH₃ |
| D-Phenylalanine | —H | —CH₂Ph |
| D-Proline | —H | -2-(pyrrolidine) |
| D-Serine | —H | —CH₂OH |
| D-Threonine | —H | —CH₂CH(OH)(CH₃) |
| D-Tryptophan | —H | —CH₂-3-(1H-indole) |
| D-Tyrosine | —H | —CH₂-(p-hydroxyphenyl) |
| D-Valine | —H | -isopropyl |
| Di-vinyl | —CH═CH₂ | —CH═CH₂ |

| | R and R′ are equal to: |
|---|---|
| α-methyl-Alanine | —CH₃, —CH₃ |
| (Aib) | |
| α-methyl-Arginine | —CH_{3,} —CH₂CH₂CH₂—NHC(═NH)NH₂ |
| α-methyl-Asparagine | —CH_{3,} —CH₂C(═O)NH₂ |
| α-methyl-Aspartic | —CH_{3,} —CH₂CO₂H |
| acid | |
| α-methyl-Cysteine | —CH_{3,} —CH₂SH |
| α-methyl-Glutamic | —CH_{3,} —CH₂CH₂CO₂H |
| acid | |
| α-methyl-Glutamine | —CH₃, —CH₂CH₂C(═O)NH₂ |
| α-methyl-Histidine | —CH_{3,} —CH₂-2-(1H-imidazole) |
| α-methyl-Isoleucine | —CH_{3,} -sec-butyl |
| α-methyl-Leucine | —CH_{3,} -iso-butyl |
| α-methyl-Lysine | —CH_{3,} —CH₂CH₂CH₂CH₂NH₂ |
| α-methyl- | —CH_{3,} —CH₂CH₂SCH₃ |
| Methionine | |
| α-methyl- | —CH_{3,} —CH₂Ph |
| Phenylalanine | |
| α-methyl-Proline | —CH_{3,} -2-(pyrrolidine) |
| α-methyl-Serine | —CH_{3,} —CH₂OH |
| α-methyl-Threonine | —CH_{3,} —CH₂CH(OH)(CH₃) |
| α-methyl- | —CH_{3,} —CH₂-3-(1H-indole) |
| Tryptophan | |
| α-methyl-Tyrosine | —CH_{3,} —CH₂-(p-hydroxyphenyl) |
| α-methyl-Valine | —CH_{3,} -isopropyl |
| Di-vinyl | —CH═CH_{2,} —CH═CH₂ |
| Norleucine | —H, —CH₂CH₂CH₂CH₃ |

There are many known unnatural amino acids any of which may be included in the polypeptides of the present invention. See, for example, S. Hunt, The Non-Protein Amino Acids: In Chemistry and Biochemistry of the Amino Acids, edited by G. C. Barrett, Chapman and Hall, 1985; incorporated by reference in its entirety. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; —CH₃), disubstituted phenylalanines, substituted tyrosines (e.g., further substituted with —C(═O)C₆H₅; —CF₃; —CN; -halo; —NO₂; —CH₃), and statine.

Certain unnatural amino acids are included into the polypeptide chain for peptide stapling or stitching. These unnatural amino acids include a terminal unsaturated moiety, such as a double or triple bond. Exemplary amino acids with terminal olefinic unsaturation include, but are not limited to, —(CH₂)_{g}—S—(CH₂)_{g}CH═CH₂; —(CH₂)_{g}—O—(CH₂)_{g}CH═CH₂; —(CH₂)_{g}—NH—(CH₂)_{g}CH═CH₂; —(CH₂)_{g}—(C═O)—S—(CH₂)_{g}CH═CH₂; —(CH₂)_{g}—(C═O)—O—(CH₂)_{g}CH═CH₂; —(CH₂)_{g}—(C═O)—NH—(CH₂)_{g}CH═CH₂; —CH₂CH₂CH₂CH₂—NH—(CH₂)_{g}CH═CH₂; —(C₆H₅)-p-O—(CH₂)_{g}CH═CH₂; —CH(CH₃)—O—(CH₂)_{g}CH═CH₂; —CH₂CH(—O—CH═CH₂)(CH₃); -histidine-N((CH₂)_{g}CH═CH₂); -tryptophan-N((CH₂)_{g}CH═CH₂); and 13 (CH₂)_{g+1}(CH═CH₂), wherein each instance of g is, independently, 0 to 10, inclusive. Specific amino acids with terminal unsaturation are further described and depicted herein.

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

“Protein,” “peptide” and “polypeptide” are terms used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. Polypeptides contain unnatural amino acids comprising terminal unsaturated side chains which may be joined via ring closing metathesis to form one or more staples, natural amino acids, and optionally one or more unnatural amino acids such as depicted in Table 2. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

The terms “peptidomimetic,” “peptide mimetic,” “polypeptide mimetic,” etc., refer to a peptide-like or polypeptide-like molecule. A peptidomimetic may contain amino acids and/or non-amino acid components. Examples of peptidomimitecs include chemically modified peptides/polypeptides, peptoids (side chains are appended to the nitrogen atom of the peptide backbone, rather than to the α-carbons), β-peptides (amino group bonded to the 1 carbon rather than the α carbon), etc.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:

1) Alanine (A) and Glycine (G);

2) Aspartic acid (D) and Glutamic acid (E);

3) Asparagine (N) and Glutamine (Q);

4) Arginine (R) and Lysine (K);

5) Isoleucine (I), Leucine (L), Methionine (M), and Valine (V);

6) Phenylalanine (F), Tyrosine (Y), and Tryptophan (W);

7) Serine (S) and Threonine (T); and

8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions with respect to that reference sequence. For example, a sequence having at least 90% sequence identity with SEQ ID NO:Z, which is 101 amino acids in length, may have up to 10 substitutions relative to SEQ ID NO:Z, and may therefore also be expressed as having 10 or fewer substitutions relative to SEQ ID NO:Z.

As used herein, when two entities are “associated with” one another they are linked by a direct or indirect covalent or non-covalent interaction. In certain embodiments, the association is covalent, and the entities are said to be “conjugated” to one another. In other embodiments, the association is non-covalent. Non-covalent interactions include hydrogen bonding, van der Waals interactions, hydrophobic interactions, magnetic interactions, electrostatic interactions, pi stacking, etc. An indirect covalent interaction is when two entities are covalently associated through a linker.

As used herein, a “label” refers to a moiety that has at least one element, isotope, or functional group incorporated into the moiety which enables detection of the polypeptide to which the label is attached. Labels can be directly attached (i.e., via a bond) or can be attached by a tether (such as, for example, an optionally substituted alkylene; an optionally substituted alkenylene; an optionally substituted alkynylene; an optionally substituted heteroalkylene; an optionally substituted heteroalkenylene; an optionally substituted heteroalkynylene; an optionally substituted arylene; an optionally substituted heteroarylene; or an optionally substituted acylene, or any combination thereof, which can make up a tether). It will be appreciated that the label may be attached to or incorporated into the polypeptide at any position.

In general, a label can fall into any one (or more) of five classes: a) a label which contains isotopic moieties, which may be radioactive or heavy isotopes, including, but not limited to, ²H, ³H, ¹³C, ¹⁴C, ¹⁵N, ¹⁸F, ³¹P, ³²P, ³⁵S, ⁶⁷Ga, ⁷⁶Br, ^{99m}Tc (Tc-99m), ¹¹¹In, ¹²³I, ¹²⁵I, ¹³¹I, ¹⁵³Gd, ¹⁶⁹Yb, and ¹⁸⁶Re; b) a label which contains an immune moiety, which may be antibodies or antigens, which may be bound to enzymes (e.g., such as horseradish peroxidase); c) a label which is a colored, luminescent, phosphorescent, or fluorescent moieties (e.g., such as the fluorescent label fluoresceinisothiocyanat (FITC); d) a label which has one or more photo affinity moieties; and e) a label which is a ligand for with one or more known binding partners (e.g., biotin-streptavidin, FK506-FKBP). In certain embodiments, a label comprises a radioactive isotope, preferably an isotope which emits detectable particles, such as β particles. In certain embodiments, the label comprises a fluorescent moiety. In certain embodiments, the label is the fluorescent label fluoresceinisothiocyanat (FITC). In certain embodiments, the label comprises a ligand moiety with one or more known binding partners. In certain embodiments, the label comprises biotin.

### DETAILED DESCRIPTION OF THE INVENTION

The present invention relates to peptides and protein mimetics and their therapeutic and research use. In particular, the present invention provides synthetic, stabilized DNA binding domain peptides and methods of using such peptides as therapeutic agents.

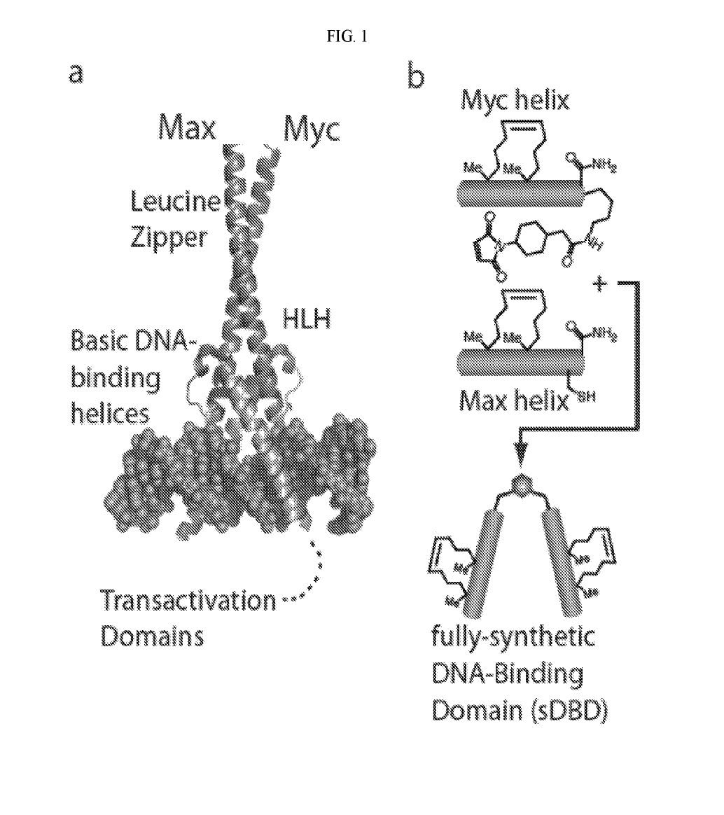

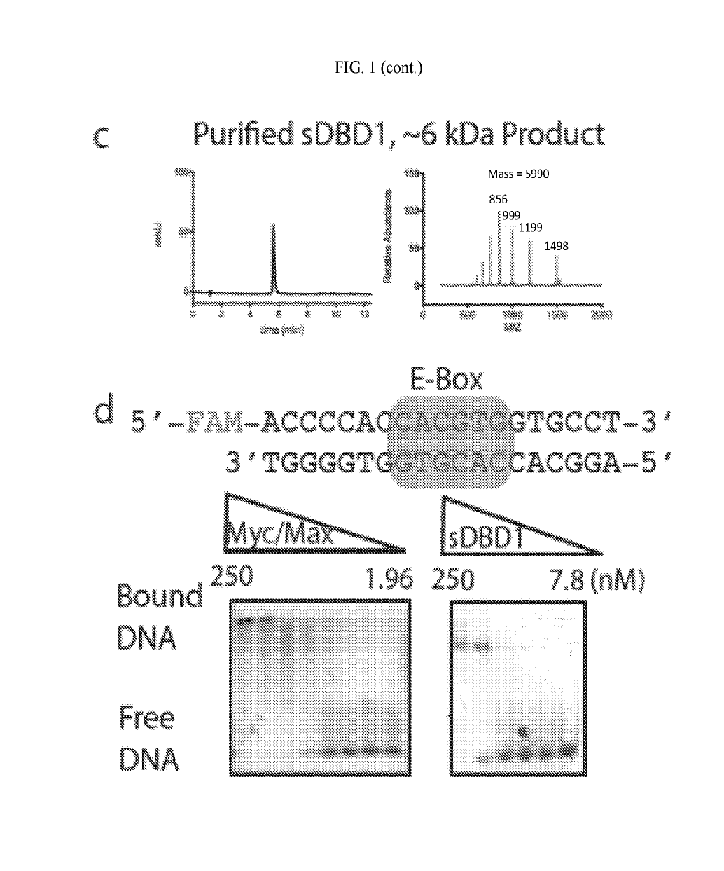

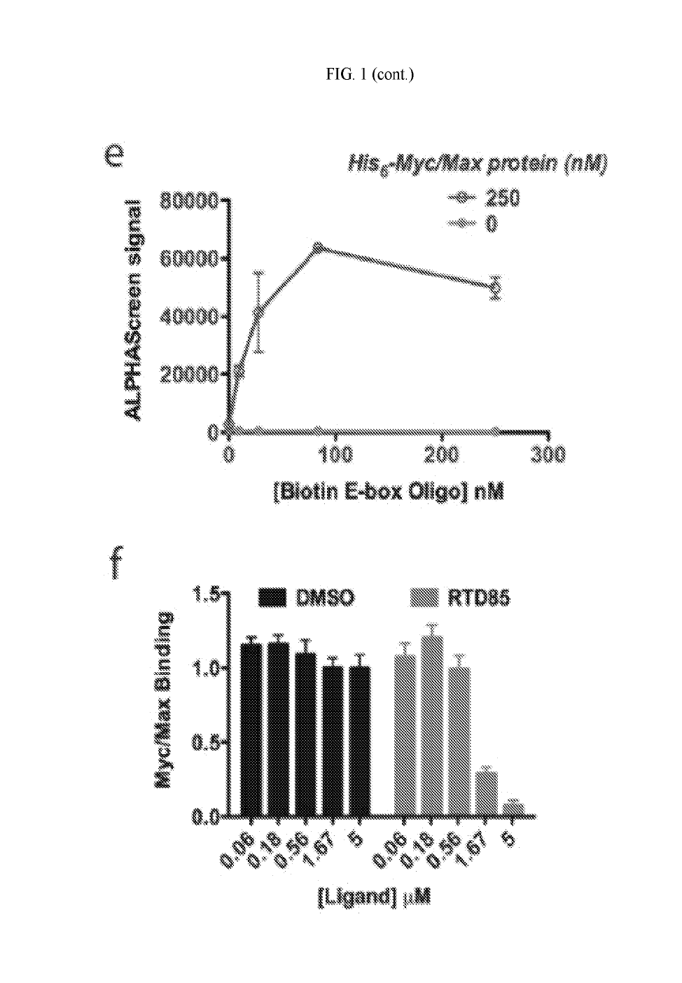

In light of the need for new approaches to target TFs, experiments described herein resulted in the development of a new class of pharmacologic agents, stapled peptides, that have proven capable of targeting difficult protein-protein interactions involved in TF function. This technology endows polypeptides with improved pharmacologic properties (e.g., binding affinity, cell penetration and in vivo stability), and has been used to develop inhibitors of several oncogenes, including β-Catenin (Ref. 2; incorporated by reference in its entirety), NOTCH1 (Ref. 3; incorporated by reference in its entirety) and RAB25 (Ref. 4; incorporated by reference in its entirety). Furthermore, optimized stapled peptides are currently being evaluated for human therapeutic potential in clinical trials for oncology and endocrine disorders (Lyer et al., Org. Biomol. Chem., 2015, 13, 3856; incorporated by reference in its entirety). Even with the successes of this approach, many TF classes remain resistant to pharmacologic modulation. Characteristic among these are the basic Helix-loop-Helix (bHLH) TFs, which by forming homo- or heterodimers bind specific DNA sequences through oriented α-helices that interrogate the major groove (FIG. 1a). The Myc/Max TF is a well-studied bHLH heterodimer implicated in driving gene expression and cell growth in a majority of cancers. Cellular, murine and human genetic experiments highlight the critical role that Myc plays in cancer. Therefore, approaches to target Myc, bHLH-TFs and related TF classes are urgently needed.

In some embodiments, provided herein are peptides comprising an alpha helical segment that comprises a DNA binding domain. In some embodiments, two amino acids (e.g., i and i+4, I and i+7, etc.) within the alpha helical segment are modified to allow hydrocarbon stapling between the two amino acids. In some embodiments, the hydrocarbon stapling stabilizes the alpha helix and allows for DNA binding by the peptide in the absence of a larger polypeptide. In some embodiments, the peptides further comprise one or two (or more linker residues. Linker residues may be natural (e.g., cysteine) or unnatural (e.g., displaing a thiol, azide, maleimide, alkyne, etc.) amino acids that facilitate the formation of linkages (e.g., covalent linkages) between the peptide and a second peptide comprising complementary linker residues. In some embodiments, the second peptide also comprises an alph helical segment modified to allow hydrocarbon stapling. In some embodiments, peptides comprise a first linker residue at the N terminal residue (e.g., azide or alkyne) and a second linker residue (e.g., thiol of maleimide) at a position 1-10 (e.g., 1, 2, 3, 4, 5, 6, 7, 8, 9, 10, or ranges therebetween) amino acids from the N-terminus. In some embodiments, hydrocarbon stabling within the alph helix stabilizes the alph helix, while linkage of the two peptides together (e.g., at two positons) provides proper (e.g., optimized) orientation of the two peptides (e.g., with respect to a DNA binding site).

Provided herein are cell-permeable, synthetic DNA-binding Domains (sDBDs) with comparable/equivalent DNA-binding specificity as endogenous, full-length TFs. sDBDs mimic the DNA-recognition architecture of bHLH proteins through synthetic preservation of: (1) dimeric, oriented DNA-binding helices and (2) α-helix stabilization to maximize affinity and specificity (FIG. 1b). Libraries of sDBDs derived from Myc/Max have been synthesized, purified (FIG. 1c), and tested for their ability to specifically bind E-box containing DNA (FIG. 1d). Strikingly, several sDBDs have binding affinities comparable to full length Myc/Max (KD<100 nM) and inhibit Myc/Max binding to E-box sequences in FRET-based proximity assays (FIG. 1e, f). In addition, it was found that sDBDs are cell permeable via active macropinocytosis, which is in agreement with recent mechanistic analyses of cell penetration by a large library of stapled peptides (Refs. 5-6; incorporated by reference in their entireties). In light of these data, sDBDs represent a new class of Myc antagonists and a general technology for the development of non-vector-based, programmable, artificial transcription factors.

Basic helix-loop-helix leucine-zipper (bHLH-LZ) transcription factors assemble via a combination of protein-protein interactions and half-site recognition of specific DNA sequences by each alpha-helix. LZ and loop regions confer affinity, stability, monomer specificity orientation of the overall protein architecture. Myc/Max is a canonical bHLH-LZ TF with regulatory roles in development, stem cell maintenance and cancer. Other bHLH-LZ) transcription factors in disease are shown below.

Notable bHLH-LZ TFs in disease

| | |
|---|---|
| Myc/Max | cancer, stem cell maintenance, immune |
| | disorders |
| HIF1α/β | Cancer, tissue injury (neurologic, cardiac etc), |
| | stem cells |
| MITF | Cancer, stem cells |
| MyoD | Cancer, muscle-related illness, stem cells |
| HES-family | Cancer, stem cells |
| HEY-family | Cancer, stem cells |
| ID1/2/3 | Cancer, stem cells |
| E2 family | Cancer |
| Twist | Cancer, metastasis, EMT progression |

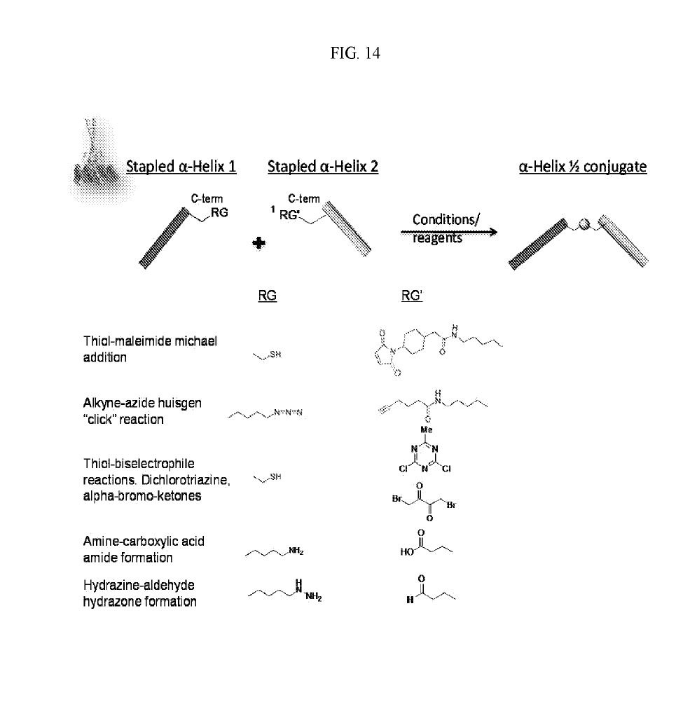

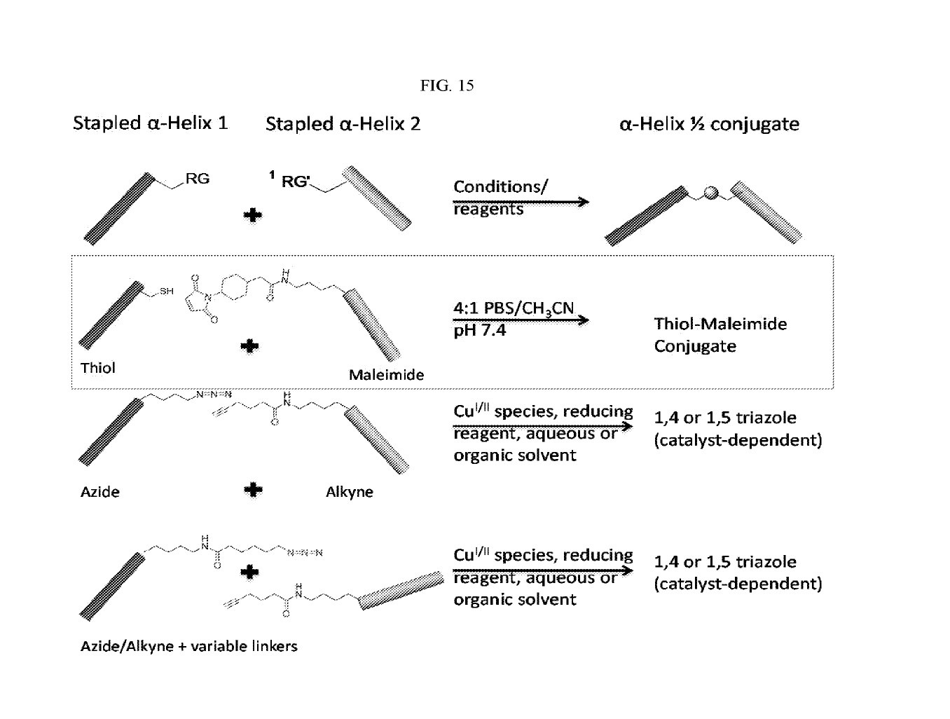

The present disclosure is not limited to particular sDBD peptides. Examples of specific peptides include, but are not limited to, AcW-βKRRTHNVLERQRRNELKRSβ-C (SEQ ID NO: 1), AcW-βKRAHHNALERKRRDHIKDSβ-K(Mmt) (SEQ ID NO: 2), AcW-βKRAHHNALERKRRDHIKDSβ-K(Mmt) (SEQ ID NO: 3), AcW-βKRRTHN*LER*RRNELKRSβ-C (SEQ ID NO: 4), AcW-βKRRTHNVLER*RRN*LKRSβ-C (SEQ ID NO: 5), AcW-βKR*THN*LERQRRNELKRSβ-C (SEQ ID NO: 6), AcW-βKRAHHN*LER*RRDHIKDSβ-K(Mmt) (SEQ ID NO: 7), AcW-βKRAHHNALER*RRD*IKDSβ-K(Mmt) (SEQ ID NO: 8), AcW-βKRAHHNALER*RRD*IKDSβ-K(Mmt) (SEQ ID NO: 9), AcW-βKR*HHN*LERKRRDHIKDSβ-K(Mmt) (SEQ ID NO: 10), AcW-GKRRTHN*LER*RRNELKRSG-C (SEQ ID NO: 11), AcW-GKR*HHN*LERKRRDHIKDSG-K(Mmt) (SEQ ID NO: 12), AcW-βKRAHHNALER*RRD*IKDS-K(Mmt) (SEQ ID NO: 13), AcW-βKR*HHN*LERKRRDHIKDS-K(Mmt) (SEQ ID NO: 14), AcW-βKR*HHN*LERKRRDHIKDS-K(Mmt) (SEQ ID NO: 15), AcW-βKRRTHN*LER*RRNELKRS-C (SEQ ID NO: 16), AcW-βKRRTHNVLER*RRN*LKRS-C (SEQ ID NO: 17), AcW-βKRAHHNALER*RRD*IKDS-K(Mmt) (SEQ ID NO: 18), AcW-βKRAHHNALER*RRD*IKDS-K(Mmt) (SEQ ID NO: 19), AcW-βKRAHHNALER*RRD*IKDS-K(Mmt) (SEQ ID NO: 20), FITC-PEG3-βKRRTHNVLERQRRNELKRSβ-C (SEQ ID NO: 21), FITC-PEG3-βKRRTHN*LER*RRNELKRSβ-C (SEQ ID NO: 22), FITC-PEG3-βKRRTHNVLER*RRN*LKRSβ-C (SEQ ID NO: 23), Biotin-PEG3-W-βKRRTHN*LER*RRNELKRSβ-C (SEQ ID NO: 24), Biotin-PEG3-W-βKRRTHN*LER*RRNELKRS-C (SEQ ID NO: 25), Fmoc-βKRRTHNVLERQRRNELKRSβ-C (SEQ ID NO: 26), Fmoc-βKRAHHNALERKRRDHIKDSβ-K(Mmt) (SEQ ID NO: 27), Fmoc-βKRRTHN*LER*RRNELKRSβ-C (SEQ ID NO: 28), Fmoc-βKRRTHN*LER*RRNELKRSβ-C (SEQ ID NO: 29), Fmoc-βKRRTHNVLER*RRN*LKRSβ-C (SEQ ID NO: 30), Fmoc-βKRRTHNVLER*RRN*LKRSβ-C (SEQ ID NO: 31), Fmoc-βKRAHHNALER*RRD*IKDSβ-K(Mmt) (SEQ ID NO: 32), Fmoc-βKRAHHNALER*RRD*IKDSβ-K(Mmt) (SEQ ID NO: 33), Fmoc-βKR*HHN*LERKRRDHIKDSβ-K(Mmt) (SEQ ID NO: 34), Fmoc-βKR*HHN*LERKRRDHIKDSβ-K(Mmt) (SEQ ID NO: 35), Fmoc-βKRRTHN*LER*RRNELKRSG-C (SEQ ID NO: 36), Fmoc-βKRRTHN*LER*RRNELKRSG-C (SEQ ID NO: 37), Fmoc-βKR*HHN*LERKRRDHIKDSG-K(Mmt) (SEQ ID NO: 38), Fmoc-βKR*HHN*LERKRRDHIKDSG-K(Mmt) (SEQ ID NO: 39), Fmoc-βKRAHHNALER*RRD*IKDS-K(Mmt) (SEQ ID NO: 40), Fmoc-βKRAHHNALER*RRD*IKDS-K(Mmt) (SEQ ID NO: 41), Fmoc-βKR*HHN*LERKRRDHIKDS-K(Mmt) (SEQ ID NO: 42), Fmoc-βKR*HHN*LERKRRDHIKDS-K(Mmt) (SEQ ID NO: 43), Fmoc-βKRRTHN*LER*RRNELKRS-C (SEQ ID NO: 44), Fmoc-βKRRTHN*LER*RRNELKRS-C (SEQ ID NO: 45), Fmoc-βKRRTHNVLER*RRN*LKRS-C (SEQ ID NO: 46), Fmoc-βKRRTHNVLER*RRN*LKRS-C (SEQ ID NO: 47), Fmoc-βKRRTHN*LER*RRNELKRSβ-K(Mmt) (SEQ ID NO: 48), Fmoc-βKRRTHNVLER*RRN*LKRSβ-K(Mmt) (SEQ ID NO: 49), Fmoc-βKRRTHN*LER*RRNELKRSβ-K(Mmt) (SEQ ID NO: 50), Fmoc-βKRRTHNVLER*RRN*LKRSβ-K(Mmt) (SEQ ID NO: 51), the peptides shown in FIGS. 14 and 15, and variants thereof (e.g., variants that have at least 70% sequence identity (e.g., 70%, 75%, 80%, 85%, 90%, 95%, 99%, or ranges therebetween) to such peptides), mimetics, or modified versions thereof.

Percent sequence identity can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

In some embodiments, 1, 2, 3, or 4 amino acids from the peptides described herein may be deleted. In some embodiments, 1, 2, 3, or 4 amino acids may be inserted into the peptides or added to either the C or N terminal end. In some embodiments, 1, 2, 3, or 4 amino acids within the peptides may be replaced with other amino acids. Suitable amino acid substitutions include conservative, semi-conservative, or non-conservative amino acid substitutions. For example, individual amino acid substitutions can be selected from any one of the following: 1) the set of amino acids with nonpolar sidechains, for example, Ala, Cys, Ile, Leu, Met, Phe, Pro, Val; 2) the set of amino acids with negatively charged side chains, for example, Asp, Glu; 3) the set of amino acids with positively charged sidechains, for example, Arg, His, Lys; and 4) the set of amino acids with uncharged polar sidechains, for example, Asn, Cys, Gln, Gly, His, Met, Phe, Ser, Thr, Trp, Tyr, to which are added Cys, Gly, Met and Phe.

A naturally occurring amino acid can also be replaced with, for example, a non-naturally occurring amino acid such as, for example, norleucine, omithine, norvaline, homoserine, and other amino acid residue analogues such as those described in Ellman et al., Meth. Enzym., 1991, 202, 301-336; incorporated by reference in its entirety. To generate such non-naturally occurring amino acid residues, the procedures of Noren et al., Science, 1989, 244, 182 and Ellman et al., supra (incorporated by reference in their entireties), can be used. Other suitable methods are described in White et al., Methods, 2013, 60, 70-74; Gentilucci et al., Curr Pharm Des 2010, 16, 3195-3203; Hodgson & Sanderson, Chem Soc Rev 2004, 33, 422-430 and Krebs et al., Chemistry 2004, 10:544-553; incorporated by reference in their entireties.

The present invention provides stapled polypeptides, and unstapled precursors thereof. In some embodiments, the stapled polypeptides comprise a stapled amino acid sequence —[X₁₋₂₃]—. The sequence —[X₁₋₂₃]— comprises one or more staples, e.g., one, two, three, or four staples, wherein the amino acids which participate in the staple are separated by two or more amino acids. In certain embodiments, the amino acid sequence is alpha helical. In certain embodiments, two stapled polypetides are conjugated together to form a homodimer or heterodimer, which, in certain embodiments, interferes with binding of a natural DNA binding domain (e.g., Myc/Max, Fos/Jun, etc.) to its DNA target sequence.

In one aspect, provided is an unstapled polypeptide of Formula (I):

R^{f-}[X_{AA}]ₛ—[X₁₋₂₃]—[X_{AA}]ₜ—R^{e} (I)

or a pharmaceutically acceptable salt thereof;
wherein:

each instance of X_{AA} is a natural amino acid or unnatural amino acid;

s is 0 or an integer between 1 and 100, inclusive;

t is 0 or an integer between 1 and 100, inclusive;

R^{f} is an N-terminal group selected from the group consisting of hydrogen; optionally substituted aliphatic; optionally substituted heteroaliphatic; optionally substituted aryl; optionally substituted heteroaryl; acyl; a resin; an amino protecting group; and a label optionally joined by a linker, wherein the linker is selected from the group consisting of optionally substituted alkylene; optionally substituted alkenylene; optionally substituted alkynylene; optionally substituted heteroalkylene; optionally substituted heteroalkenylene; optionally substituted heteroalkynylene; optionally substituted arylene; optionally substituted heteroarylene; and acylene;

R^{e} is a C-terminal group selected from the group consisting of hydrogen; optionally substituted aliphatic; optionally substituted heteroaliphatic; optionally substituted aryl; optionally substituted heteroaryl; —OR^{E}, —N(R^{E})₂, or —SR^{E}, wherein each instance of R^{E} is, independently, hydrogen; optionally substituted aliphatic; optionally substituted heteroaliphatic; optionally substituted aryl; optionally substituted heteroaryl; acyl; a resin; a protecting group; or two R^{E} groups taken together form an optionally substituted heterocyclic or optionally substituted heteroaryl ring; and

—[X₁₋₂₃]— is an unstapled amino sequence of the Formula (SEQ ID NO: 52):

-[X₁-X₂-X₃-X₄-X₅-X₆-X₇-X₈-X₉-X₁₀-X₁₁-X₁₂-X₁₃-X₁₄-X₁₅-X₁₆-
X₁₇-X₁₈-X₁₉-X₂₀-X₂₁-(X₂₂)ₙ-X₂₃]-

wherein:

X₁ is amino acid W;

X₂ is absent or is a natural amino acid or unnatural amino acid;

X₃ is amino acid K;

X₄ is amino acid R;

X₅ is an amino acid selected from the group consisting of A, R, and an amino acid of Formula (i);

X₆ is an amino acid selected from the group consisting of H and T;

X₇ is amino acid H;

X₈ is amino acid N;

X₉ is an amino acid selected from the group consisting of A, V, and an amino acid of Formula (i) or (ii);

X₁₀ is amino acid L;

X₁₁ is amino acid E;

X₁₂ is amino acid R;

X₁₃ is an amino acid selected from the group consisting of K, Q, and an amino acid of Formula (i) or (ii);

X₁₄ is amino acid R;

X₁₅ is amino acid R;

X₁₆ is an amino acid selected from the group consisting of D and N;

X₁₇ is an amino acid selected from the group consisting of H, E, and an amino acid of Formula (i) or (ii);

X₁₈ is an amino acid selected from the group consisting of I and L;

X₁₉ is amino acid K;

X₂₀ is an amino acid selected from the group consisting of D and R;

X₂₁ is amino acid S;

each instance of X₂₂ is independently a natural or unnatural amino acid, and n is 0 or an integer between 1 and 10 inclusive; and

X₂₃ is a natural or unnatural amino acid of the Formula (v) or (vi):

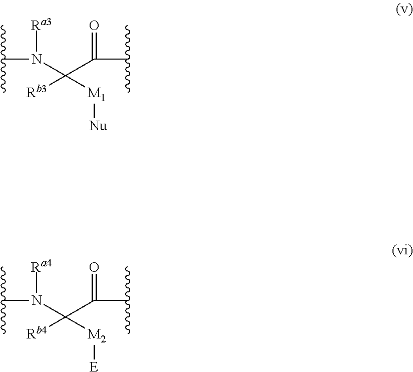

wherein:

each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of each hydrogen; optionally substituted aliphatic; optionally substituted heteroaliphatic; optionally substituted aryl; optionally substituted heteroaryl;

Nu is —SH, —OH, —NHR^{b5}, —NH—NHR^{b5}, —N═NH, —N═C, —N₃, or

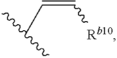

wherein R^{b10} is hydrogen, optionally substituted aliphatic, or optionally substituted heteroaliphatic; and R^{b5} is hydrogen, optionally substituted aliphatic, optionally substituted heteroaliphatic, or an amino protecting group;

E is a leaving group, —CHO, —CO₂Rⁿ⁶, —COX^{b7},

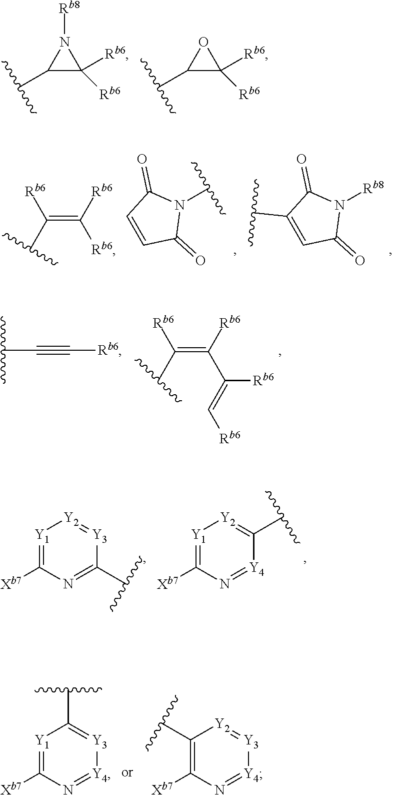

wherein:

R^{b6} is hydrogen, optionally substituted aliphatic, or optionally substituted heteroaliphatic, or wherein two R^{b6} groups are joined to form an optionally substituted carbocyclic or optionally substituted heterocyclic ring;

X^{b7} is a leaving group;

each instance of Y₁, Y₂, Y₃, and Y₄ is independently selected from —N— or —C(R^{b6})—; and

R^{b8} is hydrogen, optionally substituted aliphatic, optionally substituted heteroaliphatic, optionally substituted aryl, optionally substituted heteroaryl, or an amino protecting group;

provided that the amino acid sequence comprises at least two independent occurrences of an amino acid of Formula (i) or (ii);

the amino acid of Formula (i) is:

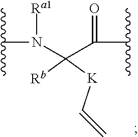

and the amino acid of Formula (ii) is:

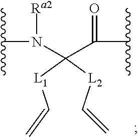

wherein:

each instance of K, L₁, and L₂, is, independently, optionally substituted alkylene; optionally substituted heteroalkylene; optionally substituted arylene; or optionally substituted heteroarylene;

each instance of R^{a1} and R^{a2} is, independently, hydrogen; optionally substituted aliphatic; optionally substituted heteroaliphatic; optionally substituted aryl; optionally substituted heteroaryl; acyl; or an amino protecting group; and

each instance of R^{b} is, independently, hydrogen; optionally substituted aliphatic; optionally substituted heteroaliphatic; optionally substituted aryl; optionally substituted heteroaryl.

In another aspect, provided is a stapled polypeptide of Formula (II):

R^{f}—[X_{AA}]ₛ—[X₁₋₂₃]—[X_{AA}]ₜ—R^{e} (II)

or a pharmaceutically acceptable salt thereof;
wherein:

each instance of X_{AA} is a natural or unnatural amino acid;

s is 0 or an integer between 1 and 100, inclusive;

t is 0 or an integer between 1 and 100, inclusive;

R^{f} is an N-terminal group selected from the group consisting of hydrogen; optionally substituted aliphatic; optionally substituted heteroaliphatic; optionally substituted aryl; optionally substituted heteroaryl; acyl; a resin; an amino protecting group; and a label optionally joined by a linker, wherein the linker is selected from the group consisting of optionally substituted alkylene; optionally substituted alkenylene; optionally substituted alkynylene; optionally substituted heteroalkylene; optionally substituted heteroalkenylene; optionally substituted heteroalkynylene; optionally substituted arylene; optionally substituted heteroarylene; and acylene;

R^{e} is a C-terminal group selected from the group consisting of hydrogen; optionally substituted aliphatic; optionally substituted heteroaliphatic; optionally substituted aryl; optionally substituted heteroaryl; —OR^{E}, —N(R^{E})₂, or —SR^{E}, wherein each instance of R^{E} is, independently, hydrogen; optionally substituted aliphatic; optionally substituted heteroaliphatic; optionally substituted aryl; optionally substituted heteroaryl; acyl; a resin; a protecting group; or two R^{E} groups taken together form an optionally substituted heterocyclic or optionally substituted heteroaryl ring; and

—[X₁₋₂₃]— is a stapled amino sequence of the Formula (SEQ ID NO: 53):

-[X₁-X₂-X₃-X₄-X₅-X₆-X₇-X₈-X₉-X₁₀-X₁₁-X₁₂-X₁₃-X₁₄-
X₁₅-X₁₆-X₁₇-X₁₈-X₁₉-X₂₀-X₂₁-(X₂₂)ₙ-X₂₃]-

wherein:

X₁ is amino acid W;

X₂ is absent or is a natural or unnatural amino acid;

X₃ is amino acid K;

X₄ is amino acid R;

X₅ is an amino acid selected from the group consisting of A and R, or X₅ and X₉ are stapled amino acids of Formula (iii), or X₅, X₉, and X₁₃ are stapled amino acids of Formula (iv);

X₆ is an amino acid selected from the group consisting of H and T;

X₇ is amino acid H;

X₈ is amino acid N;

X₉ is an amino acid selected from the group consisting of A and V, or X₅ and X₉ are stapled amino acids of Formula (iii), or X₉ and X₁₃ are stapled amino acids of Formula (iii), or X₅, X₉, and X₁₃ are stapled amino acids of Formula (iv), or X₉, X₁₃, and X₁₇ are stapled amino acids of Formula (iv);

X₁₀ is amino acid L;

X₁₁ is amino acid E;

X₁₂ is amino acid R;

X₁₃ is an amino acid selected from the group consisting of K and Q, or X₉ and X₁₃ are stapled amino acids of Formula (iii), or X₁₃ and X₁₇ are stapled amino acids of Formula (iii), or X₉, X₁₃, and X₁₇ are stapled amino acids of Formula (iv);

X₁₄ is amino acid R;

X₁₅ is amino acid R;

X₁₆ is an amino acid selected from the group consisting of D and N;

X₁₇ is an amino acid selected from the group consisting of H and E, or X₁₃ and X₁₇ are stapled amino acids of Formula (iii), or X₉, X₁₃, and X₁₇ are stapled amino acids of Formula (iv);

X₁₈ is an amino acid selected from the group consisting of I and L;

X₁₉ is amino acid K;

X₂₀ is an amino acid selected from the group consisting of D and R;

X₂₁ is amino acid S;

each instance of X₂₂ is independently a natural amino acid or an unnatural amino acid, and n is 0 or an integer between 1 and 10 inclusive; and

X₂₃ is an amino acid of the Formula (iii) or (iv):

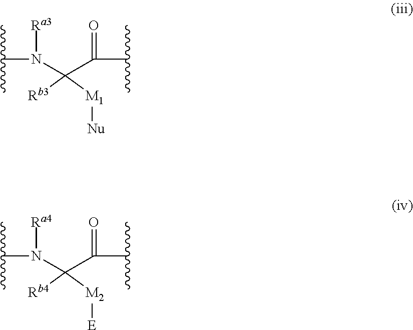

wherein:

each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of each hydrogen; optionally substituted aliphatic; optionally substituted heteroaliphatic; optionally substituted aryl; optionally substituted heteroaryl;

Nu is —SH, —OH, —NHR^{b5}, —NH—NHR^{b5}, —N═NH, —N═C, —N₃, or

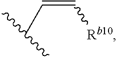

wherein R^{b10} is hydrogen, optionally substituted aliphatic, or optionally substituted heteroaliphatic; and R^{b5} is hydrogen, optionally substituted aliphatic, optionally substituted heteroaliphatic, or an amino protecting group;

E is a leaving group, —CHO, —CO₂R^{b6}, —COX^{b7},

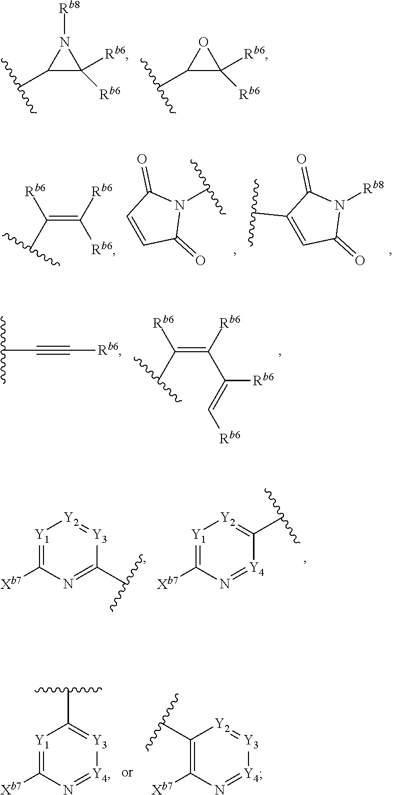

wherein:

R^{b6} is hydrogen, optionally substituted aliphatic, or optionally substituted heteroaliphatic, or wherein two R^{b6} groups are joined to form an optionally substituted carbocyclic or optionally substituted heterocyclic ring;

X^{b7} is a leaving group;

each instance of Y₁, Y₂, Y₃, and Y₄ is independently selected from —N— or —C(R^{b6})—; and

R^{b8} is hydrogen, optionally substituted aliphatic, optionally substituted heteroaliphatic, optionally substituted aryl, optionally substituted heteroaryl, or an amino protecting group;

provided that the amino acid sequence comprises at least one occurrence of stapled amino acids of Formula (iii) or (iv);

wherein the stapled amino acids of Formula (iii) are:

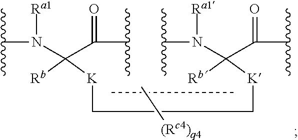

and wherein the stapled amino acids of Formula (iv) are:

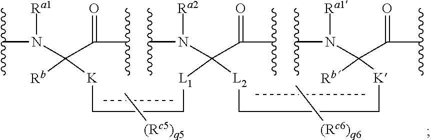

wherein:

each instance of K, K′, L₁, and L₂, is, independently, optionally substituted alkylene; optionally substituted heteroalkylene; optionally substituted arylene; or optionally substituted heteroarylene;
each instance of R^{a1}, R^{a1′}, and R^{a2} is, independently, hydrogen; optionally substituted aliphatic; optionally substituted heteroaliphatic; optionally substituted aryl; optionally substituted heteroaryl; acyl; or an amino protecting group;

each instance of R^{b} and R^{b′} is, independently, hydrogen; optionally substituted aliphatic; optionally substituted heteroaliphatic; optionally substituted aryl; optionally substituted heteroaryl;

each instance of [symbol] independently represents a single or double bond;

each instance of R^{c4}, R^{c5}, and R^{c6} is independently hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; or nitro; and

each instance of q^{c4}, q^{c6}, and q^{c6} is independently 0, an integer between 1 and 2 when [symbol] represents a double bond, or an integer between 1 and 4 when [symbol] represents a single bond.

In yet another aspect, provided is a conjugated polypeptide of Formula (III) comprising:

a first stapled polypeptide of Formula (IIIa):

R^{e1}—[X_{AA}]ₛ₁—[X₁₋₂₃]—[X_{AA}]ₜ₁—R^{f1} (IIIa)

conjugated to a second stapled polypeptide of Formula (IIIb):

R^{e2}—[X_{AA°}]ₛ₂—[X_{1-23°}]—[X_{AA°}]ₜ₂—R^{f2} (IIIb)

or a pharmaceutically acceptable salt thereof;
wherein:

each instance of X_{AA} and X_{AA°} is a natural amino acid or unnatural amino acid;

each instance of s1 and s2 is independently 0 or an integer between 1 and 100, inclusive;

each instance of t1 and t2 is independently 0 or an integer between 1 and 100, inclusive;

each instance of R^{f1} and R^{f2} is independently an N-terminal group selected from the group consisting of hydrogen; optionally substituted aliphatic; optionally substituted heteroaliphatic; optionally substituted aryl; optionally substituted heteroaryl; acyl; a resin; an amino protecting group; and a label optionally joined by a linker, wherein the linker is selected from the group consisting of optionally substituted alkylene; optionally substituted alkenylene; optionally substituted alkynylene; optionally substituted heteroalkylene; optionally substituted heteroalkenylene; optionally substituted heteroalkynylene; optionally substituted arylene; optionally substituted heteroarylene; and acylene;

each instance of R^{e1} and R^{e2} is independently a C-terminal group selected from the group consisting of hydrogen; optionally substituted aliphatic; optionally substituted heteroaliphatic; optionally substituted aryl; optionally substituted heteroaryl; —OR^{E}, —N(R^{E})₂, or —SR^{E}, wherein each instance of R^{E} is, independently, hydrogen; optionally substituted aliphatic; optionally substituted heteroaliphatic; optionally substituted aryl; optionally substituted heteroaryl; acyl; a resin; a protecting group; or two R^{E} groups taken together form an optionally substituted heterocyclic or optionally substituted heteroaryl ring;

each instance of —[X₁₋₂₃]— is a first amino sequence of the Formula (IIIa′) (SEQ ID NO: 54):

-[X₁-X₂-X₃-X₄-X₅-X₆-X₇-X₈-X₉-X₁₀-X₁₁-X₁₂-X₁₃-X₁₄-
X₁₅-X₁₆-X₁₇-X₁₈-X₁₉-X₂₀-X₂₁-(X₂₂)ₙ₁-X₂₃]-

and

each instance of —[X₁₋₂₃]°— is a second amino sequence of the Formula (IIIb′) (SEQ ID NO: 54):

-[X₁°-X₂°-X₃°-X₄°-X₅°-X₆°-X₇°-X₈°-X₉°-X₁₀°-X₁₁°-
X₁₂°-X₁₃°-X₁₄°-X₁₅°-X₁₆°-X₁₇°-X₁₈°-X₁₉°-X₂₀°-X₂₁°-
(X₂₂°)ₙ₂-X₂₃°]-

wherein:

each instance of X₁ and X₁° is amino acid W;

each instance of X₂ and X₂° is independently absent or is a natural amino acid or unnatural amino acid;

each instance of X₃ and X₃° is amino acid K;

each instance of X₄ and X₄° is amino acid R;

each instance of X₅ and X₅° is independently an amino acid selected from the group consisting of A and R, or X₅ and X₉ are stapled amino acids of Formula (iii), or X₅, X₉, and X₁₃ are stapled amino acids of Formula (iv), or X₅° and X₉° are stapled amino acids of Formula (iii), or X₅°, X₉°, and X₁₃° are stapled amino acids of Formula (iv);

each instance of X₆ and X₆° is independently an amino acid selected from the group consisting of H and T;

each instance of X₇ and X₇° is amino acid H;

each instance of X₈ and X₈° is amino acid N;

each instance of X₉ and X₉° is independently an amino acid selected from the group consisting of A and V, or X₅ and X₉ are stapled amino acids of Formula (iii), or X₉ and X₁₃ are stapled amino acids of Formula (iii), or X₅, X₉, and X₁₃ are stapled amino acids of Formula (iv), or X₉, X₁₃, and X₁₇ are stapled amino acids of Formula (iv), or X₅° and X₉° are stapled amino acids of Formula (iii), or X₉° and X₁₃° are stapled amino acids of Formula (iii), or X₅°, X₉°, and X₁₃° are stapled amino acids of Formula (iv), or X₉°, X₁₃°, and X₁₇° are stapled amino acids of Formula (iv);

each instance of X₁₀ and X₁₀° is amino acid L;

each instance of X₁₁ and X₁₁° is amino acid E;

each instance of X₁₂ and X₁₂° is amino acid R;

each instance of X₁₃ and X₁₃° is independently an amino acid selected from the group consisting of K and Q, or X₉ and X₁₃ are stapled amino acids of Formula (iii), or X₁₃ and X₁₇ are stapled amino acids of Formula (iii), or X₉, X₁₃, and X₁₇ are stapled amino acids of Formula (iv), or X₉° and X₁₃° are stapled amino acids of Formula (iii), or X₁₃° and X₁₇° are stapled amino acids of Formula (iii), or X₉°, X₁₃°, and X₁₇° are stapled amino acids of Formula (iv);

each instance of X₁₄ and X₁₄° is amino acid R;

each instance of X₁₅ and X₁₅° is amino acid R;

each instance of X₁₆ and X₁₆° is independently an amino acid selected from the group consisting of D and N;

each instance of X₁₇ and X₁₇° is independently an amino acid selected from the group consisting of H and E, or X₁₃ and X₁₇ are stapled amino acids of Formula (iii), or X₉, X₁₃, and X₁₇ are stapled amino acids of Formula (iv), or X₁₃° and X₁₇° are stapled amino acids of Formula (iii), or X₉°, X₁₃°, and X₁₇° are stapled amino acids of Formula (iv);

each instance of X₁₈ and X₁₈° is independently an amino acid selected from the group consisting of I and L;

each instance of X₁₉ and X₁₉° is amino acid K;

each instance of X₂₀ and X₂₀° is independently an amino acid selected from the group consisting of D and R;

each instance of X₂₁ and X₂₁° is amino acid S; and

each instance of X₂₂ and X₂₂° is independently a natural amino acid or an unnatural amino acid, n1 is 0 or an integer between 1 and 10 inclusive, and n2 is 0 or an integer between 1 and 10 inclusive; and

wherein amino acid X₂₃ of the first stapled amino acid sequence of Formula (IIIa) and amino acid X₂₃° of the second stapled amino acid sequence of Formula (IIIb) are joined to form a group of the Formula:

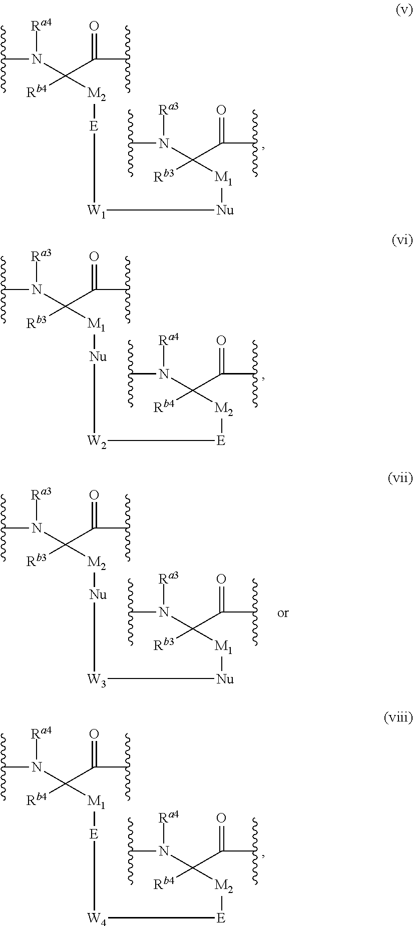

wherein:

each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of each hydrogen; optionally substituted aliphatic; optionally substituted heteroaliphatic; optionally substituted aryl; optionally substituted heteroaryl;

each instance of -Nu-W₁-E- and -Nu-W₂-E- independently represents any one of the following groups:

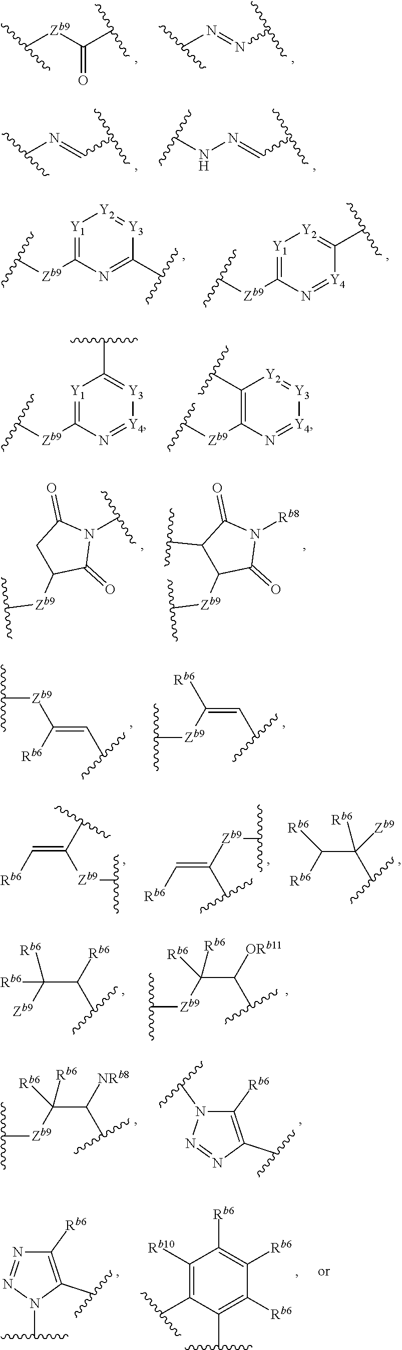

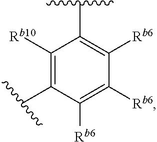

wherein:

Z^{b9} is —O—, —S—, —N(R^{b5})—, —NH—N(R^{bb5})—, —N═N—, or —NC—; and R^{b5} is hydrogen, optionally substituted aliphatic, optionally substituted heteroaliphatic, or an amino protecting group;

R^{b6} is hydrogen, optionally substituted aliphatic, or optionally substituted heteroaliphatic, or two R^{b6} groups are joined to form an optionally substituted carbocyclic or optionally substituted heterocyclic ring;

each instance of Y₁, Y₂, Y₃, and Y₄ is independently selected from —N— or —C(R^{b6})—;

R^{b8} is hydrogen, optionally substituted aliphatic, optionally substituted heteroaliphatic, optionally substituted aryl, optionally substituted heteroaryl, or an amino protecting group;

R^{b10} is hydrogen, optionally substituted aliphatic, or optionally substituted heteroaliphatic;

R^{b11} is hydrogen, optionally substituted aliphatic, optionally substituted heteroaliphatic, optionally substituted aryl, optionally substituted heteroaryl, or an oxygen protecting group;

each instance of -Nu-W₃-Nu- independently represents

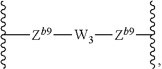

wherein
Z^{b9} is —O—, —S—, —N(R^{b5})—, —NH—N(R^{b5})—, —N═N—, or —N═C—; R^{b5} is hydrogen, optionally substituted aliphatic, optionally substituted heteroaliphatic, or an amino protecting group; and W₃ is selected from the group consisting of optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene; and

each instance of -E-W₄-E- independently represents optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

provided that the polypeptide comprises at least one occurrence of stapled amino acids of Formula (iii) or (iv);

wherein the stapled amino acids of Formula (iii) is:

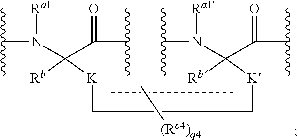

and wherein the stapled amino acids of Formula (iv) is:

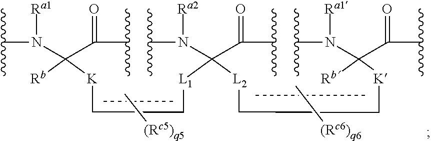

wherein:

each instance of K, K′, L₁, and L₂, is, independently, optionally substituted alkylene; optionally substituted heteroalkylene; optionally substituted arylene; or optionally substituted heteroarylene;

each instance of R^{a1}, R^{a1′}, and R^{a2} is, independently, hydrogen; optionally substituted aliphatic; optionally substituted heteroaliphatic; optionally substituted aryl; optionally substituted heteroaryl; acyl; or an amino protecting group;

each instance of R^{b} and R^{b′} is, independently, hydrogen; optionally substituted aliphatic; optionally substituted heteroaliphatic; optionally substituted aryl; optionally substituted heteroaryl;

each instance of [symbol] independently corresponds to a single or double bond;

each instance of R^{c4}, R^{c5}, and R^{c6} is independently hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; or nitro; and

each instance of q^{c4}, q^{c5}, and q^{c6} is independently 0, 1, or 2 when [symbol] represents a double bond, or an integer between 1 and 4, inclusive, when [symbol] represents a single bond.

In one aspect, provided is an unstapled, dual-linker polypeptide of Formula (IV):

R^{f}—[X_{AA}]ₛ—[X₁₋₂₃]—[X_{AA}]ₜ—R^{e} (IV)

or a pharmaceutically acceptable salt thereof;
wherein:

each instance of X_{AA} is a natural amino acid or unnatural amino acid;

s is 0 or an integer between 1 and 100, inclusive;

t is 0 or an integer between 1 and 100, inclusive;

R^{f} is an N-terminal group selected from the group consisting of hydrogen; optionally substituted aliphatic; optionally substituted heteroaliphatic; optionally substituted aryl; optionally substituted heteroaryl; acyl; a resin; an amino protecting group; and a label optionally joined by a linker, wherein the linker is selected from the group consisting of optionally substituted alkylene; optionally substituted alkenylene; optionally substituted alkynylene; optionally substituted heteroalkylene; optionally substituted heteroalkenylene; optionally substituted heteroalkynylene; optionally substituted arylene; optionally substituted heteroarylene; and acylene;

R^{e} is a C-terminal group selected from the group consisting of hydrogen; optionally substituted aliphatic; optionally substituted heteroaliphatic; optionally substituted aryl; optionally substituted heteroaryl; —OR^{E}, —N(R^{E})₂, or —SR^{E}, wherein each instance of R^{E} is, independently, hydrogen; optionally substituted aliphatic; optionally substituted heteroaliphatic; optionally substituted aryl; optionally substituted heteroaryl; acyl; a resin; a protecting group; or two R^{E} groups taken together form an optionally substituted heterocyclic or optionally substituted heteroaryl ring; and

—[X₁₋₂₃]— is an unstapled amino sequence of the Formula (SEQ ID NO: 55):

-[X₁-X₂-X₃-X₄-X₅-X₆-X₇-X₈-X₉-X₁₀-X₁₁-X₁₂-X₁₃-
X₁₄-X₁₅-X₁₆-X₁₇-X₁₈-X₁₉-X₂₀-X₂₁-(X₂₂)ₙ-X₂₃]-

wherein:

X₁ is amino acid W;

X₂ is absent or is a natural amino acid or unnatural amino acid;

X₃ is amino acid K;

X₄ is amino acid R;

X₅ is an amino acid selected from the group consisting of A, R, and an amino acid of Formula (i);

X₆ is an amino acid selected from the group consisting of H and T;

X₇ is amino acid H;

X₈ is amino acid N;

X₉ is an amino acid selected from the group consisting of A, V, and an amino acid of Formula (i) or (ii);

X₁₀ is amino acid L;

X₁₁ is amino acid E;

X₁₂ is amino acid R;

X₁₃ is an amino acid selected from the group consisting of K, Q, and an amino acid of Formula (i) or (ii);

X₁₄ is amino acid R;

X₁₅ is amino acid R;

X₁₆ is an amino acid selected from the group consisting of D and N;

X₁₇ is an amino acid selected from the group consisting of H, E, and an amino acid of Formula (i) or (ii);

X₁₈ is an amino acid selected from the group consisting of C and an amino acid of the Formula (v) or (vi):

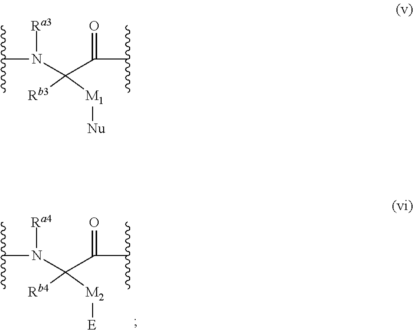

X₁₉ is amino acid K;

X₂₀ is an amino acid selected from the group consisting of D and R;

X₂₁ is amino acid S;

each instance of X₂₂ is independently a natural or unnatural amino acid, and n is 0 or an integer between 1 and 10 inclusive; and

X₂₃ is a natural or unnatural amino acid of the Formula (v) or (vi):

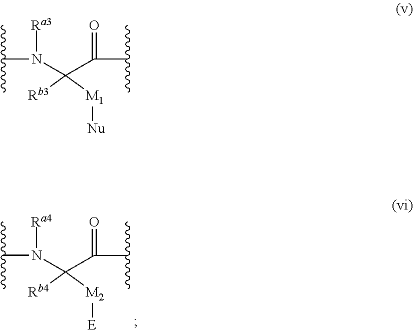

wherein:

each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of each hydrogen; optionally substituted aliphatic; optionally substituted heteroaliphatic; optionally substituted aryl; optionally substituted heteroaryl;

Nu is —SH, —OH, —NHR^{b5}, —NH—NHR^{b5}, —N═NH, —N═C, —N₃, or

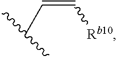

wherein R^{b10} is hydrogen, optionally substituted aliphatic, or optionally substituted heteroaliphatic; and R^{b5} is hydrogen, optionally substituted aliphatic, optionally substituted heteroaliphatic, or an amino protecting group;

E is a leaving group, —CHO, —CO₂R^{b6}, —COX^{b7},

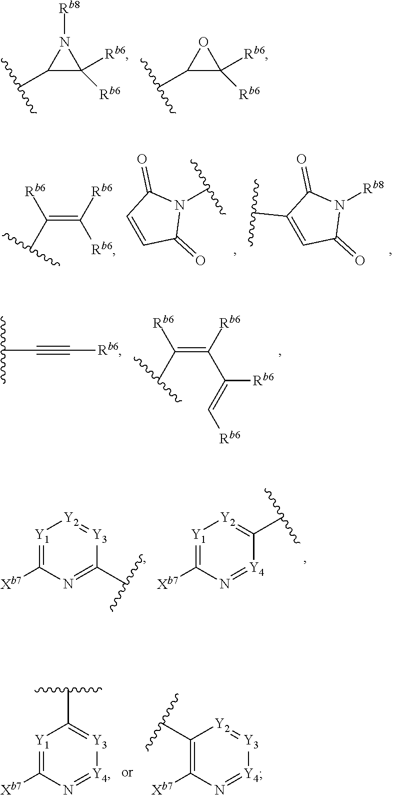

wherein:

R^{b6} is hydrogen, optionally substituted aliphatic, or optionally substituted heteroaliphatic, or wherein two R^{b6} groups are joined to form an optionally substituted carbocyclic or optionally substituted heterocyclic ring;

X^{b7} is a leaving group;

each instance of Y₁, Y₂, Y₃, and Y₄ is independently selected from —N— or —C(R^{b6})—; and

R^{b8} is hydrogen, optionally substituted aliphatic, optionally substituted heteroaliphatic, optionally substituted aryl, optionally substituted heteroaryl, or an amino protecting group;

provided that the amino acid sequence comprises at least two independent occurrences of an amino acid of Formula (i) or (ii);

the amino acid of Formula (i) is:

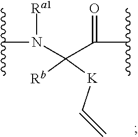

and the amino acid of Formula (ii) is:

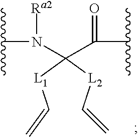

wherein:

each instance of K, L₁, and L₂, is, independently, optionally substituted alkylene; optionally substituted heteroalkylene; optionally substituted arylene; or optionally substituted heteroarylene;

each instance of R^{a1} and R^{a2} is, independently, hydrogen; optionally substituted aliphatic; optionally substituted heteroaliphatic; optionally substituted aryl; optionally substituted heteroaryl; acyl; or an amino protecting group; and

each instance of R^{b} is, independently, hydrogen; optionally substituted aliphatic; optionally substituted heteroaliphatic; optionally substituted aryl; optionally substituted heteroaryl.

In another aspect, provided is a stapled, dual-linker polypeptide of Formula (V):

R^{f}—[X_{AA}]ₛ—[X₁₋₂₃]—[X_{AA}]ₜ—R^{e} (V)

or a pharmaceutically acceptable salt thereof;
wherein:

each instance of X_{AA} is a natural or unnatural amino acid;

s is 0 or an integer between 1 and 100, inclusive;

t is 0 or an integer between 1 and 100, inclusive;

R^{f} is an N-terminal group selected from the group consisting of hydrogen; optionally substituted aliphatic; optionally substituted heteroaliphatic; optionally substituted aryl; optionally substituted heteroaryl; acyl; a resin; an amino protecting group; and a label optionally joined by a linker, wherein the linker is selected from the group consisting of optionally substituted alkylene; optionally substituted alkenylene; optionally substituted alkynylene; optionally substituted heteroalkylene; optionally substituted heteroalkenylene; optionally substituted heteroalkynylene; optionally substituted arylene; optionally substituted heteroarylene; and acylene;

R^{e} is a C-terminal group selected from the group consisting of hydrogen; optionally substituted aliphatic; optionally substituted heteroaliphatic; optionally substituted aryl; optionally substituted heteroaryl; —OR^{E}, —N(R^{E})₂, or —SR^{E}, wherein each instance of R^{E} is, independently, hydrogen; optionally substituted aliphatic; optionally substituted heteroaliphatic; optionally substituted aryl; optionally substituted heteroaryl; acyl; a resin; a protecting group; or two R^{E} groups taken together form an optionally substituted heterocyclic or optionally substituted heteroaryl ring; and

—[X₁₋₂₃]— is a stapled amino sequence of the Formula (SEQ ID NO: 56):

-[X₁-X₂-X₃-X₄-X₅-X₆-X₇-X₈-X₉-X₁₀-X₁₁-X₁₂-X₁₃-X₁₄-
X₁₅-X₁₆-X₁₇-X₁₈-X₁₉-X₂₀-X₂₁-(X₂₂)ₙ-X₂₃]-

wherein:

X₁ is amino acid W;

X₂ is absent or is a natural or unnatural amino acid;

X₃ is amino acid K;

X₄ is amino acid R;

X₅ is an amino acid selected from the group consisting of A and R, or X₅ and X₉ are stapled amino acids of Formula (iii), or X₅, X₉, and X₁₃ are stapled amino acids of Formula (iv);

X₆ is an amino acid selected from the group consisting of H and T;

X₇ is amino acid H;

X₈ is amino acid N;

X₉ is an amino acid selected from the group consisting of A and V, or X₅ and X₉ are stapled amino acids of Formula (iii), or X₉ and X₁₃ are stapled amino acids of Formula (iii), or X₅, X₉, and X₁₃ are stapled amino acids of Formula (iv), or X₉, X₁₃, and X₁₇ are stapled amino acids of Formula (iv);

X₁₀ is amino acid L;

X₁₁ is amino acid E;

X₁₂ is amino acid R;

X₁₃ is an amino acid selected from the group consisting of K and Q, or X₉ and X₁₃ are stapled amino acids of Formula (iii), or X₁₃ and X₁₇ are stapled amino acids of Formula (iii), or X₉, X₁₃, and X₁₇ are stapled amino acids of Formula (iv);

X₁₄ is amino acid R;

X₁₅ is amino acid R;

X₁₆ is an amino acid selected from the group consisting of D and N;

X₁₇ is an amino acid selected from the group consisting of H and E, or X₁₃ and X₁₇ are stapled amino acids of Formula (iii), or X₉, X₁₃, and X₁₇ are stapled amino acids of Formula (iv);

X₁₈ is an amino acid selected from the group consisting of C and an amino acid of the Formula (iii) or (iv):

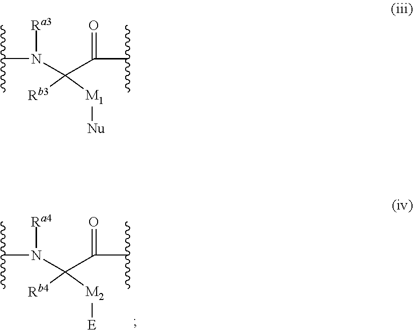

X₁₉ is amino acid K;

X₂₀ is an amino acid selected from the group consisting of D and R;

X₂₁ is amino acid S;

each instance of X₂₂ is independently a natural amino acid or an unnatural amino acid, and n is 0 or an integer between 1 and 10 inclusive; and

X₂₃ is an amino acid of the Formula (iii) or (iv):

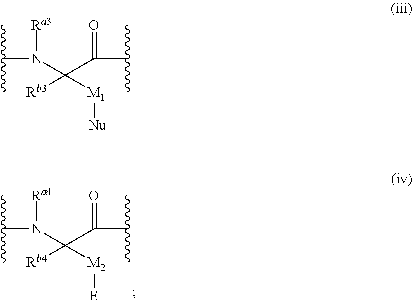

wherein:

each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of each hydrogen; optionally substituted aliphatic; optionally substituted heteroaliphatic; optionally substituted aryl; optionally substituted heteroaryl;

Nu is —SH, —OH, —NHR^{b}, —NH—NHR^{b5}, —N═NH, —N═C, —N₃, or,

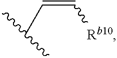

wherein R^{b10} is hydrogen, optionally substituted aliphatic, or optionally substituted heteroaliphatic; and R^{b5} is hydrogen, optionally substituted aliphatic, optionally substituted heteroaliphatic, or an amino protecting group;

E is a leaving group, —CHO, —CO₂R^{b6}, —COX^{b7},

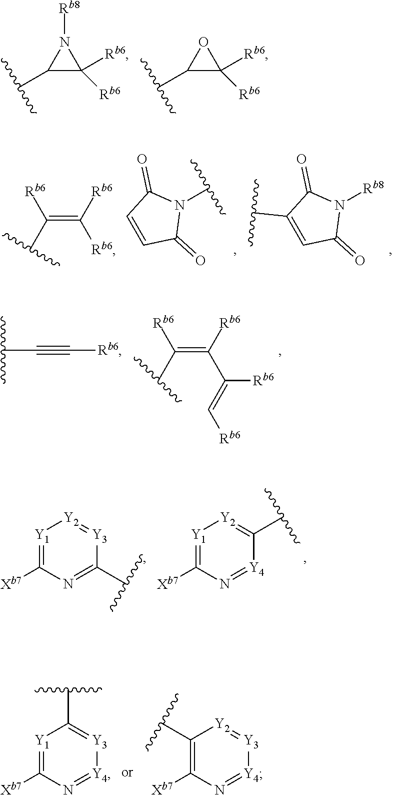

wherein:

R^{b6} is hydrogen, optionally substituted aliphatic, or optionally substituted heteroaliphatic, or wherein two R^{b6} groups are joined to form an optionally substituted carbocyclic or optionally substituted heterocyclic ring;

X^{b7} is a leaving group;

each instance of Y₁, Y₂, Y₃, and Y₄ is independently selected from —N— or —C(R^{b6})—; and

R^{b8} is hydrogen, optionally substituted aliphatic, optionally substituted heteroaliphatic, optionally substituted aryl, optionally substituted heteroaryl, or an amino protecting group;

provided that the amino acid sequence comprises at least one occurrence of stapled amino acids of Formula (iii) or (iv);

wherein the stapled amino acids of Formula (iii) are:

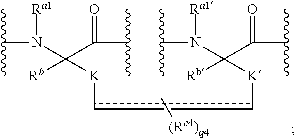

and wherein the stapled amino acids of Formula (iv) are:

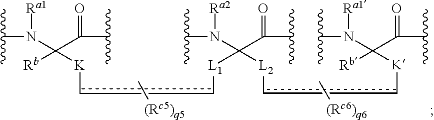

wherein:

each instance of K, K′, L₁, and L₂, is, independently, optionally substituted alkylene; optionally substituted heteroalkylene; optionally substituted arylene; or optionally substituted heteroarylene;

each instance of R^{a1}, R^{a1′}, and R^{a2} is, independently, hydrogen; optionally substituted aliphatic; optionally substituted heteroaliphatic; optionally substituted aryl; optionally substituted heteroaryl; acyl; or an amino protecting group;

each instance of R^{b} and R^{b′} is, independently, hydrogen; optionally substituted aliphatic; optionally substituted heteroaliphatic; optionally substituted aryl; optionally substituted heteroaryl;

each instance of [symbol] independently represents a single or double bond;

each instance of R^{c4}, R^{c5}, and R^{c6} is independently hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; or nitro; and

each instance of q^{c4}, q^{c5}, and q^{c6} is independently 0, an integer between 1 and 2 when [symbol] represents a double bond, or an integer between 1 and 4 when [symbol] represents a single bond.

In yet another aspect, provided is a dual-linker conjugated polypeptide of Formula (VI) comprising:

a first stapled polypeptide of Formula (VIa):

R^{e1}—[X_{AA}]ₛ₁—[X₁₋₂₃]—[X_{AA}]ₜ₁—R^{f1} (VIa)

conjugated to a second stapled polypeptide of Formula (VIb):

R^{e2}—[X_{AA°}]ₛ₂—[X_{1-23°}]—[X_{AA°}]ₜ₂—R^{f2} (VIb)

or a pharmaceutically acceptable salt thereof;
wherein:

each instance of X_{AA} and X_{AA°} is a natural amino acid or unnatural amino acid;

each instance of s1 and s2 is independently 0 or an integer between 1 and 100, inclusive;

each instance of t1 and t2 is independently 0 or an integer between 1 and 100, inclusive;

each instance of R^{f1} and R^{f2} is independently an N-terminal group selected from the group consisting of hydrogen; optionally substituted aliphatic; optionally substituted heteroaliphatic; optionally substituted aryl; optionally substituted heteroaryl; acyl; a resin; an amino protecting group; and a label optionally joined by a linker, wherein the linker is selected from the group consisting of optionally substituted alkylene; optionally substituted alkenylene; optionally substituted alkynylene; optionally substituted heteroalkylene; optionally substituted heteroalkenylene; optionally substituted heteroalkynylene; optionally substituted arylene; optionally substituted heteroarylene; and acylene;

each instance of R^{e1} and R^{e2} is independently a C-terminal group selected from the group consisting of hydrogen; optionally substituted aliphatic; optionally substituted heteroaliphatic; optionally substituted aryl; optionally substituted heteroaryl; —OR^{E}, —N(R^{E})₂, or —SR^{E}, wherein each instance of R^{E} is, independently, hydrogen; optionally substituted aliphatic; optionally substituted heteroaliphatic; optionally substituted aryl; optionally substituted heteroaryl; acyl; a resin; a protecting group; or two R^{E} groups taken together form an optionally substituted heterocyclic or optionally substituted heteroaryl ring;

each instance of —[X₁₋₂₃]— is a first amino sequence of the Formula (VIa′) (SEQ ID NO: 57):

-[X₁-X₂-X₃-X₄-X₅-X₆-X₇-X₈-X₉-X₁₀-X₁₁-X₁₂-X₁₃-X₁₄-
X₁₅-X₁₆-X₁₇-X₁₈-X₁₉-X₂₀-X₂₁-(X₂₂)ₙ₁-X₂₃]-

and

each instance of —[X₁₋₂₃]°— is a second amino sequence of the Formula (VIb′) (SEQ ID NO: 56):

-[X₁°-X₂°-X₃°-X₄°-X₅°-X₆°-X₇°-X₈°-X₉°-X₁₀°-X₁₁°-
X₁₂°-X₁₃°-X₁₄°-X₁₅°-X₁₆°-X₁₇°-X₁₈°-X₁₉°-X₂₀°-X₂₁°-
(X₂₂°)ₙ₂-X₂₃°]-

wherein:

each instance of X₁ and X₁° is amino acid W;

each instance of X₂ and X₂° is independently absent or is a natural amino acid or unnatural amino acid;

each instance of X₃ and X₃° is amino acid K;

each instance of X₄ and X₄° is amino acid R;

each instance of X₅ and X₅° is independently an amino acid selected from the group consisting of A and R, or X₅ and X₉ are stapled amino acids of Formula (iii), or X₅, X₉, and X₁₃ are stapled amino acids of Formula (iv), or X₅° and X₉° are stapled amino acids of Formula (iii), or X₅°, X₉°, and X₁₃° are stapled amino acids of Formula (iv);

each instance of X₆ and X₆° is independently an amino acid selected from the group consisting of H and T;

each instance of X₇ and X₇° is amino acid H;

each instance of X₈ and X₈° is amino acid N;

each instance of X₉ and X₉° is independently an amino acid selected from the group consisting of A and V, or X₅ and X₉ are stapled amino acids of Formula (iii), or X₉ and X₁₃ are stapled amino acids of Formula (iii), or X₅, X₉, and X₁₃ are stapled amino acids of Formula (iv), or X₉, X₁₃, and X₁₇ are stapled amino acids of Formula (iv), or X₅° and X₉° are stapled amino acids of Formula (iii), or X₉° and X₁₃° are stapled amino acids of Formula (iii), or X₅°, X₉°, and X₁₃° are stapled amino acids of Formula (iv), or X₉°, X₁₃°, and X₁₇° are stapled amino acids of Formula (iv);

each instance of X₁₀° and X₁₀° is amino acid L;

each instance of X₁₁ and X₁₁° is amino acid E;

each instance of X₁₂ and X₁₂° is amino acid R;

each instance of X₁₃ and X₁₃° is independently an amino acid selected from the group consisting of K and Q, or X₉ and X₁₃ are stapled amino acids of Formula (iii), or X₁₃ and X₁₇ are stapled amino acids of Formula (iii), or X₉, X₁₃, and X₁₇ are stapled amino acids of Formula (iv), or X₉° and X₁₃° are stapled amino acids of Formula (iii), or X₁₃° and X₁₇° are stapled amino acids of Formula (iii), or X₉°, X₁₃°, and X₁₇° are stapled amino acids of Formula (iv);

each instance of X₁₄ and X₁₄° is amino acid R;

each instance of X₁₅ and X₁₅° is amino acid R;

each instance of X₁₆ and X₁₆° is independently an amino acid selected from the group consisting of D and N;

each instance of X₁₇ and X₁₇° is independently an amino acid selected from the group consisting of H and E, or X₁₃ and X₁₇ are stapled amino acids of Formula (iii), or X₉, X₁₃, and X₁₇ are stapled amino acids of Formula (iv), or X₁₃° and X₁₇° are stapled amino acids of Formula (iii), or X₉°, X₁₃°, and X₁₇° are stapled amino acids of Formula (iv);

wherein amino acid X₁₈ of the first stapled amino acid sequence of Formula (VIa) and amino acid X₁₈° of the second stapled amino acid sequence of Formula (VIb) are joined to form a group of the Formula:

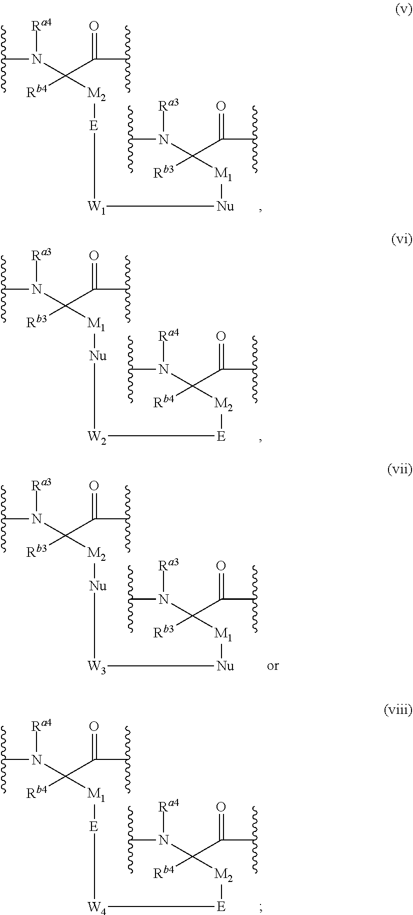

each instance of X₁₉ and X₁₉° is amino acid K;

each instance of X₂₀ and X₂₀° is independently an amino acid selected from the group consisting of D and R;

each instance of X₂₁ and X₂₁° is amino acid S; and

each instance of X₂₂ and X₂₂° is independently a natural amino acid or an unnatural amino acid, n1 is 0 or an integer between 1 and 10 inclusive, and n2 is 0 or an integer between 1 and 10 inclusive; and

wherein amino acid X₂₃ of the first stapled amino acid sequence of Formula (VIa) and amino acid X₂₃° of the second stapled amino acid sequence of Formula (VIb) are joined to form a group of the Formula:

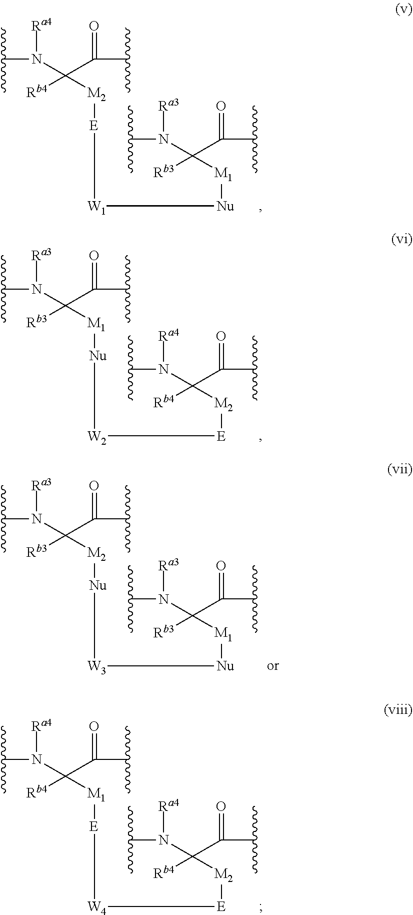

wherein:

each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of each hydrogen; optionally substituted aliphatic; optionally substituted heteroaliphatic; optionally substituted aryl; optionally substituted heteroaryl;

each instance of -Nu-W₁-E- and -Nu-W₂-E- independently represents any one of the following groups:

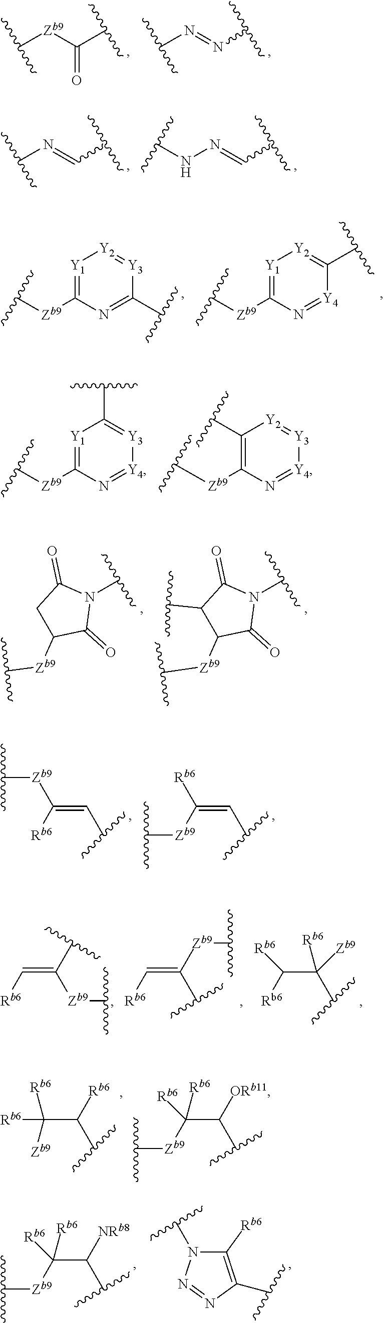

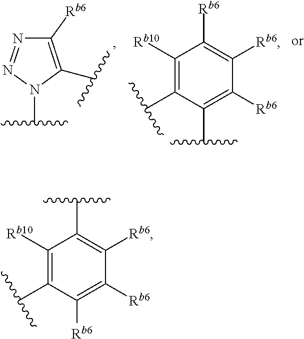

wherein:

Z^{b9} is —O—, —S—, —N(R^{b5})—, —NH—N(R^{b5})—, —N═N—, or —NC—; and R^{b5} is hydrogen, optionally substituted aliphatic, optionally substituted heteroaliphatic, or an amino protecting group;

R^{b6} is hydrogen, optionally substituted aliphatic, or optionally substituted heteroaliphatic, or two R^{b6} groups are joined to form an optionally substituted carbocyclic or optionally substituted heterocyclic ring;

each instance of Y₁, Y₂, Y₃, and Y₄ is independently selected from —N— or —C(R^{b6})—;

R^{b8} is hydrogen, optionally substituted aliphatic, optionally substituted heteroaliphatic, optionally substituted aryl, optionally substituted heteroaryl, or an amino protecting group;

R^{b10} is hydrogen, optionally substituted aliphatic, or optionally substituted heteroaliphatic;

R^{b11} is hydrogen, optionally substituted aliphatic, optionally substituted heteroaliphatic, optionally substituted aryl, optionally substituted heteroaryl, or an oxygen protecting group;

each instance of -Nu-W₃-Nu- independently represents

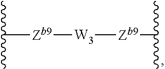

wherein

Z^{b9} is —O—, —S—, —N(R^{b5})—, —NH—N(R^{b5})—, —N═N—, or —N═C—; R^{b5} is hydrogen, optionally substituted aliphatic, optionally substituted heteroaliphatic, or an amino protecting group; and W₃ is selected from the group consisting of optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene; and

each instance of -E-W₄-E- independently represents optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

provided that the polypeptide comprises at least one occurrence of stapled amino acids of Formula (iii) or (iv);

wherein the stapled amino acids of Formula (iii) is:

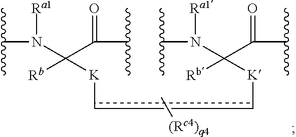

and wherein the stapled amino acids of Formula (iv) is:

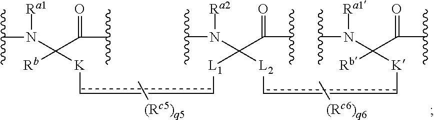

wherein:

each instance of K, K′, L₁, and L₂, is, independently, optionally substituted alkylene; optionally substituted heteroalkylene; optionally substituted arylene; or optionally substituted heteroarylene;

each instance of R^{a1}, R^{a1′}, and R^{a2} is, independently, hydrogen; optionally substituted aliphatic; optionally substituted heteroaliphatic; optionally substituted aryl; optionally substituted heteroaryl; acyl; or an amino protecting group;

each instance of R^{b} and R^{b′} is, independently, hydrogen; optionally substituted aliphatic; optionally substituted heteroaliphatic; optionally substituted aryl; optionally substituted heteroaryl;

each instance of [symbol] independently corresponds to a single or double bond;

each instance of R^{c4}, R^{c5}, and R^{c6} is independently hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; or nitro; and

each instance of q^{c4}, q^{c5}, and q^{c6} is independently 0, 1, or 2 when [symbol] represents a double bond, or an integer between 1 and 4, inclusive, when [symbol] represents a single bond.

In one aspect, provided is an unstapled, dual-linker polypeptide comprising a peptide of Formula (VII) (SEQ ID NO: 58):

[X₁₋₁₁] (VII)

wherein [X₁₋₁₁] is:

[(X₁)ₙ-X₂-X₃-X₄-X₅-(X₆)_{z}-X₇-(X₈)ₓ-X₉-(X₁₀)_{y}-X₁₁]

wherein:

each instance of X₁ is independently a natural or unnatural amino acid, and n is 0 or an integer between 1 and 25 inclusive;

X₂ is selected from the group consisting of a natural or unnatural amino acid, and an amino acid of Formula (i) or (ii);

X₃ is any a natural or unnatural amino acid;

X₄ is any a natural or unnatural amino acid;

X₅ is any a natural or unnatural amino acid;

each instance of X₆ is independently a natural or unnatural amino acid, and z is 0 or 3;

X₇ is selected from the group consisting of a natural or unnatural amino acid, and an amino acid of Formula (i) or (ii);

each instance of X₈ is independently a natural or unnatural amino acid, and x is 0 or an integer between 1 and 10 inclusive;

X₉ is an amino acid selected from the group consisting of C and an amino acid of the Formula (v) or (vi):

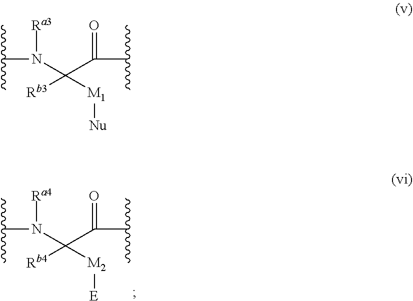

each instance of X₁₀ is independently a natural or unnatural amino acid, and y is 0 or an integer between 1 and 10 inclusive;

X₁₁ is an amino acid selected from the group consisting of C and an amino acid of the Formula (v) or (vi):

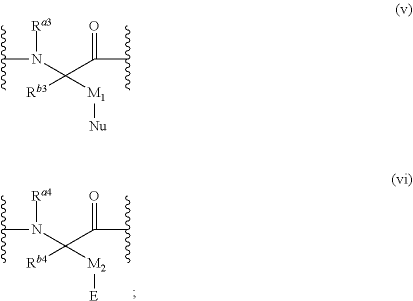

wherein:

each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of each hydrogen; optionally substituted aliphatic; optionally substituted heteroaliphatic; optionally substituted aryl; optionally substituted heteroaryl;

Nu is —SH, —OH, —NHR^{b5}, —NH—NHR^{b5}, —N═NH, —N═C, —N₃, or

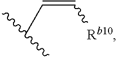

wherein R^{b10} is hydrogen, optionally substituted aliphatic, or optionally substituted heteroaliphatic; and R^{b5} is hydrogen, optionally substituted aliphatic, optionally substituted heteroaliphatic, or an amino protecting group;

E is a leaving group, —CHO, —CO₂R^{b6}, —COX^{b7},

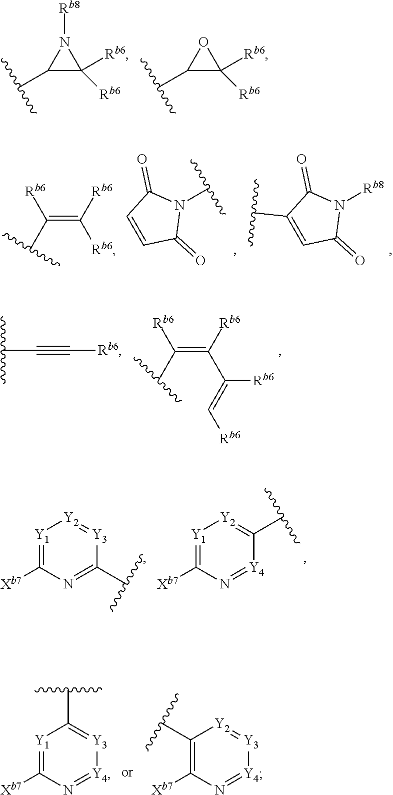

wherein:

R^{b6} is hydrogen, optionally substituted aliphatic, or optionally substituted heteroaliphatic, or wherein two R^{b6} groups are joined to form an optionally substituted carbocyclic or optionally substituted heterocyclic ring;

X^{b7} is a leaving group;

each instance of Y₁, Y₂, Y₃, and Y₄ is independently selected from —N— or —C(R^{b6})—; and

R^{b8} is hydrogen, optionally substituted aliphatic, optionally substituted heteroaliphatic, optionally substituted aryl, optionally substituted heteroaryl, or an amino protecting group;

provided that the amino acid sequence comprises at least two independent occurrences of an amino acid of Formula (i) or (ii);

the amino acid of Formula (i) is:

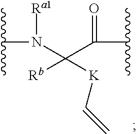

and the amino acid of Formula (ii) is:

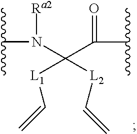

wherein:

each instance of K, L₁, and L₂, is, independently, optionally substituted alkylene; optionally substituted heteroalkylene; optionally substituted arylene; or optionally substituted heteroarylene;

each instance of R^{a1} and R^{a2} is, independently, hydrogen; optionally substituted aliphatic; optionally substituted heteroaliphatic; optionally substituted aryl; optionally substituted heteroaryl; acyl; or an amino protecting group; and

each instance of R^{b} is, independently, hydrogen; optionally substituted aliphatic; optionally substituted heteroaliphatic; optionally substituted aryl; optionally substituted heteroaryl.

In one aspect, provided is an unstapled, dual-linker polypeptide comprising a peptide of Formula (VIII) (SEQ ID NO: 59):

[X₁₋₁₁] (VIII)

wherein [X₁₋₁₁] is:

[(X₁)ₙ-X₂-X₃-X₄-X₅-(X₆)_{z}-X₇-(X₈)ₓ-X₉-(X₁₀)_{y}-X₁₁]

wherein:

each instance of X₁ is independently a natural or unnatural amino acid, and n is 0 or an integer between 1 and 25 inclusive;

X₂ and X₇ are stapled amino acids of Formula (iii);

X₃ is any a natural or unnatural amino acid;

X₄ is any a natural or unnatural amino acid;

X₅ is any a natural or unnatural amino acid;

each instance of X₆ is independently a natural or unnatural amino acid, and z is 0 or 3;

each instance of X₈ is independently a natural or unnatural amino acid, and x is 0 or an integer between 1 and 10 inclusive;

X₉ is an amino acid selected from the group consisting of C and an amino acid of the Formula (v) or (vi):

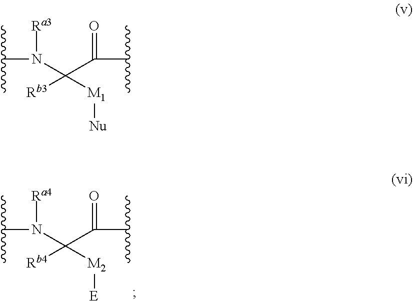

each instance of X₁₀ is independently a natural or unnatural amino acid, and y is 0 or an integer between 1 and 10 inclusive;

X₁₁ is an amino acid selected from the group consisting of C and an amino acid of the Formula (v) or (vi):

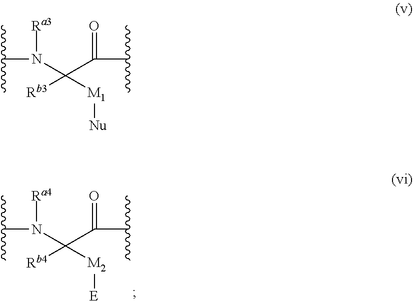

wherein:

each instance of M₁ and M₂ is independently optionally substituted alkylene, optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of each hydrogen; optionally substituted aliphatic; optionally substituted heteroaliphatic; optionally substituted aryl; optionally substituted heteroaryl;

Nu is —SH, —OH, —NHR^{b5}, —NH—NHR^{b5}, —N═NH, —N═C, —N₃, or,

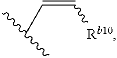

wherein R^{b10} is hydrogen, optionally substituted aliphatic, or optionally substituted heteroaliphatic; and R^{b5} is hydrogen, optionally substituted aliphatic, optionally substituted heteroaliphatic, or an amino protecting group;

E is a leaving group, —CHO, —CO₂R^{b6}, —COX^{b7},

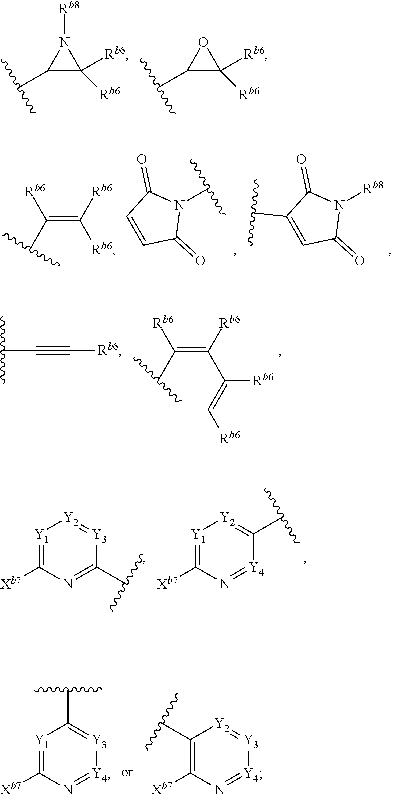

wherein:

R^{b6} is hydrogen, optionally substituted aliphatic, or optionally substituted heteroaliphatic, or wherein two R^{b6} groups are joined to form an optionally substituted carbocyclic or optionally substituted heterocyclic ring;

X^{b7} is a leaving group;

each instance of Y₁, Y₂, Y₃, and Y₄ is independently selected from —N— or —C(R^{b6})—; and

R^{b8} is hydrogen, optionally substituted aliphatic, optionally substituted heteroaliphatic, optionally substituted aryl, optionally substituted heteroaryl, or an amino protecting group;

provided that the amino acid sequence comprises at least two independent occurrences of an amino acid of Formula (i) or (ii);

the amino acid of Formula (i) is:

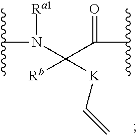

and the amino acid of Formula (ii) is:

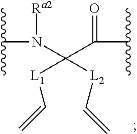

wherein:

each instance of K, L₁, and L₂, is, independently, optionally substituted alkylene; optionally substituted heteroalkylene; optionally substituted arylene; or optionally substituted heteroarylene;

each instance of R^{a1} and R^{a2} is, independently, hydrogen; optionally substituted aliphatic; optionally substituted heteroaliphatic; optionally substituted aryl; optionally substituted heteroaryl; acyl; or an amino protecting group; and

each instance of R^{b} is, independently, hydrogen; optionally substituted aliphatic; optionally substituted heteroaliphatic; optionally substituted aryl; optionally substituted heteroaryl;

wherein Formula (iii) is:

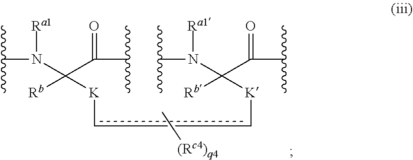

wherein:

K and K′ are independently, optionally substituted alkylene; optionally substituted heteroalkylene; optionally substituted arylene; or optionally substituted heteroarylene;

R^{a1} and R^{a1′} are independently, hydrogen; optionally substituted aliphatic; optionally substituted heteroaliphatic; optionally substituted aryl; optionally substituted heteroaryl; acyl; or an amino protecting group;

R^{b} and R^{b′} are, independently, hydrogen; optionally substituted aliphatic; optionally substituted heteroaliphatic; optionally substituted aryl; optionally substituted heteroaryl;

each instance of [symbol] independently represents a single or double bond;

R^{c4} is hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; or nitro; and

q⁴ is independently 0, an integer between 1 and 2 when [symbol] represents a double bond, or an integer between 1 and 4 when [symbol] represents a single bond.

In yet another aspect, provided is a dual-linker conjugated polypeptide of Formula (IX) comprising:

a first stapled polypeptide of comprising a peptide of Formula (IXa) (SEQ ID NO: 60):

[X₁₋₁₁] (IXa)

conjugated to a second stapled polypeptide comprising a peptide of Formula (IXb) (SEQ ID NO: 60):

[X_{1°-11°}] (IXb);

wherein [X₁₋₁₁] is:

[(X₁)ₙ-X₂-X₃-X₄-X₅-(X₆)_{z}-X₇-(X₈)ₓ-X₉-(X₁₀)_{y}-X₁₁];

wherein [X_{1°-11°}] is:

[(X_{1°})ₙ-X_{2°}-X_{3°}-X_{4°}-X_{5°}-(X_{6°})_{z}-X_{7°}-(X_{8°})ₓ-X_{9°}-
(X_{10°})_{y}-X_{11°}];

wherein:

each instance of X₁ and X_{1°} is independently a natural or unnatural amino acid, and each n is independently 0 or an integer between 1 and 25 inclusive;

X₂ and X₇, and X_{2°} and X_{7°}, are respectively stapled amino acids of Formula (iii);

X₃ and X_{3°} are independently any a natural or unnatural amino acid;

X₄ and X_{4°} are independently any a natural or unnatural amino acid;

X₅ and X_{5°} are independently any a natural or unnatural amino acid;

each instance of X₆ and X_{6°} are independently a natural or unnatural amino acid, and each z is independently 0 or 3;

each instance of X₈ and X_{8°} is independently a natural or unnatural amino acid, and each x is independently 0 or an integer between 1 and 10 inclusive;

wherein amino acid X₉ of the first stapled amino acid sequence of Formula (IXa) and amino acid X₉° of the second stapled amino acid sequence of Formula (IXb) are joined to form a group of the Formula:

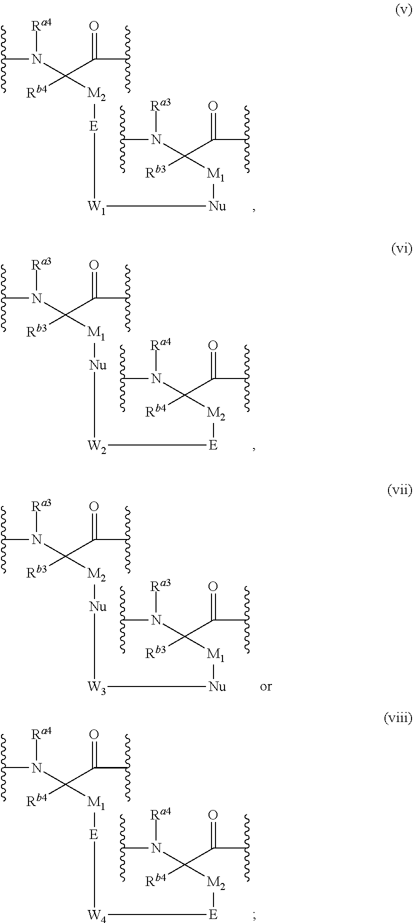

each instance of X₁₀ and X_{10°} are independently a natural or unnatural amino acid, and each independently is 0 or an integer between 1 and 10 inclusive;

wherein amino acid X₁₁ of the first stapled amino acid sequence of Formula (IXa) and amino acid X_{11°} of the second stapled amino acid sequence of Formula (IXb) are joined to form a group of the Formula:

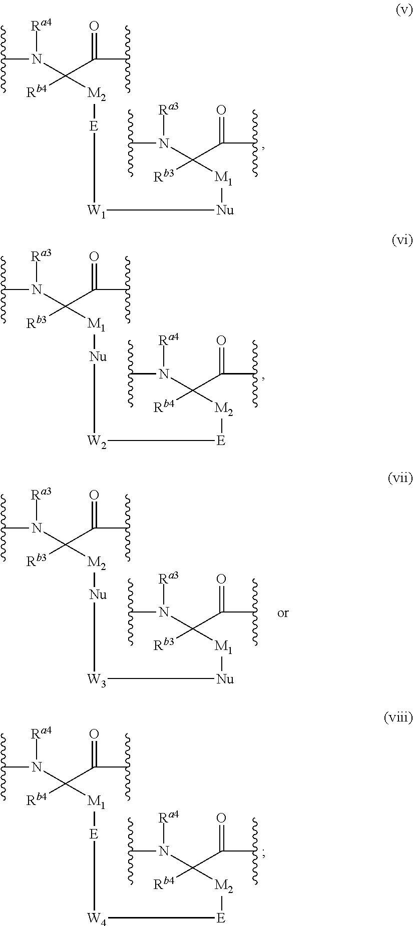

wherein:

each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of each hydrogen; optionally substituted aliphatic; optionally substituted heteroaliphatic; optionally substituted aryl; optionally substituted heteroaryl;

each instance of -Nu-W₁-E- and -Nu-W₂-E- independently represents any one of the following groups:

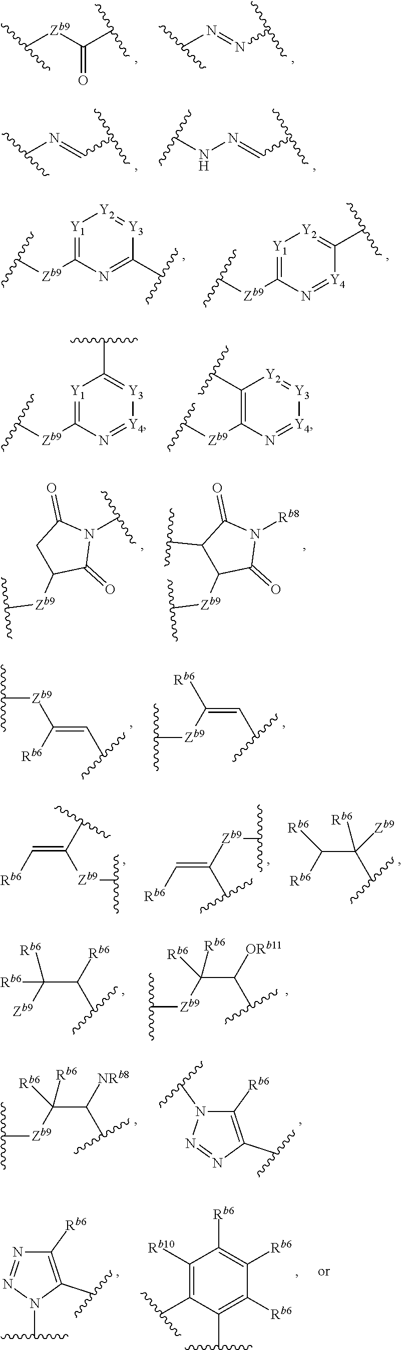

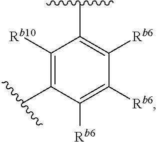

wherein:

Z^{b9} is —O—, —S—, —N(R^{b5})—, —NH—N(R^{b5})—, —N═N—, or —NC—; and R^{b5} is hydrogen, optionally substituted aliphatic, optionally substituted heteroaliphatic, or an amino protecting group;

R^{b6} is hydrogen, optionally substituted aliphatic, or optionally substituted heteroaliphatic, or two R^{b6} groups are joined to form an optionally substituted carbocyclic or optionally substituted heterocyclic ring;

each instance of Y₁, Y₂, Y₃, and Y₄ is independently selected from —N— or —C(R^{b6})—;

R^{b8} is hydrogen, optionally substituted aliphatic, optionally substituted heteroaliphatic, optionally substituted aryl, optionally substituted heteroaryl, or an amino protecting group;

R^{b10} is hydrogen, optionally substituted aliphatic, or optionally substituted heteroaliphatic;

R^{b11} is hydrogen, optionally substituted aliphatic, optionally substituted heteroaliphatic, optionally substituted aryl, optionally substituted heteroaryl, or an oxygen protecting group;

each instance of -Nu-W₃-Nu- independently represents

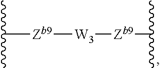

wherein
Z^{b9} is —O—, —S—, —N(R^{b5})—, —NH—N(R^{b5})—, —N═N—, or —N═C—; R^{b5} is hydrogen, optionally substituted aliphatic, optionally substituted heteroaliphatic, or an amino protecting group; and W₃ is selected from the group consisting of optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene; and

each instance of -E-W₄-E- independently represents optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

provided that the polypeptide comprises at least one occurrence of stapled amino acids of Formula (iii) or (iv);

wherein the stapled amino acids of Formula (iii) is:

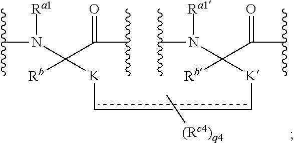

and wherein the stapled amino acids of Formula (iv) is:

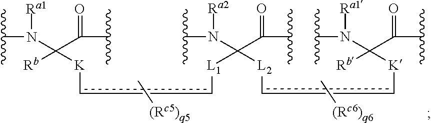

wherein:

each instance of K, K′, L₁, and L₂, is, independently, optionally substituted alkylene; optionally substituted heteroalkylene; optionally substituted arylene; or optionally substituted heteroarylene;

each instance of R^{a1}, R^{a1′}, and R^{a2} is, independently, hydrogen; optionally substituted aliphatic; optionally substituted heteroaliphatic; optionally substituted aryl; optionally substituted heteroaryl; acyl; or an amino protecting group;

each instance of R^{b} and R^{b′} is, independently, hydrogen; optionally substituted aliphatic; optionally substituted heteroaliphatic; optionally substituted aryl; optionally substituted heteroaryl; each instance of [symbol] independently corresponds to a single or double bond;

each instance of R^{c4}, R^{c5}, and R^{c6} is independently hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; or nitro; and

each instance of q^{c4}, q^{c5}, and q^{c6} is independently 0, 1, or 2 when [symbol] represents a double bond, or an integer between 1 and 4, inclusive, when [symbol] represents a single bond.

In one aspect, provided is an unstapled, (dual- or single-) linker polypeptide comprising a peptide of Formula (X) (SEQ ID NO: 61):

[X₁₋₁₃] (X)

wherein [X₁₋₁₃] is:

[(X₁)ₙ-X₂-X₃-X₄-X₅-(X₆)_{z}-X₇-(X₈)ₘ-X₉-(X₁₀)_{y}-X₁₁-
(X₁₂)ₓ-X₁₃]

wherein

each instance of X₁ is independently a natural or unnatural amino acid, and n is 0 or an integer between 1 and 25 inclusive;

X₂ is a modified or unnatural amino acid comprising hindered α-methyl, α-alkenyl substituents;

X₃ is any a natural or unnatural amino acid;

X₄ is any a natural or unnatural amino acid;

X₅ is any a natural or unnatural amino acid;

each instance of X₆ is independently a natural or unnatural amino acid, and z is 0, 3, or 6;

X₇ is a modified or unnatural amino acid comprising hindered α-methyl, α-alkenyl substituents;

each instance of X₈ is independently a natural or unnatural amino acid, and m is 0 or 6, wherein m is 0 if z is 6;

X₉ is absent or a modified or unnatural amino acid comprising hindered α-methyl, α-alkenyl substituents, wherein X₉ is absent if z is 6 or m is 0;

each instance of X₁₀ is independently a natural or unnatural amino acid, and y is 0 or an integer between 1 and 10 inclusive;

X₁₁ is a modified or unnatural amino acid displaying a linker group (e.g., thiol, maleimide, azide, alkyne, etc.);

each instance of X₁₂ is independently a natural or unnatural amino acid, and x is 0 or an integer between 1 and 10 inclusive;

X₁₃ absent or is a modified or unnatural amino acid displaying a linker group (e.g., thiol, maleimide, azide, alkyne, etc.).

In certain embodiments, provided is a stapled, (dual- or single-) linker polypeptide comprising a peptide of Formula (XI) (SEQ ID NO: 62):

[X₁₋₁₃] (XI)

wherein [X₁₋₁₃] is:

[(X₁)ₙ-X₂-X₃-X₄-X₅-(X₆)_{z}-X₇-(X₈)ₘ-X₉-(X₁₀)_{y}-X₁₁-
(X₁₂)ₓ-X₁₃]

wherein

each instance of X₁ is independently a natural or unnatural amino acid, and n is 0 or an integer between 1 and 25 inclusive;

X₂ is a modified or unnatural amino acid linked to X₇ via a hydrocarbon staple resulting from a ring-closing olefin metathesis (RCM) of hindered α-methyl, α-alkenyl amino acids;

X₃ is any a natural or unnatural amino acid;

X₄ is any a natural or unnatural amino acid;

X₅ is any a natural or unnatural amino acid;

each instance of X₆ is independently a natural or unnatural amino acid, and z is 0, 3, or 6;

X₇ is a modified or unnatural amino acid linked to X₂ via a hydrocarbon staple resulting from a ring-closing olefin metathesis (RCM) of hindered α-methyl, α-alkenyl amino acids;

each instance of X₈ is independently a natural or unnatural amino acid, and m is 0 or 6, wherein m is 0 if z is 6;

X₉ is absent or a modified or unnatural amino acid comprising hindered α-methyl, α-alkenyl substituents, wherein X₉ is absent if z is 6 or m is 0;

each instance of X₁₀ is independently a natural or unnatural amino acid, and y is 0 or an integer between 1 and 10 inclusive;

X₁₁ is a modified or unnatural amino acid displaying a linker group (e.g., thiol, maleimide, azide, alkyne, etc.);

each instance of X₁₂ is independently a natural or unnatural amino acid, and x is 0 or an integer between 1 and 10 inclusive;

X₁₃ absent or is a modified or unnatural amino acid displaying a linker group (e.g., thiol, maleimide, azide, alkyne, etc.).

In certain embodiments, provided herein is a dual- or single-linker conjugated polypeptide of Formula (XII), comprising:

a first stapled polypeptide of comprising a peptide of Formula (XIIa) (SEQ ID NO: 63):

[X₁₋₁₃] (XIIa)

conjugated to a second stapled polypeptide comprising a peptide of Formula (XIIb) (SEQ ID NO: 63):

[X_{1°-13°}] (XIIb);

wherein [X₁₋₁₃] is:

[(X₁)ₙ-X₂-X₃-X₄-X₅-(X₆)_{z}-X₇-(X₈)ₘ-X₉-(X₁₀)_{y}-X₁₁-
(X₁₂)ₓ-X₁₃],

wherein [X₁₋₁₃] is:

[(X_{1°})ₙ-X_{2°}-X_{3°}-X_{4°}-X_{5°}-(X_{6°})_{z}-X_{7°}-(X_{8°})ₘ-X_{9°}-
(X_{10°})_{y}-X_{11°}-(X_{12°})ₓ-X_{13°}];

wherein

each instance of X₁ and X_{1°} are independently a natural or unnatural amino acid, and each n is independently 0 or an integer between 1 and 25 inclusive;

X₂ and X_{2°} are independently modified or unnatural amino acid linked to X₇ and X_{7°}, respectively, via a hydrocarbon staple resulting from a ring-closing olefin metathesis (RCM) of hindered α-methyl, α-alkenyl amino acids;

X₃ and X_{3°} are independently any a natural or unnatural amino acid;

X₄ and X_{5°} are independently any a natural or unnatural amino acid;

X₄ and X_{5°} are independently any a natural or unnatural amino acid;

each instance of X₆ and X_{6°} are independently a natural or unnatural amino acid, and each z is independently 0, 3, or 6;

X₇ and X_{7°} are independently any modified or unnatural amino acid linked to X₂ and X_{2°}, respectively, via a hydrocarbon staple resulting from a ring-closing olefin metathesis (RCM) of hindered α-methyl, α-alkenyl amino acids;

each instance of X₈ and X_{8°} are independently a natural or unnatural amino acid, and each m is 0 or 6, wherein m is 0 if z is 6;

X₉ and X_{9°} are independently absent or a modified or unnatural amino acid comprising hindered α-methyl, α-alkenyl substituents, wherein each X₉ is independently absent if z is 6 or m is 0;

each instance of X₁₀ and X_{10°} are independently a natural or unnatural amino acid, and each y is independently 0 or an integer between 1 and 10 inclusive;

X₁₁ and X_{11°} are independently a modified or unnatural amino acid displaying a linker group (e.g., thiol, maleimide, azide, alkyne, etc.);

each instance of X₁₂ and X_{12°} are independently a natural or unnatural amino acid, and each x is independently 0 or an integer between 1 and 10 inclusive;

X₁₃ and X_{13°} are independently absent or are a modified or unnatural amino acid displaying a linker group (e.g., thiol, maleimide, azide, alkyne, etc.).

In certain embodiments, the amino acid of Formula (i) is selected from the group consisting of:

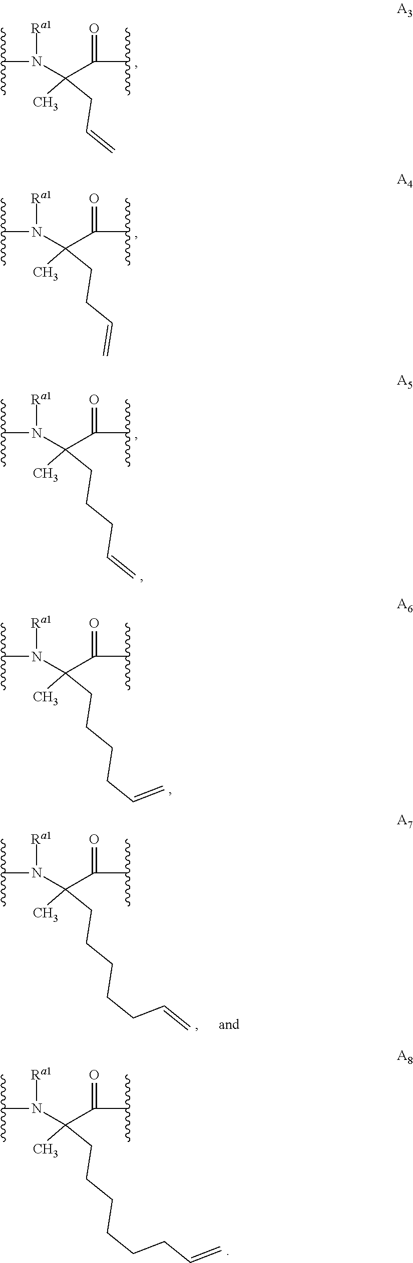

In certain embodiments, each instance of the amino acid of Formula (i) is A₅. In certain embodiments, each instance of the amino acid of Formula (i) is A₈.

In certain embodiments, the alpha carbon of the amino acid of Formula (i) is in the (S) configuration. In certain embodiments, the alpha carbon of the amino acid of Formula (i) is in the (R) configuration.

In certain embodiments, the amino acid of Formula (i) is selected from the group consisting of:

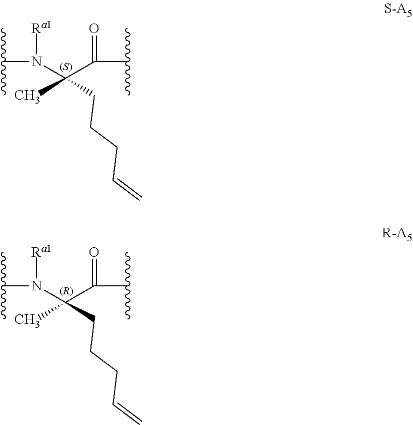

(S)-2-amino-2-methylhept-6-enoic acid, (R)-2-amino-2-methylhept-6-enoic acid,

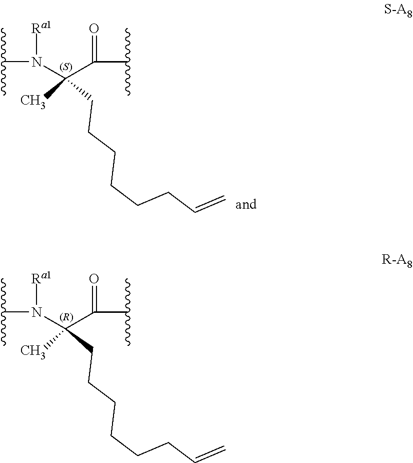

(S)-2-amino-2-methyldec-9-enoic acid, (R)-2-amino-2-methyldec-9-enoic acid.

In certain embodiments, each instance of the amino acid of Formula (i) is S-A₅ (also referred to herein as S₅). In certain embodiments, each instance of the amino acid of Formula (i) is S-A₈ (also referred to herein as S₈).
Exemplary amino acids of Formula (ii) include, but are not limited to,

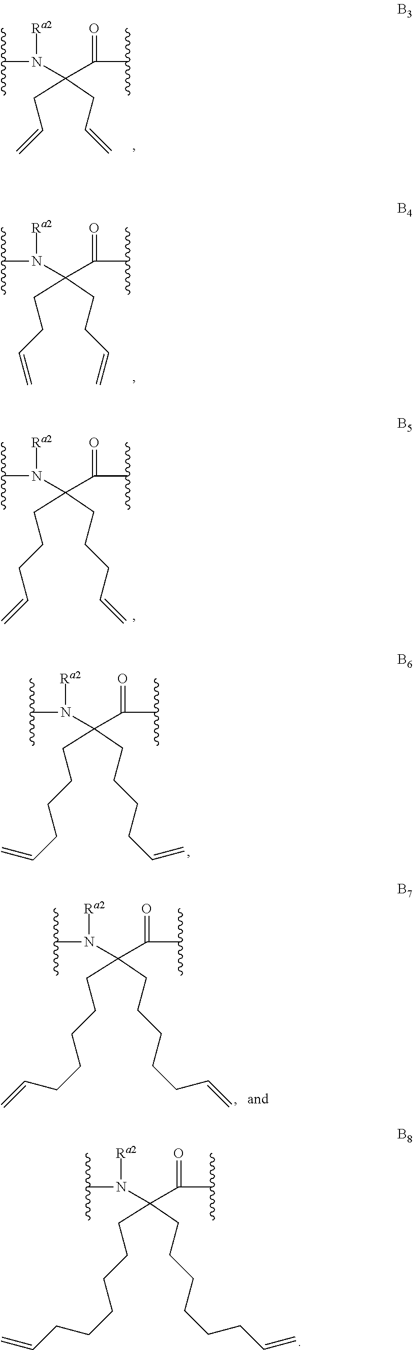

Polypeptides of Formula (I) and (II) are unconjugated monomeric polypeptides wherein amino acid X₂₃ is an unconjugated amino acid of the Formula (iii) or (iv):

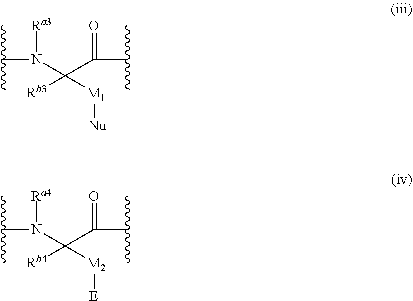

wherein:

each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of hydrogen, optionally substituted aliphatic, and optionally substituted heteroaliphatic;

Nu is —SH, —OH, —NHR^{b5}, —NH—NHR^{b5}, —N═NH, —N═C, —N₃, or

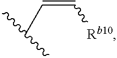

wherein R^{b10} is hydrogen, optionally substituted aliphatic, or optionally substituted heteroaliphatic; and R^{b5} is hydrogen, optionally substituted aliphatic, optionally substituted heteroaliphatic, or an amino protecting group;

E is a leaving group, —CHO, —CO₂R^{b6}, —COX^{b7},

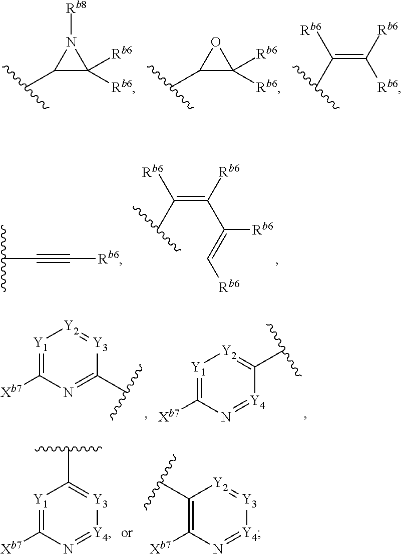

wherein:

R^{b6} is hydrogen, optionally substituted aliphatic, or optionally substituted heteroaliphatic, or wherein two R^{b6} groups are joined to form an optionally substituted carbocyclic or optionally substituted heterocyclic ring;

X^{b7} is a leaving group;

each instance of Y₁, Y₂, Y₃, and Y₄ is independently selected from —N— or —C(R^{b6})—; and

R^{b8} is hydrogen, optionally substituted aliphatic, optionally substituted heteroaliphatic, optionally substituted aryl, optionally substituted heteroaryl, or an amino protecting group.

In certain embodiments, —[X₂₃]— is an amino acid of Formula (iii):

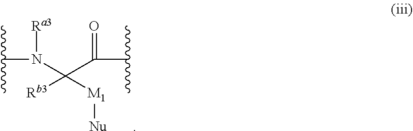

In certain embodiments, —[X₂₃]— is an amino acid of Formula (iv):

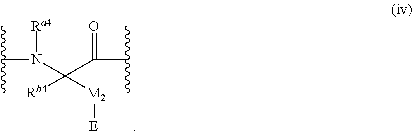

In certain embodiments, —[X₂₃]— is of Formula (iii) and —[X₂₃]— is of Formula (iv) of two polypeptides of Formula (II) are conjugated to form a polypeptide of Formula (III):

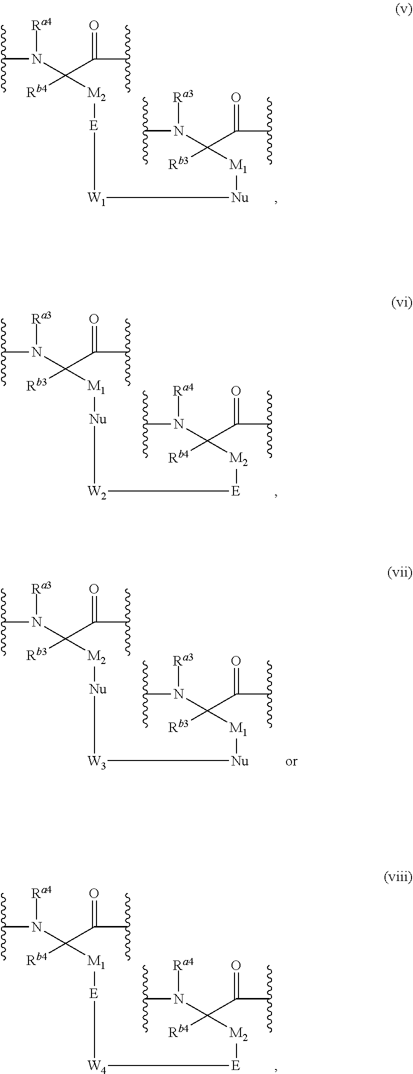

wherein:

each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;

each instance of R^{b3} and R^{b4} is independently selected from the group consisting of hydrogen; optionally substituted aliphatic; optionally substituted heteroaliphatic; optionally substituted aryl; optionally substituted heteroaryl;

each instance of -Nu-W₁-E- and -Nu-W₂-E- independently represents any one of the following groups:

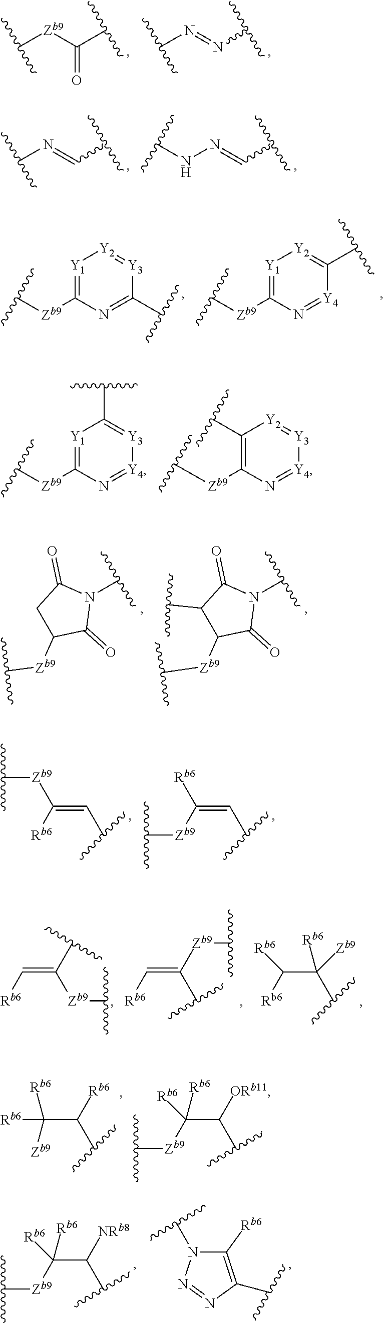

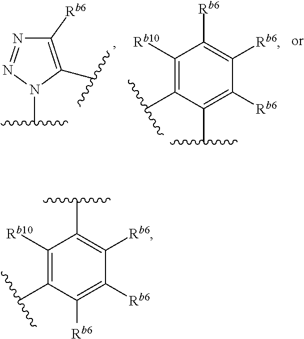

wherein:

Z^{b9} is —O—, —S—, —N(R^{b5})—, —NH—N(R^{b5})—, —N═N—, or —NC—; and R^{b5} is hydrogen, optionally substituted aliphatic, optionally substituted heteroaliphatic, or an amino protecting group;

R^{b6} is hydrogen, optionally substituted aliphatic, or optionally substituted heteroaliphatic, or two R^{b6} groups are joined to form an optionally substituted carbocyclic or optionally substituted heterocyclic ring;

each instance of Y₁, Y₂, Y₃, and Y₄ is independently selected from —N— or —C(R^{b6})—;

R^{b8} is hydrogen, optionally substituted aliphatic, optionally substituted heteroaliphatic, optionally substituted aryl, optionally substituted heteroaryl, or an amino protecting group;

R^{b10} is hydrogen, optionally substituted aliphatic, or optionally substituted heteroaliphatic;

R^{b11} is hydrogen, optionally substituted aliphatic, optionally substituted heteroaliphatic, optionally substituted aryl, optionally substituted heteroaryl, or an oxygen protecting group;

each instance of -Nu-W₃-Nu- independently represents

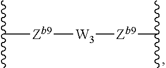

wherein
Z^{b9} is —O—, —S—, —N(R^{b5})—, —NH—N(R^{b5})—, —N═N—, or —N═C—; R^{b5} is hydrogen, optionally substituted aliphatic, optionally substituted heteroaliphatic, or an amino protecting group; and W₃ is selected from the group consisting of optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene; and each instance of -E-W₄-E- independently represents optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene.

In certain embodiments of Formula (I), (II) and (III), each instance of M₁ and M₂ is independently optionally substituted alkylene. In certain embodiments, each instance of M₁ and M₂ is independently optionally substituted heteroalkylene. In certain embodiments, each instance of M₁ and M₂ is independently optionally substituted arylene. In certain embodiments, each instance of M₁ and M₂ is independently optionally substituted heteroarylene. In certain embodiments, each instance of M₁ and M₂ is independently optionally substituted C₁₋₆ alkylene, e.g., optionally substituted C₂₋₆ alkylene, optionally substituted C₃₋₆ alkylene, optionally substituted C₄₋₆ alkylene, optionally substituted C₅₋₆ alkylene, optionally substituted C₂ alkylene, optionally substituted C₃ alkylene, optionally substituted C₄ alkylene, optionally substituted C₅ alkylene, or an optionally substituted C₆ alkylene. In certain embodiments, each instance of each instance of M₁ and M₂ is independently an unsubstituted group. For example, in certain embodiments, each instance of M₁ and M₂ is independently an unsubstituted C₁₋₆ alkylene, e.g., unsubstituted C₂₋₆ alkylene, unsubstituted C₃₋₆ alkylene, unsubstituted C₄₋₆ alkylene, unsubstituted C₅₋₆ alkylene, unsubstituted C₂ alkylene, unsubstituted C₃ alkylene, unsubstituted C₄ alkylene, unsubstituted C₅ alkylene, or an unsubstituted C₆ alkylene.

In certain embodiments of Formula (I) and (II), Nu is —SH.
In certain embodiments of Formula (I) and (II), Nu is selected from the group consisting of —NHR^{b5}, —NH—NHR^{b5}, and —N═NH. In certain embodiments of Formula (I) and (II), Nu is —NHR^{b5}. In certain embodiments of Formula (I) and (II), Nu is —NH—NHR^{b5}. In certain embodiments of Formula (I) and (II), Nu is —N═NH.
In certain embodiments of Formula (I) and (II), Nu is —N═C.
In certain embodiments of Formula (I) and (II), Nu is —N₃.
In certain embodiments of Formula (I) and (II), Nu is

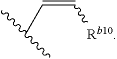

In certain embodiments of Formula (I) and (II), E is halo, —CHO, —CO₂R^{b6}, Or —COX^{b7}.
In certain embodiments of Formula (I) and (II), E is

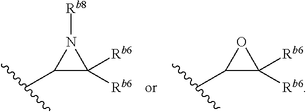

In certain embodiments of Formula (I) and (II), E is

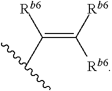

In certain embodiments of Formula (I) and (II), E is

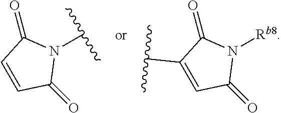

In certain embodiments of Formula (I) and (II), E is

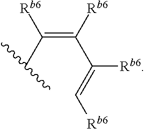

In certain embodiments of Formula (I) and (II), E is

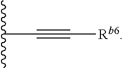

In certain embodiments of Formula (I) and (II), E is

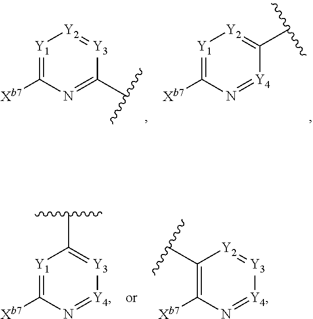

wherein X^{b7} is a leaving group (e.g., —Br, —Cl, —I), each instance of Y₁, Y₂, Y₃, and Y₄ is independently selected from —N— or —C(R^{b6})—, and R^{b6} is hydrogen, optionally substituted aliphatic, or optionally substituted heteroaliphatic, or two R^{b6} groups are joined to form an optionally substituted carbocyclic or optionally substituted heterocyclic ring. In certain embodiments, X^{b7} is —Cl. In certain embodiments, each instance of Y₁, Y₂, Y₃, and Y₄ is independently selected from —C(R^{b6})—. In certain embodiments, Y₁ is —N— and Y₂, Y₃, and Y₄ are independently —C(R^{b6})—. In certain embodiments, Y₂ is —N— and Y₁, Y₃, and Y₄ are independently —C(R^{b6})—. In certain embodiments, Y₃ is —N— and Y₁, Y₂, and Y₄ are independently —C(R^{b6})—. In certain embodiments, Y₄ is —N— and Y₁, Y₂, and Y₃ are independently —C(R^{b6})—. In certain embodiments, each instance of Y₁ and Y₂ is —N— and Y₃ and Y₄ are independently —C(R^{b6})—. In certain embodiments, each instance of Y₁ and Y₃ is —N— and Y₂ and Y₄ are independently —C(R^{b6})—. In certain embodiments, each instance of Y₁ and Y₄ is —N— and Y₂ and Y₃ are independently —C(R^{b6})—. In certain embodiments, each instance of Y₂ and Y₃ is —N— and Y₁ and Y₄ are independently —C(R^{b6})—. In certain embodiments, each instance of Y₃ and Y₄ is —N— and Y₂ and Y₃ are independently —C(R^{b6})—. In certain embodiments, R^{b6} is hydrogen, optionally substituted aliphatic, or optionally substituted heteroaliphatic. In certain embodiments, R^{b6} is hydrogen or C₁₋₆alkyl. In certain embodiments, R^{b6} is hydrogen or —CH₃.

Two stapled polypeptides, appropriately functionalized (e.g., with Nu and/or E groups, as defined herein) may be covalently conjugated using a variety of reaction conditions. Conjugation of the Nu and E groups together or with bis-nucleophiles or bis-electrophiles is described herein, and, in certain embodiments may be classified as “Click chemistry.” Click chemistry is a chemical philosophy introduced by Sharpless in 2001 and describes chemistry tailored to generate substances quickly and reliably by joining small units together (see, e.g., Kolb, Finn and Sharpless Angewandte Chemie International Edition (2001) 40: 2004-2021; Evans, Australian Journal of Chemistry (2007) 60: 384-395). The reactions in Click chemistry should be modular, wide in scope, give high chemical yields, generate inoffensive byproducts, be stereospecific, be physiologically stable, exhibit a large thermodynamic driving force and/or have high atom economy. Several reactions have been identified which fit this concept:

(1) The Huisgen 1,3-dipolar cycloaddition (e.g., the Cu(I)-catalyzed stepwise variant, often referred to simply as the “click reaction”; see, e.g., Tornoe et al., Journal of Organic Chemistry (2002) 67: 3057-3064). Copper and ruthenium are the commonly used catalysts in the reaction. The use of copper as a catalyst results in the formation of 1,4-regioisomer whereas ruthenium results in formation of the 1,5- regioisomer;

(2) Other cycloaddition reactions, such as the Diels-Alder reaction;

(3) Nucleophilic addition to small strained rings like epoxides and aziridines;

(4) Nucleophilic addition to activated carbonyl groups; and

(4) Addition reactions to carbon-carbon double or triple bonds.

In certain embodiments, two stapled polypeptides of Formula (II), when Nu is —SH, —OH, —NHR^{b5}, —NH—NHR^{b5}, and —N═NH, and E is

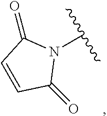 [SMILES: *N1C(=O)C=CC1=O]

are conjugated to form a homodimer or a heterodimer polypeptide of Formula (III) wherein Nu and E are joined to form a conjugated group of the Formula:

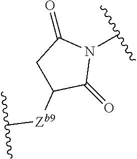

wherein Z^{b9} is —S—, —O—, —N(R^{b5})—, —NH—N(R^{b5})—, or —N═N—. In certain embodiments, Nu is —SH and Z^{b9} is —S—. In certain embodiments, Nu is —OH and Z^{b9} is —O—. In certain embodiments, Nu is —NHR^{b5} and Z^{b9} is —N(R^{5b})—. In certain embodiments, Nu is —NH—NHR^{b5} and Z^{b9} is —NH—N(R^{b5})—. In certain embodiments, Nu is —N═NH and Z^{b9} is —N═N—. In certain embodiments, R^{b5} is hydrogen.
In certain embodiments, two stapled polypeptides of Formula (II), when Nu is —SH, —OH, —NHR^{b}, —NH—NHR^{b5}, and —N═NH, and E is

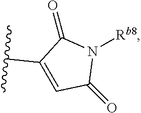

are conjugated to form a homodimer or a heterodimer polypeptide of Formula (III) wherein Nu and E are joined to form a conjugated group of the Formula:

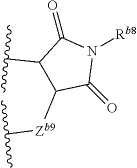

wherein Z^{b9} is —S—, —O—, —N(R^{b5})—, —NH—N(R^{b5})—, or —N═N—. In certain embodiments, Nu is —SH and Z^{b9} is —S—. In certain embodiments, Nu is —OH and Z^{b9} is —O—. In certain embodiments, Nu is —NHR^{b5} and Z^{b9} is —N(R^{b5})—. In certain embodiments, Nu is —NH—NHR^{b5} and Z^{b9} is —NH—N(R^{b5})—. In certain embodiments, Nu is —N═NH and Z^{b9} is —N═N—. In certain embodiments, R^{b5} is hydrogen.
In certain embodiments, two stapled polypeptides of Formula (II), when Nu is —SH, —OH, —NHR^{b5}, —NH—NHR^{b5}, and —N═NH, and E is

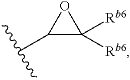

are conjugated to form a homodimer or a heterodimer polypeptide of Formula (III) wherein Nu and E are joined to form a conjugated group of the Formula:

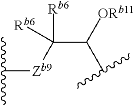

wherein Z^{b9} is —S—, —O—, —N(R^{5b})—, —NH—N(R^{b5})—, or —N═N—. In certain embodiments, Nu is —SH and Z^{b9} is —S—. In certain embodiments, Nu is —OH and Z^{b9} is —O—. In certain embodiments, Nu is —NHR^{b5} and Z^{b9} is —N(R^{b5})—. In certain embodiments, Nu is —NH—NHR^{b5} and Z^{b9} is —NH—N(R^{b5})—. In certain embodiments, Nu is —N═NH and Z^{b9} is —N═N—. In certain embodiments, R^{b5} is hydrogen. In certain embodiments, R^{b6} is hydrogen, optionally substituted aliphatic, or optionally substituted heteroaliphatic. In certain embodiments, R^{b6} is hydrogen or C₁₋₆alkyl. In certain embodiments, R^{b6} is hydrogen or —CH₃. In certain embodiments, R^{b11} is hydrogen. In certain embodiments, R^{b1} is an oxygen protecting group. In certain embodiments, two stapled polypeptides of Formula (II), when Nu is —SH, —OH, —NHR^{b5}, —NH—NHR^{b5}, and —N═NH, and E is —CO₂R^{b6}, —COX^{b7}, are conjugated to form a homodimer or a heterodimer polypeptide of Formula (III) wherein Nu and E are joined to form a conjugated group of the Formula:

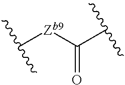

wherein Z^{b9} is —S—, —O—, —N(R^{b5})—, —NH—N(R^{b5})—, or —N═N—. In certain embodiments, Nu is —SH and Z^{b9} is —S—. In certain embodiments, Nu is —OH and Z^{b9} is —O—. In certain embodiments, Nu is —NHR^{b5} and Z^{b9} is —N(R^{b5})—. In certain embodiments, Nu is —NH—NHR^{b5} and Z^{b9} is —NH—N(R^{b5})—. In certain embodiments, Nu is —N═NH and Z^{b9} is —N═N—. In certain embodiments, R^{b5} is hydrogen.
In certain embodiments, two stapled polypeptides of Formula (II), when Nu is —SH, —OH, —NHR^{b}, —NH—NHR^{b}, and —N═NH, and E is

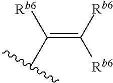

are conjugated to form a homodimer or a heterodimer polypeptide of Formula (III) wherein Nu and E are joined to form a conjugated group of the Formula:

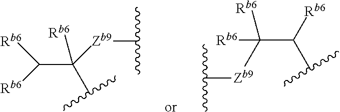

wherein Z^{b9} is —S—, —O—, —N(R^{5b})—, —NH—N(R^{b5})—, or —N═N—. In certain embodiments, Nu is —SH and Z^{b9} is —S—. In certain embodiments, Nu is —OH and Z^{b9} is —O—. In certain embodiments, Nu is —NHR^{b5} and Z^{b9} is —N(R^{b5})—. In certain embodiments, Nu is —NH—NHR^{b5} and Z^{b9} is —NH—N(R^{b5})—. In certain embodiments, Nu is —N═NH and Z^{b9} is —N═N—. In certain embodiments, R^{b5} is hydrogen. In certain embodiments, R^{b6} is hydrogen, optionally substituted aliphatic, or optionally substituted heteroaliphatic. In certain embodiments, R^{b6} is hydrogen or C₁₋₆alkyl. In certain embodiments, R^{b6} is hydrogen or —CH₃.
In certain embodiments, two stapled polypeptides of Formula (II), when Nu is —SH, —OH, —NHR^{b5}, —NH—NHR^{b5}, and —N═NH, and E is

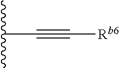

are conjugated to form a homodimer or a heterodimer polypeptide of Formula (III) wherein Nu and E are joined to form a conjugated group of the Formula:

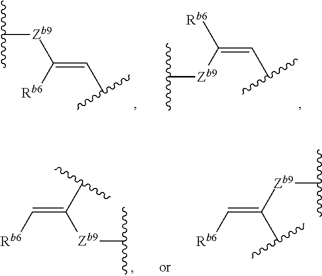

wherein Z^{b9} is —S—, —O—, —N(R^{5b})—, —NH—N(R^{b5})—, or —N═N—. In certain embodiments, Nu is —SH and Z^{b9} is —S— (a thiol-yne reaction). In certain embodiments, Nu is —OH and Z^{b9} is —O—. In certain embodiments, Nu is —NHR^{b5} and Z^{b9} is —N(R^{b}S)—. In certain embodiments, Nu is —NH—NHR^{b5} and Z^{b9} is —NH—N(R^{b5})—. In certain embodiments, Nu is —N═NH and Z^{b9} is —N═N—. In certain embodiments, R^{b5} is hydrogen. In certain embodiments, R^{b6} is hydrogen, optionally substituted aliphatic, or optionally substituted heteroaliphatic. In certain embodiments, R^{b6} is hydrogen or C₁₋₆alkyl. In certain embodiments, R^{b6} is hydrogen or —CH₃.
In certain embodiments, two stapled polypeptides of Formula (II), when Nu is Nu is —SH, —OH, —NHR^{b5}, —NH—NHR^{b5}, and —N═NH, and E is

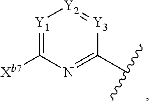

are conjugated to form a homodimer or a heterodimer polypeptide of Formula (III) wherein Nu and E are joined to form a conjugated group of the Formula:

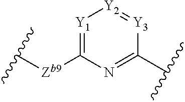

wherein Z^{b9} is —S—, —O—, —N(R^{b5})—, —NH—N(R^{b5})—, or —N═N—. In certain embodiments, Nu is —SH and Z^{b9} is —S— (a thiol-yne reaction). In certain embodiments, Nu is —OH and Z^{b9} is —O—. In certain embodiments, Nu is —NHR^{b5} and Z^{b9} is —N(R^{b5})—. In certain embodiments, Nu is —NH—NHR^{b5} and Z^{b9} is —NH—N(R^{b5})—. In certain embodiments, Nu is —N═NH and Z^{b9} is —N═N—. In certain embodiments, two stapled polypeptides of Formula (II), when Nu is Nu is —SH, —OH, —NHR^{b5}, —NH—NHR^{b5}, and —N═NH, and E is

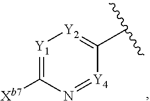

are conjugated to form a homodimer or a heterodimer polypeptide of Formula (III) wherein Nu and E are joined to form a conjugated group of the Formula:

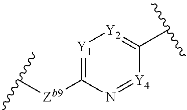

wherein Z^{b9} is —S—, —O—, —N(R^{b5})—, —NH—N(R^{b5})—, or —N═N—. In certain embodiments, Nu is —SH and Z^{b9} is —S— (a thiol-yne reaction). In certain embodiments, Nu is —OH and Z^{b9} is —O—. In certain embodiments, Nu is —NHR^{b5} and Z^{b9} is —N(R^{b5})—. In certain embodiments, Nu is —NH—NHR^{b5} and Z^{b9} is —NH—N(R^{b5})—. In certain embodiments, Nu is —N═NH and Z^{b9} is —N═N—. In certain embodiments, two stapled polypeptides of Formula (II), when Nu is —N═NH and E is —CHO, are conjugated to form a homodimer or a heterodimer polypeptide of Formula (III) wherein Nu and E are joined to form a conjugated group of the Formula:

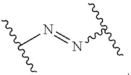

In certain embodiments, two stapled polypeptides of Formula (II), when Nu is —NHR^{b5}, R^{b5} is hydrogen, and E is —CHO, are conjugated to form a homodimer or a heterodimer polypeptide of Formula (III) wherein Nu and E are joined to form a conjugated group of the Formula:

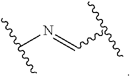

In certain embodiments, two stapled polypeptides of Formula (II), when Nu is

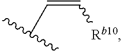

and E is

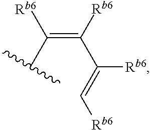

are conjugated via a Diels Alder reaction to form a homodimer or a heterodimer polypeptide of Formula (III) wherein Nu and E are joined to form a conjugated group of the Formula:

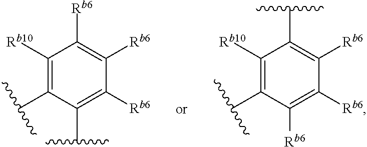

In certain embodiments, R^{b10} is hydrogen. In certain embodiments, R^{b6} is hydrogen or optionally substituted aliphatic, e.g., acyl.
In certain embodiments, two stapled polypeptides of Formula (II), when Nu is —N₃, and E is

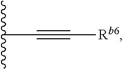

are conjugated via a Huisgen 1,3-dipolar cycloaddition reaction to form a homodimer or a heterodimer polypeptide of Formula (III) wherein Nu and E are joined to form a conjugated group of the Formula:

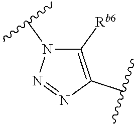

(1,4 regioisomer) or

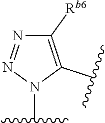

(1,5 regioisomer).
In certain embodiments, R^{b6} is hydrogen, optionally substituted aliphatic, or optionally substituted heteroaliphatic. In certain embodiments, R^{b6} is hydrogen or C₁-6alkyl. In certain embodiments, R^{b6} is hydrogen or —CH₃. In certain embodiments, R^{b6} is hydrogen. In certain embodiments, two stapled polypeptides of Formula (II), each comprising a group Nu, wherein each Nu is independently selected from —SH, —OH, —NHR^{b}, —NH—NHR^{b5}, and —N═NH, are conjugated by reacting the two polypeptides with a bis-electrophile of Formula

X^{b7}—W₃—X^{b7}

wherein X^{b7} is a leaving group, and W₃ is selected from the group consisting of optionally substituted alkylene; optionally substituted alkenylene; optionally substituted alkynylene; optionally substituted heteroalkylene; optionally substituted heteroalkenylene; optionally substituted heteroalkynylene; optionally substituted arylene; or optionally substituted heteroarylene, to provide a conjugated group of Formula:

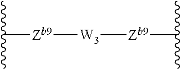

wherein Z^{b9} is —O—, —S—, —N(R^{b5})—, —NH—N(R^{b5})—, or —N═N—. In certain embodiments, each Nu is —SH and each Z^{b9} is —S—. In certain embodiments, each Nu is —OH and each Z^{b9} is —O—. In certain embodiments, each Nu is —NHR^{b5} and each Z^{b9} is —N(R^{b5})—. In certain embodiments, each Nu is —NH—NHR^{b5} and each Z^{b9} is —NH—N(R^{b5})—. In certain embodiments, each Nu is —N═NH and each Z^{b9} is —N═N—. In certain embodiments, W₃ is optionally substituted alkylene. In certain embodiments, W₃ is optionally substituted arylene. In certain embodiments, W₃ is optionally substituted heteroarylene. Various combinations of the two Nu groups and two X^{b7} groups are contemplated. In certain embodiments, the two Nu groups, and thus the two Z^{b9} groups, are the same. In certain embodiments, the two Nu groups, and thus the two Z^{b9} groups, are different. In certain embodiments, the two X^{b7} groups are the same. In certain embodiments, the two X^{b7} groups are different.
In certain embodiments, wherein W₃ is optionally substituted alkylene, the bis-electrophile is of the Formula:

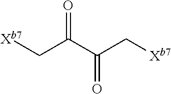

wherein X^{b7} is —Br, —Cl, or —I.
For example, when the bis-electrophile is of the Formula:

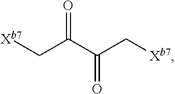

the resulting conjugated group is of the Formula

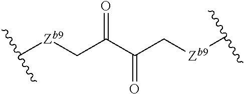

In certain embodiments, wherein W₃ is optionally substituted heteroarylene, the bis-electrophile is of the Formula:

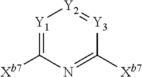

wherein X^{b7} is —Br, —Cl, or —I.

For example, when the bis-electrophile is of the Formula:

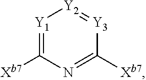

the resulting conjugated group is of the Formula

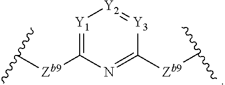

In certain embodiments, two stapled polypeptides of Formula (II), each comprising a group E, wherein each E is independently selected from a leaving group, —CHO, —CO₂R^{b6}, —COX^{b7},

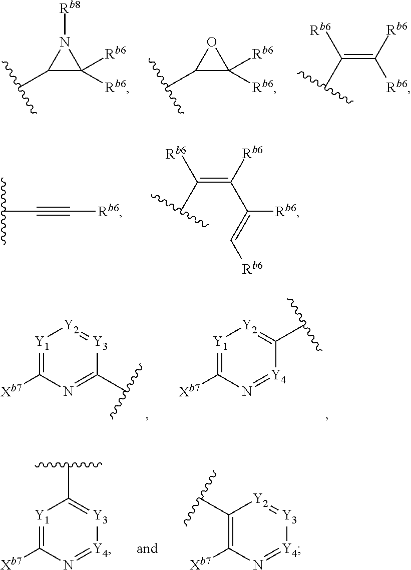

are conjugated by reacting the two polypeptides with a bis-nucleophile Nu-W₄-Nu wherein each Nu is —SH, —OH, —NHR^{b5}, —NH—NHR^{b5}, —N═NH, —N═C, —N₃, or

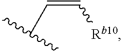

and W₄ is independently represents optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene; to provide a conjugated polypeptide. The two E groups conjugated to W₄ independently correspond to any of the above described conjugated groups, also listed below:

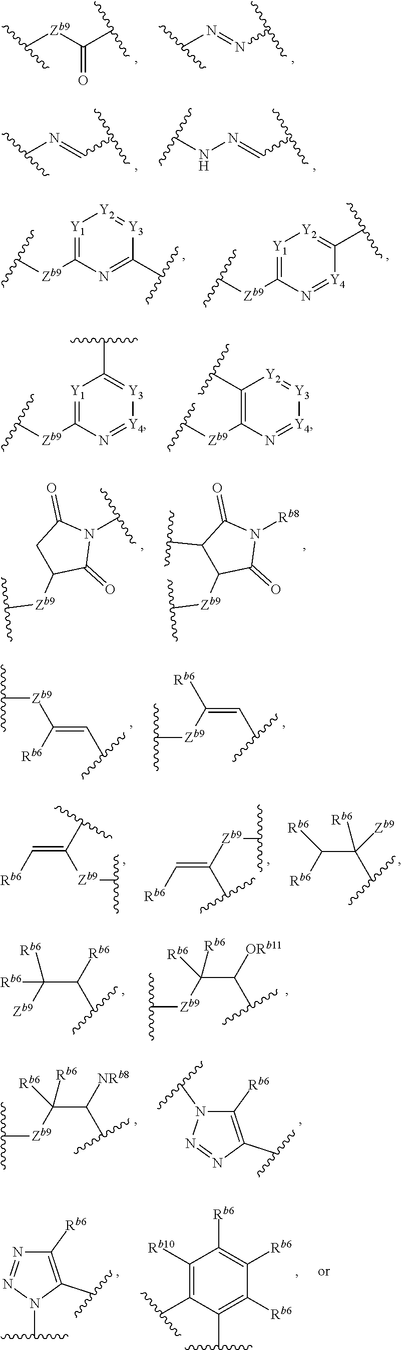

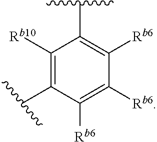

Various combinations of the two E groups are contemplated. In certain embodiments, the two E groups are the same. In certain embodiments, the two E groups are different. In certain embodiments, the two Nu groups, and thus the two Z^{b9} groups, are different. In certain embodiments, the two X^{b7} groups are the same. In certain embodiments, the two X^{b7} groups are different.
In certain embodiments, the amino acid of Formula (iii) is of the Formula:

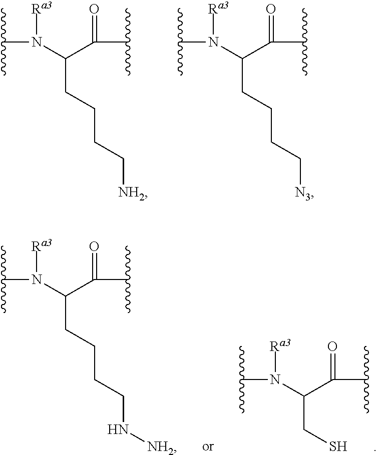

In certain embodiments, the amino acid of Formula (iv) is of the Formula:

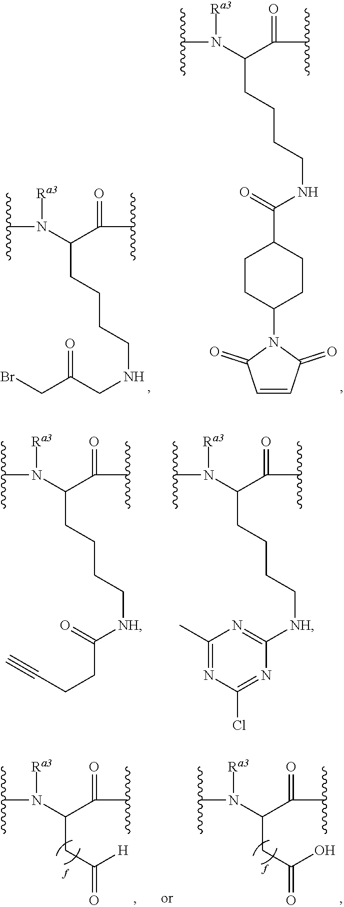

wherein f is an integer between 1 and 10, inclusive (e.g., f is 1, 2, 3, 4, 5, 6, 7, 8, 9, or 10). In certain embodiments, f is 1.

The peptides described herein can further be modified. Some modifications may increase the stability and activity of a peptide to enable reduced dosing level or frequency, as well as enable alternative routes of administration, e.g., oral or inhalation. The following are examples of modifications of peptides that may increase stability, activity, specificity, and/or efficacy.

1) Replace labile amino acids with ones that increase stability and improve activity. Such replacement can be performed based upon HPLC analysis of peptide incubation in serum or liver/lung homogenates. For example, lysines and arginines that are recognized by trypsin can be replaced with glutamine.

2) Replace one or more L-amino acids with D-amino acids. D-amino acids are unnatural amino acids which are less likely to be attacked by proteases. For example, a protease cleavage site prediction program has identified 8 cleavage sites for trypsin. To reduce the probability of this proteolysis, one or more L-arginines (R) in the peptide can be replaced with D-arginines as described by Powell et al. (Pharm. Res., 1993, 10, 1268-1273.)

3) Reduce the size of the peptide. Removing non-essential sequences or individual residues may improve entry into target cells. Use of smaller transduction domains, such as those described herein, may be carried out. An example of similar successful manipulation of somastatin is described in Harris (Gut, 1994, 35(3 Suppl), S1-4).

4) Oligomerize the peptide. The peptide molecular weight is less than 5 kDa, and hence is likely to be rapidly excreted through kidneys. Oligomerization may improve bioavailability. Oligomerization can be carried out by synthesizing repeating sequences such as dimers, trimers and polymers to increase the molecular mass so the peptide will be more stable and less easily excreted. These oligomers (n=number of repeats) may consist of repeats of the whole structure.

5) Cyclize the peptide. Cyclizing a peptide may protect it against proteolysis and degradation. As described herein, cyclizing a peptide may occur via side-chain to side-chain. Further, cyclizing a peptide may occur through commonly used coupling methods using agents such as, for example, p-nitrophenyl esters, the azide method, 2,4,5-trichlorophenyl and pentafluorophenyl esters and the mixed anhydride method. Other more direct methods of activation using N,N-dicyclohexylcarbodiimide (DCC) with catalysts such as HOBt, HONSu, and HOAt are also suitable. Use of use of a water soluble carbodiimide EDC (1-ethyl-3-(3-dimethylaminopropyl)carbodiimide hydrochloride) is also suitable. Suitable successors to the conventional azide coupling includes the use of DPPA and PyBrop. Also included are:

1-benzotriazole-tris-dimethyl aminophosphonium hexafluorophosphate (BOP), O-(benzotriazol-1-yl)-1,1,3,3-tetramethyl uronium hexafluorophosphate (HBTU), 1-benzotriazolyloxy-tris-pyrrolidino-phosphonium hexafluorophosphate (PyBOP), O-(benzotriazol-1-yl)-1,1,3,3-tetramethyl uronium tetrafluoroborate (TBTU), 7-azabenzotriazol-1-yloxytrispyrrolidino phosphonium hexafluorophosphate (PyAOP), O-(7-azabenzotriazol-1-yl)-1,1,3,3-tetramethyl uronium hexafluorophosphate (HATU), 7-azabenzotriazol-1-yloxy-tris-dimethyl aminophosphonium hexafluorophosphate (AOP), O-(7-azabenzotriazol-1-yl)-1,1,3,3-tetramethylene uronium hexafluorophosphate (HAPyU), and O-(7-azabenzotriazol-1-yl)-1,1,3,3-pentamethylene uronium hexafluorophosphate (HAPipU). See, Davies, J. Peptide Science, 2003, 9, 471-501.

6) Internal hydrocarbon “stapling”, that stabilize peptide conformations. Examples of such methods are described by Bird et al., Methods Enzymol 2008, 446, 369-386; Madden et al., Chem Commun 2009, 37, 5588-5590; Kim et al., Org Lett 2010, 12, 3046-3049; Bird et al., PNAS 2010, 107, 14093-14098; Jacobsen et al., J Org Chem 2011, 76, 1228-1238 and Verdine & Hilinski, Methods Enzymol 2012, 503, 3-33.

7) PEGylate. Adding polyethylene glycol of different sizes, e.g., 40 kDa, to the amino-, carboxy-, and/or inside of the molecule may improve its stability. An example of the latter approach in stabilizing interferon alpha is described by Ramon et al. (Pharm. Res., 2005, 22, 1374-1386).

8) Other modifications: C-terminal amidation or N-terminal acetylation as described in, for example, Brinckerhoff et al. (Int'l J. Cancer, 1999, 83, 326-334), or N-pyroglutamylation as described in, for example, Green et al. (J. Endocrinol., 2004, 180, 379-388). Other examples include conjugation of various fatty acids ranging from 4-18 chain length as described in, for example, DasGupta et al. (Biol. Pharma. Bull., 2002, 25, 29-36).

9) Biodegradable modifications: e.g., polymers of N-acetylneuraminic adid (poysialic acids) as described in, for example, Georgiadis et al. (Cell. Mol. Life Sci., 2000, 57, 1964-1969).

10) Combination with delivery systems that enable sustained release. Carriers such as liposomes, microspheres or microcapsules, poly lactic acid (PLA), poly lactic/glycolic acid (PLGA) as described in, for example, Heya et al. (J. Pharm. Sci., 1994, 83, 798-801), nanoparticles and emulsions, cyclodextrins and derivatives.

11) Formulations that protect peptides such as those containing different types of protease inhibitors. In addition, formulation containing multifunctional polymers which exhibit mucoadhesive properties as well as enzyme inhibitory activity, e.g., poly(acrylates), thiolated polymers, and polymer-enzyme inhibitor conjugates.

Other modifications may further include conjugation of the stapled polypeptide, or a synthetically modified stapled polypeptide, with a biologically active agent, label or diagnostic agent anywhere on the polypeptide scaffold, e.g., such as at the N-terminus of the polypeptide, the C-terminus of the polypeptide, on an amino acid side chain of the polypeptide, or at one or more modified or unmodified stapled sites. Such modification may be useful in delivery of the peptide or biologically active agent to a cell, tissue, or organ. Such modifications may allow for targeting to a particular type of cell or tissue. Conjugation of an agent (e.g., a label, a diagnostic agent, a biologically active agent) to the polypeptide may be achieved in a variety of different ways. The agent may be covalently conjugated, directly or indirectly, to the polypeptide at the site of stapling, or to the N-terminus or the C-terminus of the polypetide chain. Alternatively, the agent may be noncovalently conjugated, directly or indirectly, to the polypeptide at the site of stapling, or to the N-terminus or the C-terminus of the polypetide chain. Indirect covalent conjugation is by means of one or more covalent bonds. Indirect noncovalent conjugation is by means of one or more noncovalent bonds. Conjugation may also be via a combination of non-covalent and covalent forces/bonds. The agent may also be conjugated through a covalent or noncovalent linking group. Any number of covalent bonds may be used in the conjugation of a biologically active agent and/or diagnostic agent to the polypeptide present invention. Such bonds include amide linkages, ester linkages, disulfide linkages, carbon-carbon bonds, carbamate, carbonate, urea, hydrazide, and the like. In some embodiments, the bond is cleavable under physiological conditions (e.g., enzymatically cleavable, cleavable with a high or low pH, with heat, light, ultrasound, x-ray, etc.). However, in some embodiments, the bond is not cleavable.

In some embodiments, peptidomimetics of the peptides described herein are provided. The use of peptides as lead compounds, and subsequently conversion into low-molecular-weight nonpeptide molecules (peptidomimetics), have successfully led to development of small-molecule antagonists of intracellular targets (Bottger et al., J. Mol. Biol., 1997, 269, 744-56; Bottger et al., Oncogene, 1996, 13, 2141-7). Therefore, peptidomimetics have emerged as a powerful means for overcoming the obstacles inherent in the physical characteristics of peptides, improving their therapeutic potential (Kieber-Emmons et al., Curr. Opin. Biotechnol., 1997, 8, 435-41; Beeley, Trends Biotechnol., 1994, 12, 213-6; and Moore et al., Trends Pharmacol. Sci., 1994, 15, 124-9). In some embodiments, compared to native peptides, peptidomimetics possess desirable pharmacodynamic properties superior to natural peptides, including good oral activity, long duration of action, better transport through cellular membranes, decreased rate of excretion, and decreased hydrolysis by peptidases.

Development of a small molecule peptidomimetic generally involves identification of the smallest functional peptide unit capable of inhibiting the targeted interaction. A growing body of literature demonstrates that high-affinity ligands can be selected from peptide libraries displayed on bacteriophage (Sulochana et al., Curr. Pharm. Des., 2007, 13, 2074-86; Cwirla et al., Proc. Natl. Acad. Sci. USA, 1990, 87, 6378-82; Scott et al., Science, 1990, 249, 386-90; and Devlin et al., Science, 1990, 249, 404-6), and many applications have been directed toward antagonizing the function of a protein ligand (Dower, Curr. Opin. Chem. Biol., 1998, 2, 328-34; and Sidhu et al., Methods Enzymol., 2000, 328, 333-63). Because the libraries can be very large (10¹¹ or more individual members), no initial assumptions are required concerning how to bias the library, nor the selective enrichment of rare binding phage through biological amplification and rescreening. Those sequences that bind can be identified easily by sequencing their encoding DNA.

In some embodiments, peptide ligands such identified further serve as starting points for a combinatorial chemistry approach or a medicinal chemistry-based peptidomimetic approach for the development of new directed therapeutic agents. In addition, the determination of the structural basis for the high-binding affinity of these peptides for their substrate contributes to the rational design of a therapeutic agent.

In some embodiments, peptides are synthesized de novo. A variety of peptide synthesis methods may be utilzed. Examples include, but are not limited to, solid-phase peptide synthesis (SPPS), (R. B. Merrifield (1963). “Solid Phase Peptide Synthesis. I. The Synthesis of a Tetrapeptide”. J. Am. Chem. Soc. 85 (14): 2149-2154; Mitchell, A. R. K., S. B. H.; Engelhard, M.; Merrifield, R. B. (1978). “A new synthetic route to tert-butyloxycarbonylaminoacyl-4-(oxymethyl)phenylacetamidomethyl-resin, an improved support for solid-phase peptide synthesis”. J. Org. Chem. 43 (13): 2845-2852). Recent developments in synthesis methods are further described in Hojo, Curr Opin Struct Biol 2014, 26C, 16-23; Ramakers et al., Chem Soc Rev 2014, 43, 2743-2756 and Chandrudu et al., Molecules 2013, 18, 4373-4388.

In SPPS, Small solid beads, insoluble yet porous, are treated with functional units (‘linkers’) on which peptide chains can be built. The peptide will remain covalently attached to the bead until cleaved from it by a reagent such as anhydrous hydrogen fluoride or trifluoroacetic acid. The peptide is thus ‘immobilized’ on the solid-phase and can be retained during a filtration process, whereas liquid-phase reagents and by-products of synthesis are flushed away.

The general principle of SPPS is one of repeated cycles of coupling-wash-deprotection-wash. The free N-terminal amine of a solid-phase attached peptide is coupled (see below) to a single N-protected amino acid unit. This unit is then deprotected, revealing a new N-terminal amine to which a further amino acid may be attached. The superiority of this technique partially lies in the ability to perform wash cycles after each reaction, removing excess reagent with all of the growing peptide of interest remaining covalently attached to the insoluble resin.

There are two majorly used forms of SPPS-Fmoc and Boc. Unlike ribosome protein synthesis, solid-phase peptide synthesis proceeds in a C-terminal to N-terminal fashion. The N-termini of amino acid monomers is protected by either of these two groups and added onto a deprotected amino acid chain.

The present invention further provides the use and methods of using the peptides described herein in the inhibition of transcription factor activity. Such peptides find use in the treatment of disease and related conditions. Examples of diseases and conditions treated by the peptides described herein include but are not limited to cancer and stem cell related diseases.

In some embodiments, the peptides described herein are administered in combination with one or more additional agents useful in the treatment of disease (e.g., cancer).

Embodiments of the present invention further provide pharmaceutical compositions (e.g., comprising one or more of the therapeutic agents described above). The pharmaceutical compositions of the present invention may be administered in a number of ways depending upon whether local or systemic treatment is desired and upon the area to be treated.

Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

In some embodiments, peptides described herein are introduced either subcutaneously or intravenously. In some embodiments, peptides are administered using a gene delivery technique to express the peptide in taget (e.g., cardiac) cells. In some embodiments, recombinant adeno-associated virus (serotype 9) vectors carrying peptide-encoding cDNA with a cytomegalovirus promoter (AAV9) (Cutler M J, et al. Circulation. 2012; 126:2095-2104; herein incorporated by reference in its entirety) are utilized.

Pharmaceutical compositions and formulations for topical administration (e.g., to tissues, wounds, organs, etc) may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets.

Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the active agents of the formulation.

Dosing is dependent on severity and responsiveness of the disease state or condition to be treated, with the course of treatment lasting from several days to several months, or until a cure is effected or a diminution of the disease state is achieved. In some embodiments, treatment is administered in one or more courses, where each course comprises one or more doses per day for several days (e.g., 1, 2, 3, 4, 5, 6) or weeks (e.g., 1, 2, or 3 weeks, etc.). In some embodiments, courses of treatment are administered sequentially (e.g., without a break between courses), while in other embodiments, a break of 1 or more days, weeks, or months is provided between courses. In some embodiments, treatment is provided on an ongoing or maintenance basis (e.g., multiple courses provided with or without breaks for an indefinite time period). Optimal dosing schedules can be calculated from measurements of drug accumulation in the body of the patient. The administering physician can readily determine optimum dosages, dosing methodologies and repetition rates.

In some embodiments, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues.

### EXPERIMENTAL

The following examples are provided in order to demonstrate and further illustrate certain preferred embodiments and aspects of the present invention and are not to be construed as limiting the scope thereof.

### Example 1

### Design and Synthesis of sDBDs with Improved Specificity and Affinity for E-Box DNA

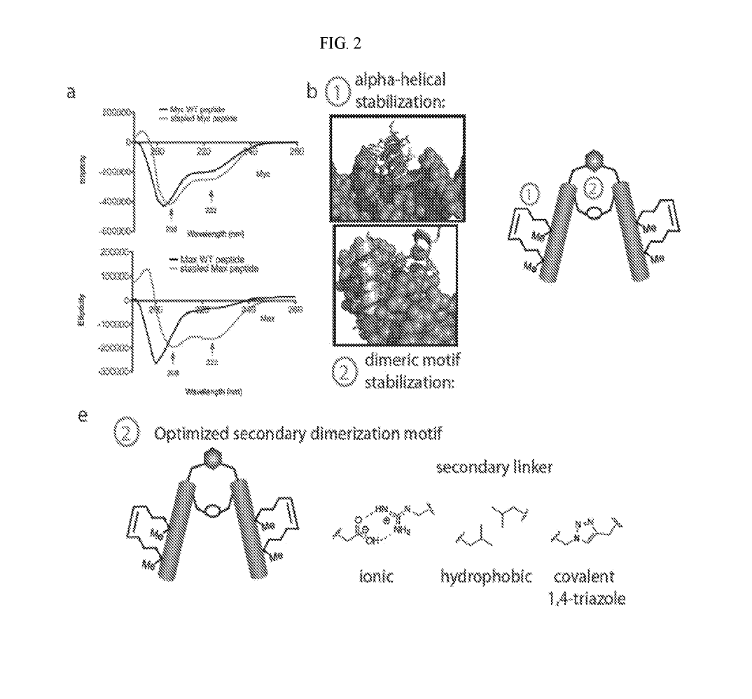

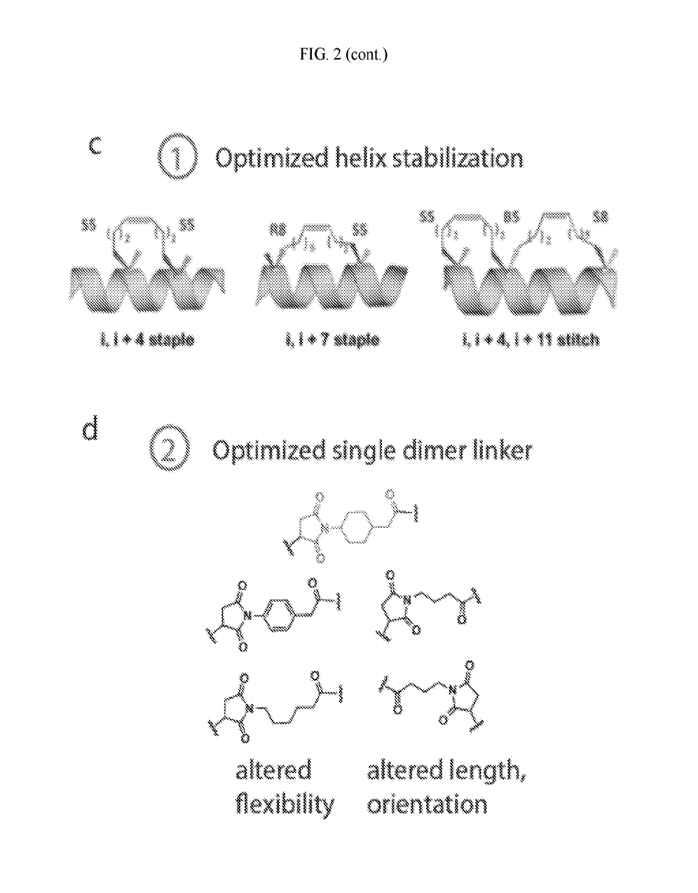

The first generation of sDBDs has proven that fully synthetic, multi-domain stapled peptides are capable of binding E-box sequences with affinity and specificity comparable to and competitive with the natural Myc/Max dimer. The position of the hydrocarbon helix-stabilization motif was systematically tested in initial experiments. This parameter revealed that the most potent sDBD exhibited the highest degree of individual helix stabilization relative to their unmodified counterparts (FIG. 2A). Other important structural features, such as the dimerization linker and chemistry, as well as the DNA-binding residues themselves, remained constant.

An iterative medicinal chemistry effort is undertaken to synthesize focused libraries of sDBDs with optimized: 1) individual helix stabilization; 2) dimerization motif, which will include altering the length and rigidity of the current thiol-maleimide linker as well as inclusion of an additional linker to constrain the orientation of DNA-binding helices (FIG. 2B). Optimization of individual helix stabilization is accomplished by testing complimentary hydrocarbon stabilization linkers. First generation sDBDs employed a single i→i+4 hydrocarbon macrocyclic tether, and experiments will also test the incorporation of i→i+7 and i→i+4→i+11 designs (FIG. 2C), which have been shown in several studies to provide sequence-dependent variability in stabilization(7, 8). Circular dichroism spectroscopy is used to measure the relative degree of helix stabilization, and optimized individual Myc- and Max-derived stabilized peptides are carried forward to further testing.

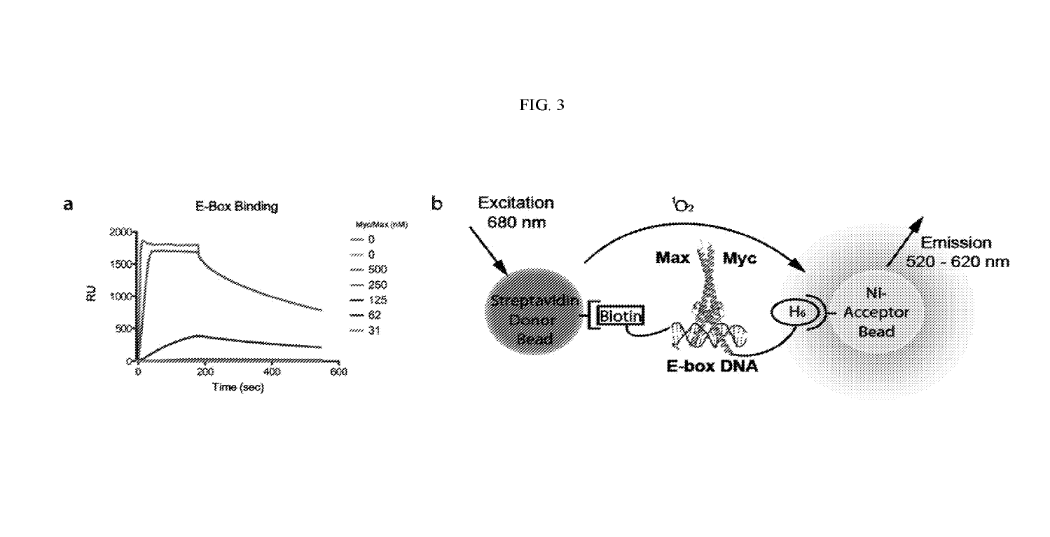

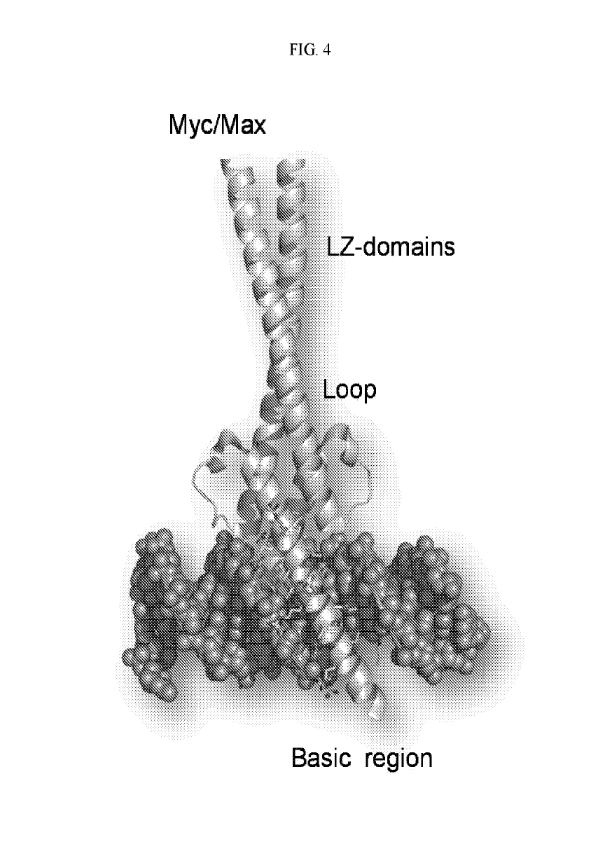

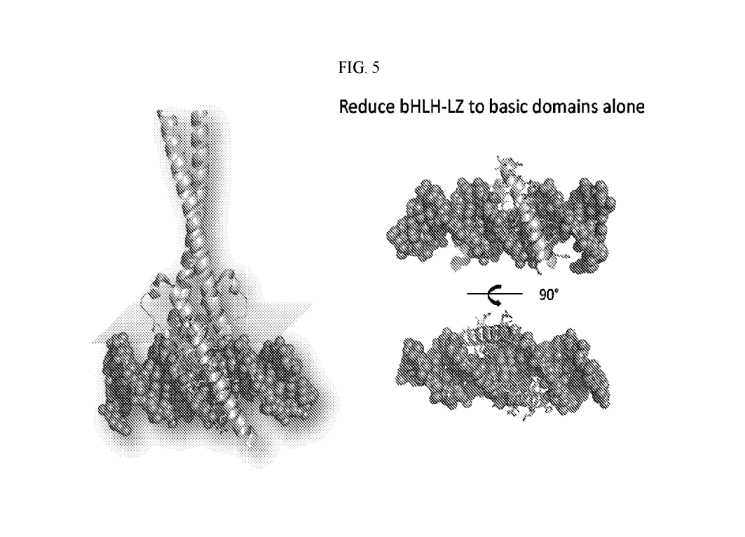

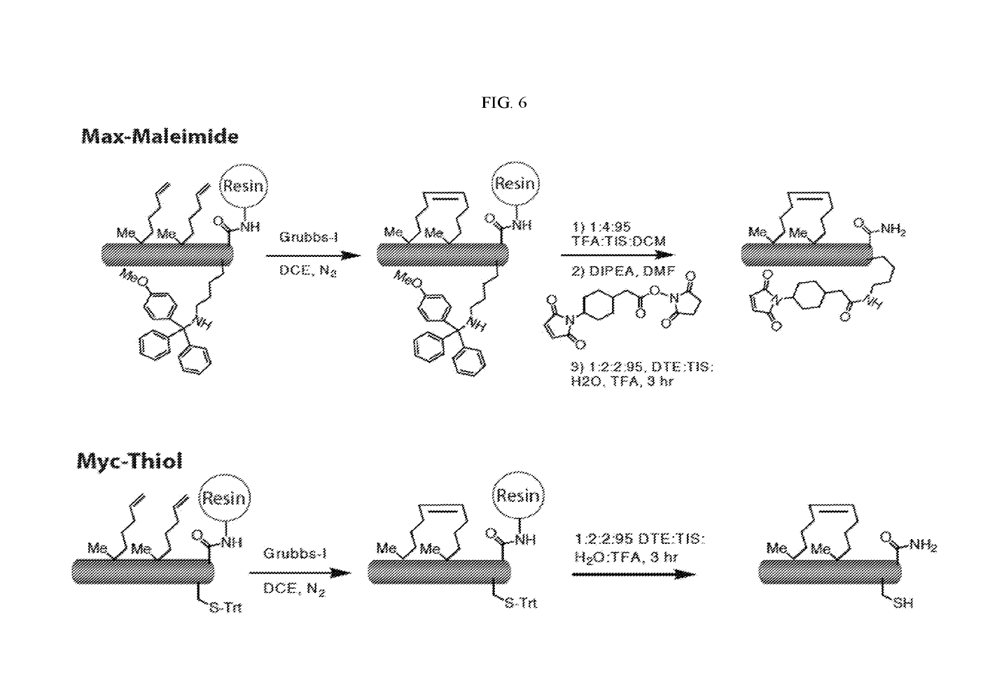

In the second phase of medicinal chemistry, alternative dimerization linkers and chemistries are tested for their effect on the tertiary structure and DNA-binding properties of the resulting sDBDs. The cyclohexyl-maleimide linker employed in the first round of sDBD synthesis was chosen based on molecular modeling, where the distance between modeled Lys and Cys residues in each helix (˜7.5 Å) closely matched the length of the linker (8.3 Å). To optimize this linker and the resulting structural constraints it imparts on the presentation of the DNA-binding helices, linkers incorporating the same chemical reactivity, but altered length and rigidity, are tested (FIG. 2D). In parallel, the effect of a second point of contact between the two helices is explored. The rationale for this design element is that in the natural bHLH structure the leucine zipper/loop domains of the protein not only juxtapose the DNA-binding helices, but also to properly orient them for DNAbinding. This preorganization is associated with both binding affinity and specificity(9, 10). By comparison, the current sDBDs have a much greater degree of flexibility around the dimerization linker and therefore are free to sample many nonproductive conformations. A second point of contact between the dimerized helices by either non-covalent interactions that mimic natural protein design (e.g. complimentary ionic bonding or hydrophobic interactions, FIG. 2E) or through inclusion of an orthogonal chemical crosslink (e.g. an triazole linker formed by alkyne- and azide-containing amino acids, FIG. 2F) is included. These dimerization motifs are tested individually and then in parallel. Primary screening for DNA-binding affinity and specificity is assessed using gel-shift assays with E-box containing and mutant oligonucleotides, with first generation sDBDs and recombinant Myc/Max protein serving as standards. Secondary screening assays include surface plasmon resonance (e.g., Biacore) binding assays (FIG. 3A), which provide both thermodynamic and kinetic binding data, as well as ALPHAscreen proximity assays, which can detect and quantify direct competition with Myc/Max for E-box DNA binding (FIG. 3B and FIGS. 1E, F). X-ray crystal structures of sDBD-DNA complexes is also performed. Cell penetration is assayed using fluorescently labeled sDBDs and fluorescence activated cell sorting (FACS) as well as confocal microscopy.

### Example 2

### Validation of Optimized sDBD E-Box Binding and Inhibition of Myc-Dependent Gene Expression in Cells

Optimized sDBDs are tested in situ for the ability to specifically bind E-box sites in the genome and subsequently repress Myc-dependent gene expression. First, sDBD binding in the genome is mapped using chromatin immunoprecipitation coupled to DNA sequencing (ChIP-seq) approaches. sDBD analogs harboring a biotin group to facilitate binding site determination in live cells are used. A number of well-characterized human and murine cell lines having a known phenotypic dependence on Myc expression are available, including transformed murine cell lines in which Myc activity can be modulated through induction of a dominant-negative Myc protein (Omomyc)(11), as well as human-derived Burkitt's Lymphoma cell lines, where Myc activation is pathologic. To gain loci-specific information on sDBD targeting, Raji Burkitt's Lymphoma cells are treated with biotinylated sDBDs identified, processed for ChIP-purification of sDBD-bound DNA and analysis of these sites through DNA sequencing.

In addition, gene expression profiling is performed to assess the downstream transcriptional effects of sDBDs on well validated Myc-responsive genes, for example CDK4, CCND2, ID2, and CUL1. One or more negative control sDBDs having a scrambled basic region as well as the inducible expression of Omomyc to blunt Myc-driven gene expression are used. Genome-wide microarray (Affymetrix U133 Plus 2.0) and RNA-sequencing gene expression analysis is performed to assess the entire spectrum of transcriptional effects caused by treatment of Raji and Ramos cell lines with sDBDs. The effect of sDBD or Omomyc treatment on validated Myc-target genes in these cell lines is used as a first measure of pathway interferences. Additionally, these expression profiles are be used to generate gene sets representing transcripts that are reproducibly up-regulated and down-regulated upon treatment, and these gene sets are then used to query the entire library of gene sets available in the Broad Institute database Molecular Signatures Database (MSigDB), using Gene Set Enrichment Analysis(3, 12). Particularly informative is the comparison of the expression changes brought about in the Evan system between treatment of cells with doxycycline (the Omomyc inducer) versus treatment with an sDBD.

Statistical considerations that are agreed upon as best practices is applied to control for large profiling and sequencing data sets and identify enriched, statistically significant binding sites and gene expression changes associated with sDBD treatment. ChIP-seq data analysis is supported by bioinformatics support in the Institute for Genomics and Systems Biology, which has contributed directly to these practices in large scale genome profiling efforts such as the ENCODE consortium(13). Specifically, biological and technical replicates of all experimental conditions are repeated to derive Pvalues, q-values, ChIP-to-input enrichment ratios, as well as the irreproducibility discovery rate (IDR).

### Example 3

### Effects of Direct Myc Inhibition on Oncogenic Phenotypes in Myc-Dependent Burkitt's Lymphoma Cells

In cancer cells, Myc has been shown to drive expression of diverse genes leading to increased pathogenicity through augmented aerobic glycolysis, protein synthesis, cell cycle progression and proliferation(14). Therefore, sDBDs exhibiting the ability to specifically modulate Myc-regulated genes in expression profiling studies are expected to have profound effects on cellular proliferation and differentiation. Several established Myc-dependent phenotypes are used to identify the functional effects of direct Myc antagonism in Burkitt's lymphoma cells. Myc-driven glycolytic remodeling is explored using metabolomic profiling to monitor cellular glucose uptake, fermentation and respiration in Raji and Ramos cell lines. The effect sDBD treatment or Omomyc expression at time-points associated with inhibition of Myc-driven gene expression on protein synthesis is quantified using pulse-chase labeling of cells with 35S-Methionine. Finally, the cumulative effect of Myc-driven gene expression is augmented cell cycle progression and cell proliferation in numerous cancers. Systems where Myc-inhibition has been modeled genetically through Omomyc induction have revealed profound regression of both incipient and established lung adenocarcinoma tumors in vivo (11). Therefore pharmacologic inhibition of Myc is expected to drastically effect cell cycle progression and proliferation of Myc-dependent tumor cells. These phenotypes are measured in Raji and Ramos cells in response to dose- and time-dependent treatment of optimized sDBDs as well as induction of Omomyc expression.

### Example 4

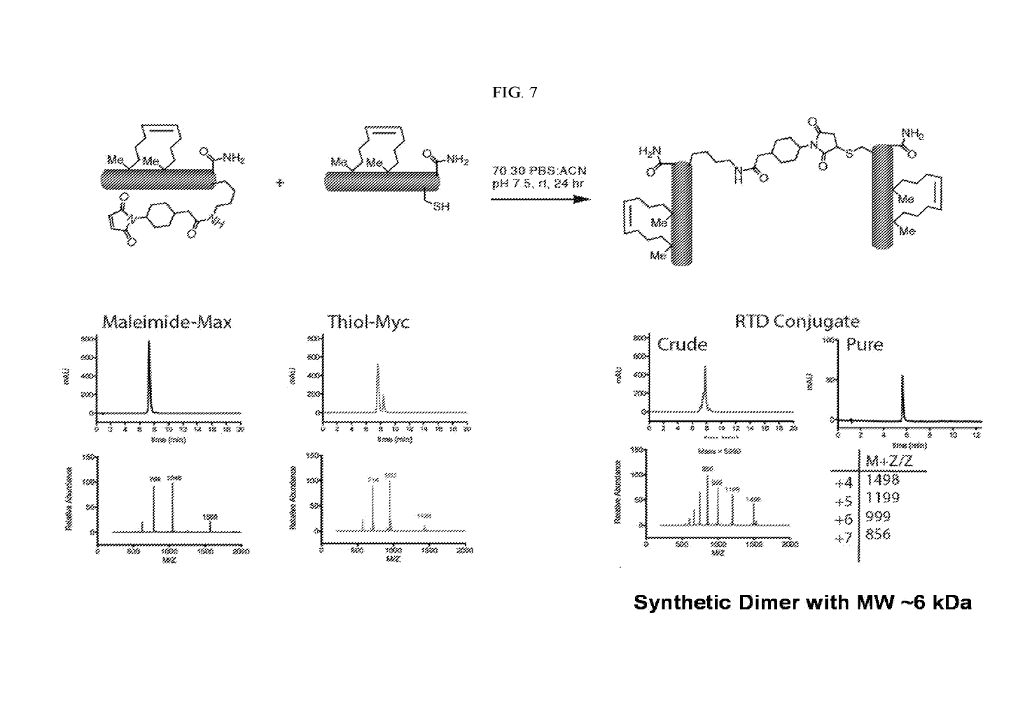

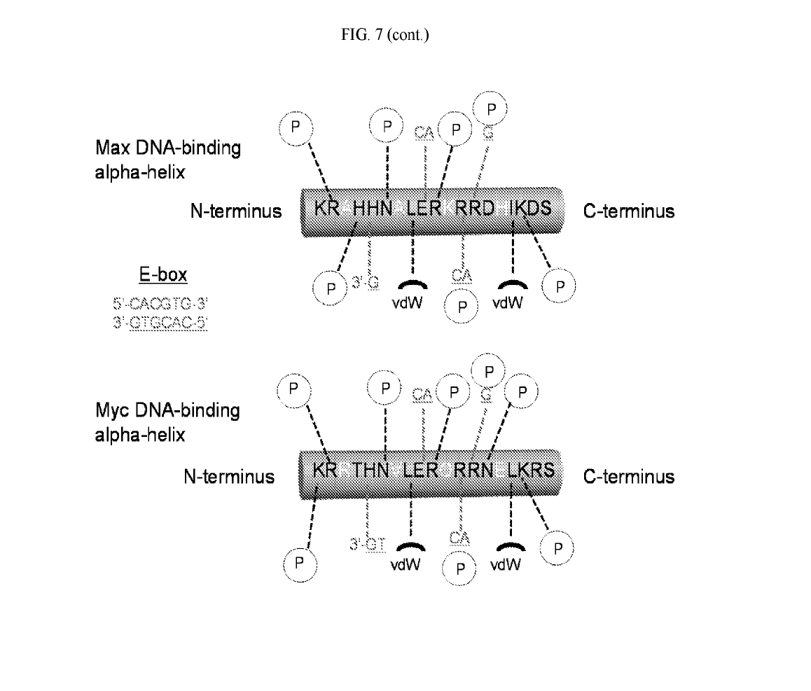

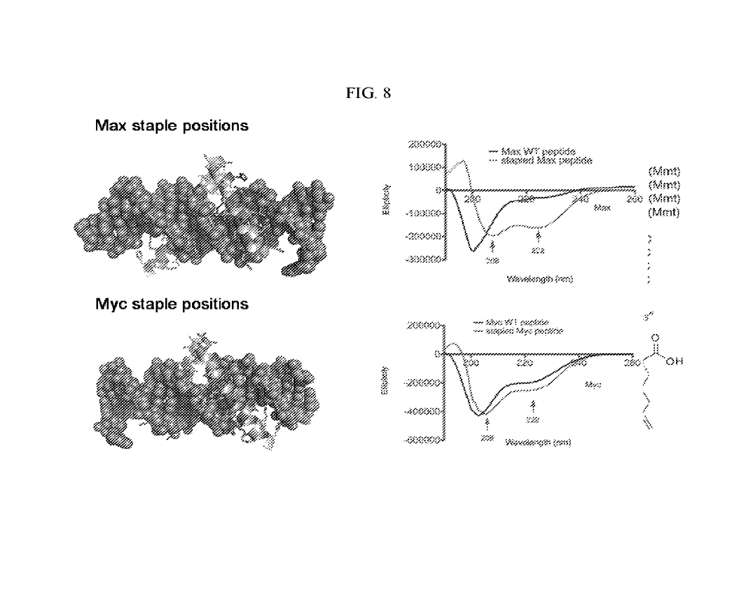

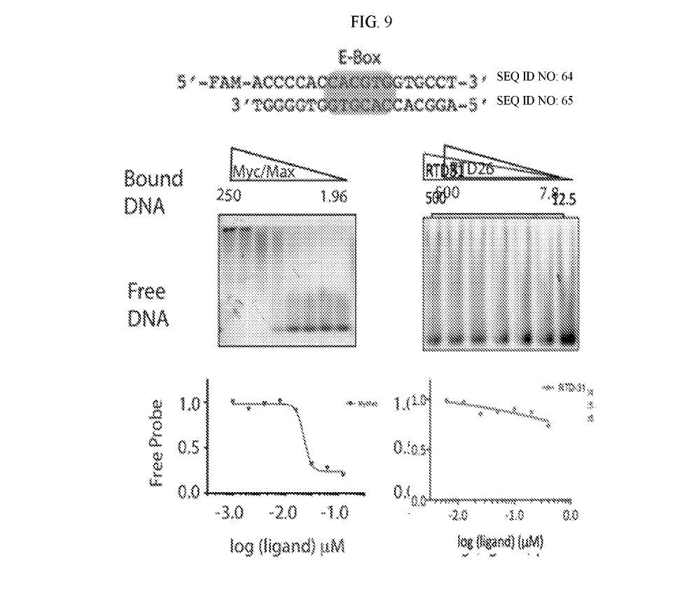

Peptides were synthesized using a variety of conjugation chemistry. Thio-maleimide (FIGS. 7-8) peptides were synthesized. Maleimide peptides were synthesized with C-terminal Fmoc-Lys(Mmt)-OH amino acid, with or without spacer amino acids. Internal bis-alkylated “S5” or “R8” amino acids (used for hydrocarbon stapling) are incorporated at defined i-i+4 or i-i+7 positions for alpha-helix stabilization. The thiol peptide was synthesized with C-terminal Fmoc-Cys(Trt)-OH amino acid, with or without spacer amino acids. Internal bis-alkylated “S5” or “R8” amino acids (used for hydrocarbon stapling) are incorporated at defined i−i+4 or i−i+7 positions for alpha-helix stabilization.

Various linkers and/or N-terminal chemical tags can be incorporated (fluorophores, fatty acids, biotin, polyethylene glycol, acetylation etc.). The completed peptide is chemically “stapled” with Grubbs-I olefin metathesis catalyst in dichloroethane. Mmt-Lysine is selectively deprotected with 1% trifluoroacetic acid, 2% dithioethane, 2% triisopropylsilane in dichloromethane. This leaves all other amino acids protected while exposing the lysine amine. The maleimide reactive group is incorporated by reacting an NHS-linker-Maleimide moiety with the lysine amine, yielding the stable maleimide-peptide cojugate, which can be cleaved and purified by standard methods.

Purified (or semi-purified) maleimide- and thiol-containing peptides are reacted in a mixture of phosphate buffered saline and acetonitrile (70% and 30%, respectively) at pH 7.5. These conditions have been found to be amenable to a wide-range of peptides over concentration ranges of 1 micromolar to 500 micromolar. This reaction is carried out at room temperature or elevated temperature for faster kinetics. Addition of non-alkylating reducing agents (such as TCEP) can prevent disulfide formation between thiol peptides. This synthetic route enables oriented dimerization of the peptide C-termini, which is useful to mimic the DNA binding architecture of bHLH-LZ TF proteins. The resulting crude dimers are purified by traditional reverse-phase HPLC methods.

FIG. 7 shows LCMS chromatograms and mass spectra of purified monomers (maleimide-Max and thiol-Myc peptides) and the resulting product. —The crude mixture shows over 80% conversion after 24 hours and this species can be purified to >95% purity. The mass spectrum of the resulting dimer (MW=5990 da) shows characteristic m/z peaks for z=4, 5, 6 & 7 ions.

FIG. 16 shows alkyne-azide huisgen conjugation. Peptide was synthesized with C-terminal Fmoc-Lys(Mmt)-OH amino acid, with or without spacer amino acids. Internal bis-alkylated “S5” or “R8” amino acids (used for hydrocarbon stapling) are defined i-i+4 or i-i+7 positions for alpha-helix stabilization. Alkyne substituent is incorporated at the deprotected lysine with hexynoic acid or other electrophilic alkyne. Azide-containing peptide is synthesized with C-terminal azide-containing amino acids.

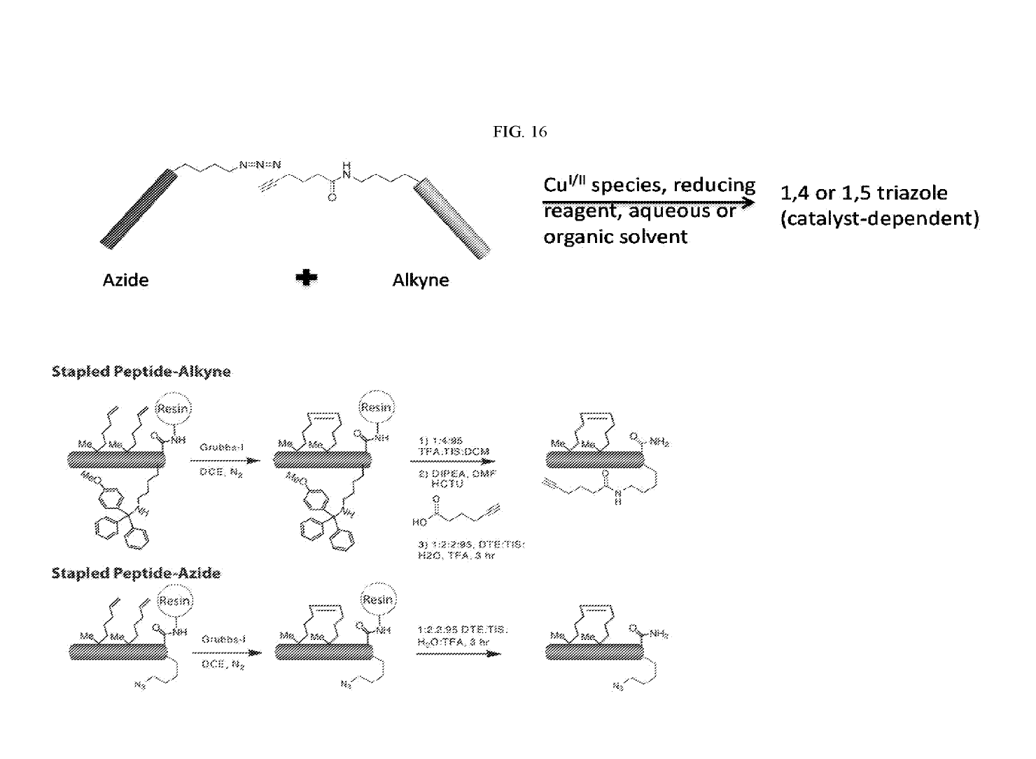

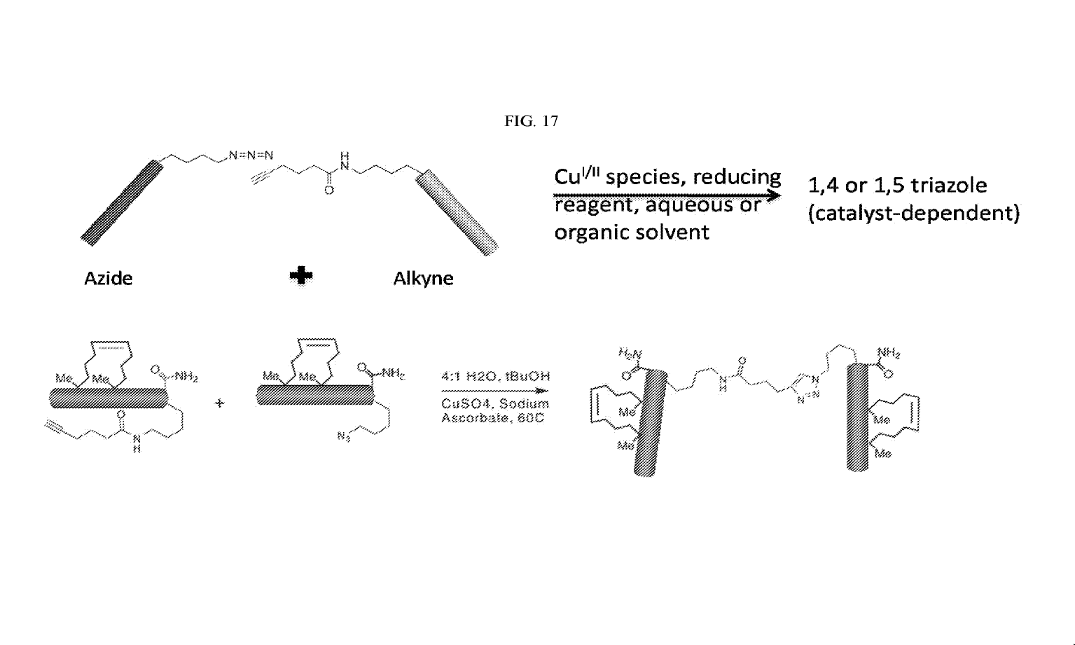

Purified (or semi-purified) alkyne- and azide-containing peptides are reacted as shows in FIG. 17. This synthetic route enables oriented dimerization of the peptide C-termini, which is useful to mimic the DNA binding architecture of bHLH-LZ TF proteins. The resulting crude dimers are purified by traditional reverse-phase HPLC methods

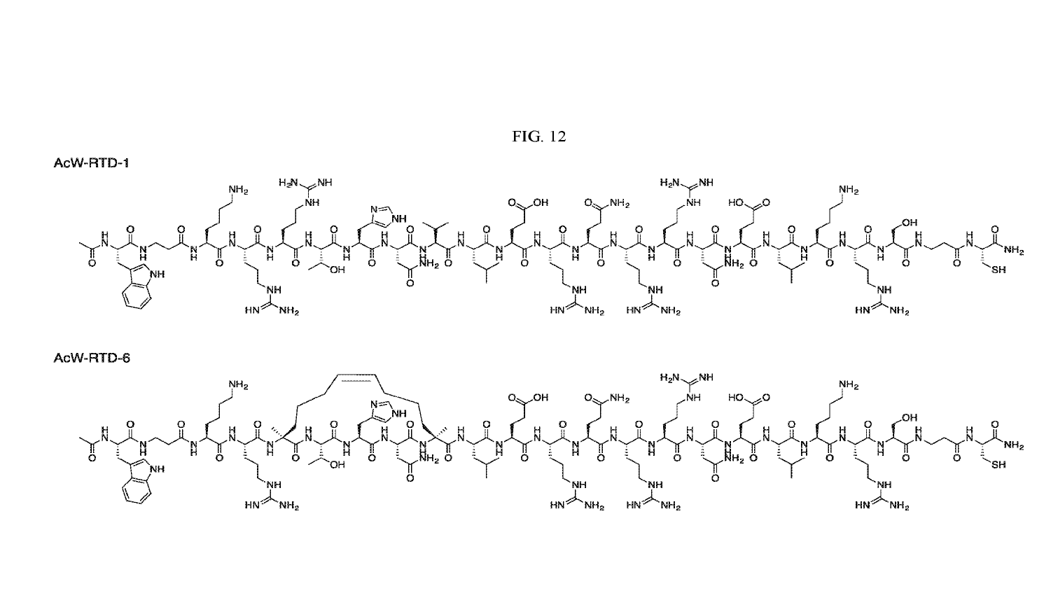

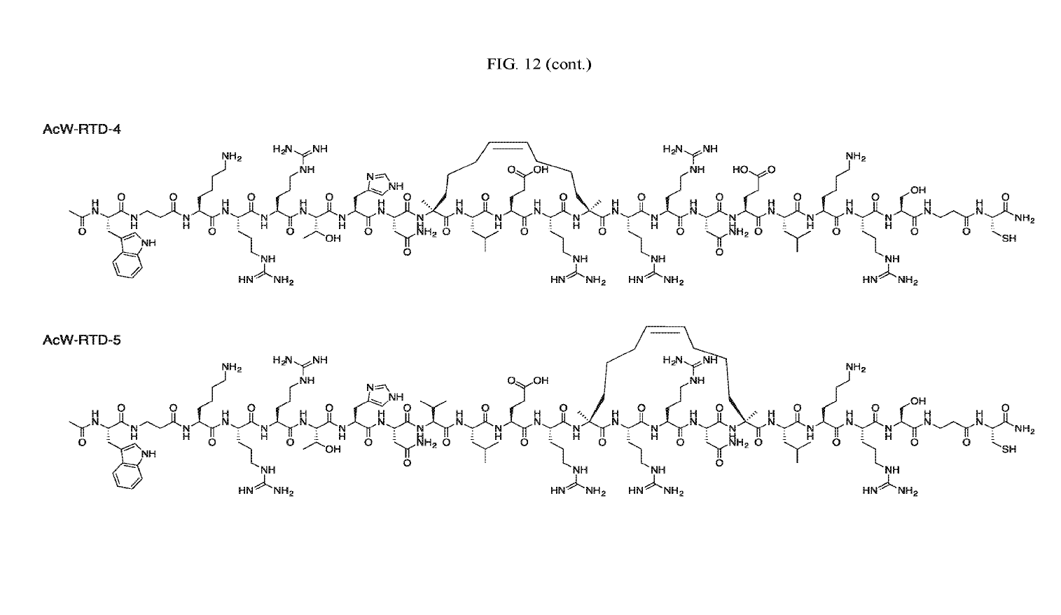

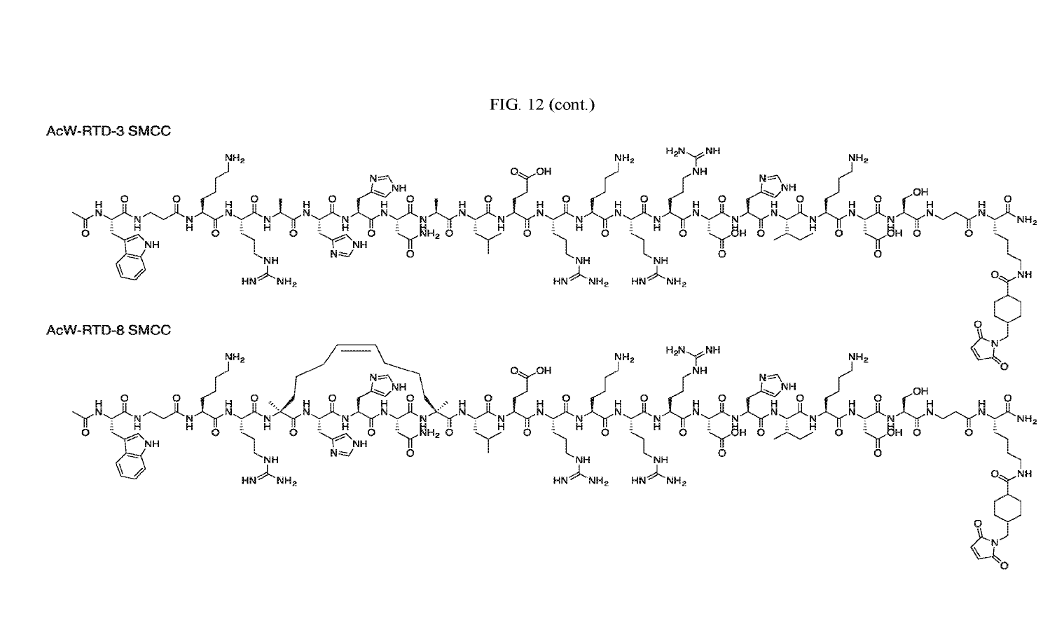

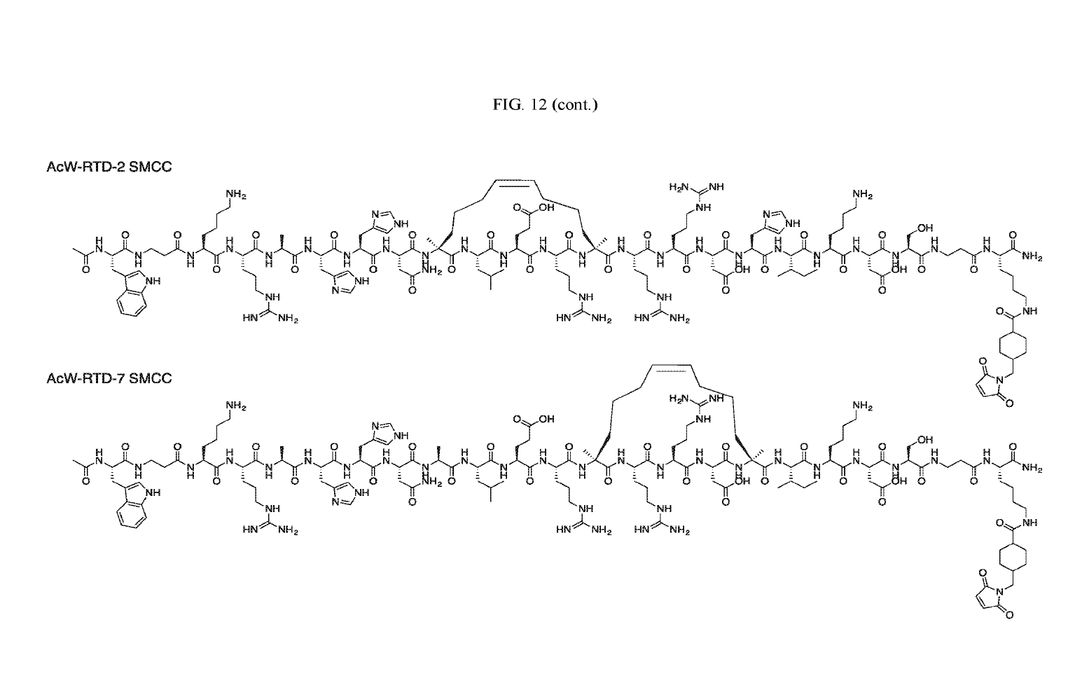

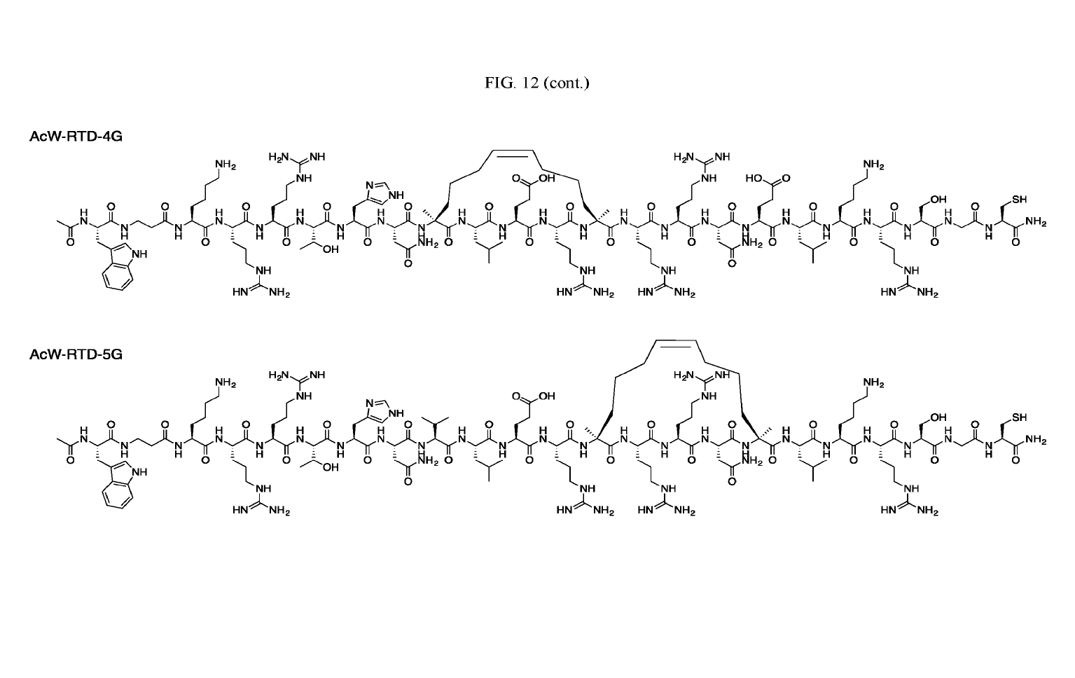

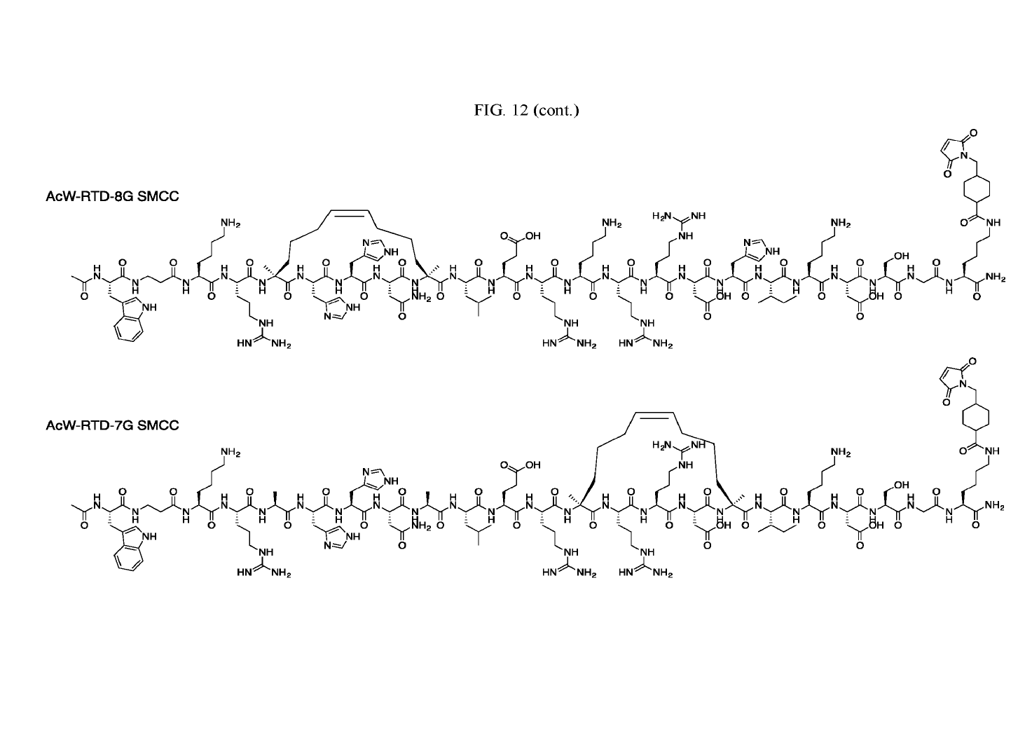

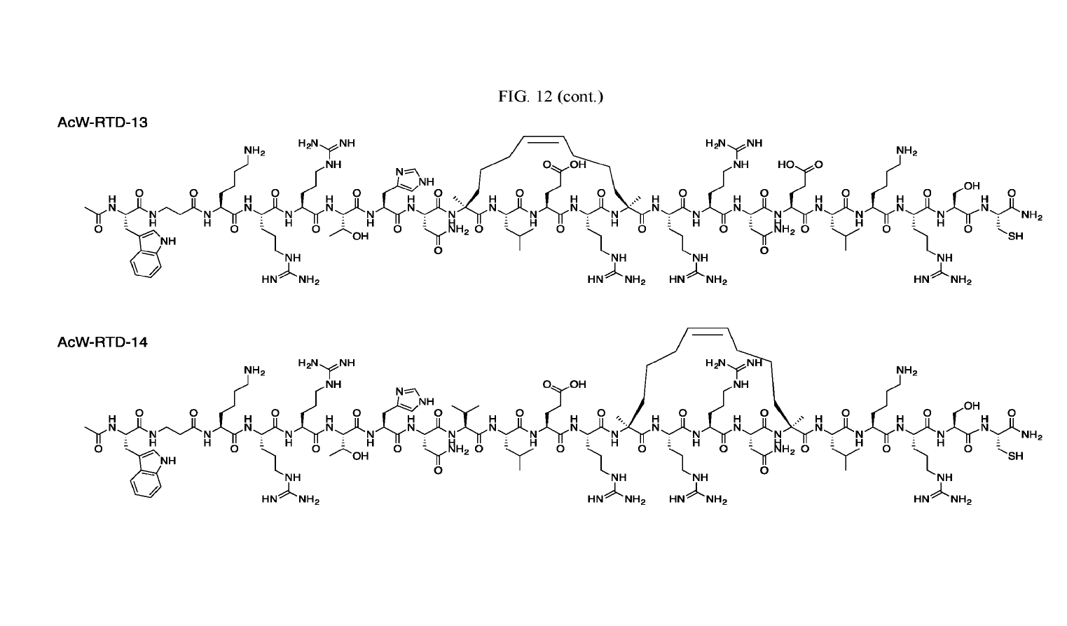

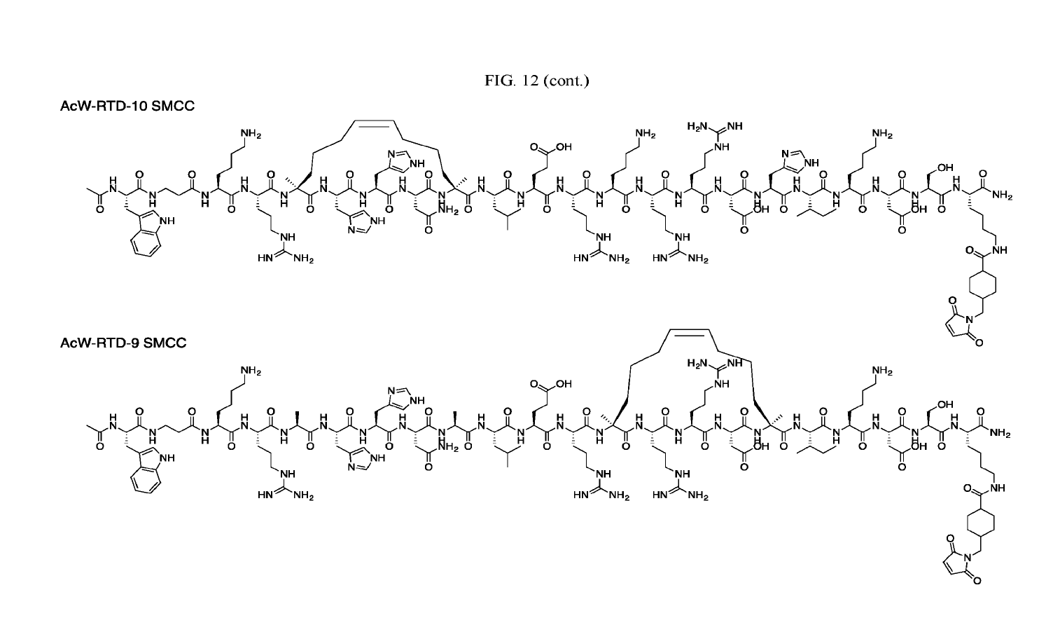

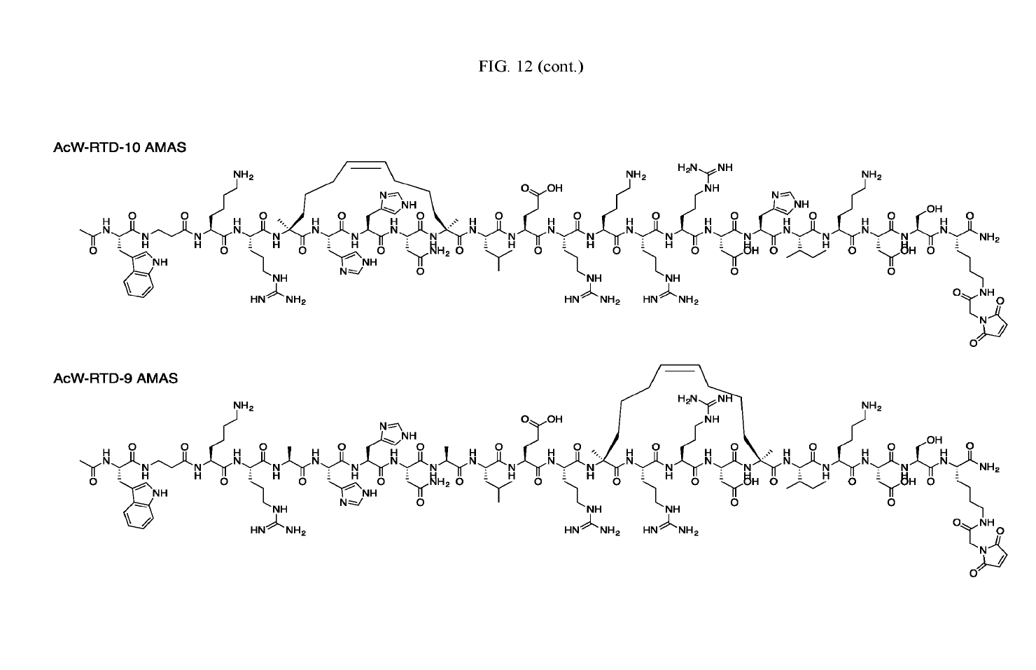

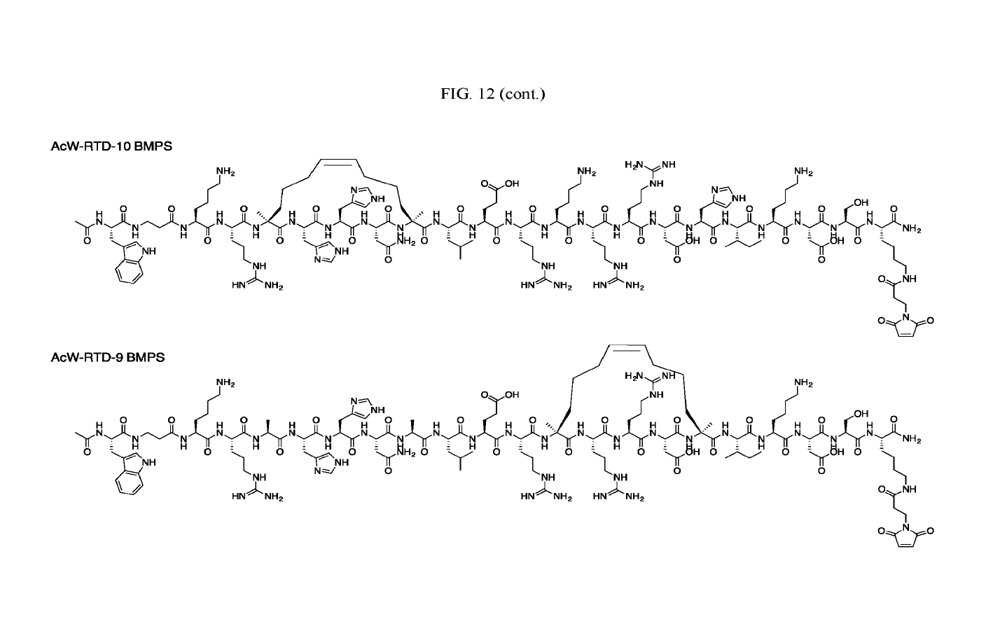

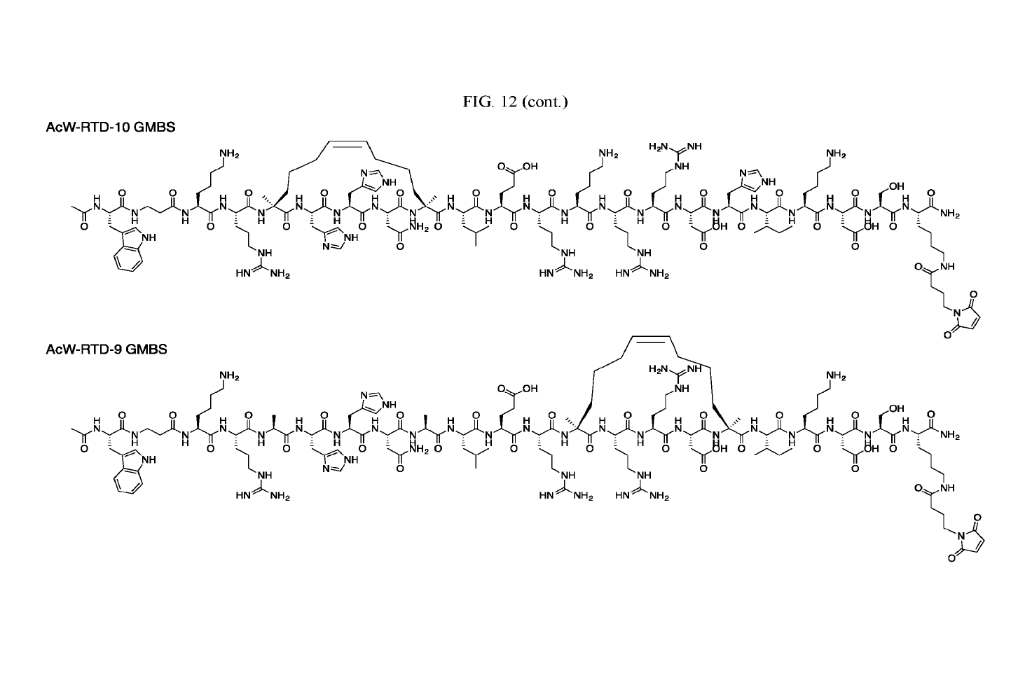

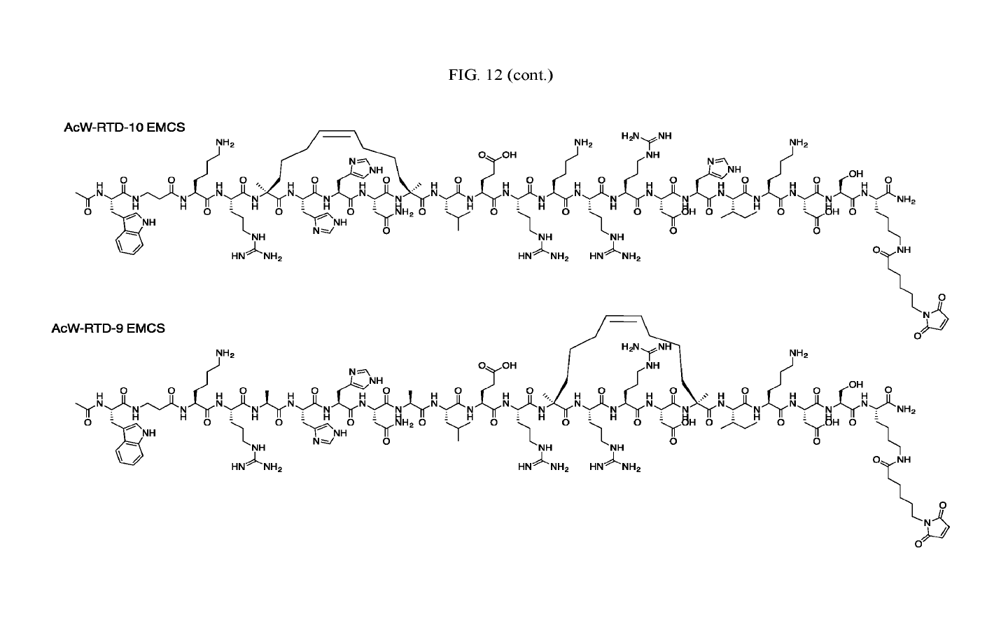

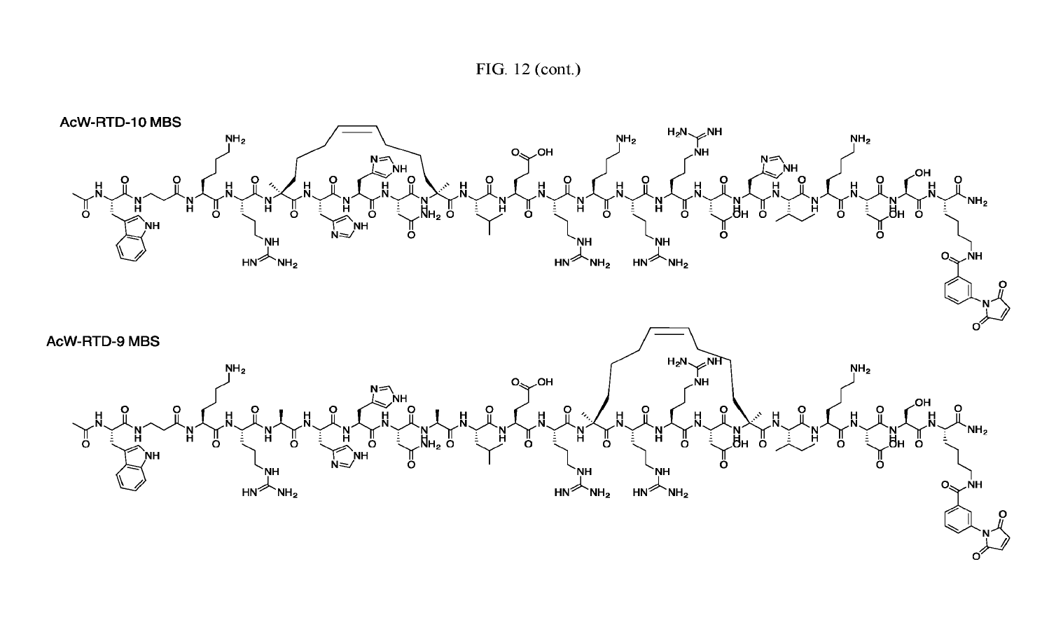

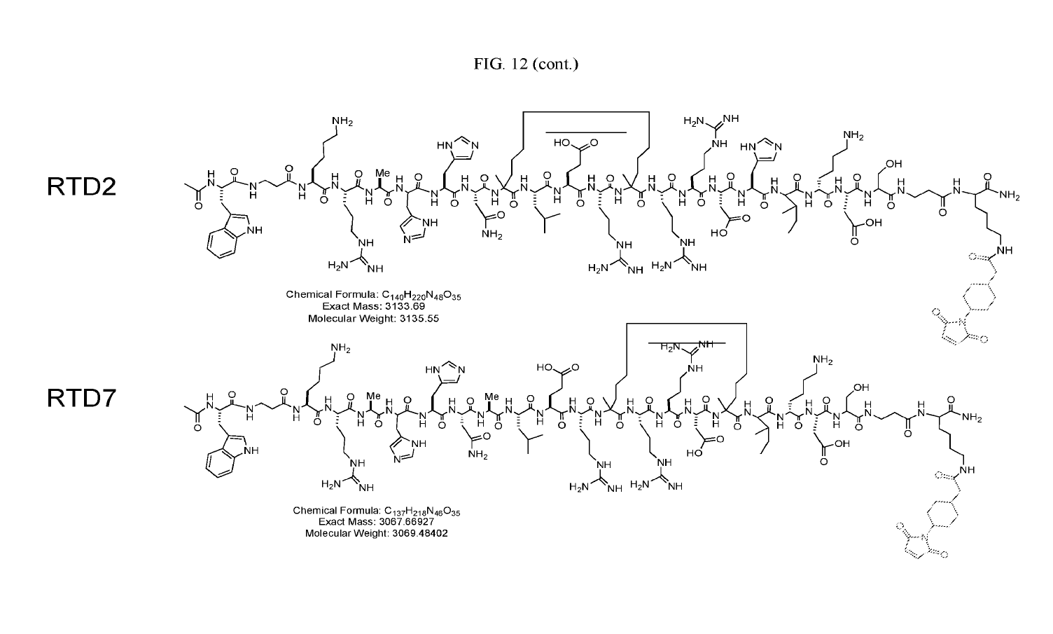

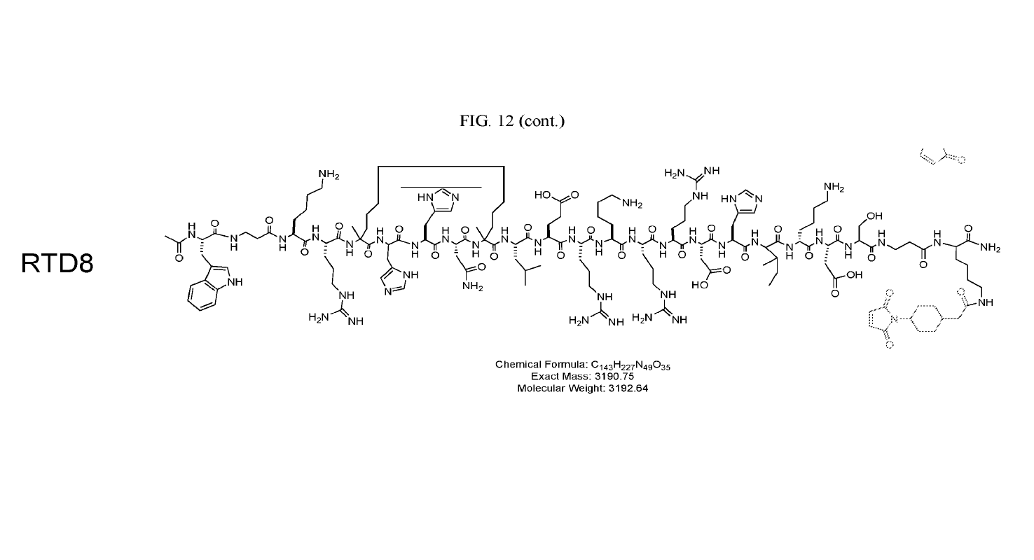

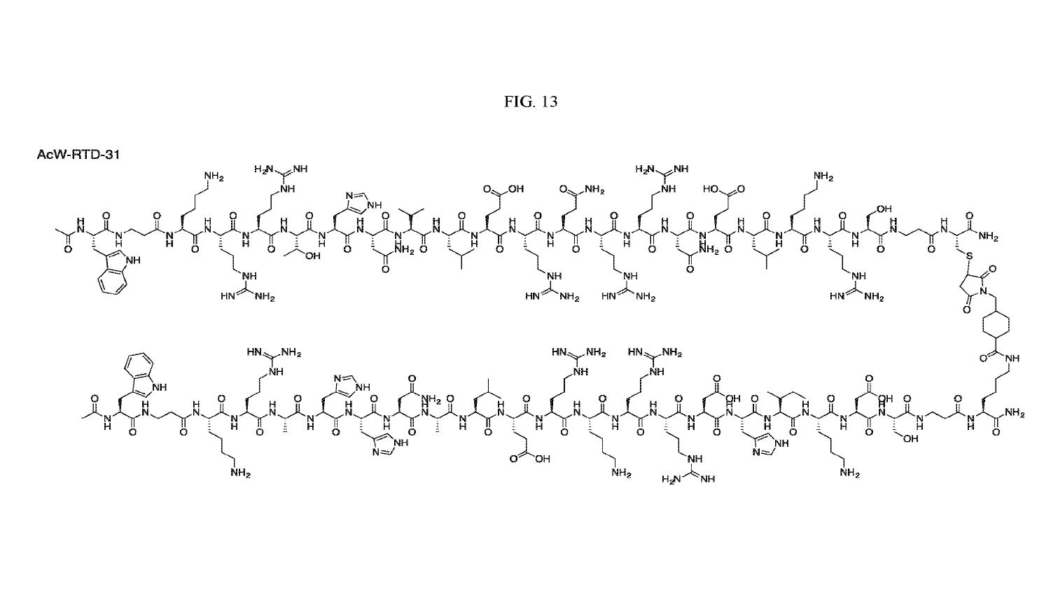

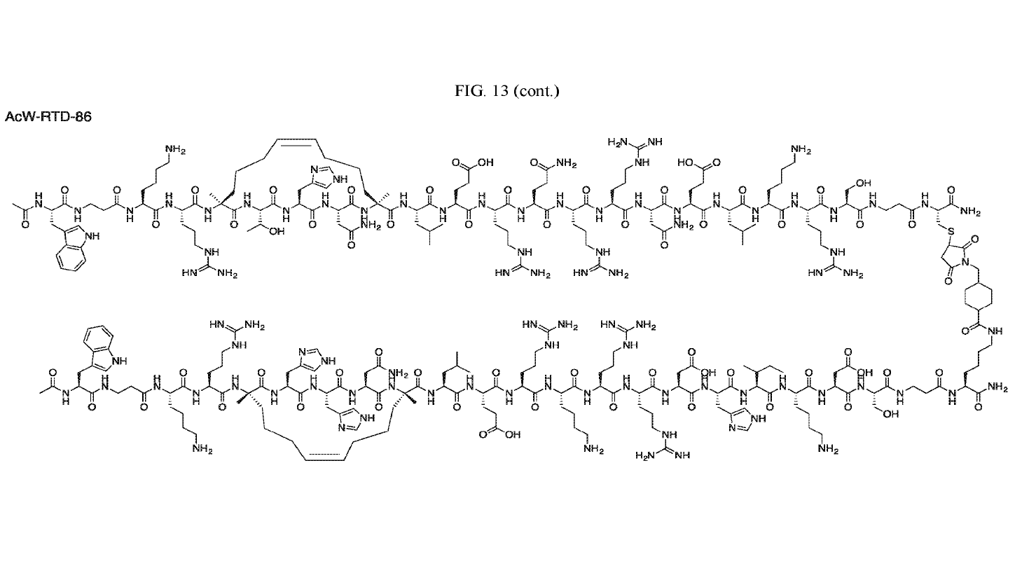

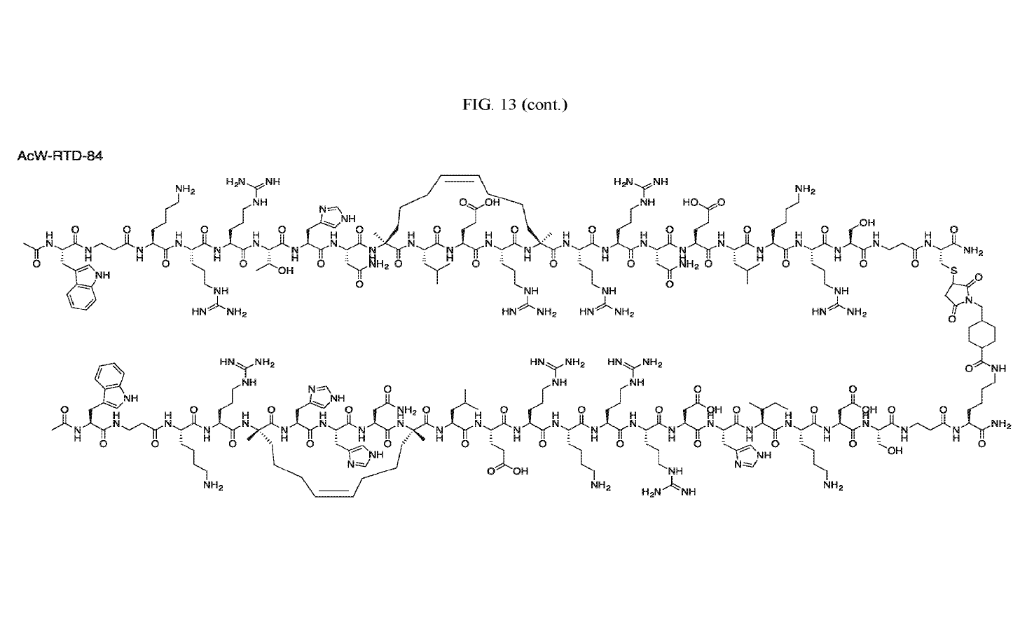

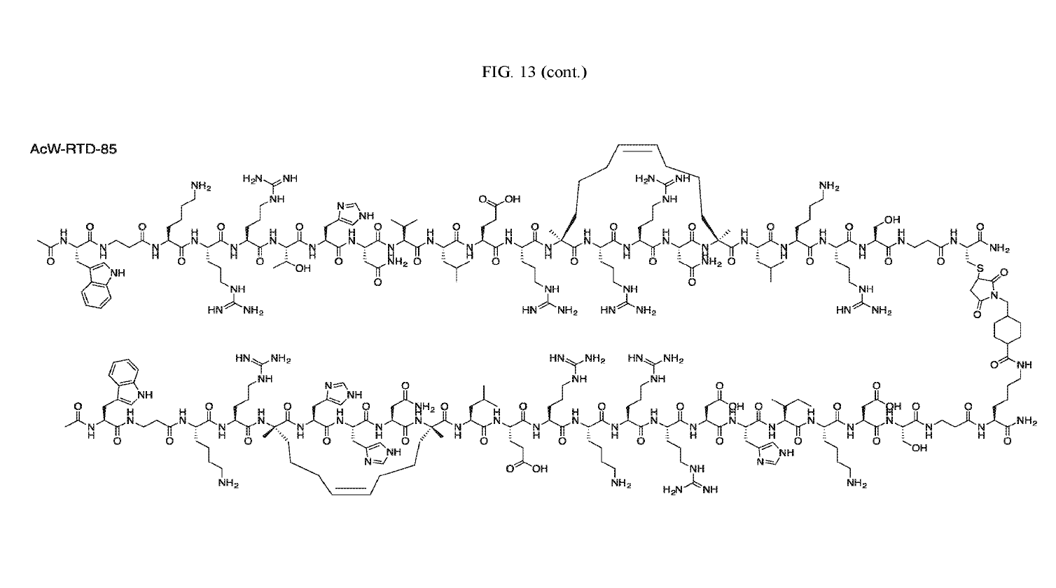

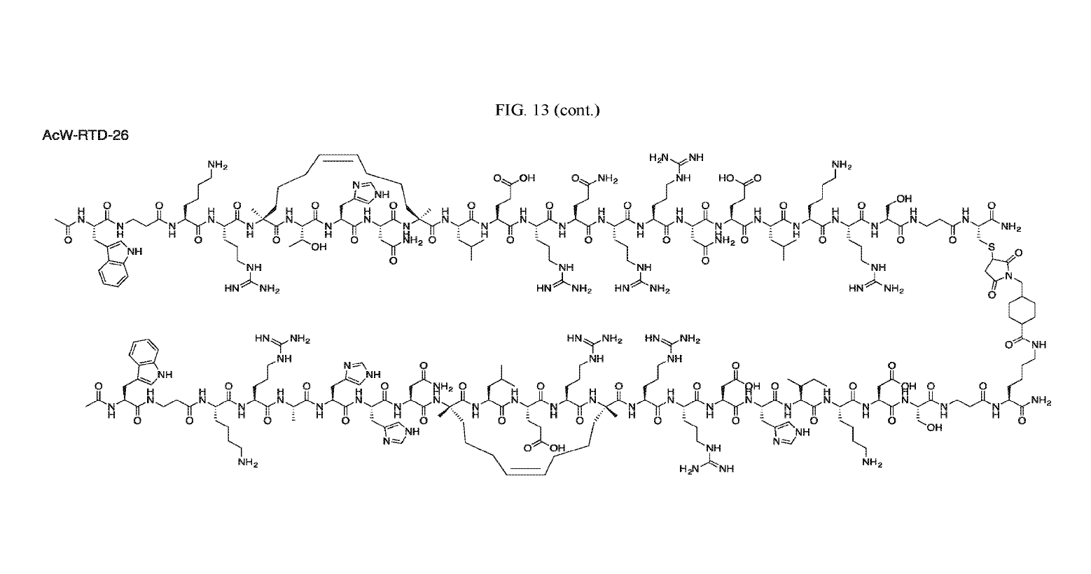

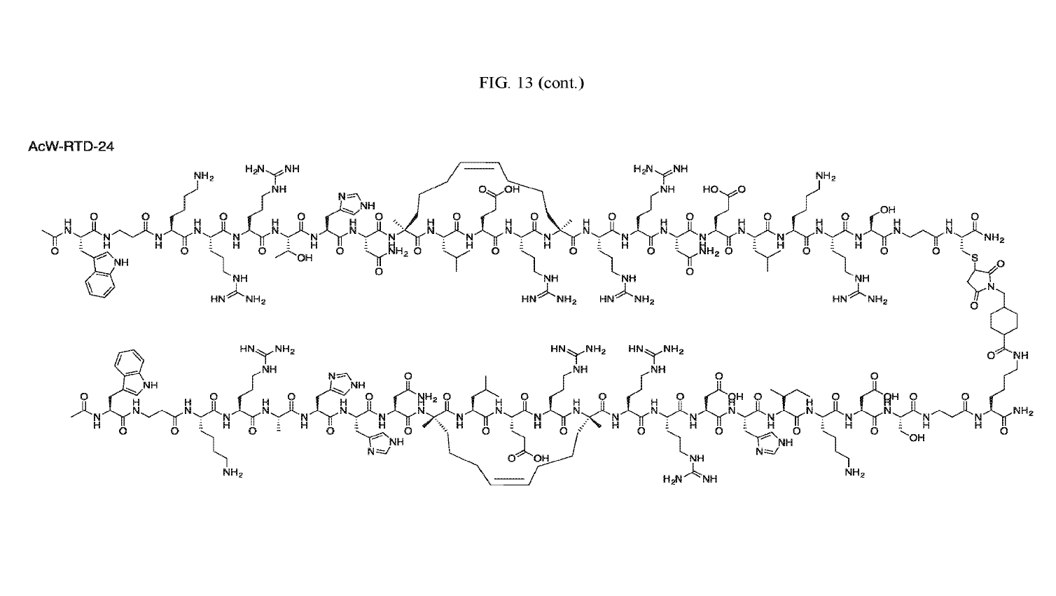

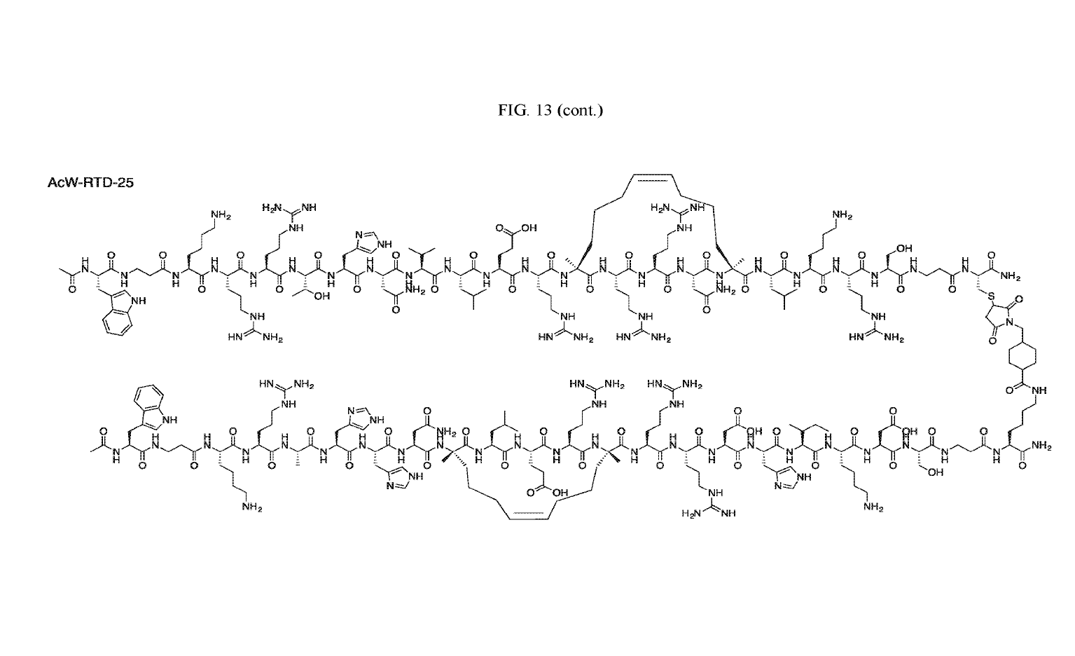

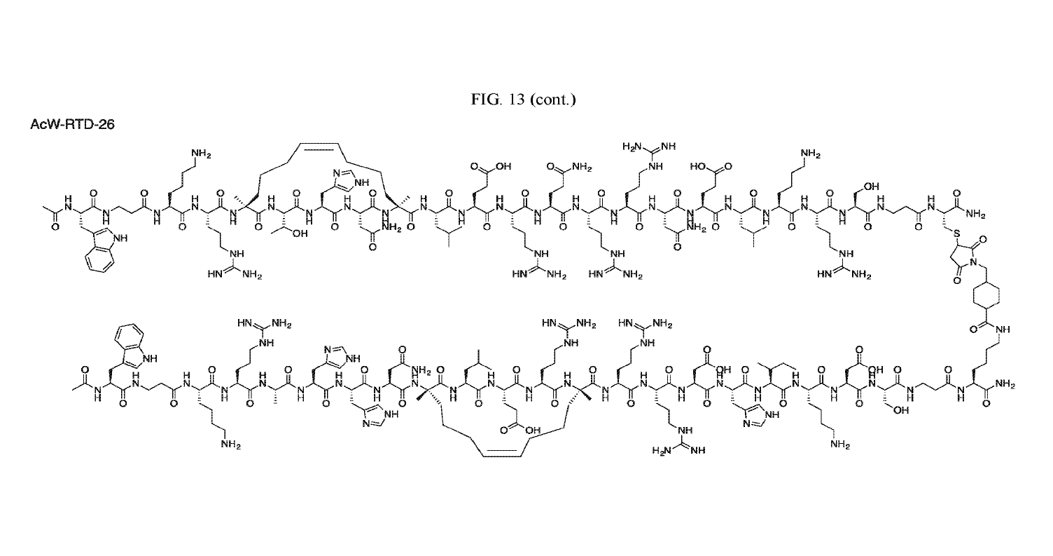

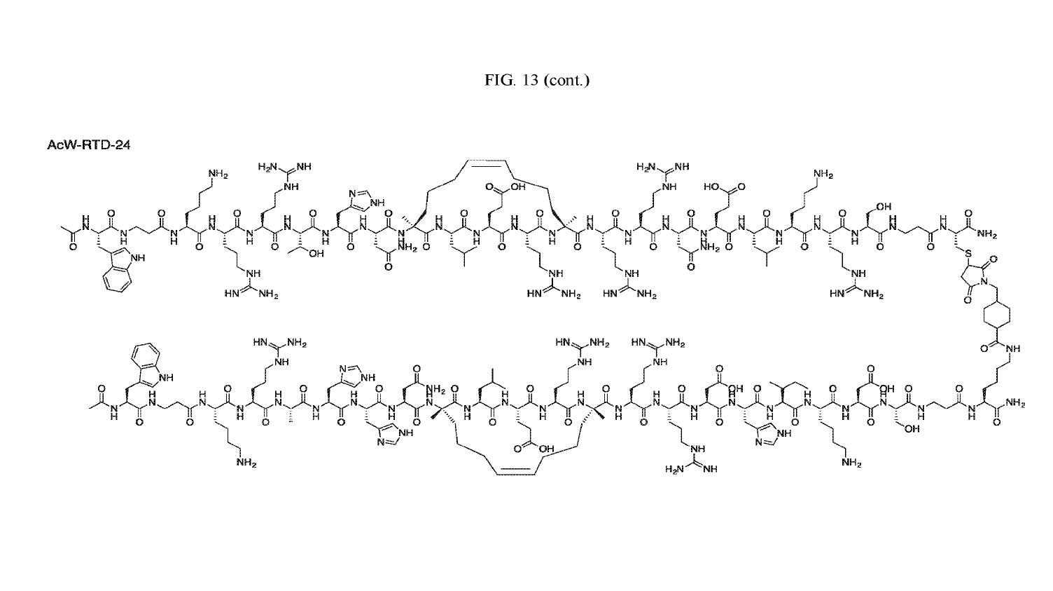

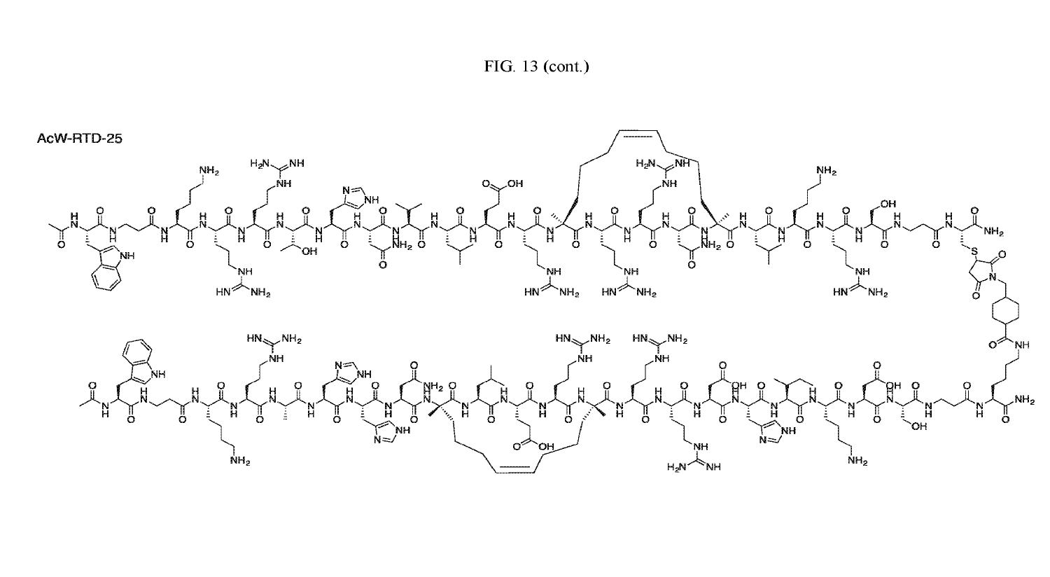

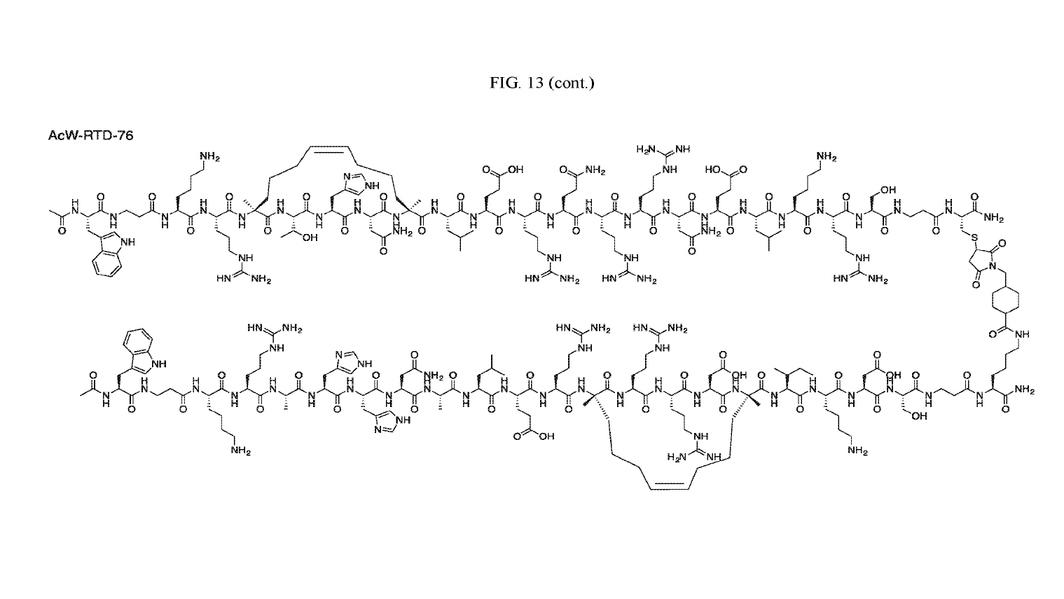

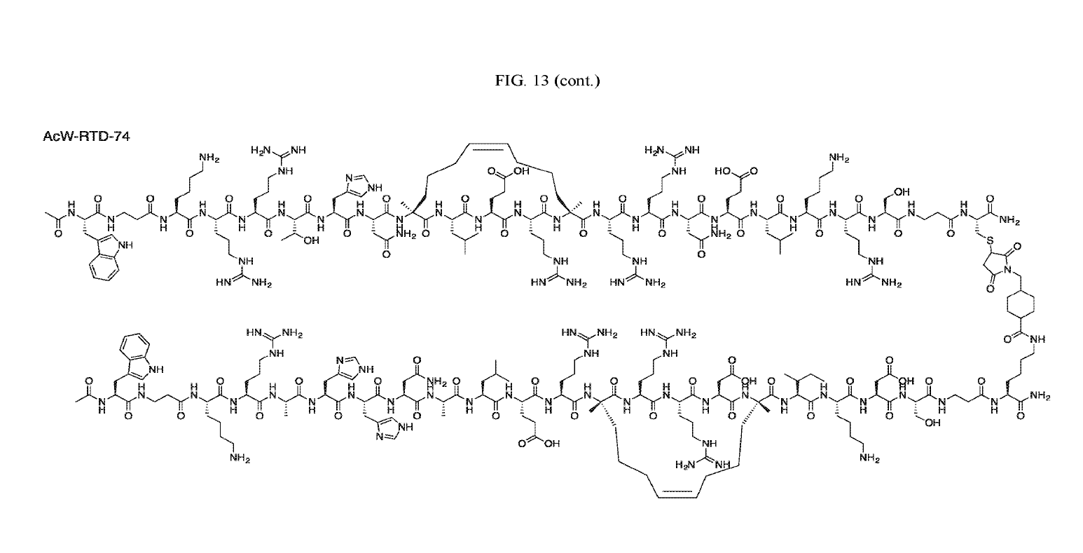

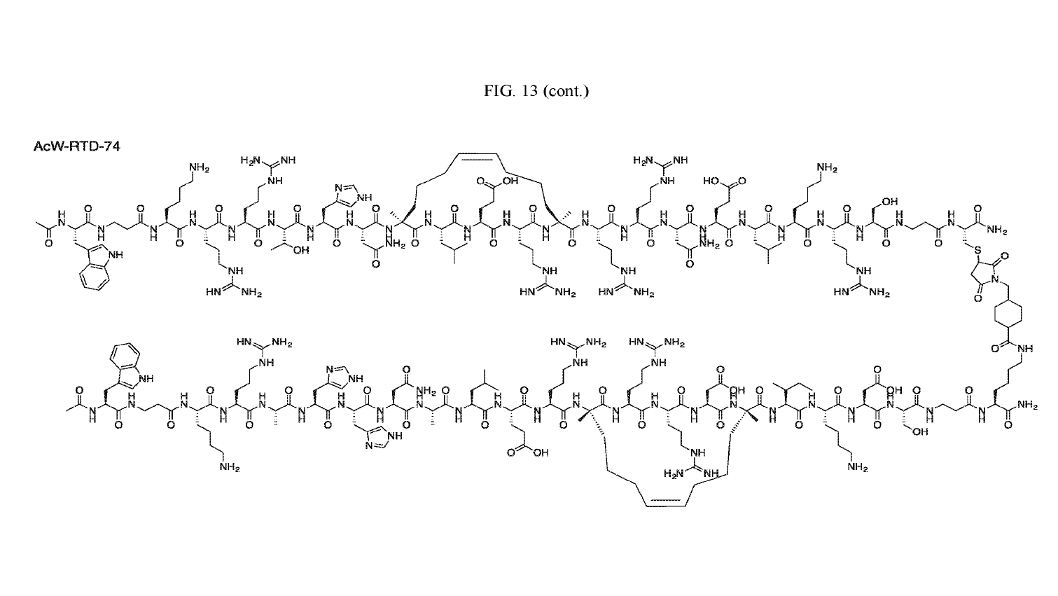

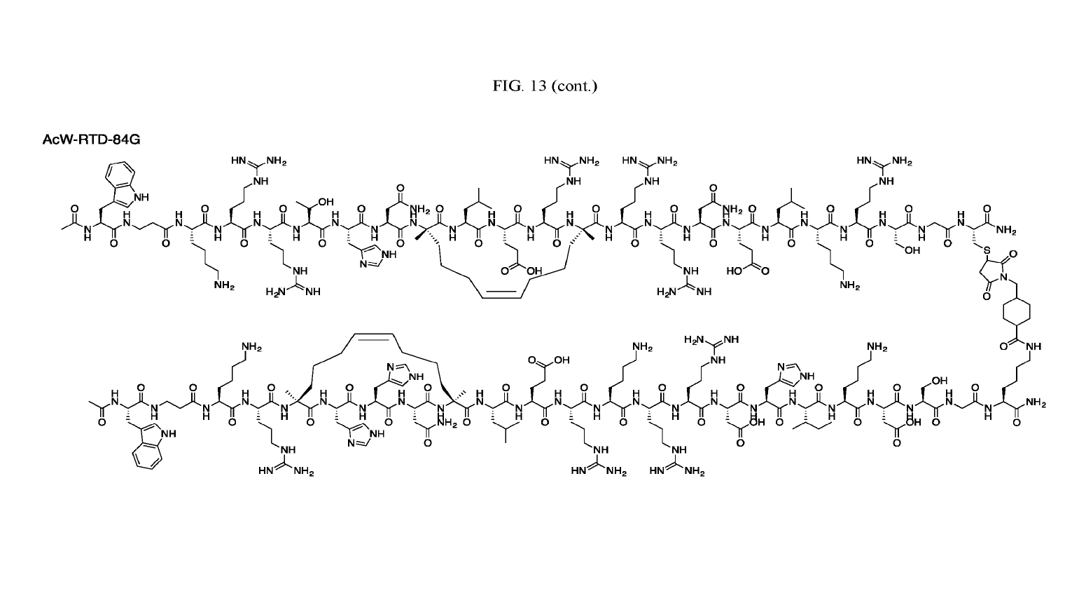

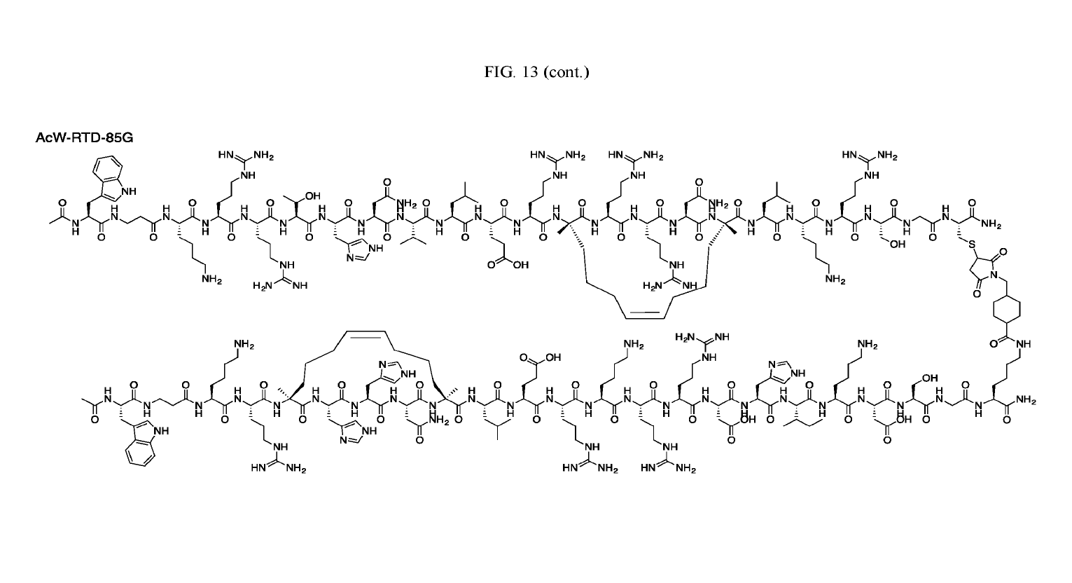

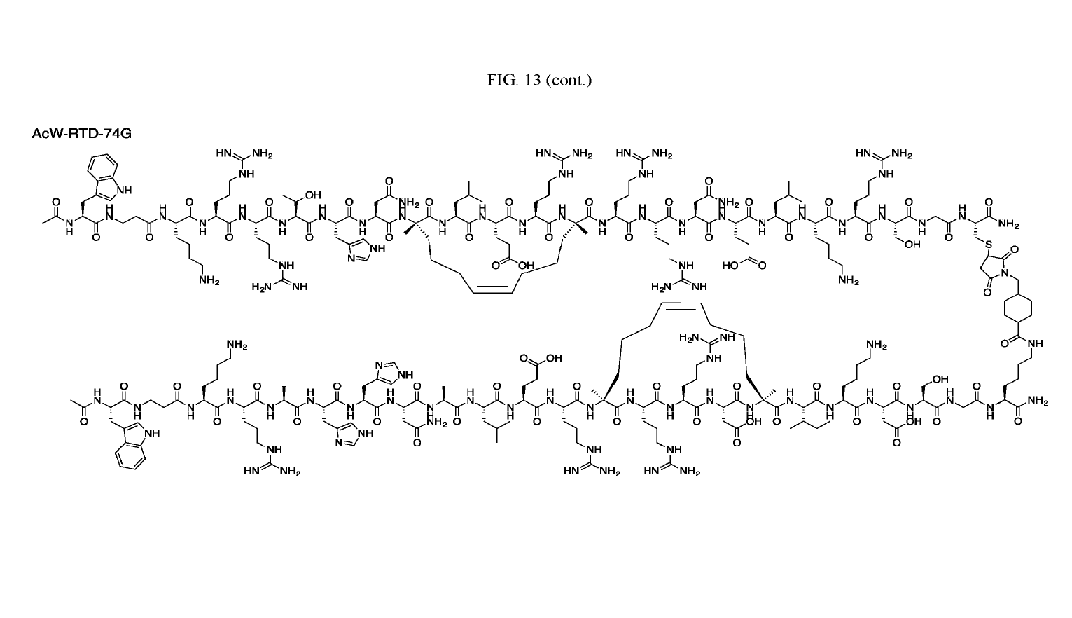

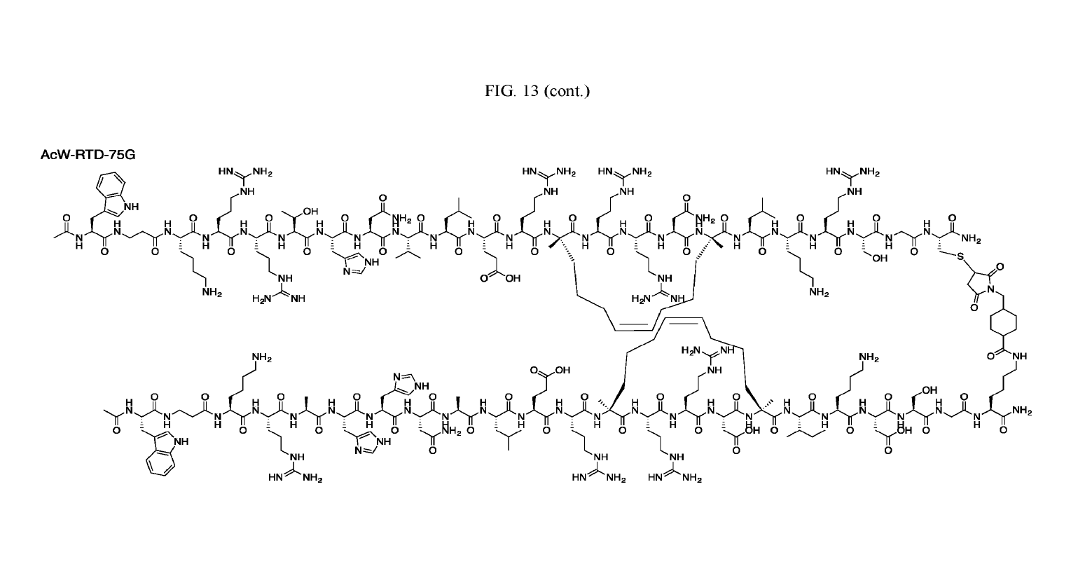

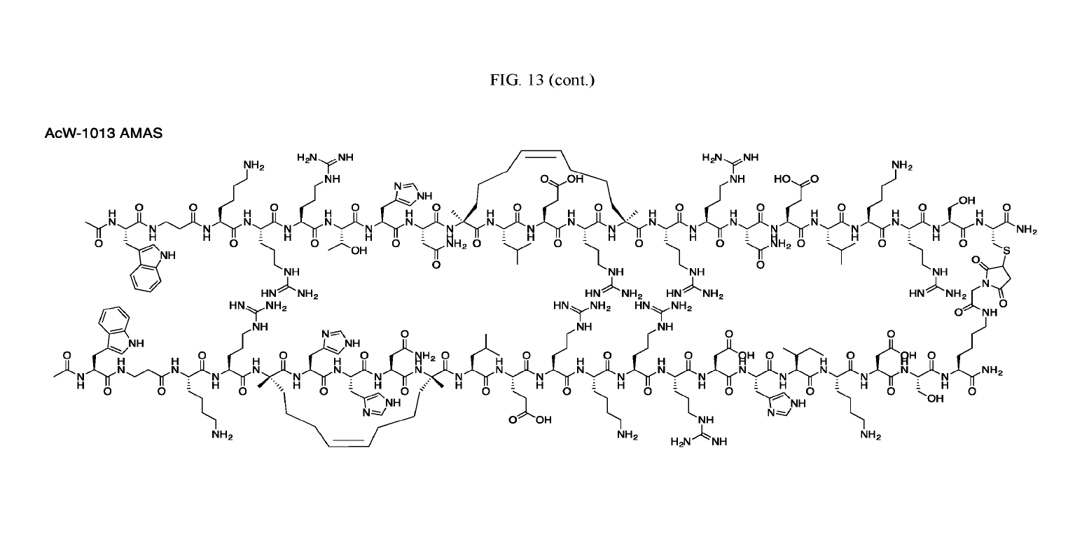

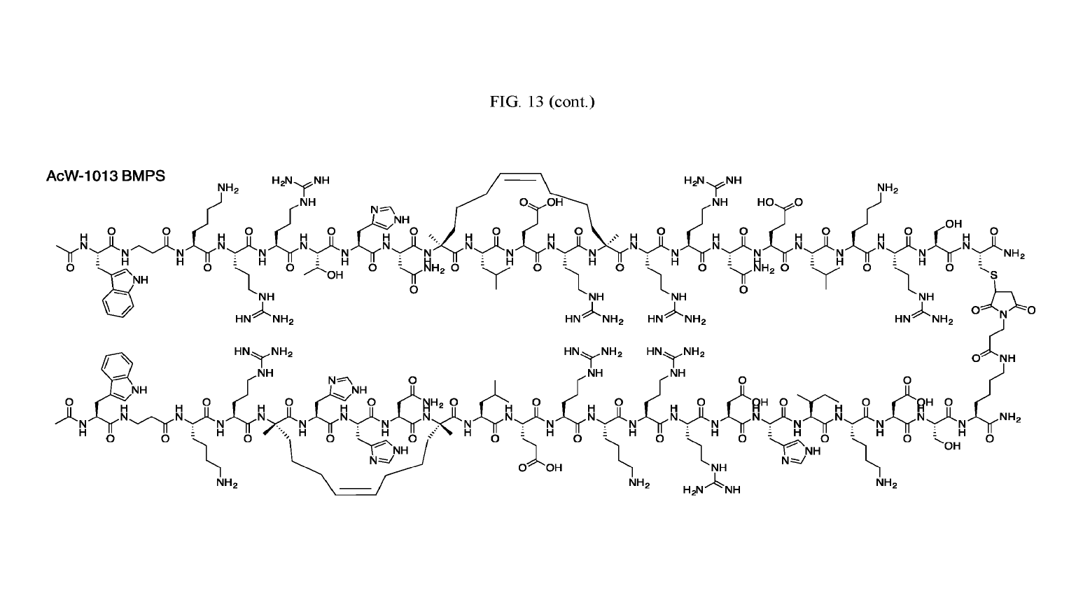

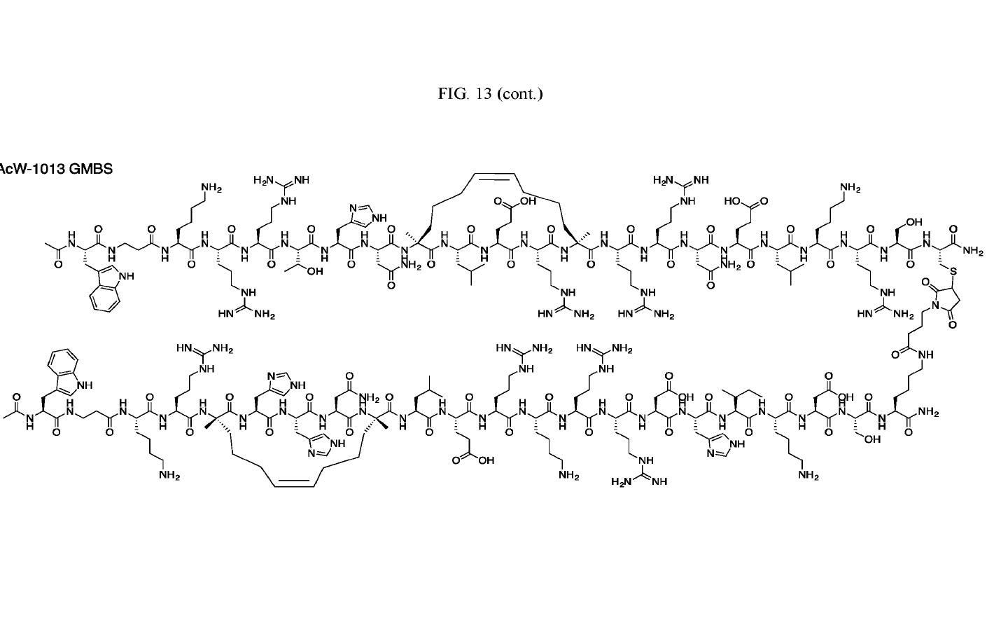

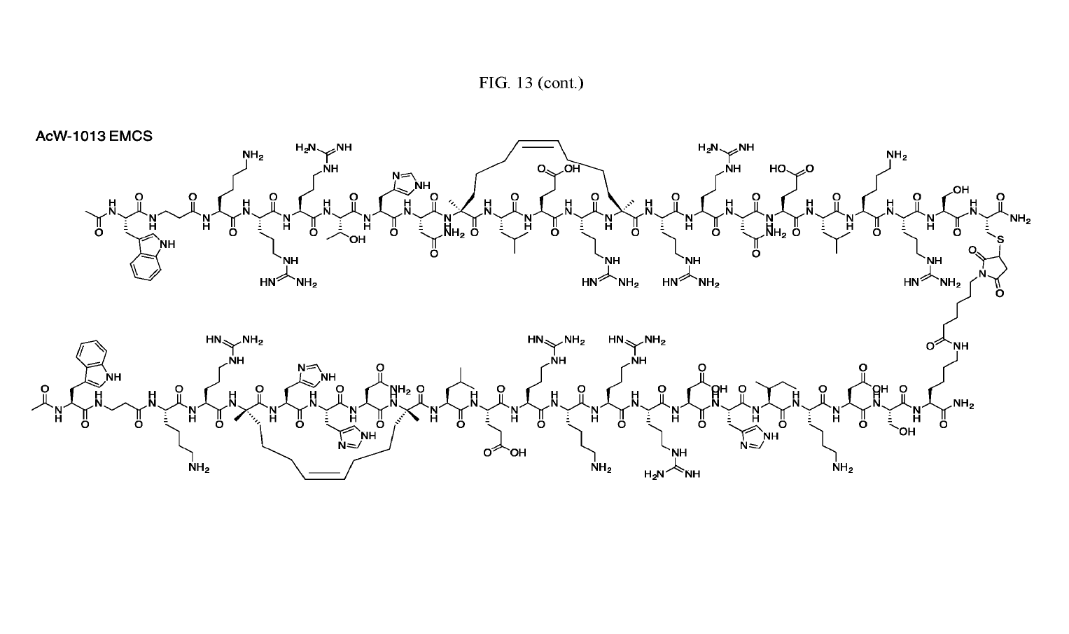

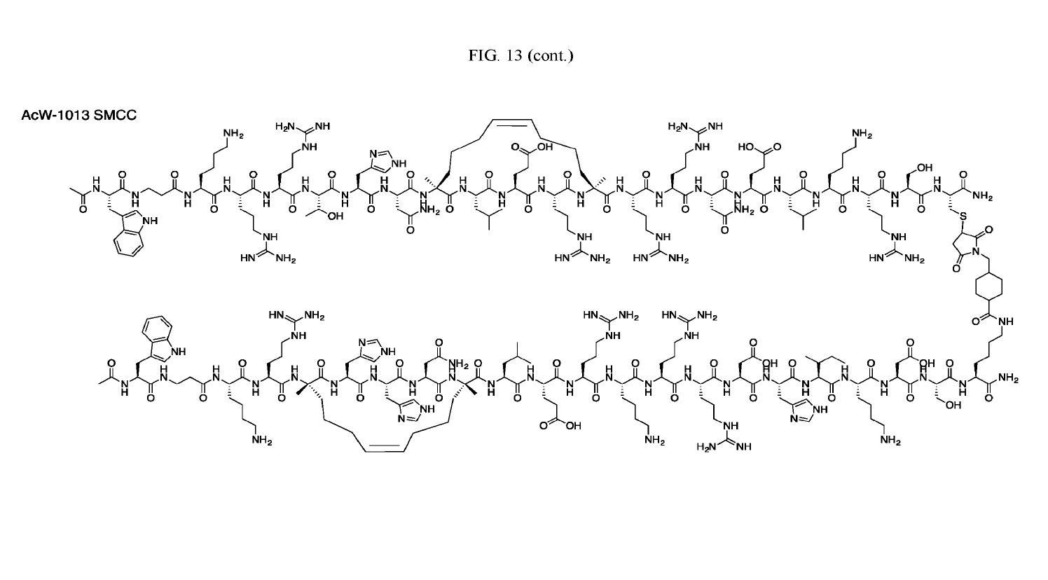

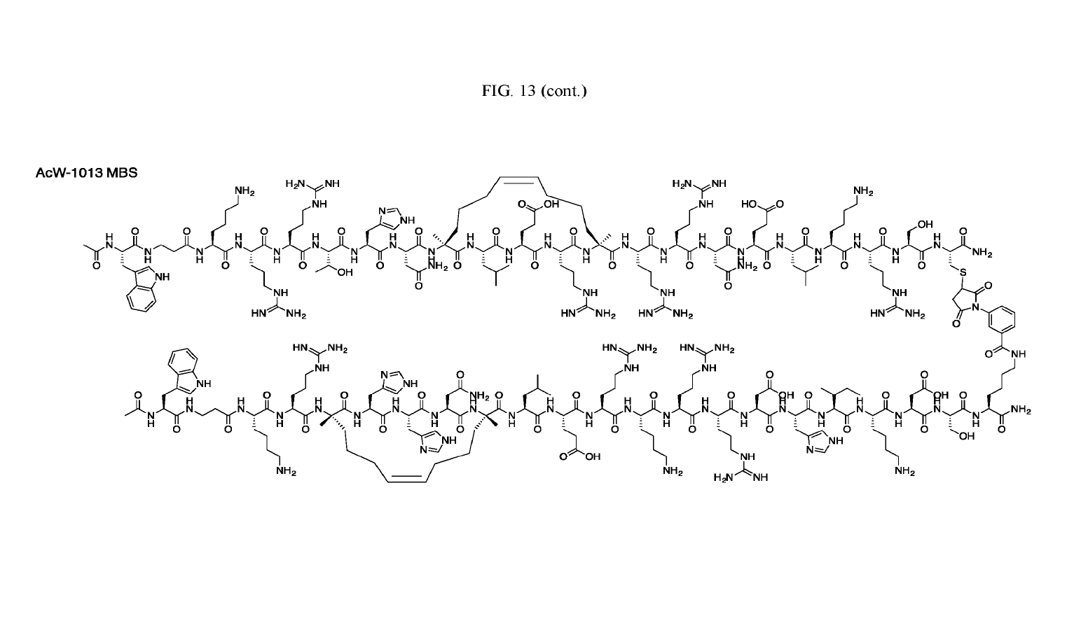

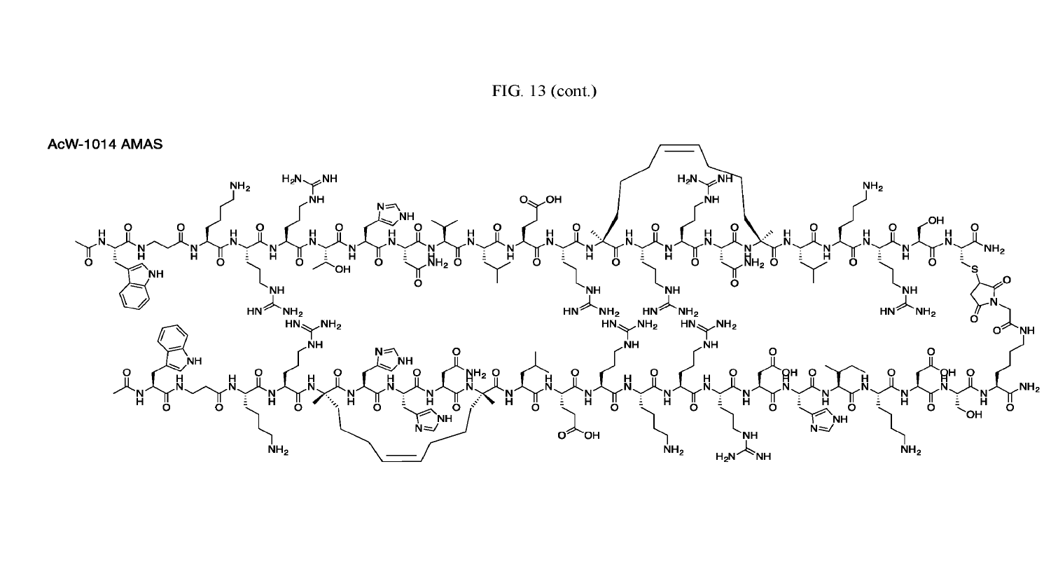

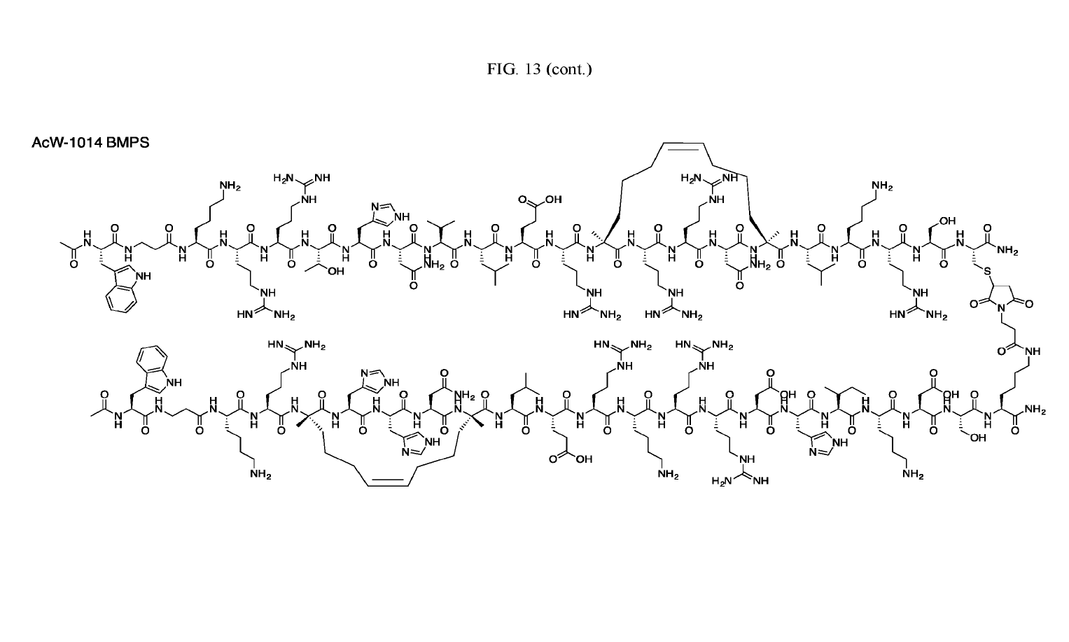

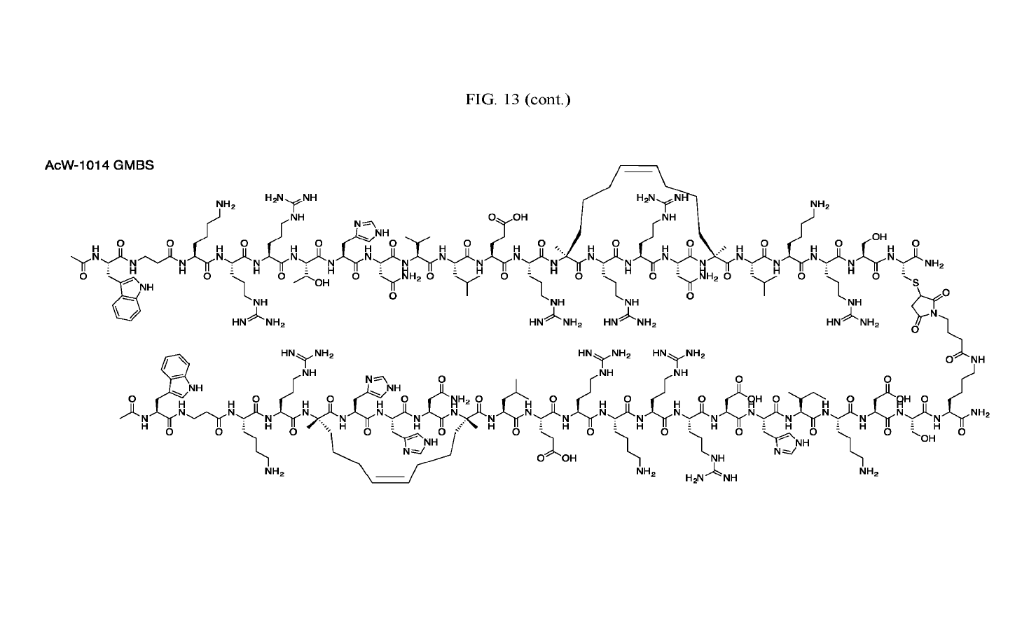

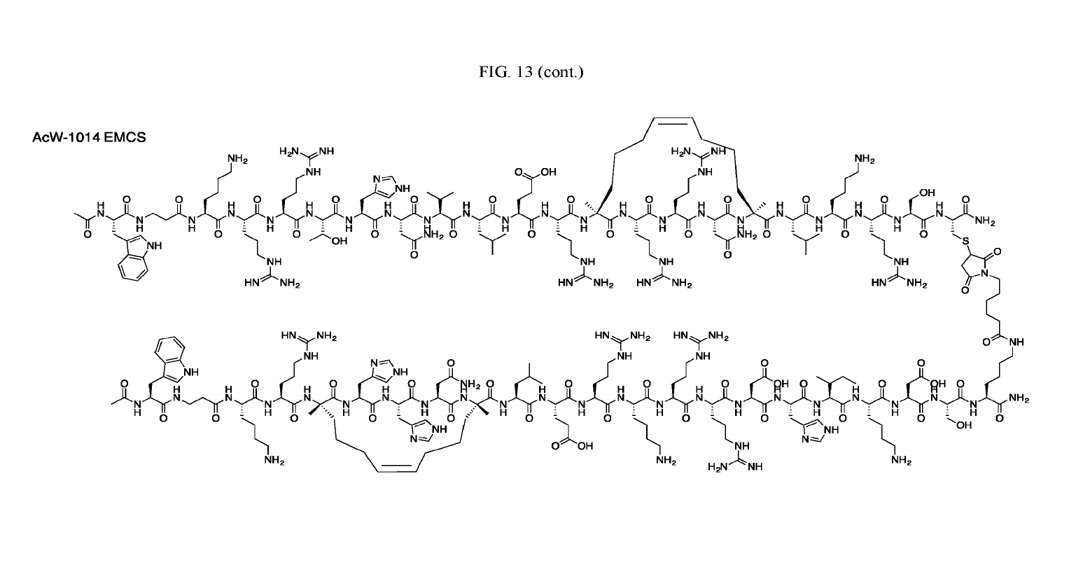

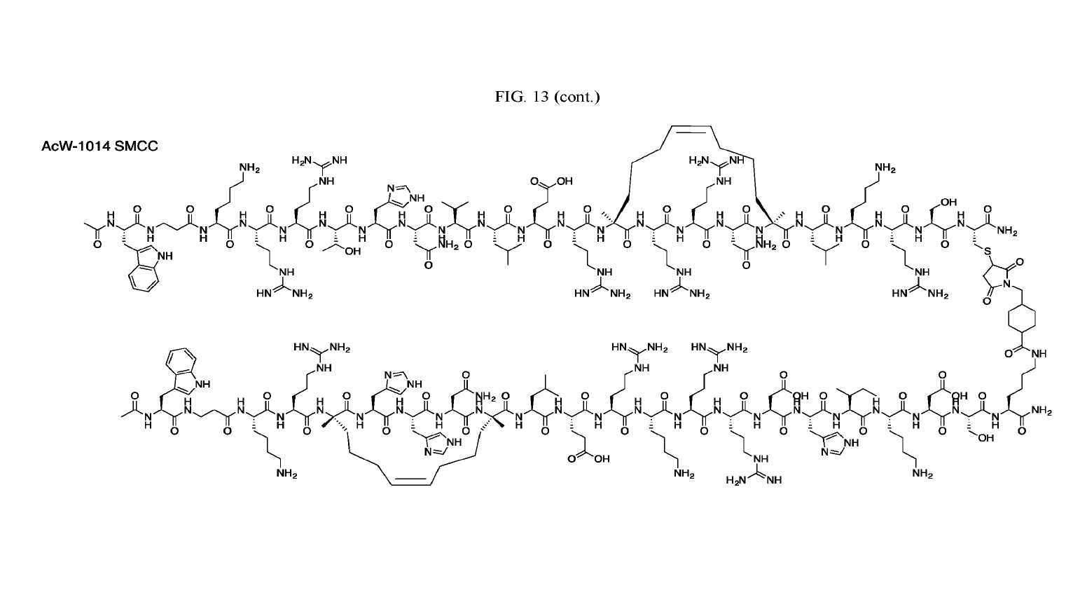

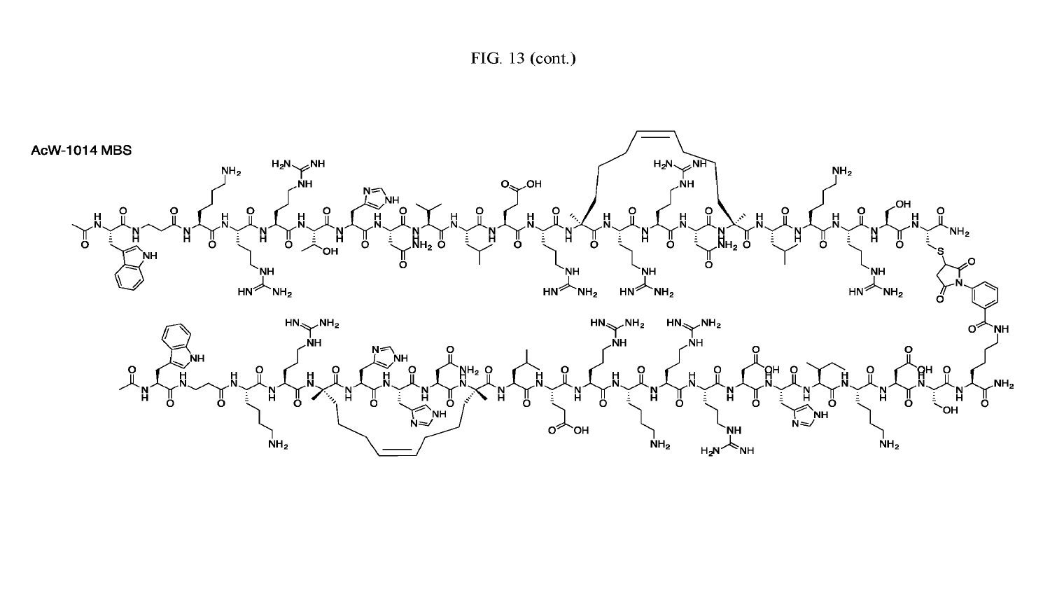

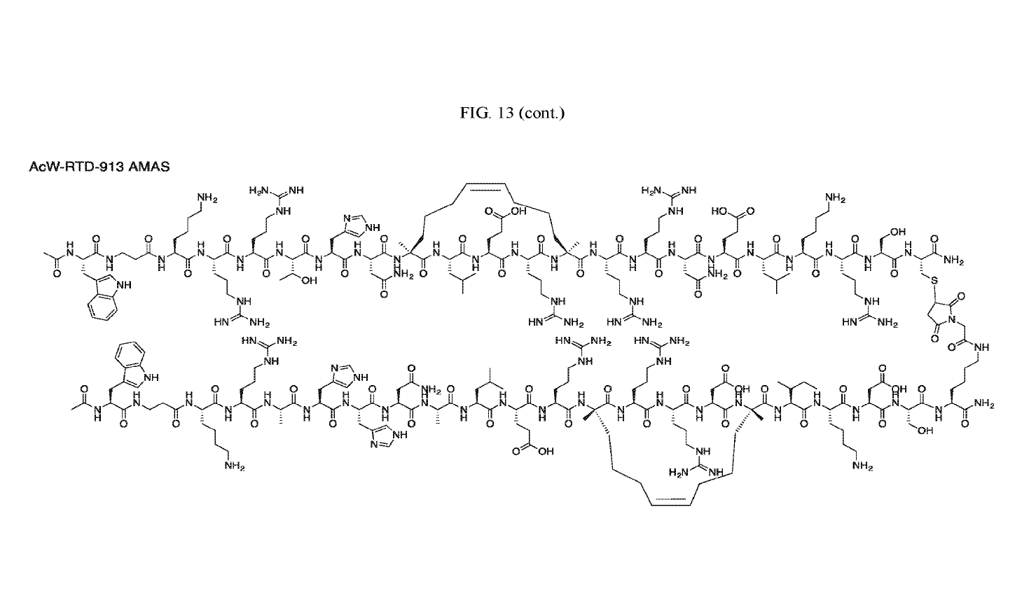

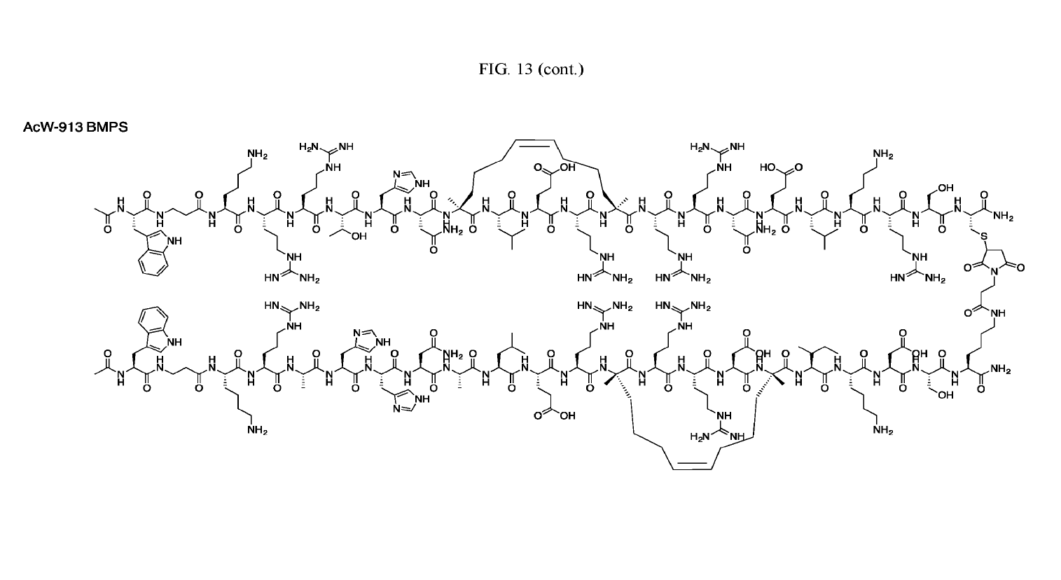

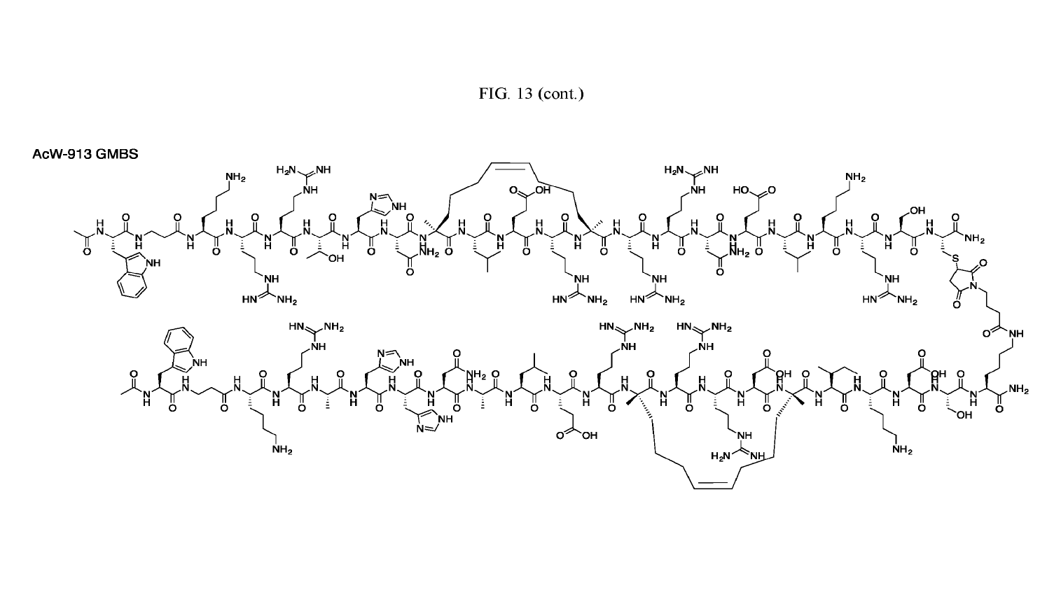

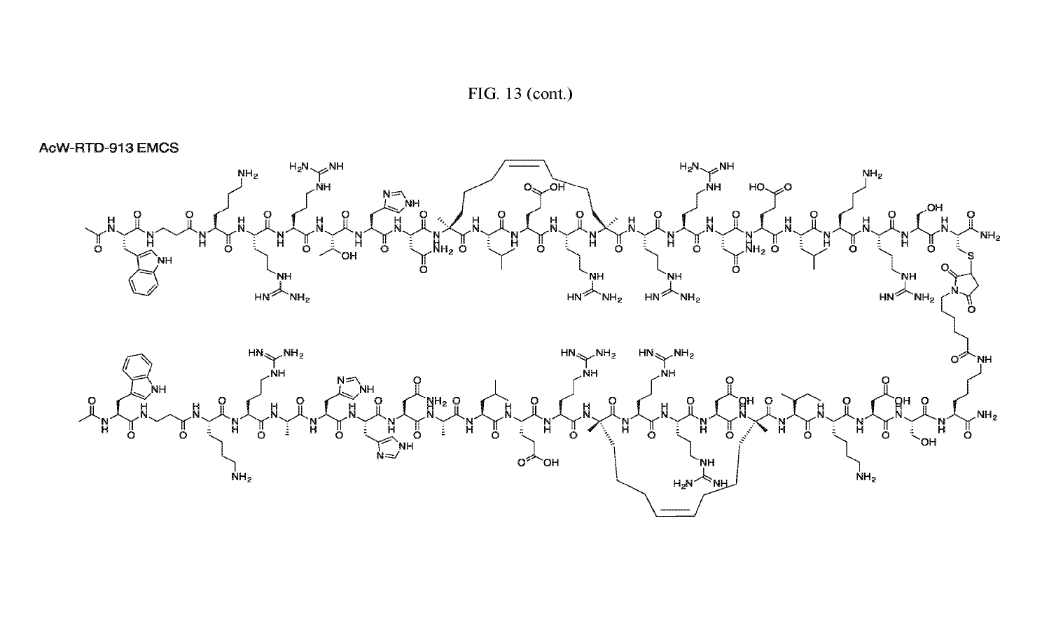

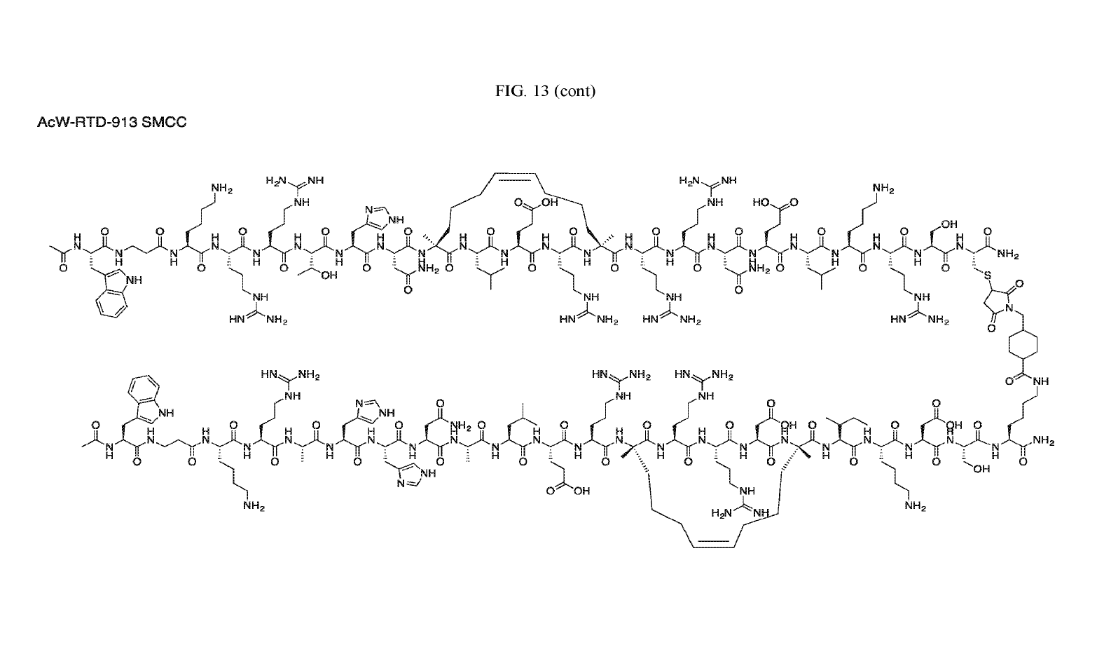

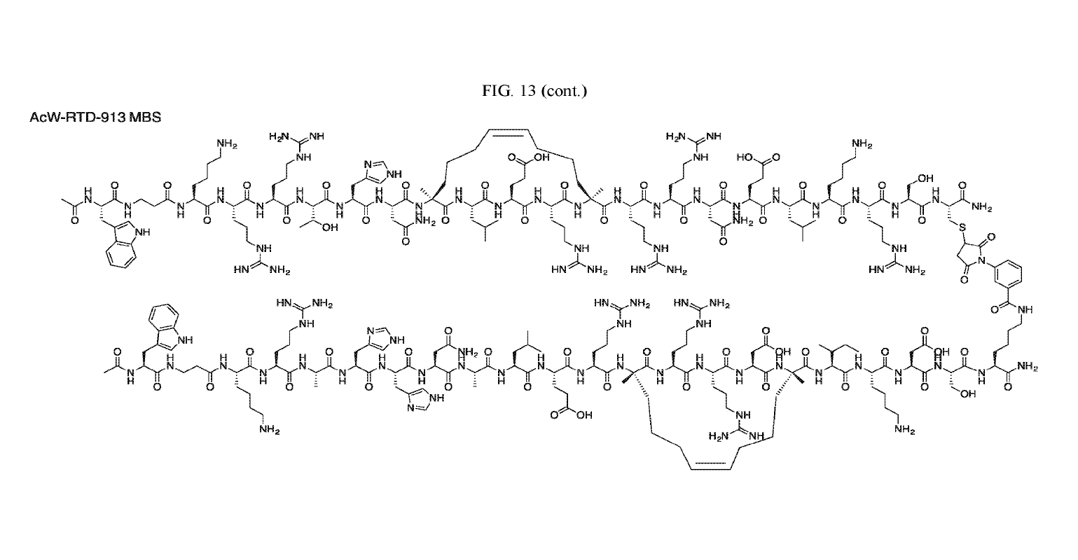

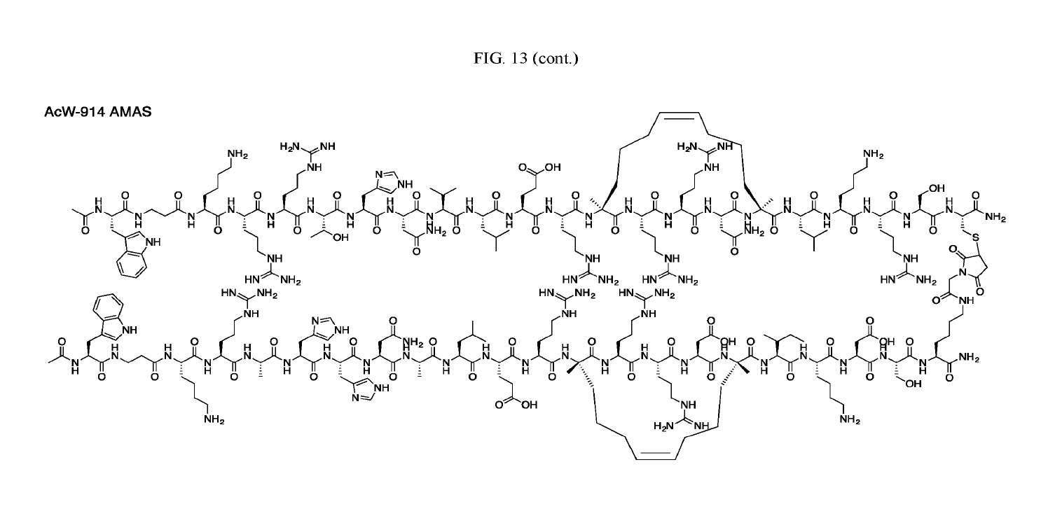

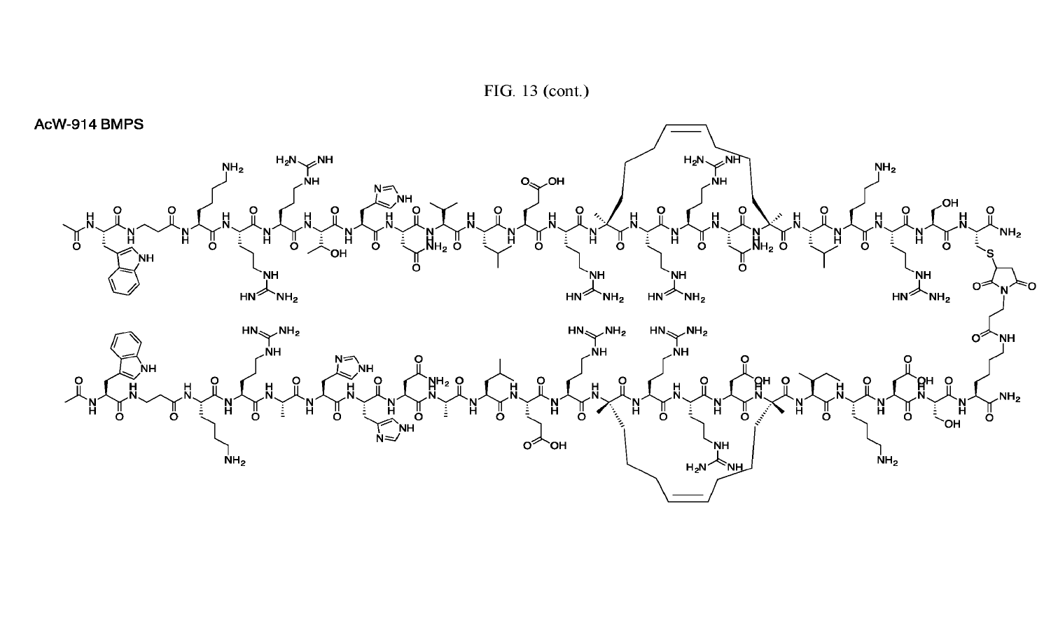

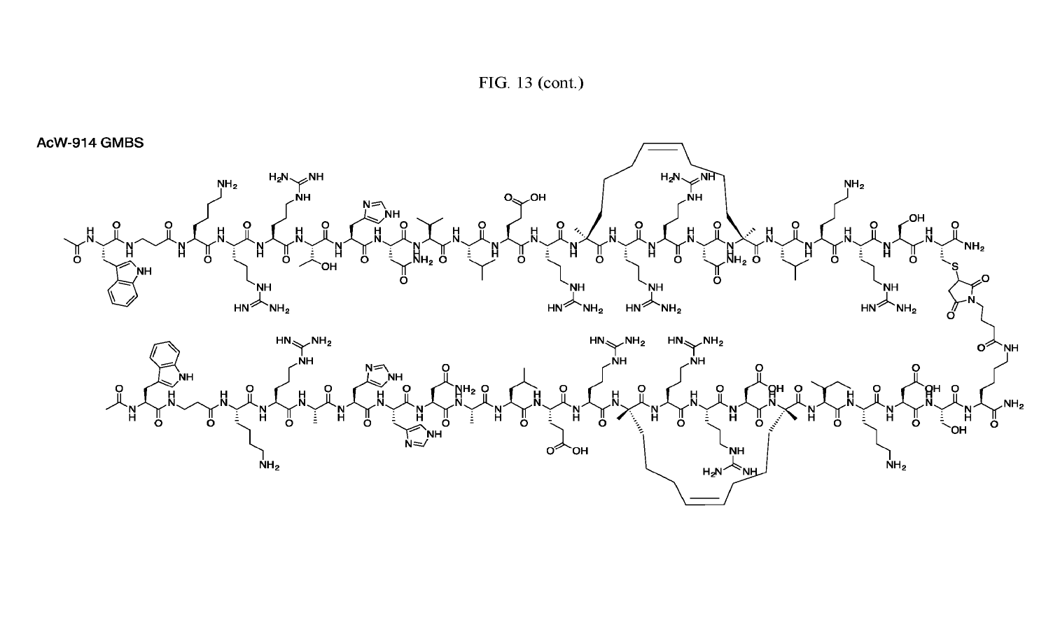

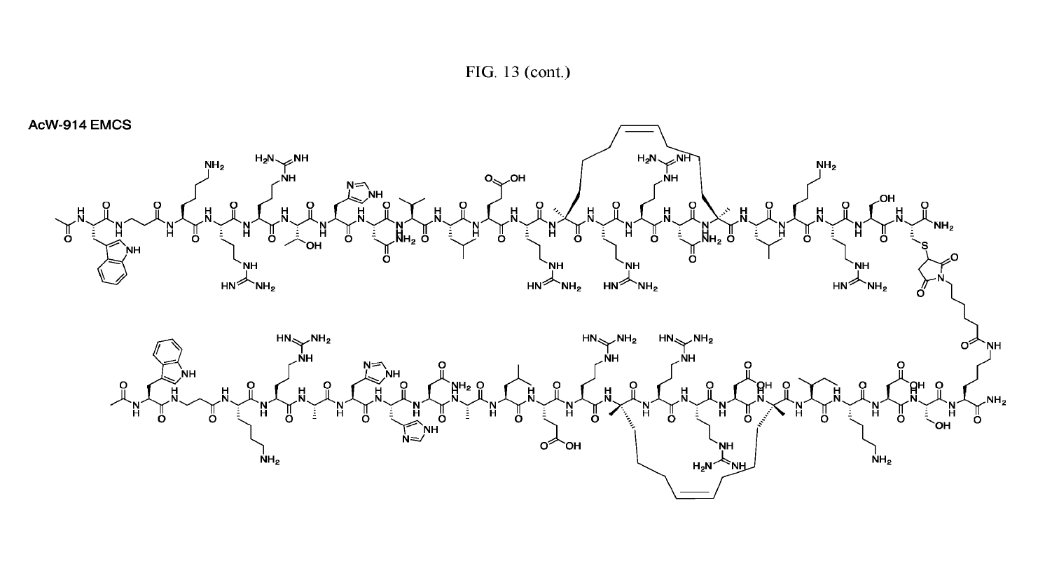

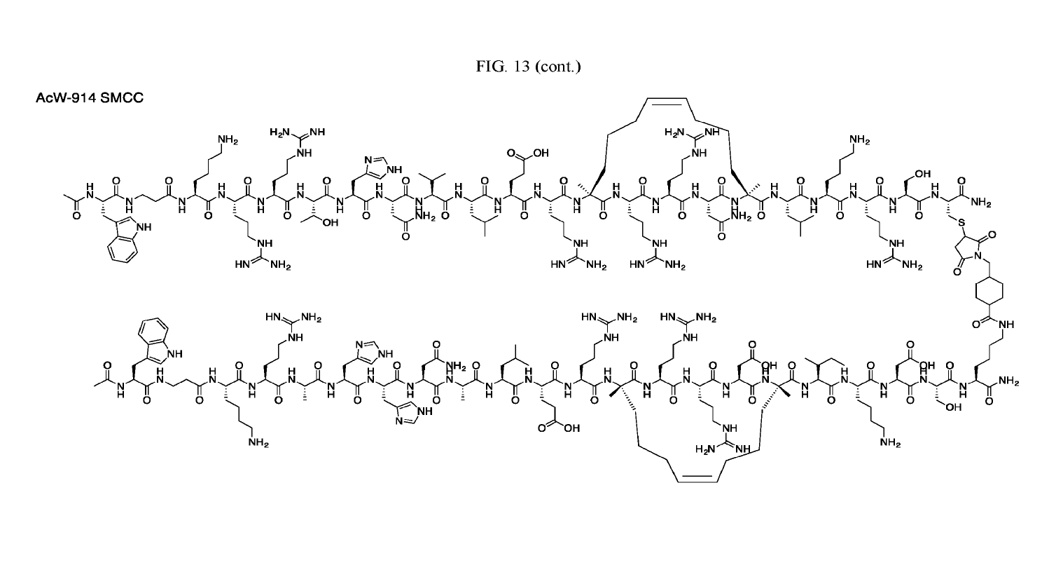

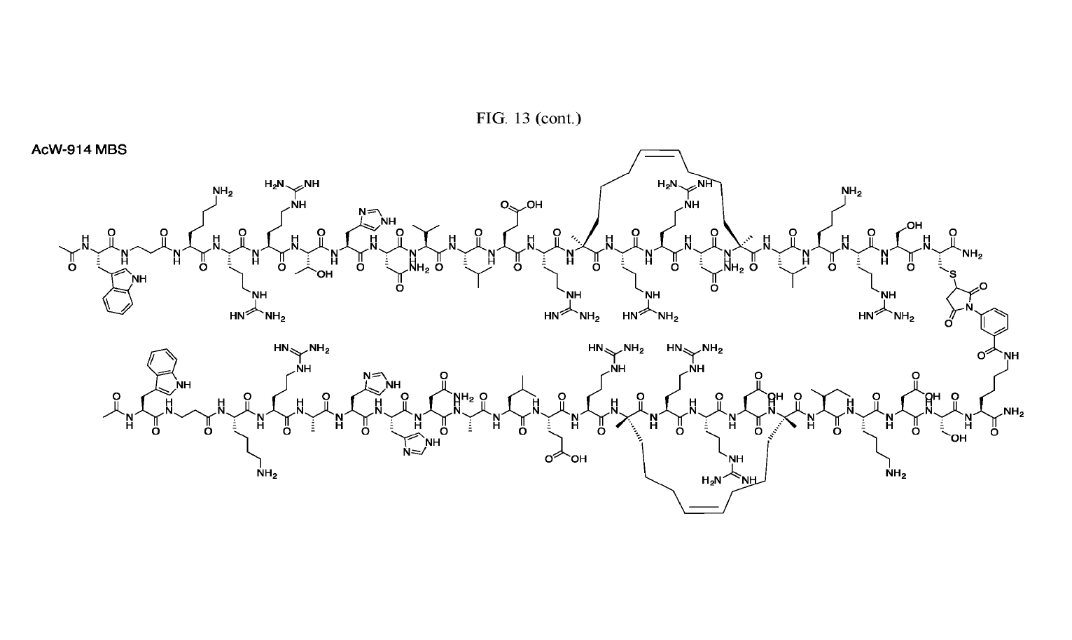

Tables 1-7 show exemplary monomeric (Tables 1-3) and dimeric (Tables 4-7) stabilized DNA-binding compounds. FIG. 12 shows monomer structures. FIG. 13 shows the dimer structures. Various derivatives of several of these that contain N-terminal functionalities such as a fluorophore (FITC for imaging) or a biotin (for IP or ChIP experiments) were generated and are shown in the Tables. The tables show information about different modifications and macrocycle locations, helical linkers and dimerization moieties.

All compounds follow the nomenclature of RTDXY where X and Y refer to the two monomers creating the dimer.

TABLE 1

| Peptide | Sequence | Linker | SEQ ID NO: |
|---|---|---|---|
| AcW-RTD-1 | AcW-•KRRTHNVLERQRRNELKRS•-C | — | 1 |
| AcW-RTD-3 | AcW-•KRAHHNALERKRRDHIKDS•-K (Mmt) | — | 2 |
| AcW-RTD-3 | AcW-•KRAHHNALERKRRDHIKDS•-K (Mmt) | SMCC | 3 |
| AcW-RTD-4 | AcW-•KRRTHN*LER*RRNELKRS•-C | — | 4 |
| AcW-RTD-5 | AcW-•KRRTHNVLER*RRN*LKRS•-C | — | 5 |
| AcW-RTD-6 | AcW-•KR*THN*LERQRRNELKRS•-C | — | 6 |
| AcW-RTD-2 | AcW-•KRAHHN*LER*RRDHIKDS•-K (Mmt) | SMCC | 7 |
| AcW-RTD-7 | AcW-•KRAHHNALER*RRD*IKDS•-K (Mmt) | — | 8 |
| AcW-RTD-7 | AcW-•KRAHHNALER*RRD*IKDS•-K (Mmt) | SMCC | 9 |
| AcW-RTD-8 | AcW-•KR*HHN*LERKRRDHIKDS•-K (Mmt) | SMCC | 10 |
| AcW-RTD- | AcW-GKRRTHN*LER*RRNELKRSG-C | — | 11 |
| 4G | | | |
| AcW-RTD- | AcW-GKR*HHN*LERKRRDHIKDSG-K (Mmt) | SMCC | 12 |
| 8G | | | |
| AcW-RTD-9 | AcW-βKRAHHNALER*RRD*IKDS-K (Mmt) | SMCC | 13 |
| AcW-RTD- | AcW-βKR*HHN*LERKRRDHIKDS-K (Mmt) | — | 14 |
| 10 | | | |
| AcW-RTD- | AcW-βKR*HHN*LERKRRDHIKDS-K (Mmt) | SMCC | 15 |
| 10 | | | |
| AcW-RTD- | AcW-βKRRTHN*LER*RRNELKRS-C | — | 16 |
| 13 | | | |
| AcW-RTD- | AcW-βKRRTHNVLER*RRN*LKRS-C | — | 17 |
| 14 | | | |
| AcW-RTD-9 | AcW-βKRAHHNALER*RRD*IKDS-K (Mmt) | AMAS | 18 |
| AcW-RTD-9 | AcW-βKRAHHNALER*RRD*IKDS-K (Mmt) | EMCS | 19 |
| AcW-RTD-9 | AcW-βKRAHHNALER*RRD*IKDS-K (Mmt) | MBS | 20 |

TABLE 2

| | | | SEQ ID |
|---|---|---|---|
| Peptide | Sequence | Linker | NO: |
| FITC-PEG3-RTD-1 | FITC-PEG3-•KRRTHNVLERQRRNELKRS•-C | — | 21 |
| FITC-PEG3-RTD-4 | FITC-PEG3-βKRRTHN*LER*RRNELKRSβ-C | — | 22 |
| FITC-PEG3-RTD-5 | FITC-PEG3-βKRRTHNVLER*RRN*LKRSβ-C | — | 23 |

TABLE 3

| Peptide | Sequence | Linker | SEQ ID NO: |
|---|---|---|---|
| Biotin-PEG3-W-RTD-4 | Biotin-PEG3-W- | — | 24 |
| | •KRRTHN*LER*RRNELKRS•-C | | |
| Biotin-PEG3-W-RTD- | Biotin-PEG3-W- | — | 25 |
| 13 | •KRRTHN*LER*RRNELKRS-C | | |

TABLE 4

| Peptide | Linker |
|---|---|
| AcW-RTD-31 | SMCC |
| AcW-RTD-74 | SMCC |
| AcW-RTD-75 | SMCC |
| AcW-RTD-84 | SMCC |
| AcW-RTD-85 | SMCC |
| AcW-RTD-84G | SMCC |
| AcW-RTD-913 | SMCC |
| AcW-RTD-914 | SMCC |
| AcW-RTD-1013 | SMCC |
| AcW-RTD-1014 | SMCC |
| AcW-RTD-913 | AMAS |
| AcW-RTD-913 | EMCS |
| AcW-RTD-913 | MBS |
| AcW-RTD-44 | SMCC |
| AcW-RTD-55 | SMCC |

TABLE 5

| Peptide | Linker |
|---|---|
| FITC-PEG3-RTD-31 | SMCC |
| FITC-PEG3-RTD-85 | SMCC |
| FITC-PEG3-RTD-74 | SMCC |
| FITC-PEG3-RTD-84 | SMCC |
| FITC-PEG3-RTD-75 | SMCC |

TABLE 6

| Peptide | Linker |
|---|---|
| Biotin-RTD-913 | MBS |

TABLE 7

| | | | SEQ ID |
|---|---|---|---|
| Peptide | Sequence | Stapled? | NO: |
| Fmoc-RTD-1 | Fmoc-•KRRTHNVLERQRRNELKRS•-C | — | 26 |
| Fmoc-RTD-3 | Fmoc-•KRAHHNALERKRRDHIKDS•-K (Mmt) | — | 27 |
| Fmoc-RTD-4 | Fmoc-•KRRTHN*LER*RRNELKRS•-C | No | 28 |
| Fmoc-RTD-4 | Fmoc-•KRRTHN*LER*RRNELKRS•-C | Yes | 29 |
| Fmoc-RTD-5 | Fmoc-•KRRTHNVLER*RRN*LKRS•-C | No | 30 |
| Fmoc-RTD-5 | Fmoc-•KRRTHNVLER*RRN*LKRS•-C | Yes | 31 |
| Fmoc-RTD-7 | Fmoc-•KRAHHNALER*RRD*IKDS•-K (Mmt) | No | 32 |
| Fmoc-RTD-7 | Fmoc-•KRAHHNALER*RRD*IKDS•-K (Mmt) | Yes | 33 |
| Fmoc-RTD-8 | Fmoc-•KR*HHN*LERKRRDHIKDS•-K (Mmt) | No | 34 |
| Fmoc-RTD-8 | Fmoc-•KR*HHN*LERKRRDHIKDS•-K (Mmt) | Yes | 35 |
| Fmoc-RTD- | Fmoc-•KRRTHN*LER*RRNELKRSG-C | No | 36 |
| 4G | | | |
| Fmoc-RTD- | Fmoc-•KRRTHN*LER*RRNELKRSG-C | Yes | 37 |
| 4G | | | |
| Fmoc-RTD- | Fmoc-•KR*HHN*LERKRRDHIKDSG-K (Mmt) | No | 38 |
| 8G | | | |
| Fmoc-RTD- | Fmoc-•KR*HHN*LERKRRDHIKDSG-K (Mmt) | Yes | 39 |
| 8G | | | |
| Fmoc-RTD-9 | Fmoc-•KRAHHNALER*RRD*IKDS-K (Mmt) | No | 40 |
| Fmoc-RTD-9 | Fmoc-•KRAHHNALER*RRD*IKDS-K (Mmt) | Yes | 41 |
| Fmoc-RTD- | Fmoc-•KR*HHN*LERKRRDHIKDS-K (Mmt) | No | 42 |
| 10 | | | |
| Fmoc-RTD- | Fmoc-•KR*HHN*LERKRRDHIKDS-K (Mmt) | Yes | 43 |
| 10 | | | |
| Fmoc-RTD- | Fmoc-•KRRTHN*LER*RRNELKRS-C | No | 44 |
| 13 | | | |
| Fmoc-RTD- | Fmoc-•KRRTHN*LER*RRNELKRS-C | Yes | 45 |
| 13 | | | |
| Fmoc-RTD- | Fmoc-•KRRTHNVLER*RRN*LKRS-C | No | 46 |
| 14 | | | |
| Fmoc-RTD- | Fmoc-•KRRTHNVLER*RRN*LKRS-C | Yes | 47 |
| 14 | | | |
| Fmoc-RTD- | Fmoc-•KRRTHN*LER*RRNELKRS•-K (Mmt) | No | 48 |
| 4′ | | | |
| Fmoc-RTD- | Fmoc-•KRRTHNVLER*RRN*LKRS•-K (Mmt) | No | 49 |
| 5′ | | | |
| Fmoc-RTD- | Fmoc-•KRRTHN*LER*RRNELKRS•-K (Mmt) | Yes | 50 |
| 4′ | | | |
| Fmoc-RTD- | Fmoc-•KRRTHNVLER*RRN*LKRS•-K (Mmt) | Yes | 51 |
| 5′ | | | |

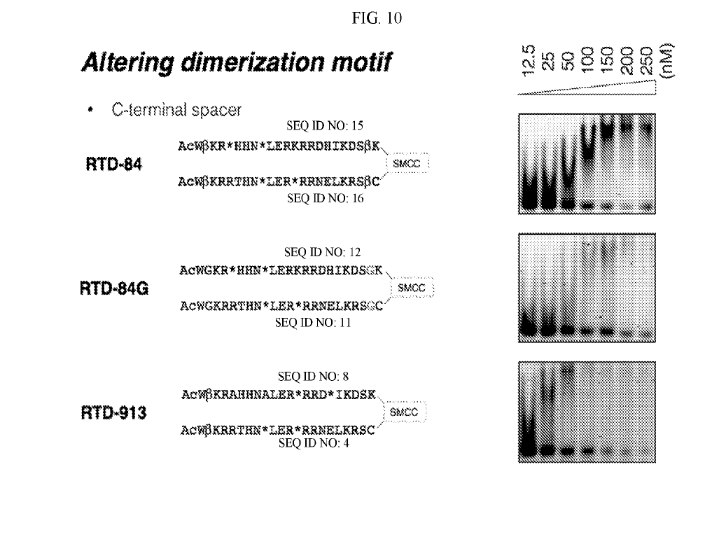

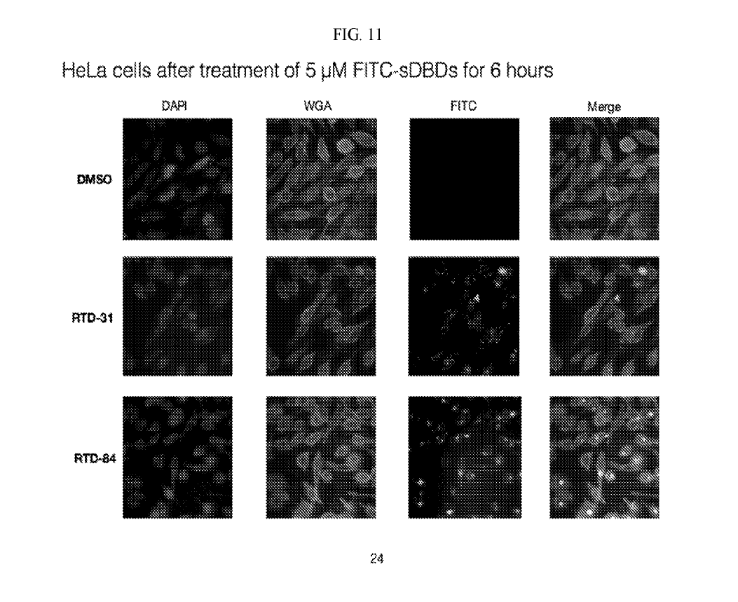

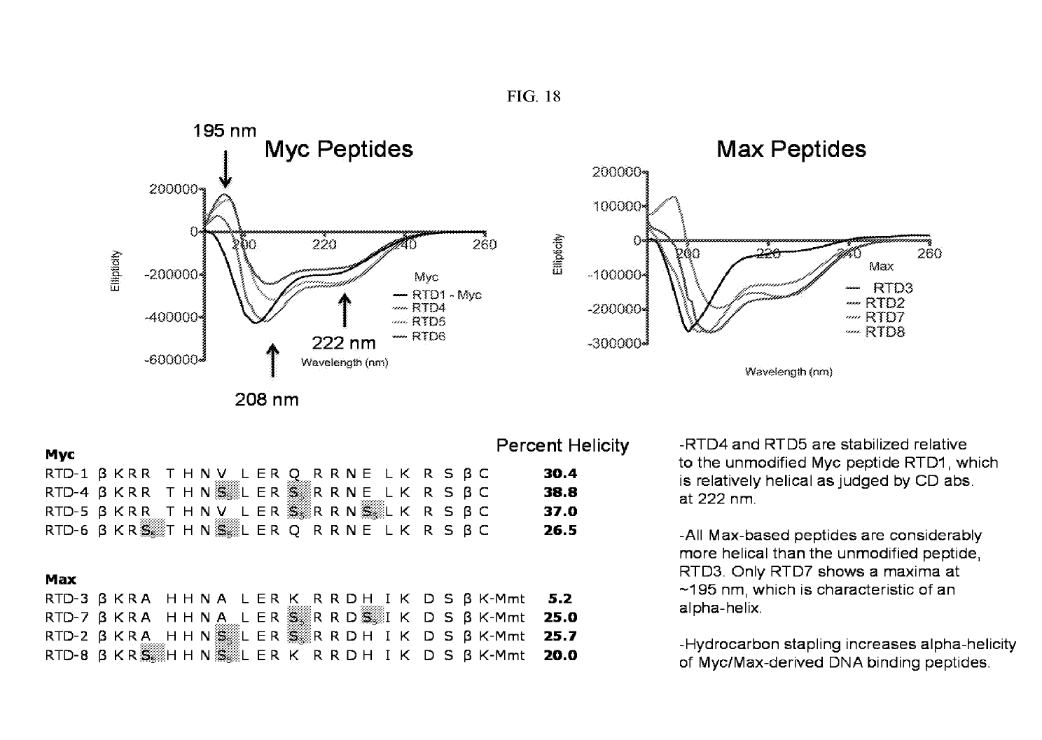

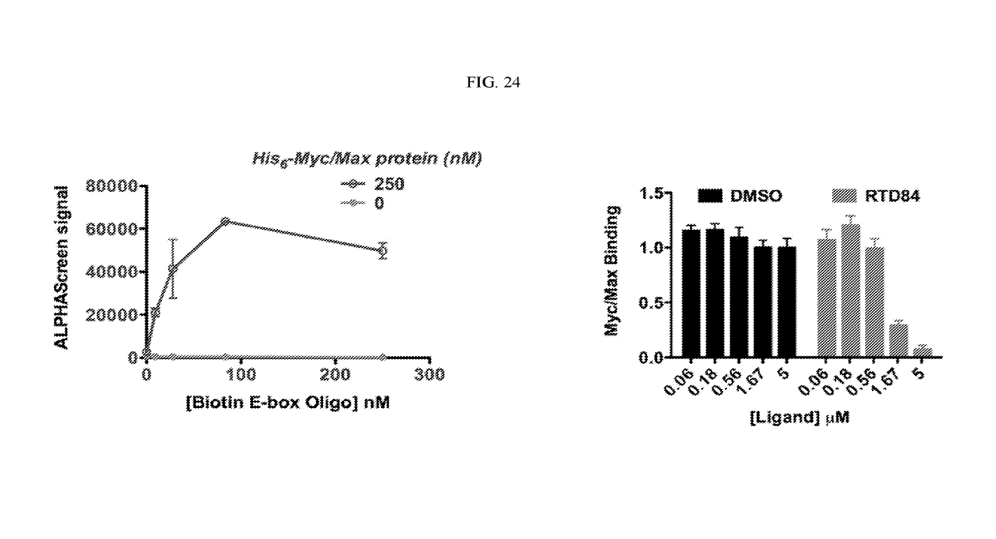

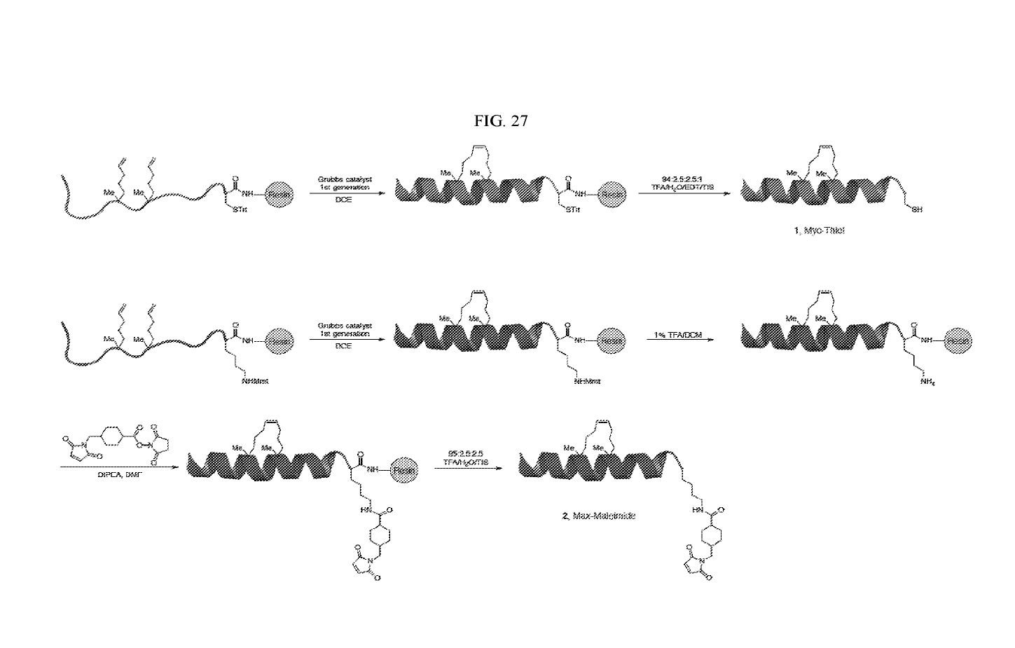

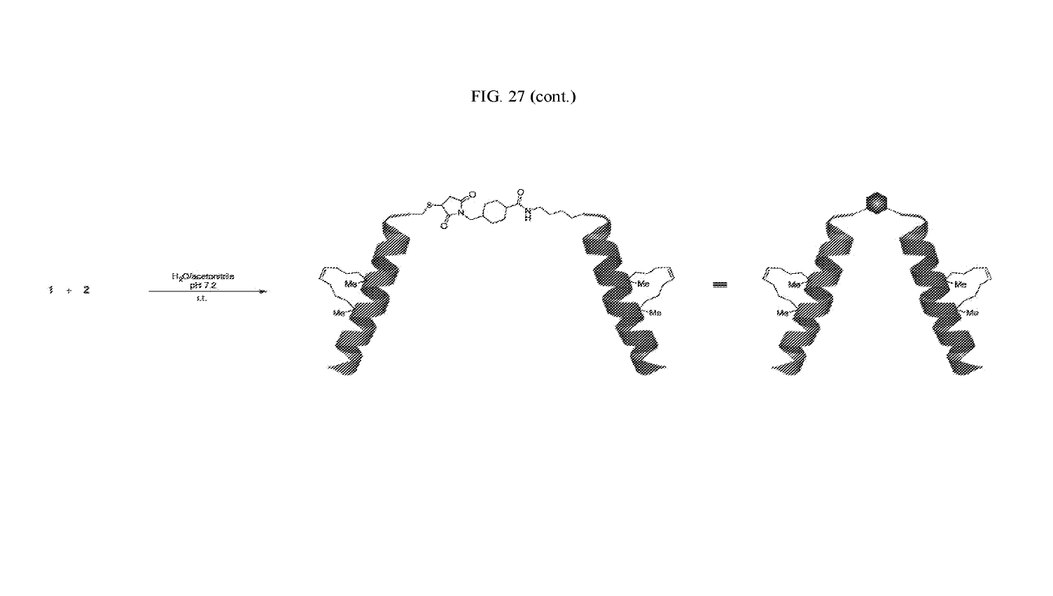

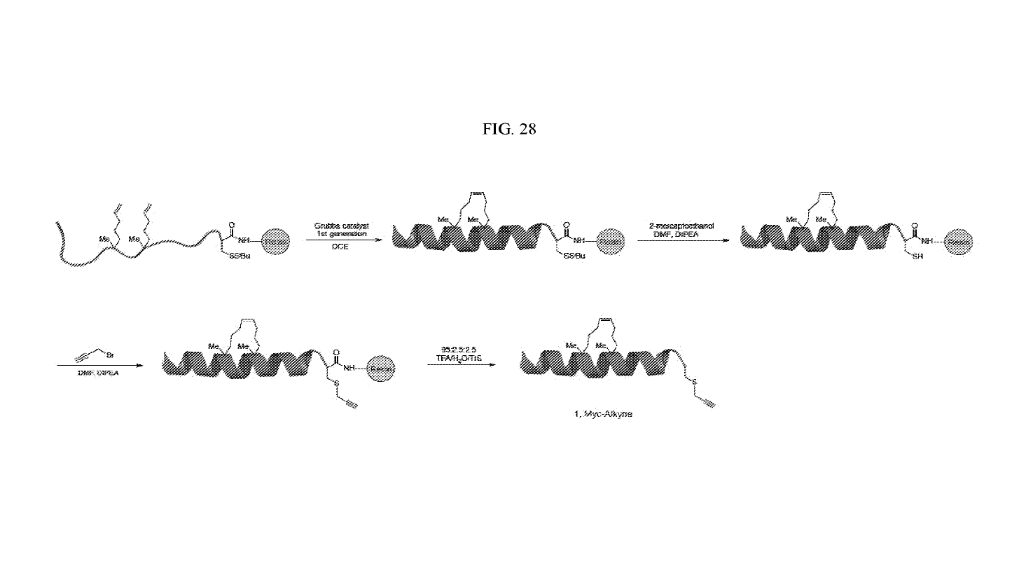

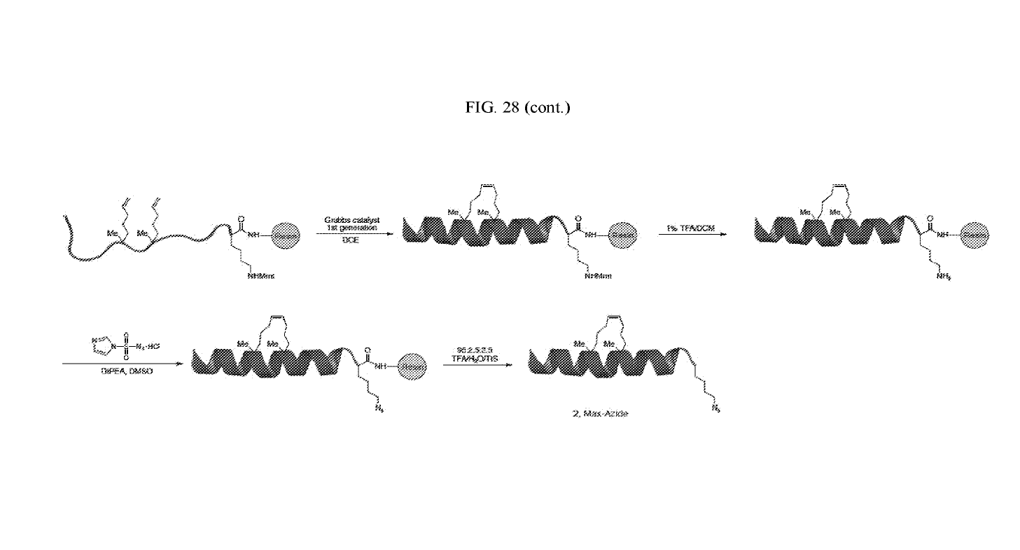

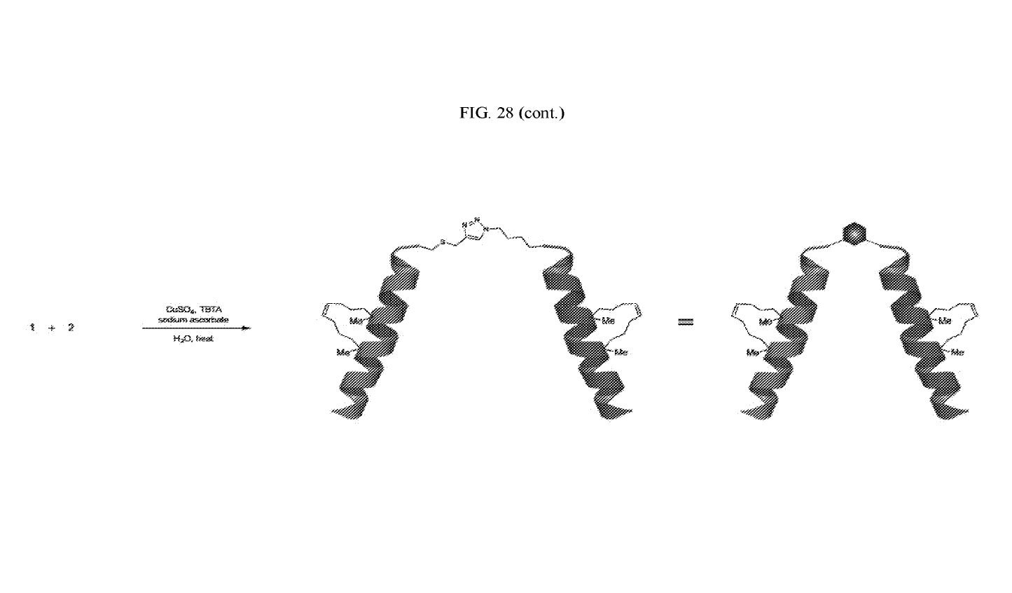

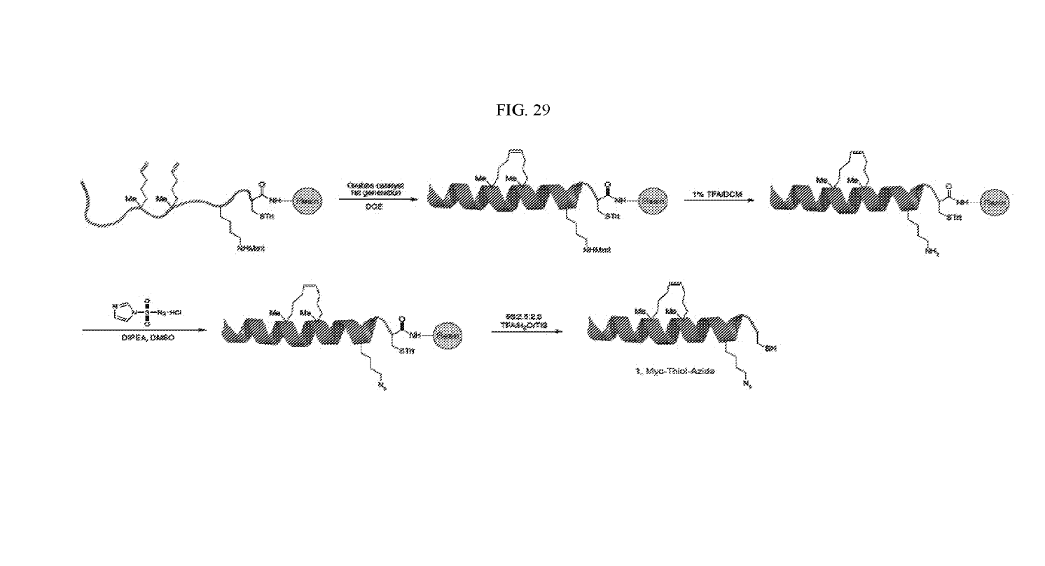

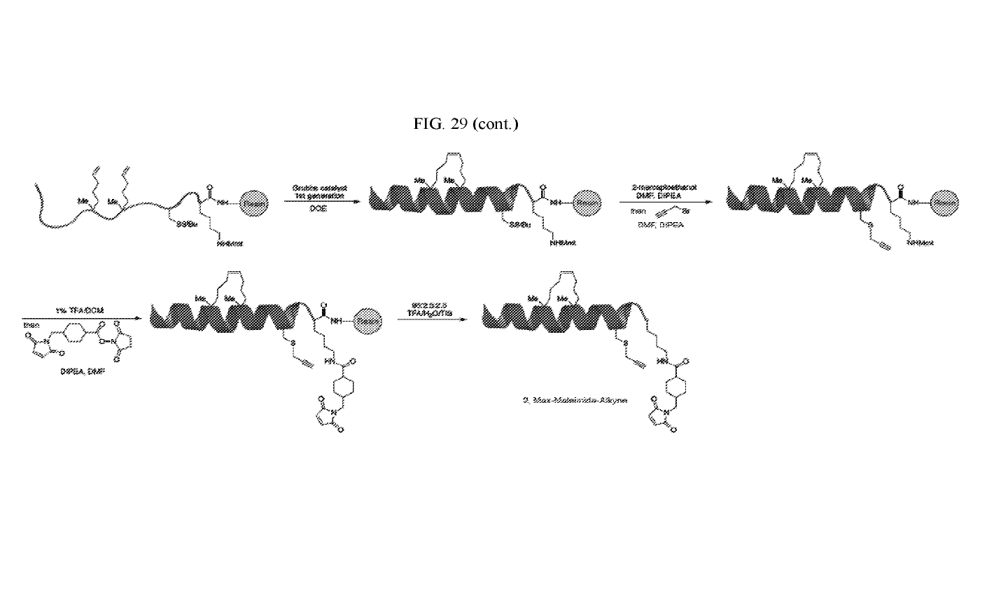

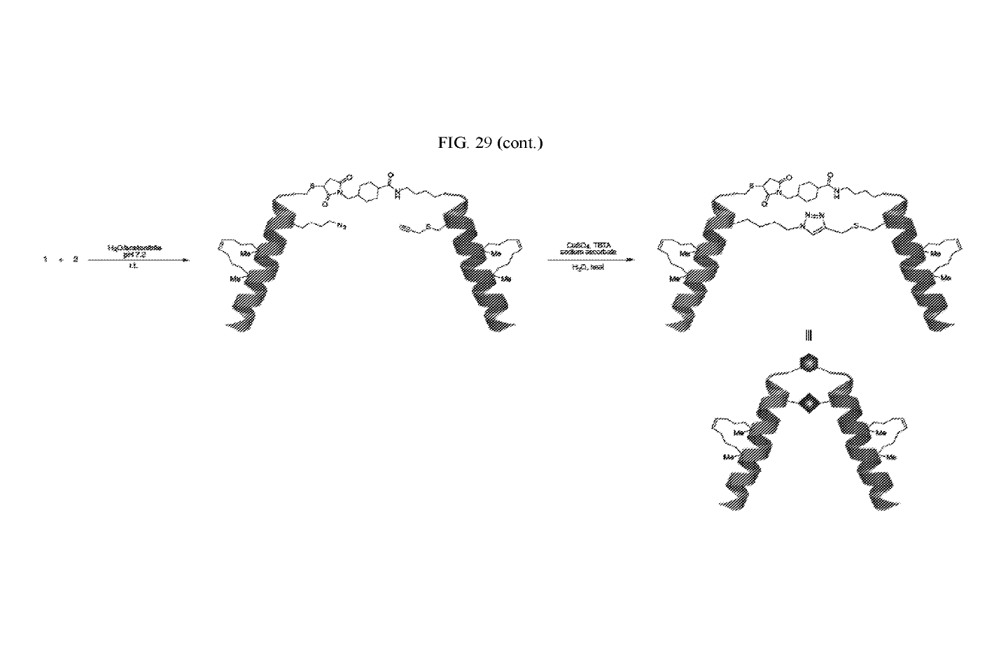

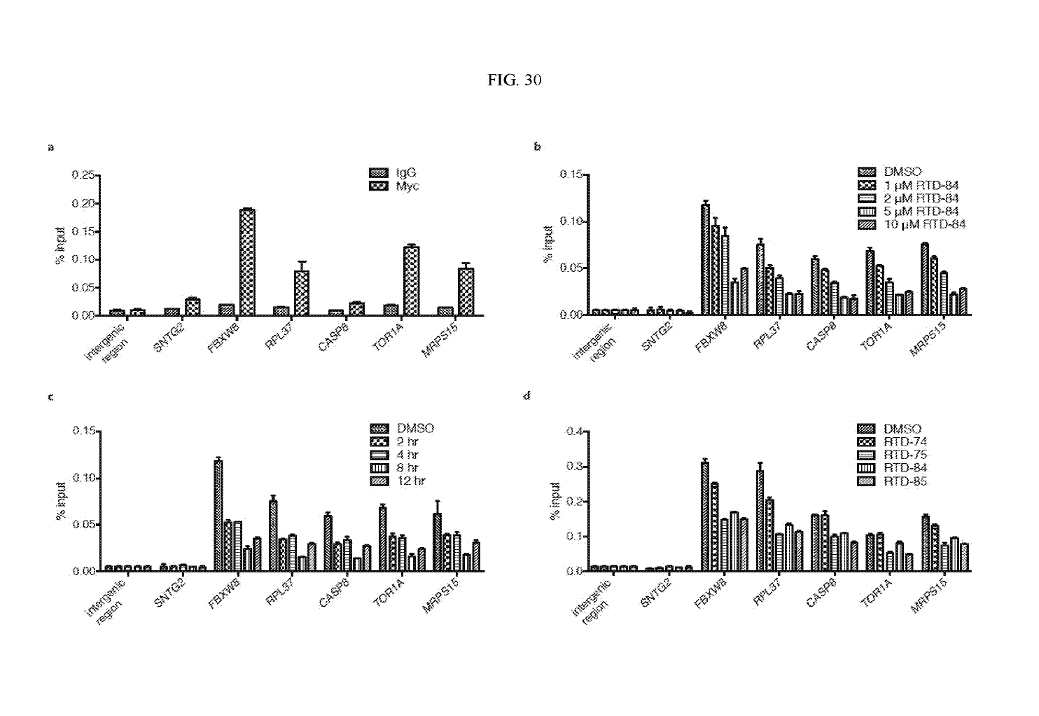

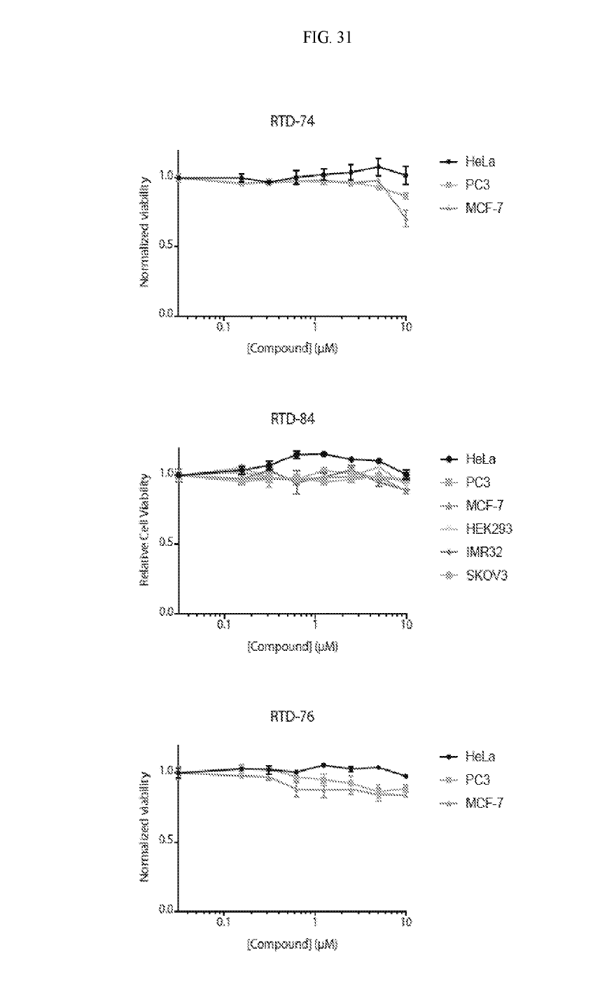

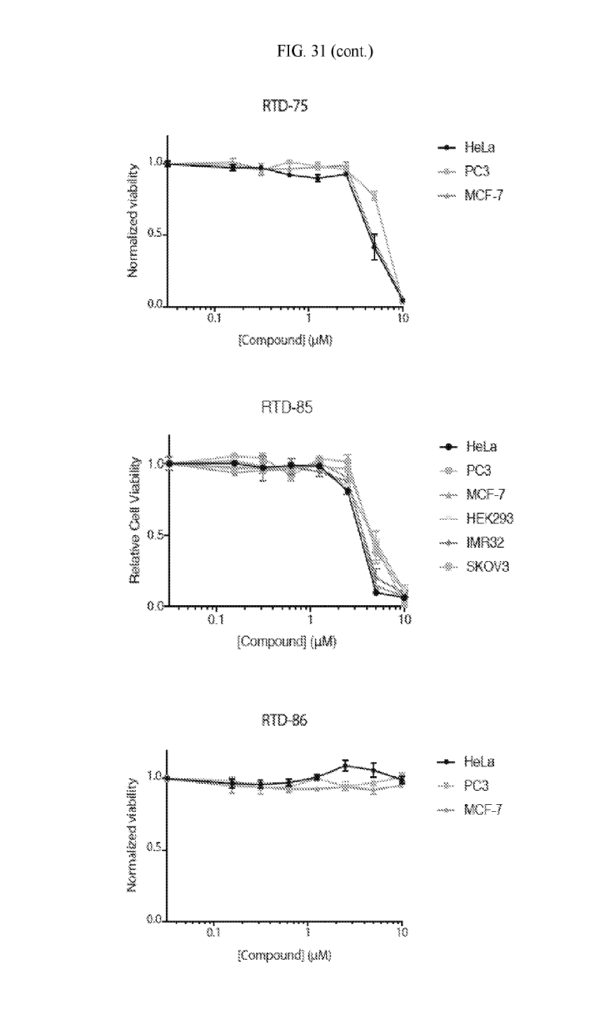

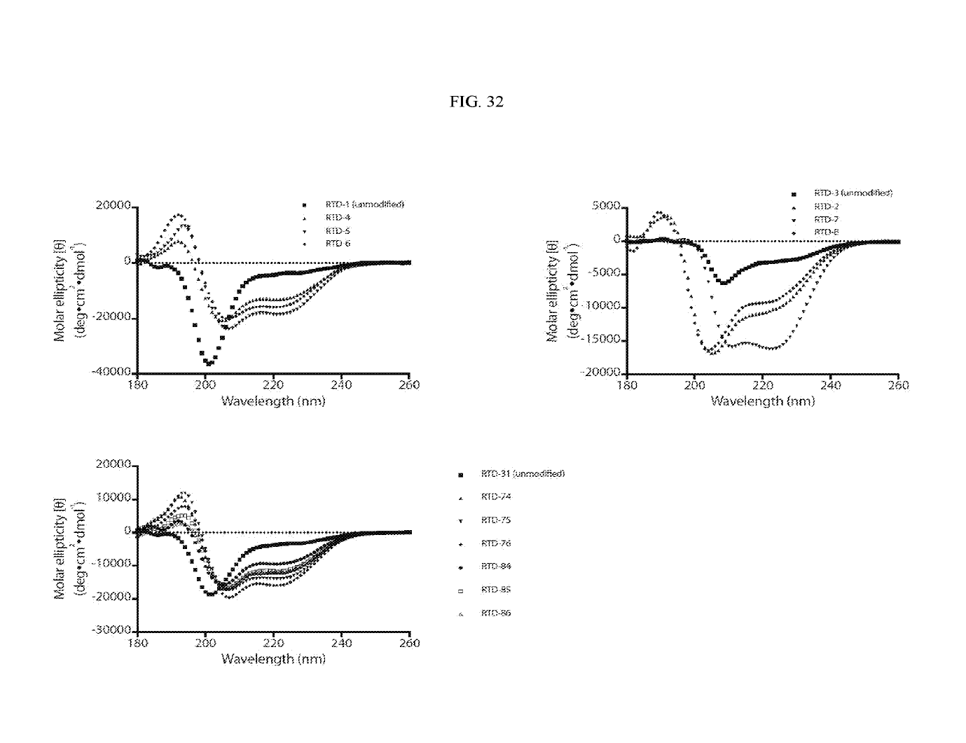

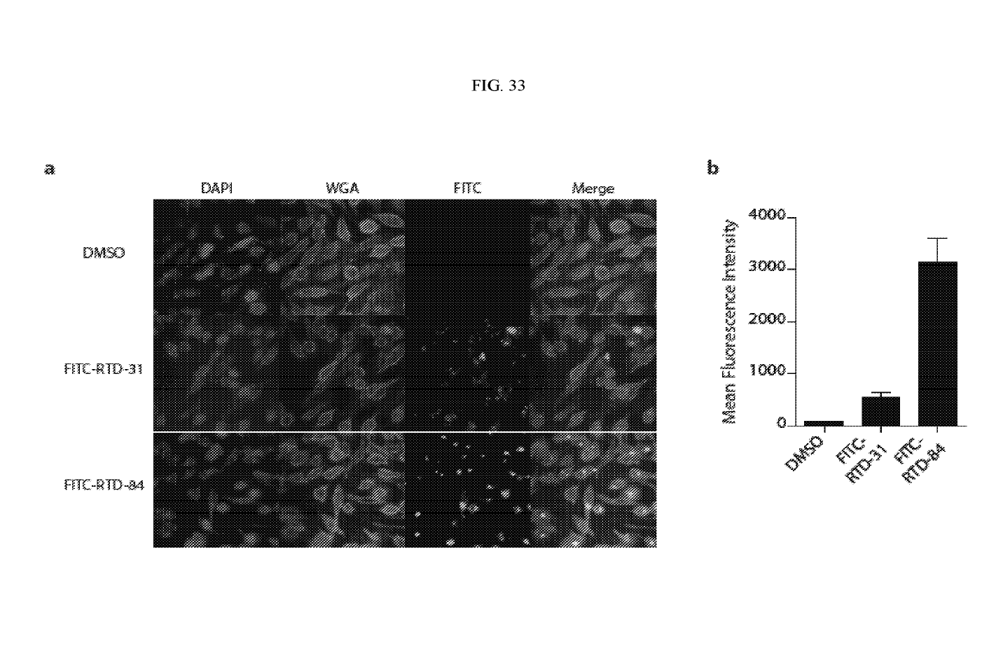

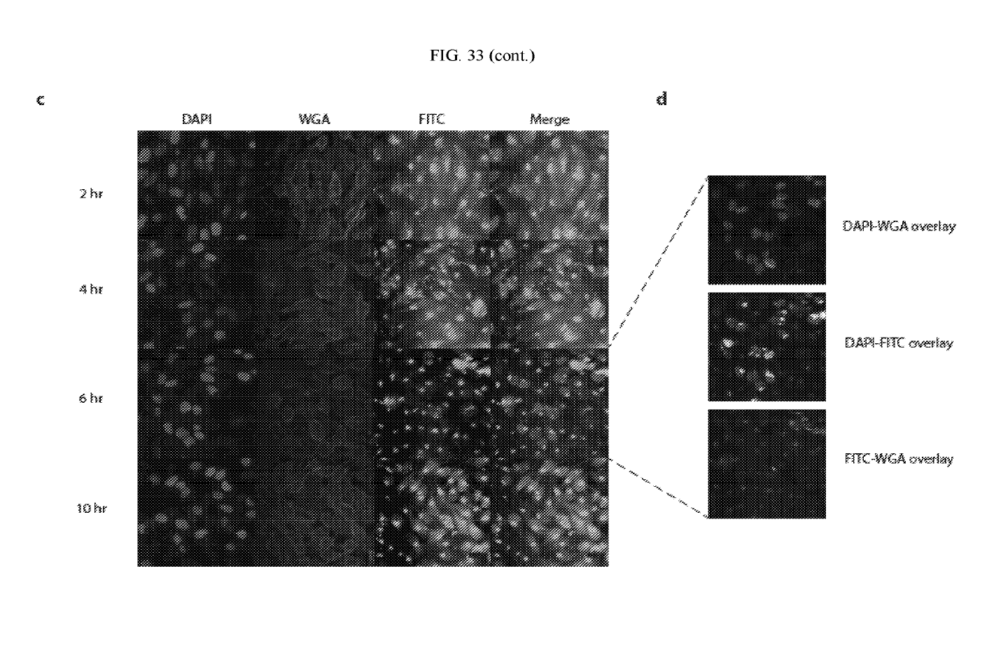

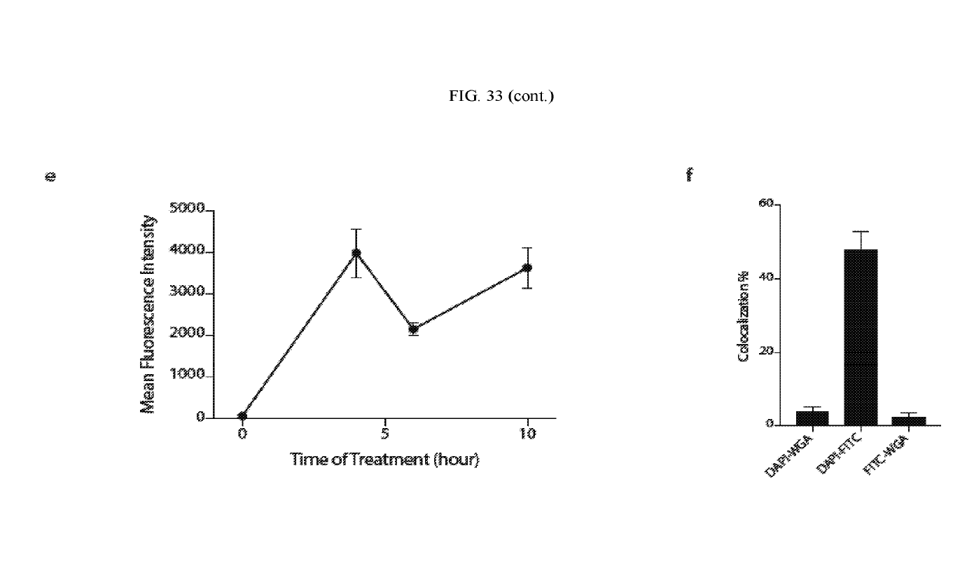

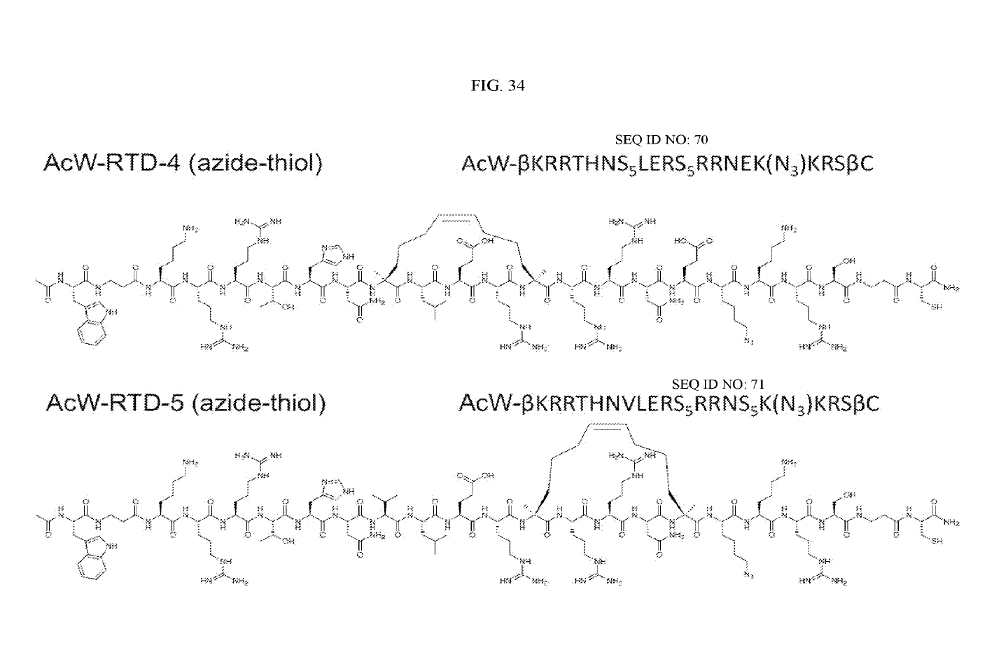

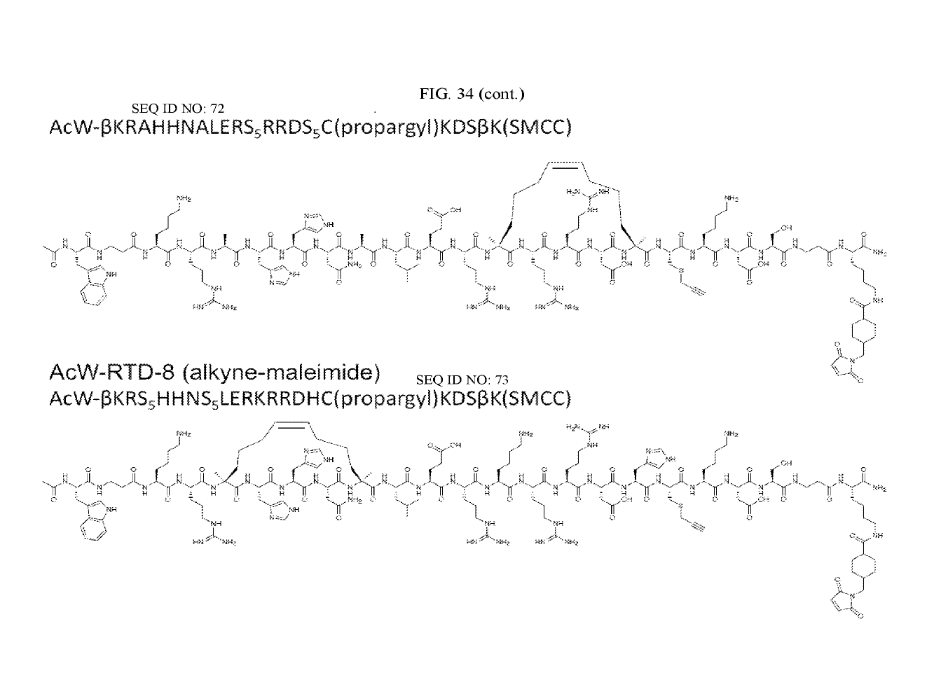

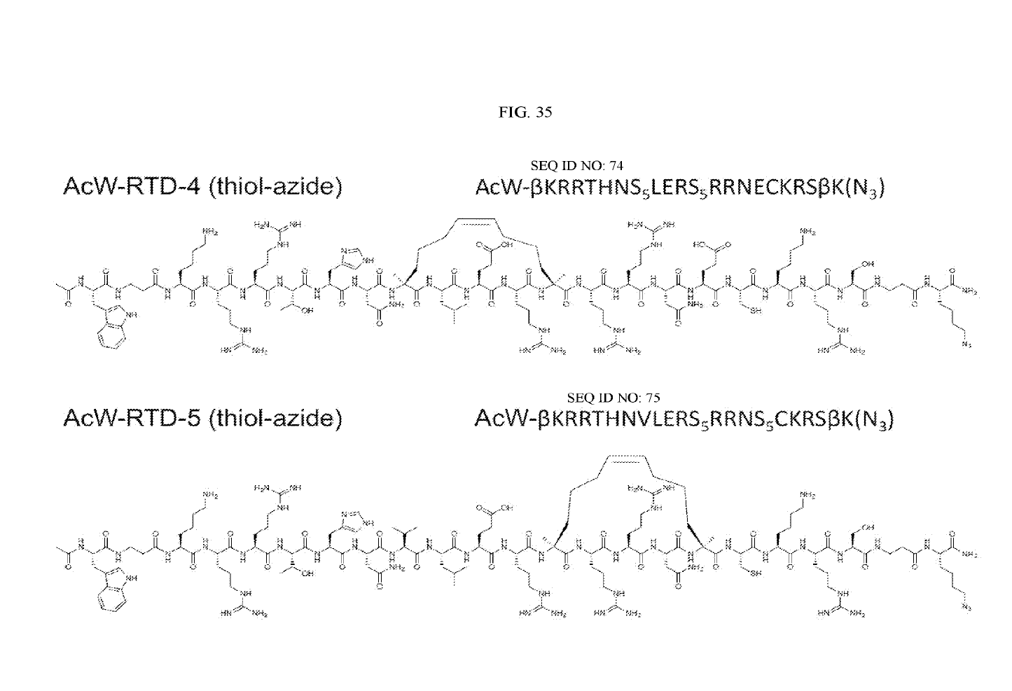

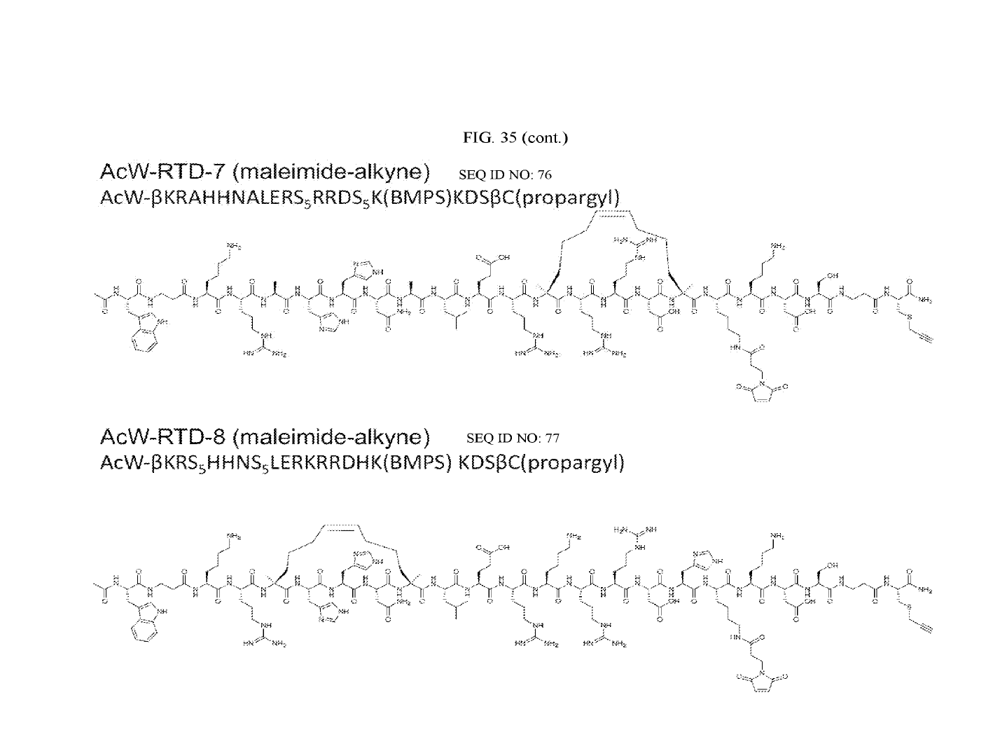

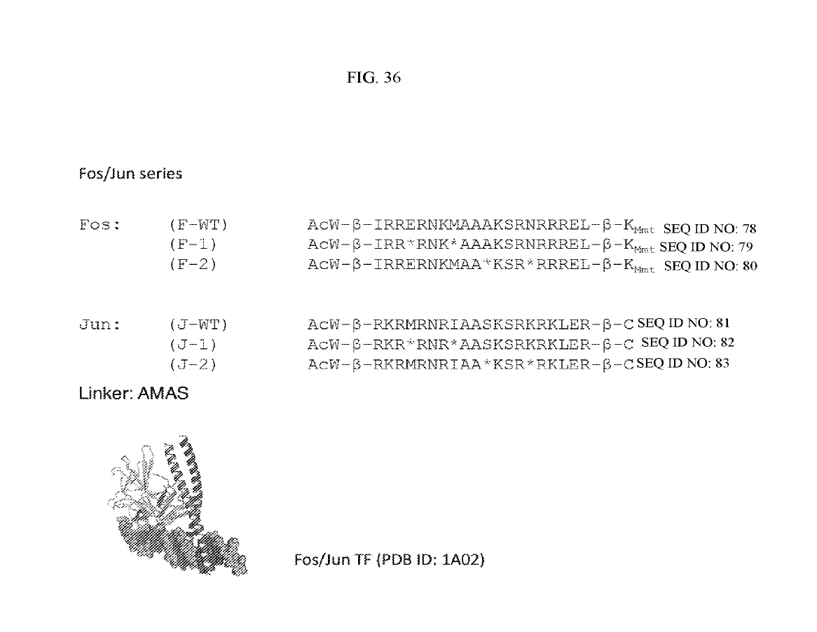

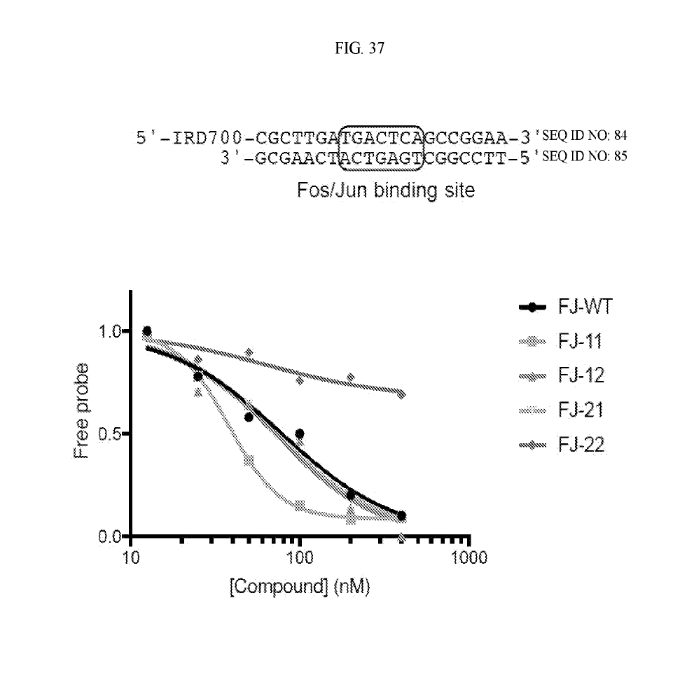

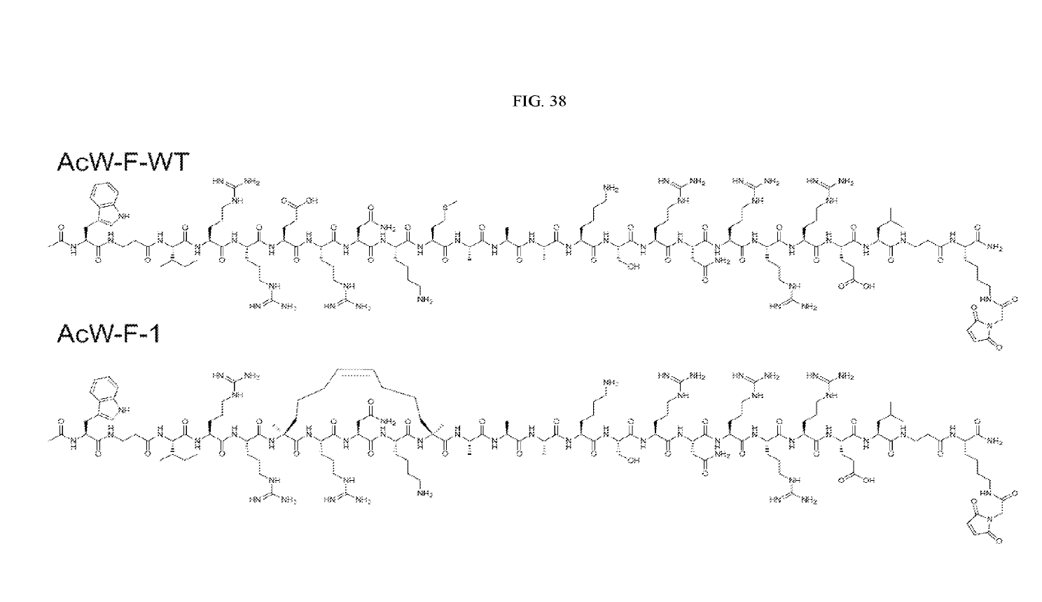

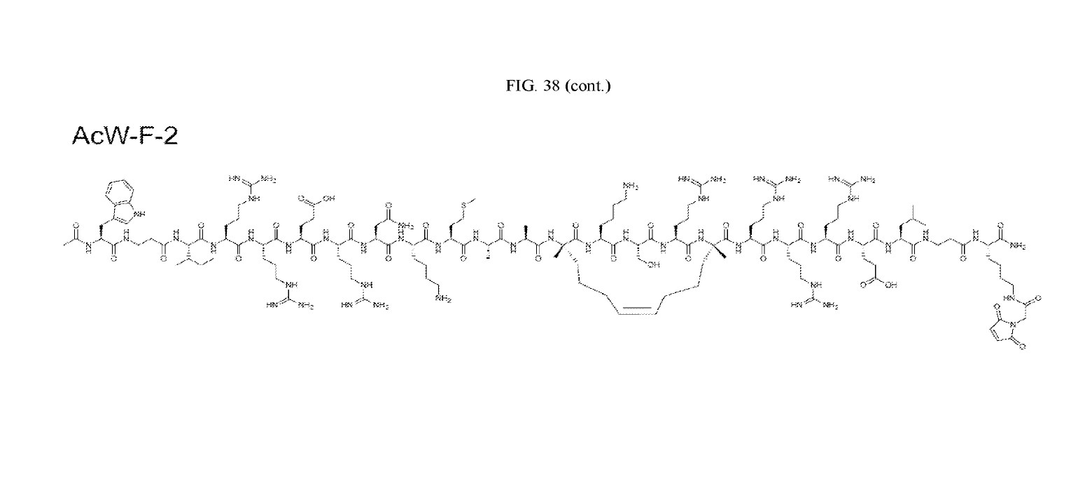

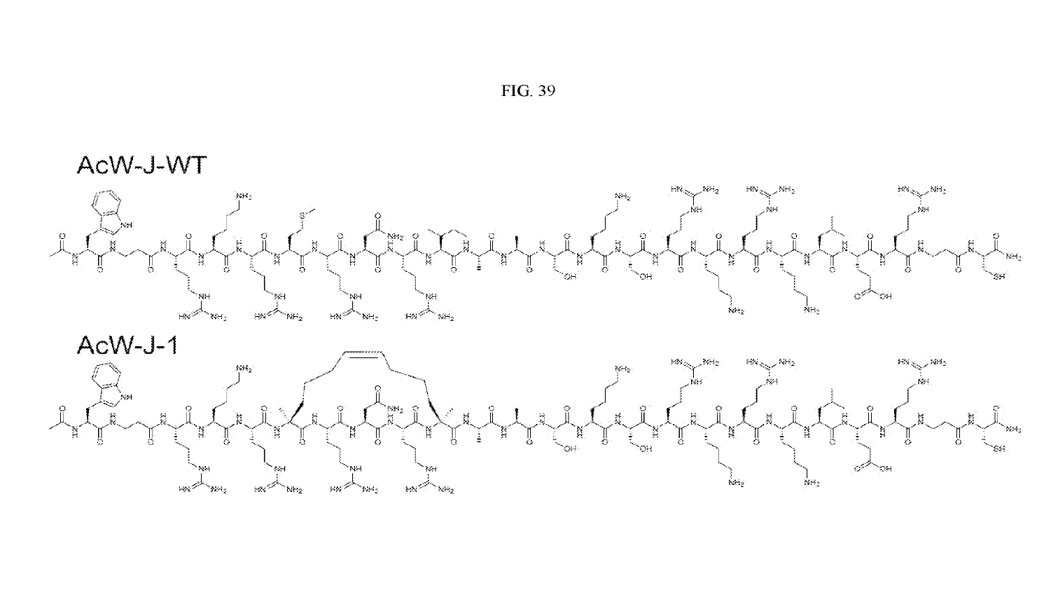

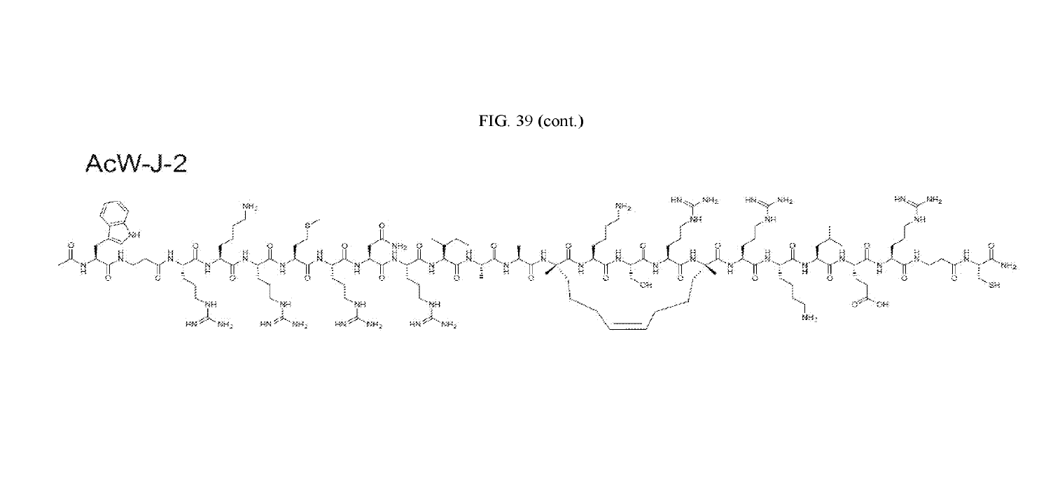

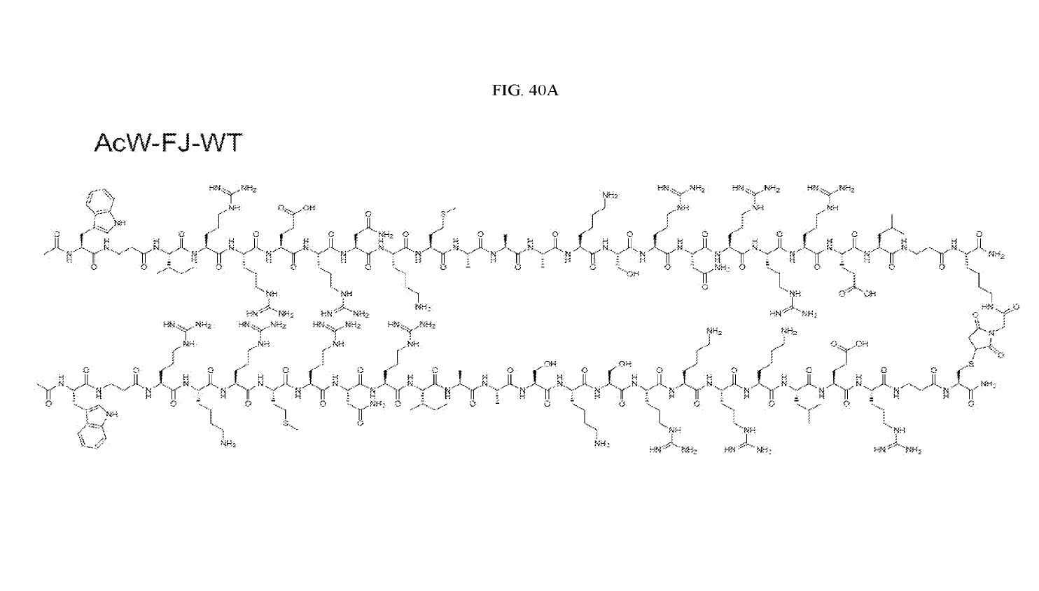

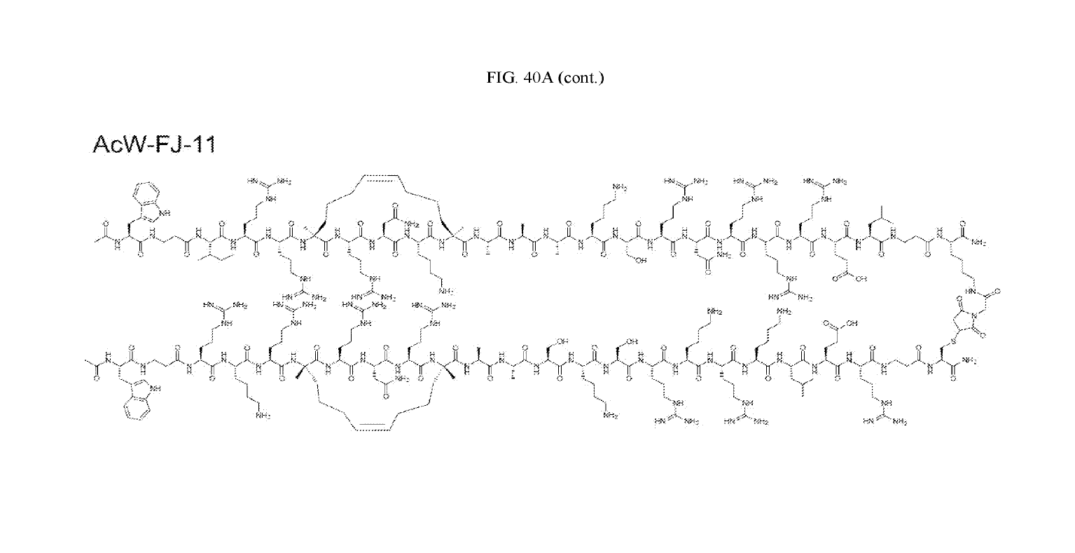

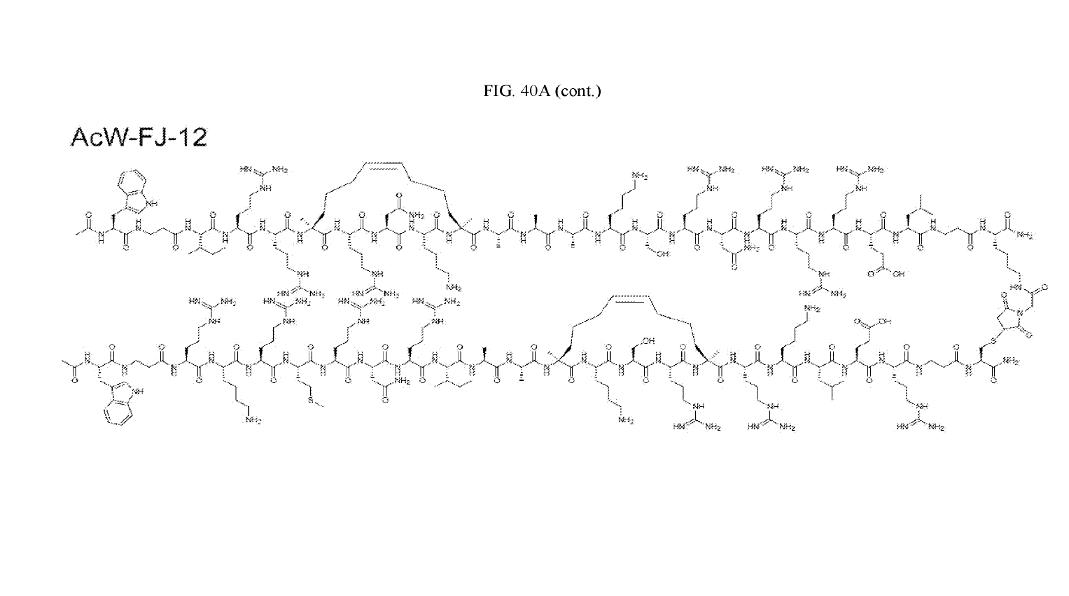

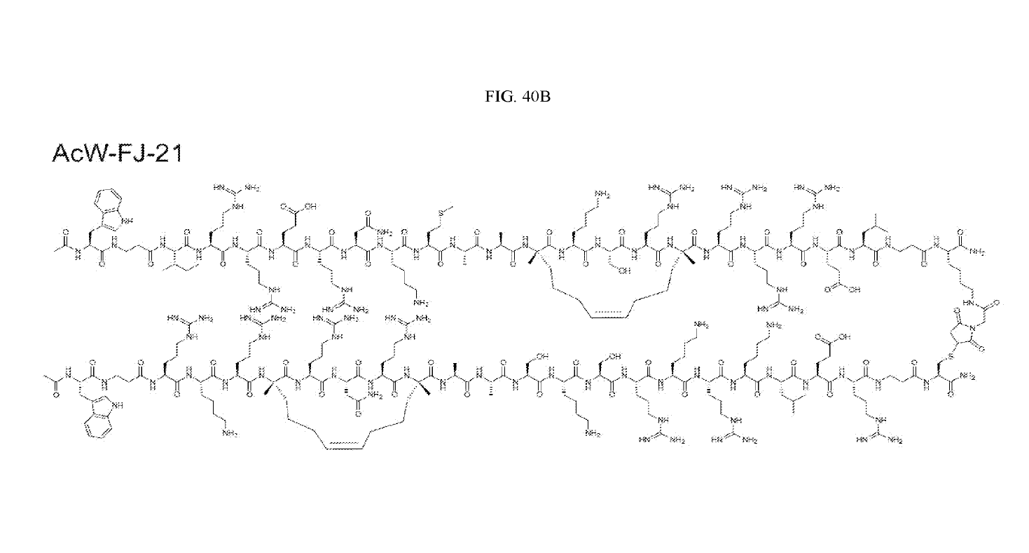

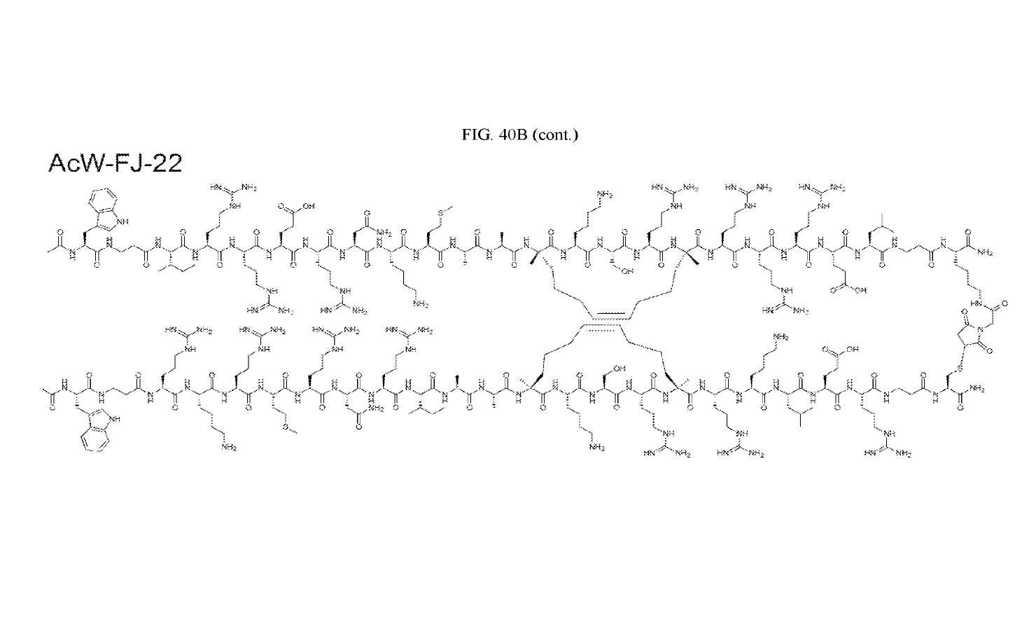

These peptides are assayed for activity using the methods described in Example 1 above. Results are shown in FIGS. 10-13 and 16-29. FIG. 18 shows circular dicrhoism of peptides. RTD4 and RTD5 are stabilized relative to the unmodified Myc peptide RTD1, which is relatively helical as judged by CD absorbance at 222 nm. All Max-based peptides are considerably more helical than the unmodified peptide, RTD3. Only RTD7 shows a maxima at ˜195 nm, which is characteristic of an alpha-helix. Hydrocarbon stapling increases alpha-helicity of Myc/Max-derived DNA binding peptides.

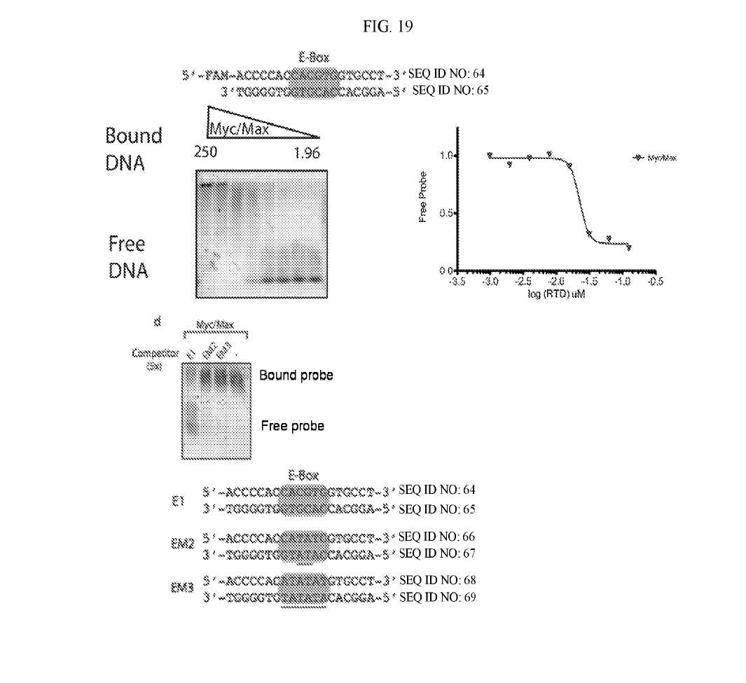

FIG. 19 shows sDBD DNA-binding activity measured by gel-shift assay. A fluorophore-labeled (5′-FAM) oligonucleotide containing a central E-box sequence was used (at 10 nM) to assess Myc/Max and DMD binding. This sequence was used in the Myc/Max/E-box crystal structure. Myc/Max protein specifically binds this oligo in a gel shift assay. The lower band is free E-box probe whereas the higher band is Myc/Max-bound E-box probe. Addition of excess non-labeled E-box probe (E1) competes this interaction. Addition of excess mutant probe (EM2 or EM3) does not significantly block Myc/Max binding. Thus, gel shift assays with the E-box probe are suitable for determining DMD (RTD) DNA-binding.

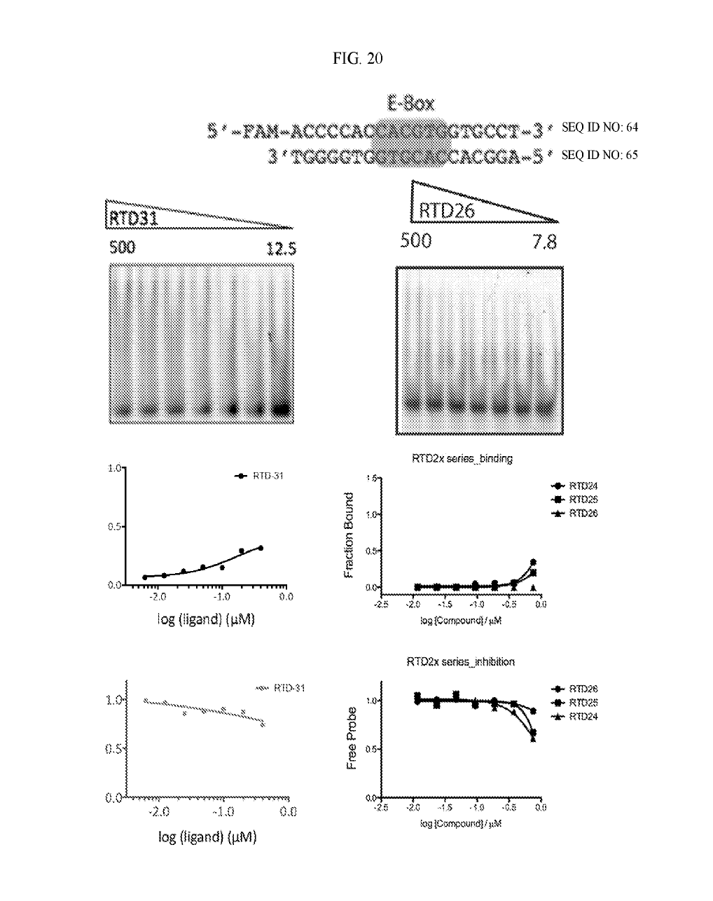

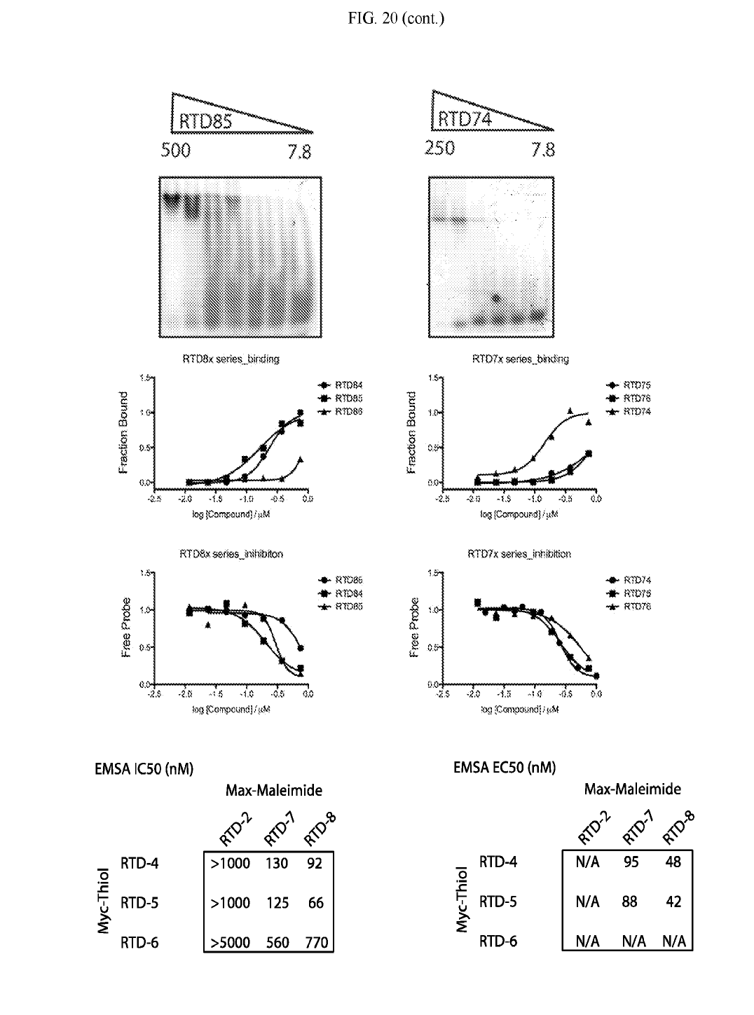

FIG. 20 shows sDBD DNA-binding activity measured by gel-shift assay. Representative gel shifts are shown for the Myc/Max heterodimer and members of the RTD2x, RTD8x and RTD7x series. Plotted are binding curves derived from the intensity of the free E-box probe (inhibition) and stable shifted band (binding). Dimerized, but not macrocycle-stabilized compound (RTD31), shows no stable binding. RTD24, 25 and 26 were largely inactive in the gel-shift assay. sDBDs in the RTD8x and RTD7x series showed EC50 values ranging from ˜200 to 600 nM. -sDBDs containing RTD6 showed the least activity among all classes, which might be correlated to its low helicity among Myc-based peptides. -sDBDs of the RTD7x and RTD8x classes are the most active.

### Example 5

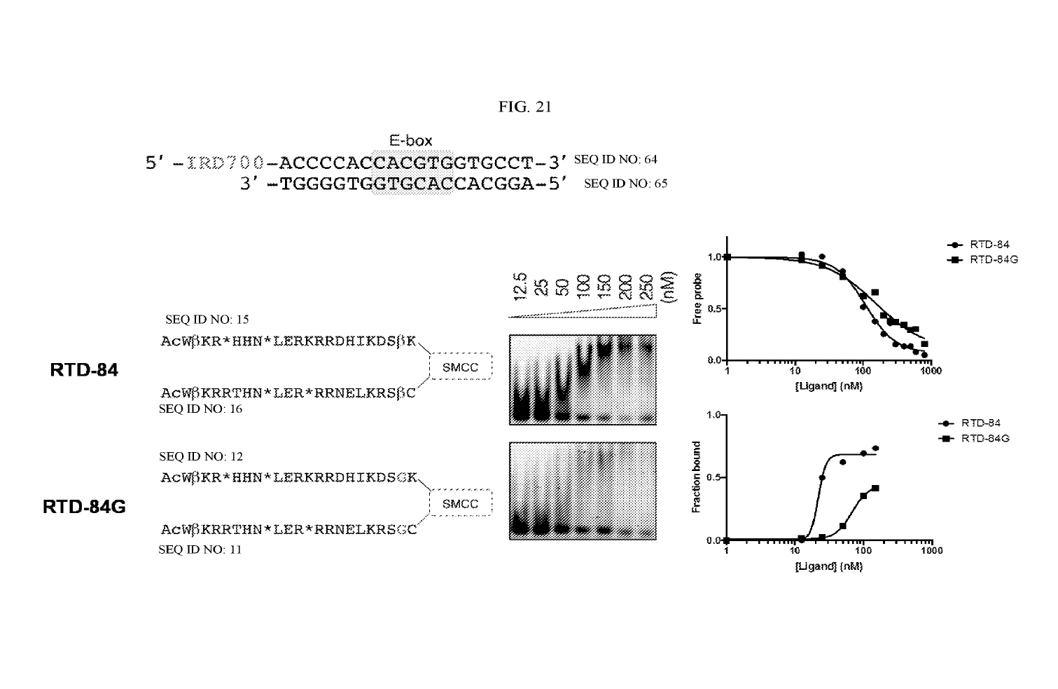

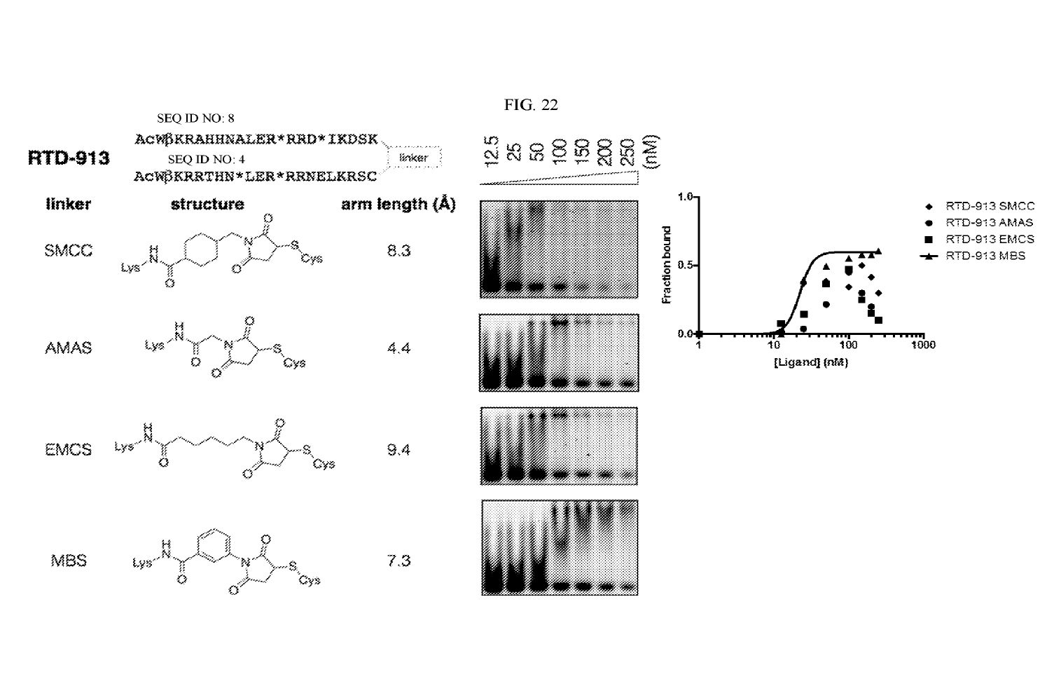

Experiments conducted during development of embodiments herein to determine the effect(s) of helix linkers within each stabilized DNA-binding helix as well as the dimerization motif between each helix on the overall ability of sDBDs to stably bind E-box DNA. Unlike wild-type bHLH proteins such as Myc/Max, sDBDs do not have the stabilizing leucine zipper and loop regions that help the correct folding of the DNA binding domain. While nature utilizes this fold to orient DNA-binding helices, their inclusion in a synthetic molecule precludes synthetic accessibility and cell-permeability. An exemplary compound, RTD-84, both stabilized DNA-binding helices are separated from the dimerization motif by a helix-breaking β-alanyl spacer; stable E-box binding is observed with an EC50 of ˜50-100 nM (FIG. 21). Altering this compound by one methylene on both helices (chaning the 3-alanine to glycine; compound RTD-84G) results in abrogation of stable DNA-binding and complex formation (FIG. 21). This is likely caused by propagation of the α-helix throughout the sequence up to the dimerization motif, and the extra glycine leads to an over-rotation of each helix by approximately 1/3.6 helical turns, misaligning the binding orientation. In a third design deletion of the glycyl spacer altogether is expected to maintain the helix to the dimerization motif but without misaligning the DNA-binding residues. This compound, RTD913, exhibits binding with an EC50 of ˜50-100 nM (FIG. 22). These data show that proper secondary structure stabilization (individual helix alteration) and tertiary structure stabilization are required and non-obvious even to enable in vitro DNA-binding.

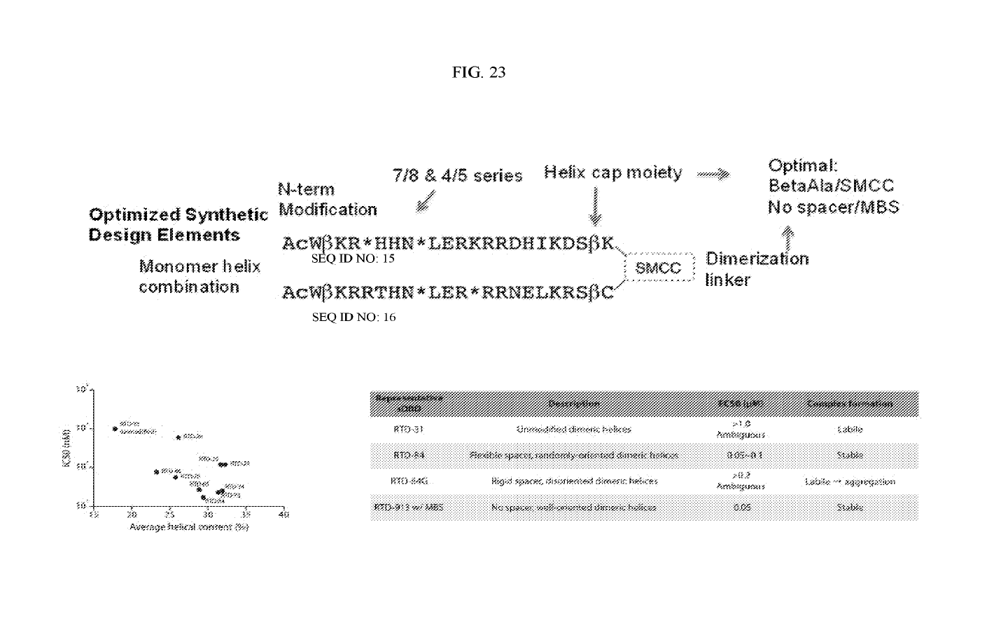

Experiments conducted during development of embodiments herein to investigate the relationship between the DNA binding activity and the structure of thiol-maleimide linkers within the RTD913 sub-class—the DNA binding sequence is identical to RTD84, but in RTD913 there is no linker breaking the helix (FIG. 22). Switching the cyclohexyl-containing SMCC linker to straight-chain counterparts, either short AMAS or long EMCS, yields less stable complex formation, due to lack of enough rigidity of either linker. However, when incorporating a more rigid meta-xylyl linker MBS with proper arm length, more stable DNA complex formation is observed at an EC50 of 50˜100 nM (FIG. 23).

The DNA binding affinity of sDBDs is highly context dependent, where location of the hydrocarbon linker must stabilize the helical structure but not interfere with DNA-binding, and specific linkers within and between helices are required for efficient and potent DNA binding.

### Example 6

### Microscopy

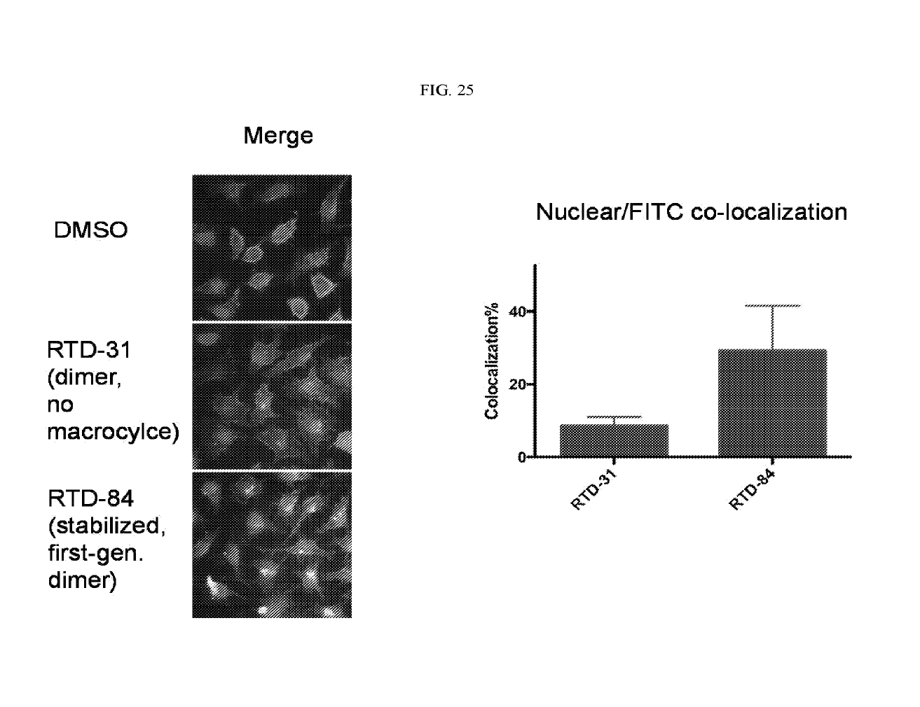

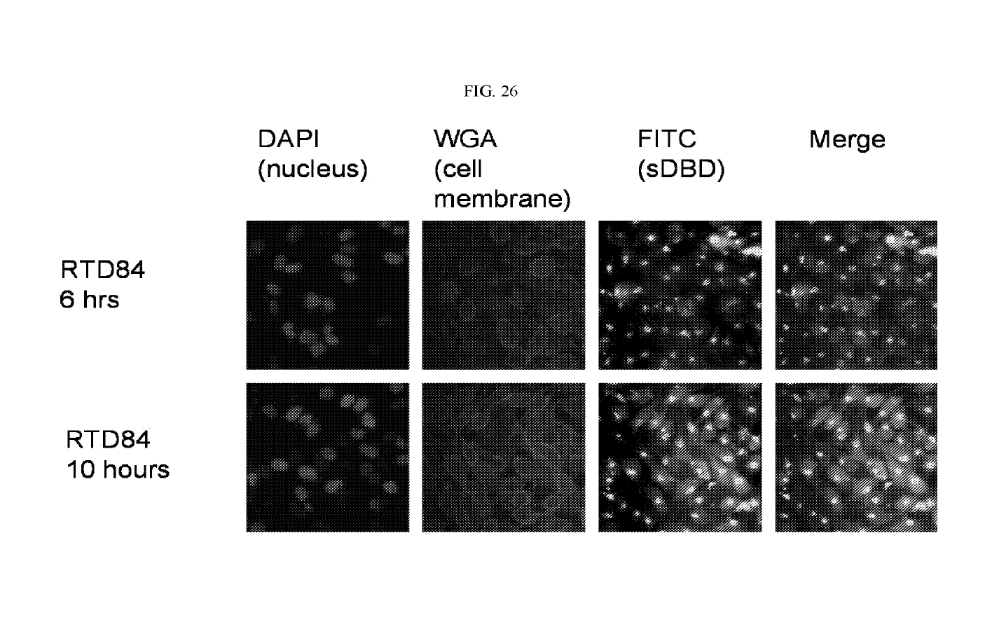

FIG. 11 shows that sDBDs are cell permeable, localize to cytoplasm and nucleus and exhibit higher cell penetration than non-macrocycle-containing dimer (RTD31). FIG. 25 shows that sDBDs are cell permeable, localize to cytoplasm and nucleus. sDBD exhibits higher total intracellular fluorescence and nuclear localization than non-macrocycle-containing dimer (RTD31). FIG. 26 shows that RTD84 shows strong cytosolic and nuclear localization at 6 and 10 hrs.

### REFERENCES

The following references, some of which may be cited above by author name or reference number, are herein incorporated by reference.
1. J. E. Damell, Jr., Transcription factors as targets for cancer therapy. Nature reviews. Cancer 2, 740-749 (2002).
2. T. N. Grossmann, J. T. Yeh, B. R. Bowman, Q. Chu, R. E. Moellering, G. L. Verdine, Inhibition of oncogenic Wnt signaling through direct targeting of beta-catenin. Proceedings of the National Academy of Sciences of the United States of America 109, 17942-17947 (2012).
3. R. E. Moellering, M. Comejo, T. N. Davis, C. Del Bianco, J. C. Aster, S. C. Blacklow, A. L. Kung, D. G. Gilliland, G. L. Verdine, J. E. Bradner, Direct inhibition of the NOTCH transcription factor complex. Nature 462, 182-188 (2009).
4. R. E. Moellering, G. L. Verdine, Stapled polypeptides as regulators of Rab-GTPase function. International Patent No. WO 2012/174423 A1. (2012).
5. Q. Chu, R. E. Moellering, G. J. Hilinski, Y. W. Kim, T. N. Grossmann, J. T. H. Yeh, G. L. Verdine, Towards understanding cell penetration by stapled peptides. Med Chem Comm 6, 111-119 (2015).
6. J. R. LaRochelle, G. B. Cobb, A. Steinauer, E. Rhoades, A. Schepartz, Fluorescence correlation spectroscopy reveals highly efficient cytosolic delivery of certain penta-arg proteins and stapled peptides. Journal of the American Chemical Society 137, 2536-2541 (2015).
7. F. Bemal, A. F. Tyler, S. J. Korsmeyer, L. D. Walensky, G. L. Verdine, Reactivation of the p53 tumor suppressor pathway by a stapled p53 peptide. Journal of the American Chemical Society 129, 2456-2457 (2007).
8. G. J. Hilinski, Y. W. Kim, J. Hong, P. S. Kutchukian, C. M. Crenshaw, S. S. Berkovitch, A. Chang, S. Ham, G. L. Verdine, Stitched alpha-helical peptides via bis ring-closing metathesis. Journal of the American Chemical Society 136.
9. S. J. Metallo, A. Schepartz, Distribution of labor among bZIP segments in the control of DNA affinity and specificity. Chemistry & biology 1, 143-151 (1994).
10. J. J. Kohler, S. J. Metallo, T. L. Schneider, A. Schepartz, DNA specificity enhanced by sequential binding of protein monomers. Proceedings of the National Academy of Sciences of the United States of America 96, 11735-11739 (1999).
11. L. Soucek, J. Whitfield, C. P. Martins, A. J. Finch, D. J. Murphy, N. M. Sodir, A. N. Kamezis, L. B. Swigart, S. Nasi, G. I. Evan, Modelling Myc inhibition as a cancer therapy. Nature 455, 679-683 (2008).
12. A. Subramanian, et al. Gene set enrichment analysis: a knowledge-based approach for interpreting genome-wide expression profiles. Proceedings of the National Academy of Sciences of the United States of America 102, 15545-15550 (2005).
13. S. G. Landt, et al. Genome research 22, 1813-1831 (2012).
14. C. V. Dang, Rethinking the Warburg effect with Myc micromanaging glutamine metabolism. Cancer research 70, 859-862 (2010).
15. R. E. Moellering, B. F. Cravatt, Functional lysine modification by an intrinsically reactive primary glycolytic metabolite. Science 341, 549-553 (2013).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A synthetic DNA binding domain peptide, comprising a modfied peptide that binds to a DNA molecule comprising an E-box transcription factor binding domain, wherein said peptide comprises a dimerization moiety configured to form a dimer with a second modified peptide.

2. The peptide of claim 1, wherein said dimerization moiety is selected from the group consisting of thiol, maliemide, an alkyne, azide, SMCC, AMAS, EMCS, and MBS.

3. The peptide of claim 1 or 2, wherien said E-box transcription factor binding domain has the sequence 5′-CACGTG-3′.

4. The peptide of any one of claims 1 to 3, wherien said peptide is derived from a basic helix-loop-helix leucine-zipper (bHLH-LZ) transcription factor.

5. The peptide of claim 4, wherein said transcription factor is selected from the group consisting of Myc/Max, Fos/Jun, HIF1α/β, MITF, MyoD, HES family, Hey family, ID1/2/3, E2 family, and Twist.

6. The peptide of any one of claims 1 to 5, wherein said peptide is a monomeric peptide.

7. The peptide of any one of claims 1 to 5, wherein said peptide is a dimeric peptide linked by said dimerization moiety.

8. The peptide of any one of claims 1 to 7, wherein said peptide is selected from the group consisting of AcW-βKRRTHNVLERQRRNELKRSβ-C (SEQ ID NO: 1), AcW-βKRAHHNALERKRRDHIKDSβ-K(Mmt) (SEQ ID NO: 2), AcW-βKRAHHNALERKRRDHIKDSβ-K(Mmt) (SEQ ID NO: 3), AcW-βKRRTHN*LER*RRNELKRSPβ-C (SEQ ID NO: 4), AcW-βKRRTHNVLER*RRN*LKRSβ-C (SEQ ID NO: 5), AcW-βKR*THN*LERQRRNELKRSβ-C (SEQ ID NO: 6), AcW-βKRAHHN*LER*RRDHIKDSβ-K(Mmt) (SEQ ID NO: 7), AcW-βKRAHHNALER*RRD*IKDSβ-K(Mmt) (SEQ ID NO: 8), AcW-βKRAHHNALER*RRD*IKDSβ-K(Mmt) (SEQ ID NO: 9), AcW-βKR*HHN*LERKRRDHIKDSβ-K(Mmt) (SEQ ID NO: 10), AcW-GKRRTHN*LER*RRNELKRSG-C (SEQ ID NO: 11), AcW-GKR*HHN*LERKRRDHIKDSG-K(Mmt) (SEQ ID NO: 12), AcW-βKRAHHNALER*RRD*IKDS-K(Mmt) (SEQ ID NO: 13), AcW-βKR*HHN*LERKRRDHIKDS-K(Mmt) (SEQ ID NO: 14), AcW-βKR*HHN*LERKRRDHIKDS-K(Mmt) (SEQ ID NO: 15), AcW-βKRRTHN*LER*RRNELKRS-C (SEQ ID NO: 16), AcW-βKRRTHNVLER*RRN*LKRS-C (SEQ ID NO: 17), AcW-βKRAHHNALER*RRD*IKDS-K(Mmt) (SEQ ID NO: 18), AcW-βKRAHHNALER*RRD*IKDS-K(Mmt) (SEQ ID NO: 19), AcW-βKRAHHNALER*RRD*IKDS-K(Mmt) (SEQ ID NO: 20), FITC-PEG3-βKRRTHNVLERQRRNELKRSβ-C (SEQ ID NO: 21), FITC-PEG3-βKRRTHN*LER*RRNELKRSβ-C (SEQ ID NO: 22), FITC-PEG3-βKRRTHNVLER*RRN*LKRSβ-C (SEQ ID NO: 23), Biotin-PEG3-W-βKRRTHN*LER*RRNELKRSβ-C (SEQ ID NO: 24), Biotin-PEG3-W-βKRRTHN*LER*RRNELKRS-C (SEQ ID NO: 25), Fmoc-βKRRTHNVLERQRRNELKRSβ-C (SEQ ID NO: 26), Fmoc-βKRAHHNALERKRRDHIKDβ-K(Mmt) (SEQ ID NO: 27), Fmoc-βKRRTHN*LER*RRNELKRSβ-C (SEQ ID NO: 28), Fmoc-βKRRTHN*LER*RRNELKRSβ-C (SEQ ID NO: 29), Fmoc-βKRRTHNVLER*RRN*LKRSβ-C (SEQ ID NO: 30), Fmoc-βKRRTHNVLER*RRN*LKRSβ-C (SEQ ID NO: 31), Fmoc-βKRAHHNALER*RRD*IKDSβ-K(Mmt) (SEQ ID NO: 32), Fmoc-βKRAHHNALER*RRD*IKDSβ-K(Mmt) (SEQ ID NO: 33), Fmoc-βKR*HHN*LERKRRDHIKDSβ-K(Mmt) (SEQ ID NO: 34), Fmoc-βKR*HHN*LERKRRDHIKDSβ-K(Mmt) (SEQ ID NO: 35), Fmoc-βKRRTHN*LER*RRNELKRSG-C (SEQ ID NO: 36), Fmoc-βKRRTHN*LER*RRNELKRSG-C (SEQ ID NO: 37), Fmoc-βKR*HHN*LERKRRDHIKDSG-K(Mmt) (SEQ ID NO: 38), Fmoc-βKR*HHN*LERKRRDHIKDSG-K(Mmt) (SEQ ID NO: 39), Fmoc-βKRAHHNALER*RRD*IKDS-K(Mmt) (SEQ ID NO: 40), Fmoc-βKRAHHNALER*RRD*IKDS-K(Mmt) (SEQ ID NO: 41), Fmoc-βKR*HHN*LERKRRDHIKDS-K(Mmt) (SEQ ID NO: 42), Fmoc-βKR*HHN*LERKRRDHIKDS-K(Mmt) (SEQ ID NO: 43), Fmoc-βKRRTHN*LER*RRNELKRS-C (SEQ ID NO: 44), Fmoc-βKRRTHN*LER*RRNELKRS-C (SEQ ID NO: 45), Fmoc-βKRRTHNVLER*RRN*LKRS-C (SEQ ID NO: 46), Fmoc-βKRRTHNVLER*RRN*LKRS-C (SEQ ID NO: 47), Fmoc-βKRRTHN*LER*RRNELKRSβ-K(Mmt) (SEQ ID NO: 48), Fmoc-βKRRTHNVLER*RRN*LKRSβ-K(Mmt) (SEQ ID NO: 49), Fmoc-βKRRTHN*LER*RRNELKRSβ-K(Mmt) (SEQ ID NO: 50), Fmoc-βKRRTHNVLER*RRN*LKRSβ-K(Mmt) (SEQ ID NO: 51).

9. The peptide of any one of claims 1 to 8, wherein said peptide inhibits the activity of said transcription factor.

10. A complex, comprising at least one peptide of any one of claims 1 to 9 bound to an E-box transcription factor binding domain.

11. The complex of claim 10, wherein said E-box transcription factor binding domain has the sequence 5′-CACGTG-3′.

12. The complex of claim 10 or 11, wherein said at least one peptide is two peptides, wherein each of said two peptides has a different dimerization moiety, and wherein said different dimerization moieties form a covalent bond when contacted.

13. The complex of claim 10 or 11, wherein said at least one peptide comprises two peptides covalently linked by one or more dimerization moieties.

14. A composition, comprising:
(a) a first synthetic peptide comprising:
(i) at least one internal hydrocarbon staple, and
(ii) a first dimerization moiety; and
(b) a second synthetic peptide comprising:
(i) at least one internal hydrocarbon staple, and
(ii) a second dimerization moiety;
wherein the first and second dimerization moieties are capable of interacting to form a stable bond, thereby forming a dimer of the first and second synthetic peptides.

15. The composition of claim 14, wherein the hydrocarbon staples are the result of ring-closing olefin metathesis (RCM) of hindered α-methyl, α-alkenyl amino acids.

16. The composition of claim 14, wherein the first and second dimerization moieties are attached to a side chain of N-terminal amino acids.

17. The composition of claim 14, wherein the first dimerization moiety comprises a thiol and the second dimerization moiety comprises a maleimide.

18. The composition of claim 14, wherein the first dimerization moiety comprises a azide and the second dimerization moiety comprises an alkyne.

19. The composition of claim 14, wherein the first synthetic peptide further comprises a third dimerization moiety and the second synthetic peptide further comprises a fourth dimerization moiety; wherein the third and fourth dimerization moieties are capable of interacting to form a stable bond, thereby forming a dimer of the first and second synthetic peptides.

20. The composition of claim 19, wherein the third and fourth dimerization moieties are attached to a side chain of an amino acid within 5 positions of the N-terminal amino acid.

21. The composition of claim 19, wherein the first dimerization moiety comprises a thiol, the second dimerization moiety comprises a maleimide, the third dimerization moiety comprises an azide, and the fourth dimerization moiety comprises an alkyne.

22. A composition comprising a dimer of the first and second synthetic peptides of one of claims 14-21.

23. A peptide or conjugate of peptides of one of Formulas I-XII.

24. A pharmaceutical composition, comprising: (i) a peptide of one of claims 1 to 9, a complex of one of claims 10-13, a composition of one of claims 14-22, or a peptide or conjugate of peptides of claim 23; and (ii) a pharmaceutically acceptable carrier.

25. The pharmaceutical composition of claim 24, wherein said at least one peptide is two peptides, wherein each of said two peptides has a different dimerization moiety, and wherein said different dimerization moieties form a covalent bond when contacted.

26. The pharmaceutical composition of claim 24 wherein said at least one peptide comprises two peptides covalently linked by one or more dimerization moieties.

27. A method of inhibiting the activity of a transcription factor, comprising:
contacting said transcription factor with a peptide of one of claims 1 to 9, a complex of one of claims 10-13, or a composition of one of claims 14-22, a peptide or conjugate of peptides of claim 23, or a pharmaceutical composition of one of claims 24-26, wherein said contacting inhibits the activity of said transcription factor.

28. The method of claim 27, wherein said transcription factor is selected from the group consisting of Myc/Max, Fos/Jun, HIF1α/β, MITF, MyoD, HES family, Hey family, ID1/2/3, E2 family, Twist, AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, CLOCK, EPAS1, FERD3L, FIGLA, HAND1, HAND2, HES1, HES2, HES3, HES4, HES5, HES6, HES7, HEY1, HEY2, HIF1A, ID1, ID2, ID3, ID4, KIAA2018, LYL1, MASH1, MATH2, MAX, MESP1, MESP2, MIST1, MITF, MLX, MLXIP, MLXIPL, MNT, MSC, MSGN1, MXD1, MXD3, MXD4, MXI1, MYC, MYCL1, MYCL2, MYCN, MYF5, MYF6, MYOD1, MYOG, NCOA1, NCOA3, NEUROD1, NEUROD2, NEUROD4, NEUROD6, NEUROG1, NEUROG2, NEUROG3, NHLH1, NHLH2, NPAS1, NPAS2, NPAS3, OAF1, OLIG1, OLIG2, OLIG3, PTF1A, SCL, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, USF2, ATF1, ATF2, ATF4, ATF5, ATF6, ATF7, BACH1, BACH2, BATF, BATF2, CREB1, CREB3, CREB3L1, CREB3L2, CREB3L3, CREB3L4, CREB5, CREBL1, CREM, E4BP4, FOSL1, FOSL2, JUN, JUNB, JUND, NFE2, NFE2L2, NFE2L3, OPAQUE2, SNFT, and CREM.

29. The method of claim 27 or 28, wherein said inhibiting treats a disease.

30. The method of claim 29, wherein said disease is cancer.

31. A method of treating a disease, comprising:
administering the pharmaceutical composition of any one of claims 24 to 26 to a subject in need thereof, wherein said administering treats said disease.

32. The method of claim 31, wherein said disease is cancer.

33. The use of the a peptide of one of claims 1 to 9, a complex of one of claims 10-13, or a composition of one of claims 14-22, a peptide or conjugate of peptides of claim 23, or a pharmaceutical composition of one of claims 24-26 to inhibit at least one activity of a transcription factor.

34. The use of claim 33, wherein said transcription factor is selected from the group consisting of Myc/Max, Fos/Jun, HIF1α/β, MITF, MyoD, HES family, Hey family, ID1/2/3, E2 family, or Twist.

35. The use of claim 33 or 34, wherein said inhibiting treats a disease.

36. The use of claim 35, wherein said disease is cancer.

37. The use of the the pharmaceutical composition of any one of claims 24 to 26 to treat a disease in a subject.

38. The use of claim 37, wherein said disease is cancer.
